(19) United States
(12) Patent Application Publication
(10) Pub. No.: US 2021/0038628 A1
(43) Pub. Date: Feb. 11, 2021

(54) NANOPARTICLE COMPOSITIONS

(71) Applicant: JANUARY THERAPEUTICS, INC., San Diego, CA (US)

(72) Inventors: Raj RAHEJA, San Diego, CA (US); Robin M. JACKMAN, San Diego, CA (US); Jason A. KAHANA, San Diego, CA (US)

(73) Assignee: January Therapeutics, Inc., San Diego, CA (US)

(21) Appl. No.: 16/976,909

(22) PCT Filed: Mar. 1, 2019
(86) PCT No.: PCT/US19/20389

§ 371 Date: Aug. 31, 2020

Related U.S. Application Data
(60) Provisional application No. 62/798,859, filed on Jan. 30, 2019.
(60) Provisional application No. 62/637,965, filed on Mar. 2, 2018.

(51) Int. Cl. A61K 31/7068; A61K 9/00; A61K 45/06; A61K 9/107; A61P 35/00; C07F 9/6558; A61K 9/51

(52) U.S. Cl. CPC A61K 31/7068; A61K 9/0019; A61K 45/06; A61K 9/5169; A61P 35/00; C07F 9/65586; A61K 9/107

(57) ABSTRACT

Provided herein are nanoparticle compositions comprising organophosphate compounds, and pharmaceutically acceptable carriers.

### CROSS-REFERENCE

This application claims benefit of U.S. Provisional Application No. 62/637,965, filed on Mar. 2, 2018, and U.S. Provisional Application No. 62/798,859, filed on Jan. 30, 2019, both of which are herein incorporated by reference in their entirety.

### BACKGROUND

Nucleoside or nucleotide derivatives are widely used in treating cancer or viral infections.

### BRIEF SUMMARY OF THE DISCLOSURE

This disclosure provides, for example, nanoparticle compositions comprising organophosphate compounds, such as the compounds of Formula (I) or Formula (II) as described herein, their use as medicinal agents, and processes for their preparation. The disclosure also provides for the use of the nanoparticle compositions described herein as medicaments and/or in the manufacture of medicaments for the treatment of a variety of diseases, including cancer and viral infections.

Provided in one aspect is a composition comprising nanoparticles, wherein the nanoparticles comprise a compound of Formula (I):

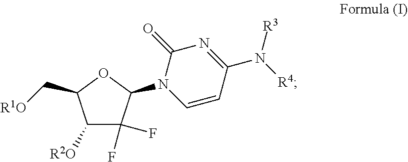

wherein:
R¹ is

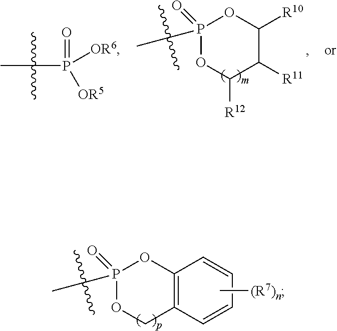

R² is —C(O)R⁸;
R³ is H, —C(O)R⁹, or —C(O)OR⁹;
R⁴ is H;
R⁵ is H, C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C alkyl-C₂₋₉heteroaryl are optionally substituted with 1, 2, 3, or 4 R¹⁴;
R⁶ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C alkyl-C₂₋₉heteroaryl are optionally substituted with 1, 2, 3, or 4 R¹⁴;
each R⁷ is independently selected from halogen, C₁₋₈alkyl, C₁₋₈haloalkyl, and C₁₋₈alkoxy;
R⁸ is C₃₋₂₂alkyl, C₃₋₂₂alkenyl, C₃₋₂₂alkynyl, C₃₋₂₂haloalkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁹ is C₁₋₁₂alkyl;
R¹⁹ and R¹¹ are each independently H or C₁₋₁₂alkyl, or R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³;
R¹² is H or C₁₋₁₂alkyl;
each R¹³ is independently selected from C₁₋₁₂alkyl;
each R¹⁴ is independently selected from halogen, C₁₋₈alkyl, C₁₋₈haloalkyl, C₁₋₈alkoxy, and —C(O)R¹³;
m is 0 or 1,
n is 0, 1, 2, 3, or 4; and
p is 0 or 1, and
   a pharmaceutically acceptable carrier; wherein the pharmaceutically acceptable carrier comprises albumin.

In some embodiments, R¹ is

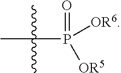

In some embodiments, R⁵ is C₃₋₁₂alkyl. In some embodiments, wherein R⁵ is C₆₋₁₀alkyl. In some embodiments, R⁵ is —C₁₋₄alkyl-OC(O)C₁₋₈alkyl. In some embodiments, R⁵ is —C₁₋₂alkyl-OC(O)C₁₋₆alkyl. In some embodiments, R⁵ is —CH₂—OC(O)C(CH₃)₃. In some embodiments, R⁵ is H. In some embodiments, R⁶ is C₃₋₁₂alkyl. In some embodiments, R⁶ is C₆₋₁₀alkyl. In some embodiments, R⁶ is —C₁₋₄alkyl-OC(O)C₁₋₈alkyl. In some embodiments, R⁶ is —C₁₋₂alkyl-OC(O)C₁₋₆alkyl. In some embodiments, R⁶ is —CH₂—OC(O)C(CH₃)₃.

In some embodiments, R¹ is

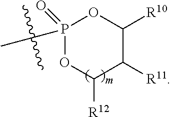

In some embodiments, m is 0. In some embodiments, m is 1. In some embodiments, R¹⁰ is H. In some embodiments, R¹⁰ is C₁₋₁₂alkyl. In some embodiments, R¹¹ is H. In some embodiments, R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In some embodiments, R¹ is

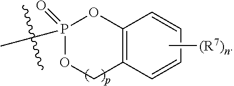

In some embodiments, each R⁷ is independently selected from C₁₋₈alkyl, C₁₋₈haloalkyl, and C₁₋₈alkoxy. In some embodiments, each R⁷ is independently selected from C₁₋₈alkyl. In some embodiments, n is 1 or 2. In some embodiments, n is 0. In some embodiments, p is 0. In some embodiments, p is 1. In some embodiments, R⁸ is C₃₋₁₅alkyl. In some embodiments, R⁸ is C₆₋₁₂alkyl. In some embodiments, R⁸ is —(CH₂)₇CH₃.

Provided in one aspect is a composition comprising nanoparticles, wherein the nanoparticles comprise a compound of Formula (II):

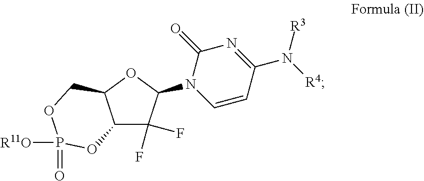

wherein:
R³ is H, —C(O)R⁹, or —C(O)OR⁹;
R⁴ is H;
R⁹ is C₁₋₈alkyl;
R¹¹ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹²;
each R¹² is independently selected from halogen, C₁₋₈alkyl, C₁₋₈haloalkyl, C₁₋₈alkoxy, and —C(O)R¹³; and
each R¹³ is independently selected from C₁₋₁₂alkyl; and
   a pharmaceutically acceptable carrier; wherein the pharmaceutically acceptable carrier comprises albumin.

In some embodiments, R¹¹ is C₃₋₁₅alkyl. In some embodiments, R¹¹ is C₆₋₁₂alkyl. In some embodiments, R¹¹ is C₈₋₁₀alkyl. In some embodiments, R¹¹ is —C₁₋₄alkyl-OC(O)C₁₋₈alkyl. In some embodiments, R¹¹ is —C₁₋₂alkyl-OC(O)C₁₋₆alkyl. In some embodiments, R¹¹ is —CH₂—OC(O)C(CH₃)₃. In some embodiments, R¹¹ is C₆₋₁₀aryl optionally substituted with 1, 2, 3, or 4 R¹². In some embodiments, R¹¹ is phenyl optionally substituted with 1, 2, or 3 R¹². In some embodiments, R¹¹ is phenyl optionally substituted with 1, 2, or 3 R¹², and each R¹² is independently selected from C₁-C₈alkoxy, and —C(O)R¹³. In some embodiments, R¹¹ is phenyl optionally substituted with 1 or 2 R¹², and each R¹² is independently selected from C₁₋₈alkyl and C₁₋₈alkoxy. In some embodiments, R¹¹ is —C₁₋₈alkyl-C₆₋₁₀aryl optionally substituted with 1, 2, 3, or 4 R¹². In some embodiments, R¹¹ is —CH₂-phenyl optionally substituted with 1, 2, or 3 R¹². In some embodiments, R¹¹ is —CH₂-phenyl optionally substituted with 1, 2, or 3 R¹², and each R¹² is independently selected from C₁₋₈alkyl, C₁₋₈alkoxy, and —C(O)R¹³. In some embodiments, R¹¹ is —CH₂-phenyl optionally substituted with 1 or 2 R¹², and each R¹² is independently selected from C₁₋₈alkyl and C₁₋₈alkoxy. In some embodiments, R³ is H. In some embodiments, R³ is —C(O)R⁹. In some embodiments, R³ is —C(O)OR⁹.

Provided in one aspect is a composition comprising nanoparticles, wherein the nanoparticles comprise a compound selected from:

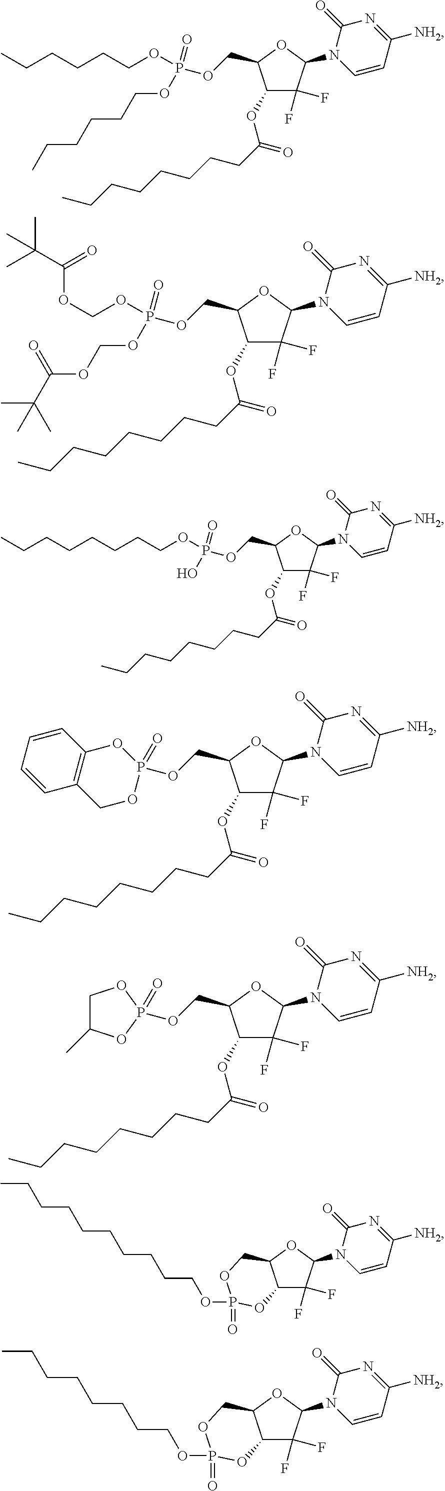

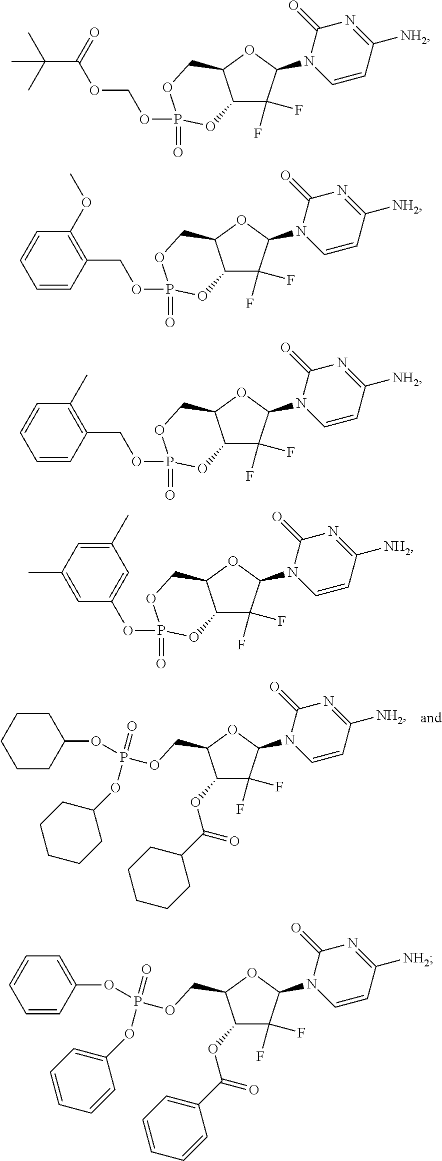

and a pharmaceutically acceptable carrier; wherein the pharmaceutically acceptable carrier comprises albumin.

Provided in another aspect is a composition comprising nanoparticles, wherein the nanoparticles comprise a compound selected from:

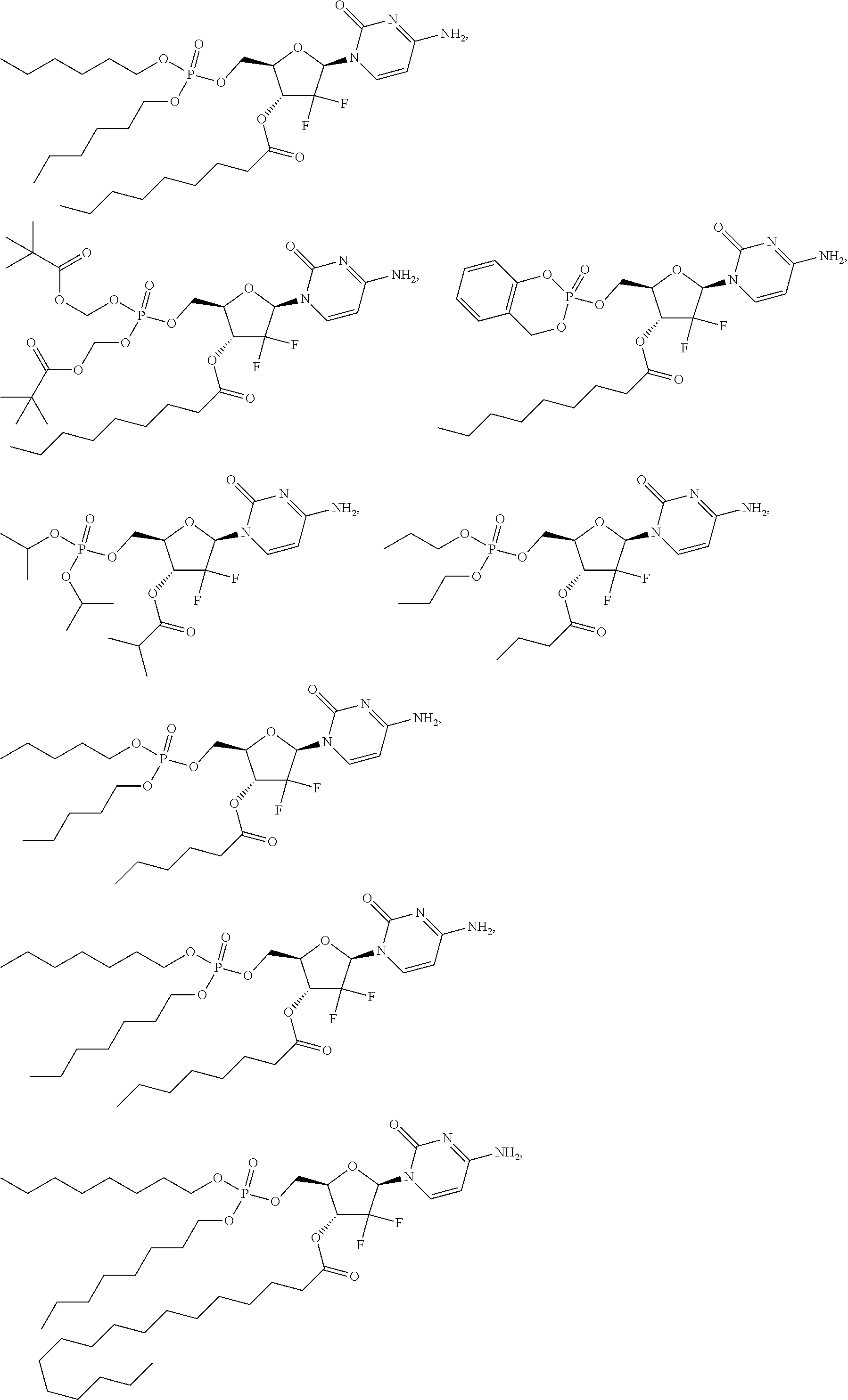

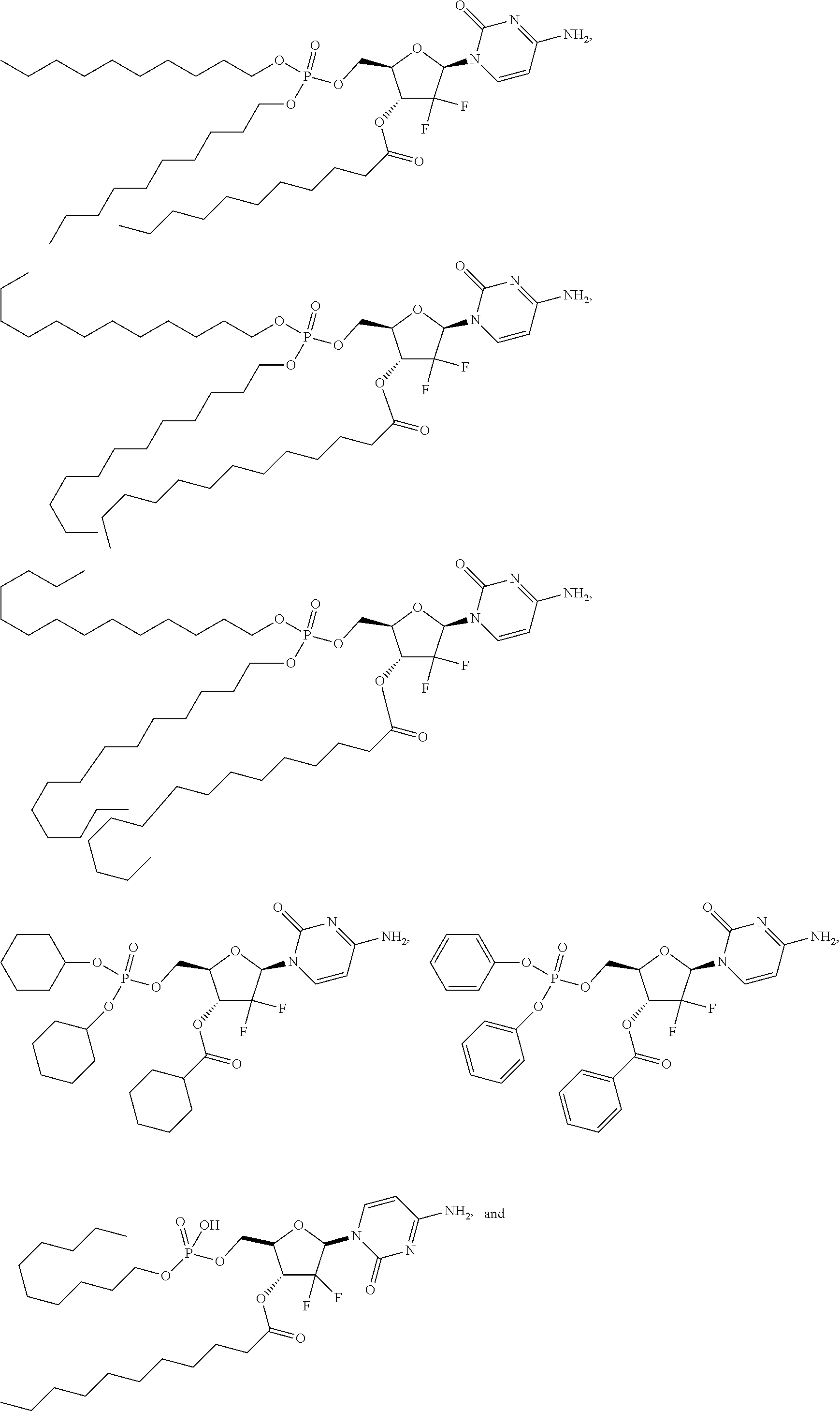

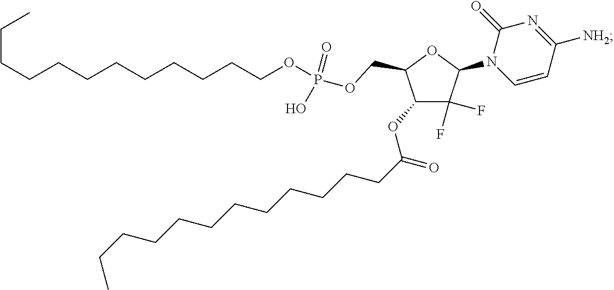 [SMILES: CCCCCCCCCCCCOP(=O)(O)OC[C@H]1O[C@@H](n2ccc(N)nc2=O)C(F)(F)[C@@H]1OC(=O)CCCCCCCCCCCC]

and a pharmaceutically acceptable carrier; wherein the pharmaceutically acceptable carrier comprises albumin.

Provided in another aspect is a composition comprising nanoparticles, wherein the nanoparticles comprise a compound selected from:

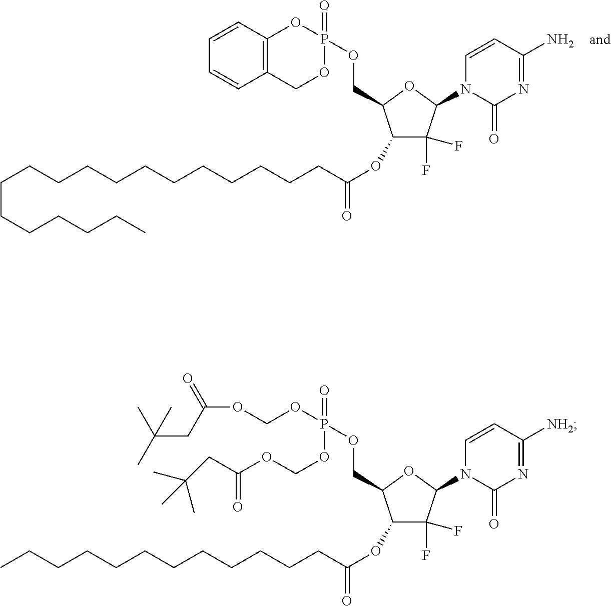 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

and a pharmaceutically acceptable carrier; wherein the pharmaceutically acceptable carrier comprises albumin.

Provided in another aspect is a composition comprising nanoparticles, wherein the nanoparticles comprise a compound selected from:

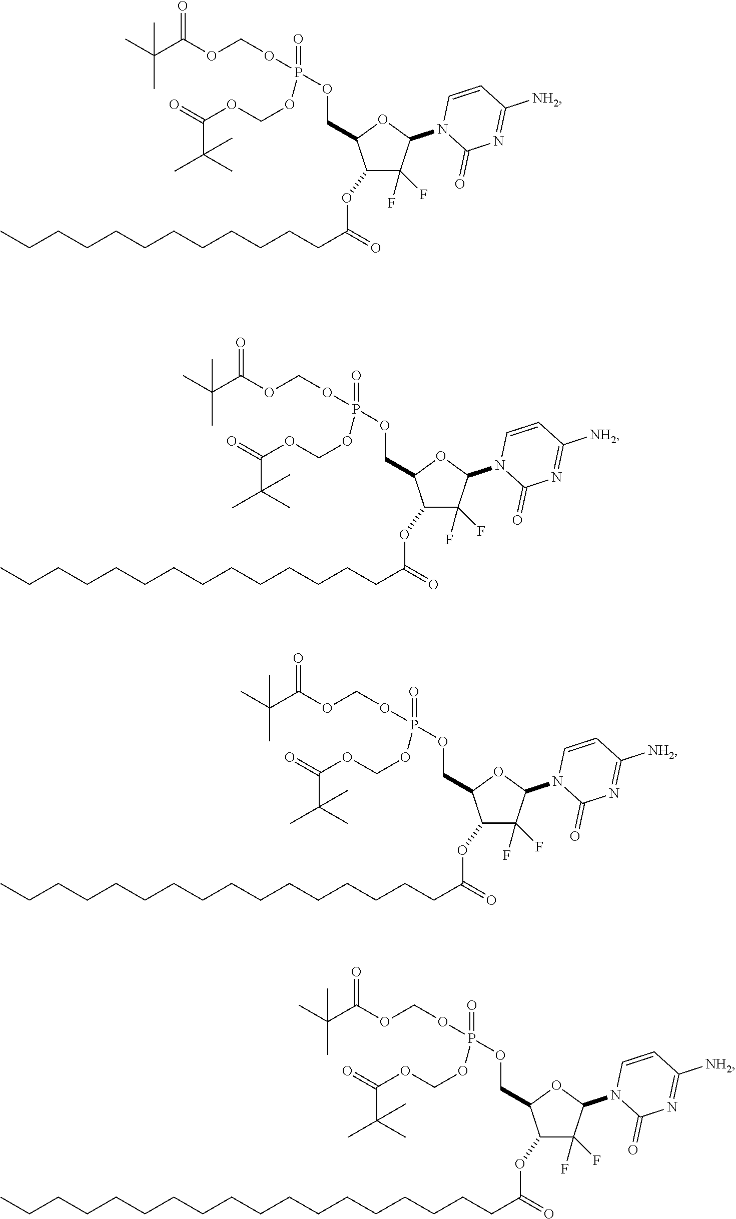 [SMILES: CCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F ; CCCCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F ; CCCCCCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F ; CCCCCCCCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F]

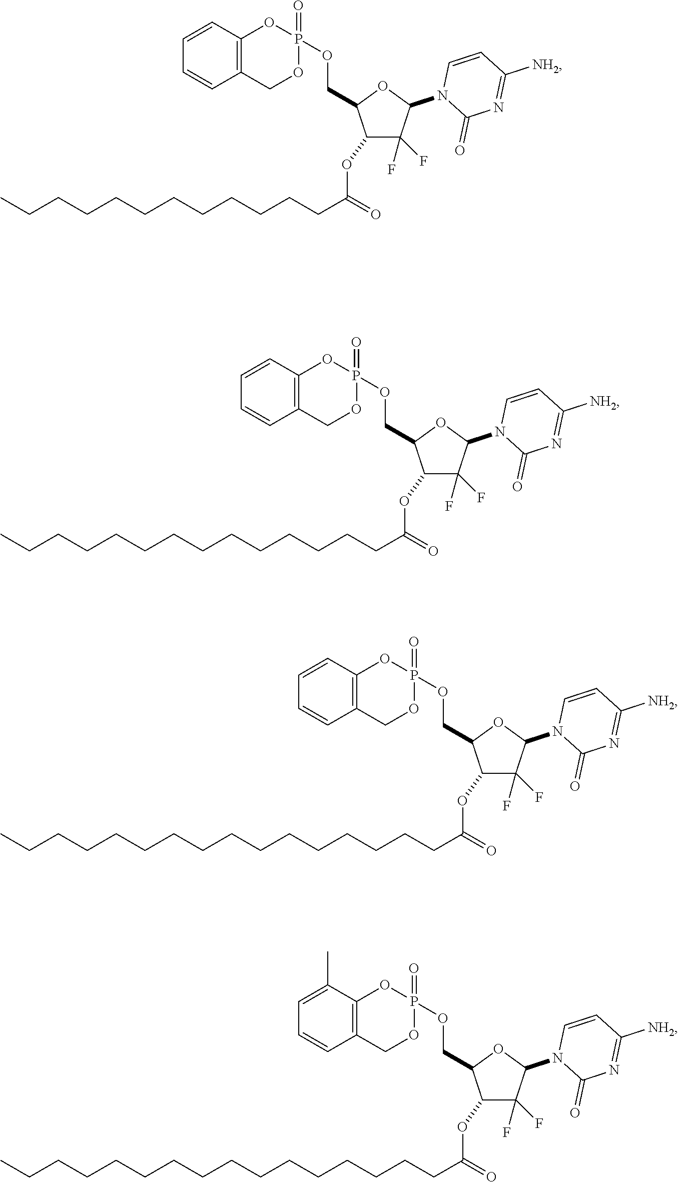 [SMILES: CCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCCCCCC(=O)O[C@@H]1[C@@H](COP2(=O)OCc3cccc(C)c3O2)O[C@@H](n2ccc(N)nc2=O)C1(F)F]

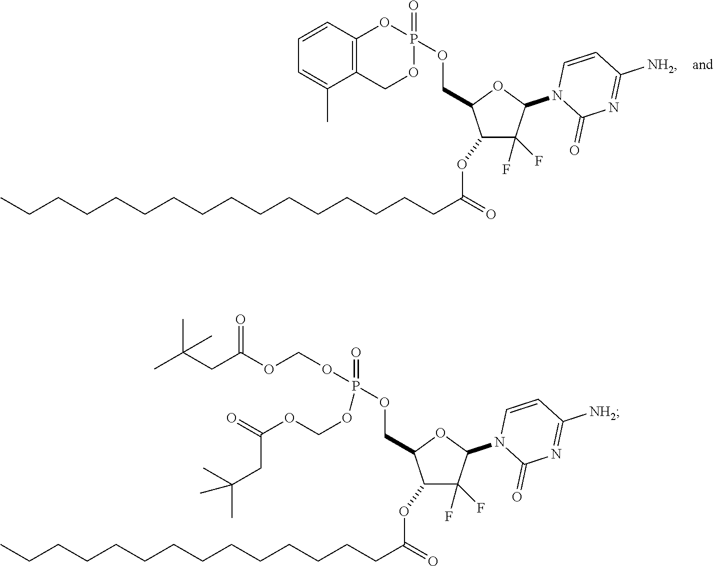 [SMILES: CCCCCCCCCCCCCCCCC(=O)O[C@@H]1[C@@H](COP2(=O)OCc3c(C)cccc3O2)O[C@@H](n2ccc(N)nc2=O)C1(F)F ; CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

and a pharmaceutically acceptable carrier; wherein the pharmaceutically acceptable carrier comprises albumin.

In some embodiments, the nanoparticles have an average diameter of about 1000 nm or less for at least about 15 minutes after nanoparticle formation. In some embodiments, the nanoparticles have an average diameter of about 10 nm or greater for at least about 15 minutes after nanoparticle formation. In some embodiments, the nanoparticles have an average diameter of from about 10 nm to about 1000 nm for at least about 15 minutes after nanoparticle formation. In some embodiments, the nanoparticles have an average diameter of about 1000 nm or less for at least about 4 hours after nanoparticle formation. In some embodiments, the nanoparticles have an average diameter of about 10 nm or greater for at least about 4 hours nanoparticle formation. In some embodiments, the nanoparticles have an average diameter of from about 10 nm to about 1000 nm for at least about 4 hours after nanoparticle formation.

In some embodiments, the nanoparticles have an average diameter of from about 10 nm to about 1000 nm. In some embodiments, the nanoparticles have an average diameter of from about 30 nm to about 250 nm. In some embodiments, the albumin is human serum albumin. In some embodiments, the molar ratio of the compound to pharmaceutically acceptable carrier is from about 1:1 to about 20:1. In some embodiments, the molar ratio of the compound to pharmaceutically acceptable carrier is from about 2:1 to about 12:1. In some embodiments, the nanoparticles are suspended, dissolved, or emulsified in a liquid. In some embodiments, the composition is sterile filterable.

In some embodiments, the composition is dehydrated. In some embodiments, the composition is a lyophilized composition. In some embodiments, the composition comprises from about 0.9% to about 24% by weight of the compound. In some embodiments, the composition comprises from about 1.8% to about 16% by weight of the compound. In some embodiments, the composition comprises from about 76% to about 99% by weight of the pharmaceutically acceptable carrier. In some embodiments, the composition comprises from about 84% to about 98% by weight of the pharmaceutically acceptable carrier.

In some embodiments, the composition is reconstituted with an appropriate biocompatible liquid to provide a reconstituted composition. In some embodiments, the appropriate biocompatible liquid is a buffered solution. In some embodiments, the appropriate biocompatible liquid is a solution comprising dextrose. In some embodiments, the appropriate biocompatible liquid is a solution comprising one or more salts. In some embodiments, the appropriate biocompatible liquid is sterile water, saline, phosphate-buffered saline, 5% dextrose in water solution, Ringer's solution, or Ringer's lactate solution. In some embodiments, the nanoparticles have an average diameter of from about 10 nm to about 1000 nm after reconstitution. In some embodiments, the nanoparticles have an average diameter of from about 30 nm to about 250 nm after reconstitution.

In some embodiments, the composition is suitable for injection. In some embodiments, the composition is suitable for intravenous administration. In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally. In some embodiments, the compound is an anticancer agent. In some embodiments, the compound is an antiviral agent.

Provided in one aspect is a method of treating a disease in a subject in need thereof comprising administering any one of the compositions described herein. In some embodiments, the disease is cancer. In some embodiments, the disease is caused by an infection. In some embodiments, the infection is viral.

Provided in another aspect is a method of delivering a compound of Formula (I) or Formula (II) to a subject in need thereof comprising administering any one of the compositions described herein.

Provided in another aspect is a process of preparing any one of the composition described herein comprising
a) dissolving a compound of Formula (I) or Formula (II) in a volatile solvent to form a solution comprising a dissolved compound of Formula (I) or Formula (II);
b) adding the solution comprising the dissolved compound of Formula (I) or Formula (II) to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form any one of the compositions described herein.

In some embodiments, the volatile solvent is a chlorinated solvent, alcohol, ketone, ester, ether, acetonitrile, or any combination thereof. In some embodiments, the volatile solvent is chloroform, ethanol, methanol, or butanol. In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the evaporation is accomplished with a rotary evaporator. In some embodiments, the evaporation is under reduced pressure.

Provided in another aspect is a compound selected from:

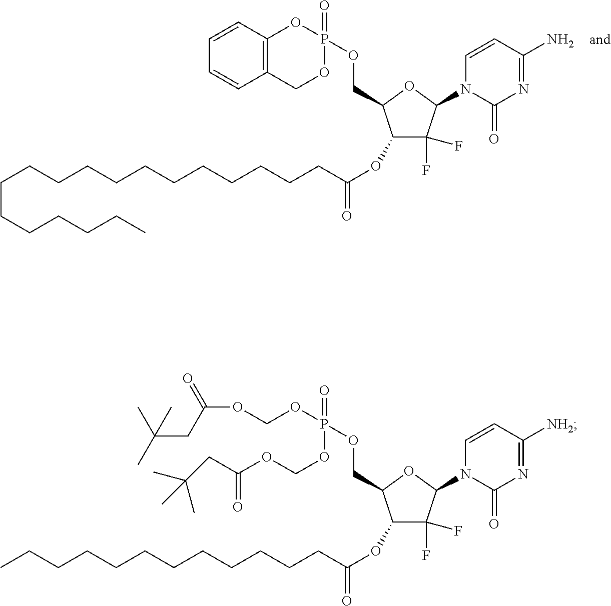 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

or a pharmaceutically acceptable salt thereof.

Provided in another aspect is a compound that is:

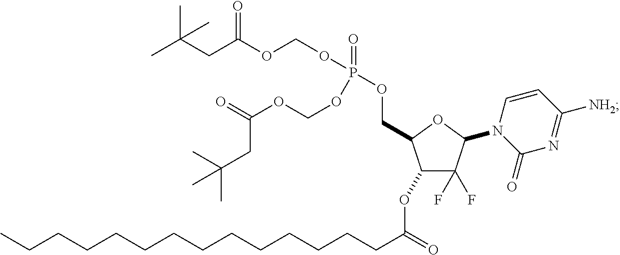 [SMILES: CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

or a pharmaceutically acceptable salt thereof.

Provided in another aspect is a pharmaceutical composition comprising a compound, or a pharmaceutically acceptable salt thereof, selected from:

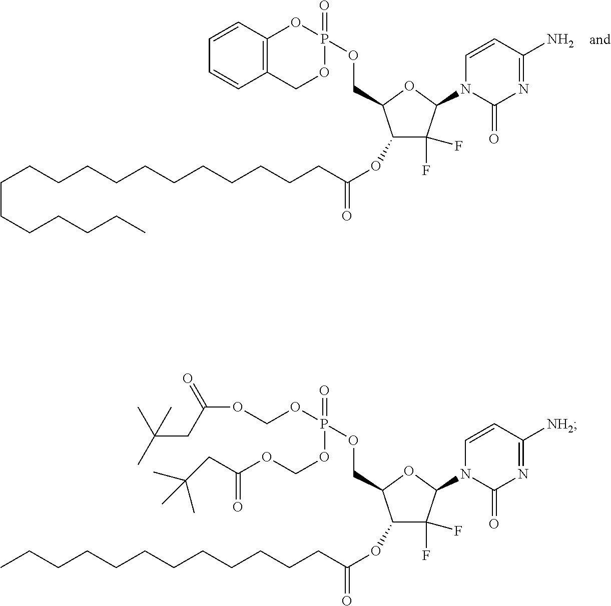 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

and at least one pharmaceutically acceptable excipient.

Provided in another aspect is a pharmaceutical composition comprising a compound, or a pharmaceutically acceptable salt thereof, that is:

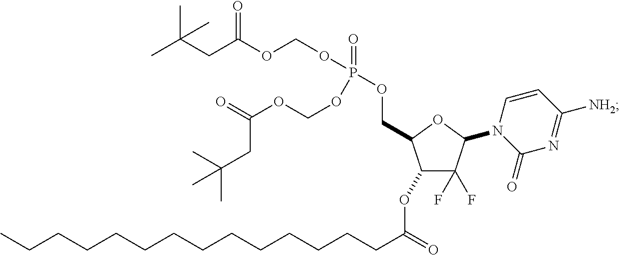 [SMILES: CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

and at least one pharmaceutically acceptable excipient.

Provided in another aspect is a method of treating cancer in a patient in need thereof, comprising administering to the patient a therapeutically effective amount of a compound, or a pharmaceutically acceptable salt thereof, selected from:

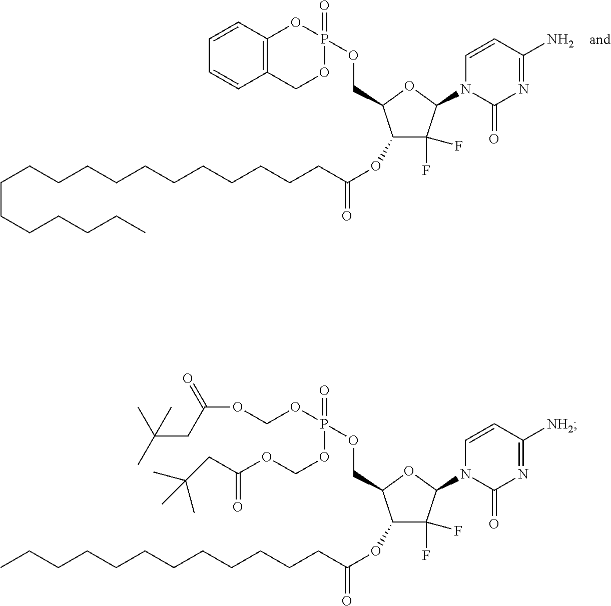 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

Provided in another aspect is a method of treating cancer in a patient in need thereof, comprising administering to the patient a therapeutically effective amount of a compound, or a pharmaceutically acceptable salt thereof, that is:

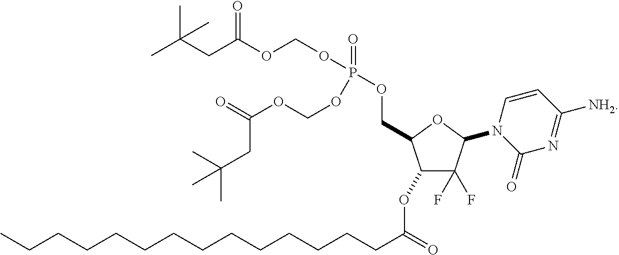 [SMILES: CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

Provided in another aspect is a method of treating an infectious disease in a patient in need thereof, comprising administering to the patient a therapeutically effective amount of a compound, or a pharmaceutically acceptable salt thereof, selected from:

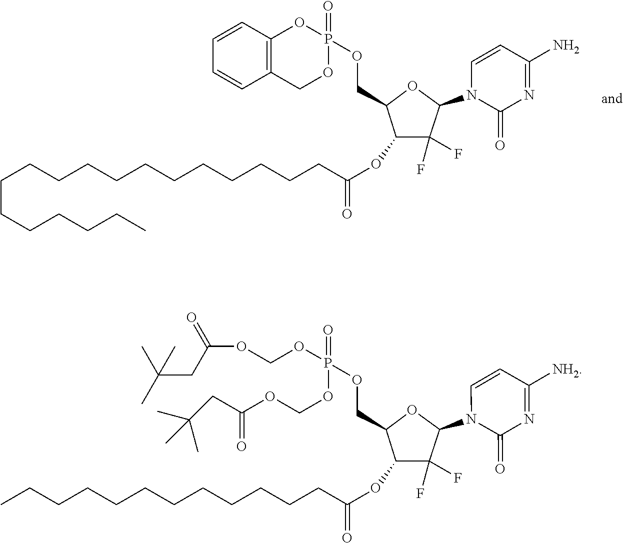 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

Provided in another aspect is a method of treating an infectious disease in a patient in need thereof, comprising administering to the patient a therapeutically effective amount of a compound, or a pharmaceutically acceptable salt thereof, that is:

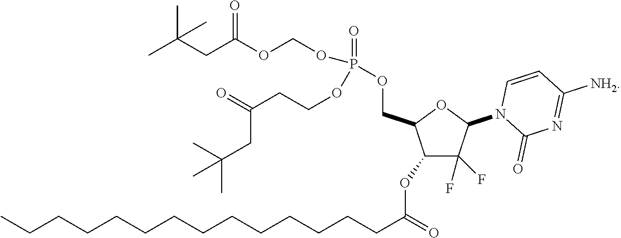 [SMILES: CCCCCCCCCCCCCCC(=O)O[C@@H]1[C@@H](COP(=O)(OCCC(=O)CC(C)(C)C)OCOC(=O)CC(C)(C)C)O[C@@H](n2ccc(N)nc2=O)C1(F)F]

### BRIEF DESCRIPTION OF THE FIGURE

FIG. 1. shows tumor volume up to Day 25 for mice treated with a nanoparticle formulation of Compound 24 (nanoparticle formulation of Example 46), a nanoparticle formulation of Compound 16 (nanoparticle formulation of Example 46), or Gemcitabine at 40 mg/kg dosing.

### DETAILED DESCRIPTION OF THE DISCLOSURE

This application recognizes that the use of nanoparticles as a drug delivery platform is an attractive approach as nanoparticles provide the following advantages: more specific drug targeting and delivery, reduction in toxicity while maintaining therapeutic effects, greater safety and biocompatibility, and faster development of new safe medicines. The use of a pharmaceutically acceptable carrier, such as a protein, is also advantageous as proteins, such as albumin, are nontoxic, non-immunogenic, biocompatible, and biodegradable.

This application also recognizes that nucleoside or nucleotide derivatives are difficult to formulate into dosage forms that achieve and/or optimize the desired therapeutic effect(s) while minimizing its adverse effects. As such, there exists a need to develop compositions that deliver nucleoside or nucleotide derivatives with improved drug delivery and efficacy.

The application also recognizes that, in a non-limiting example, that chemically modifying a nucleoside or nucleotide into the corresponding prodrug form allows for the formulation of a nanoparticle composition wherein albumin is the carrier. In some instances, a wide variety of nucleosides or nucleotides is compatible for use, regardless of nitrogenous base (either natural or non-natural base), ring structure of the sugar moiety (either cyclic or acyclic), and number of phosphate groups (either none or containing at least one phosphate group). In one aspect provided herein, suitable nucleotide derivatives, such as the monophosphate compounds described herein, are used to prepare nanoparticle formulations comprising albumin as a carrier.

Provided herein are compositions comprising nanoparticles that allow for the drug delivery of the nucleotide derivatives described herein, such as the compounds of Formula (I) or Formula (II). These nanoparticle compositions further comprise pharmaceutically acceptable carriers that interact with the nucleotide derivatives described herein to provide the compositions in a form that is suitable for administration to a subject in need thereof. In some embodiments, this application recognizes that the compounds of Formula (I) or Formula (II), which are prodrugs of gemcitabine, as described herein with specific pharmaceutically acceptable carriers, such as the albumin-based pharmaceutically acceptable carriers described herein, provide nanoparticle formulations that are stable. Also, this application recognizes that, in some instances, use of unmodified nucleoside or nucleotide (e.g. without forming the prodrug as described herein) with the albumin-based pharmaceutically acceptable carriers described herein do not result in stable nanoparticle formulations.

Nucleoside derivatives or analogs constitute a major class of chemotherapeutic agents and are used for the treatment of patients with cancer. This group of agents, known as antimetabolites, includes a variety of pyrimidine and purine nucleoside derivatives with cytotoxic activity in both hematological and solid tumors. Gemcitabine (2′,2′-difluoro-2′-deoxycytidine) is a pyrimidine nucleoside analogue, shown to be active against several solid tumor types.

Both innate and acquired resistance to nucleoside analogues is a problem in the treatment of cancer and is regarded as a driver of poor patient survival outcomes. Gemcitabine faces inherent and acquired cancer resistance mechanisms that limit its effectiveness. These include (i) poor conversion of gemcitabine into the active forms, dFdCDP and dFdCTP; (ii) rapid degradation into inactive or toxic byproducts; and (iii) limited uptake by cancer cells. These effects are due to multiple factors including the following: (i) down-regulation of the key initial phosphorylating enzyme deoxycytidine kinase (dCK) required to convert gemcitabine into the monophosphate form; (ii) expression of the key deactivating enzyme cytidine deaminase; and (iii) deficiency of nucleoside transporter proteins. In addition, increased expression and/or activity of cytidine deaminase (CDA) increases the degradation of gemcitabine into the toxic metabolite 2′,2′-difluoro-2′-deoxyuridine (dFdU). Similarly, increased expression of ribonucleoside-disphosphate reductase large subunit (RRM1) can lead to increased intracellular concentrations of endogenous nucleoside precursors, avoiding Gemcitabine incorporation. Because of these and other processes, single agent gemcitabine has limited activity in cancer treatment.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” or “alkylene” refers to a straight or branched hydrocarbon chain radical consisting solely of carbon and hydrogen atoms, containing no unsaturation, having from one to eighteen carbon atoms (e.g., C₁-C₁₈ alkyl). In certain embodiments, an alkyl comprises three to eighteen carbon atoms (e.g., C₃-C₁₈ alkyl). In certain embodiments, an alkyl comprises one to fifteen carbon atoms (e.g., C₁-C₁₅ alkyl). In certain embodiments, an alkyl comprises one to twelve carbon atoms (e.g., C₁-C₁₂ alkyl). In certain embodiments, an alkyl comprises one to eight carbon atoms (e.g., C₁-C₈ alkyl). In other embodiments, an alkyl comprises one to six carbon atoms (e.g., C₁-C₆ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to eighteen carbon atoms. In certain embodiments, an alkenyl comprises three to eighteen carbon atoms. In certain embodiments, an alkenyl comprises three to twelve carbon atoms. In certain embodiments, an alkenyl comprises six to twelve carbon atoms. In certain embodiments, an alkenyl comprises six to ten carbon atoms. In certain embodiments, an alkenyl comprises eight to ten carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkynyl” refers to a straight or branched hydrocarbon chain radical group consisting solely of carbon and hydrogen atoms, containing at least one carbon-carbon triple bond, having from two to eighteen carbon atoms. In certain embodiments, an alkynyl comprises three to eighteen carbon atoms. In certain embodiments, an alkynyl comprises three to twelve carbon atoms. In certain embodiments, an alkynyl comprises six to twelve carbon atoms. In certain embodiments, an alkynyl comprises six to ten carbon atoms. In certain embodiments, an alkynyl comprises eight to ten carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Bickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Aryloxy” refers to a radical bonded through an oxygen atom of the formula O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkyloxy” refers to a radical bonded through an oxygen atom of the formula O-aralkyl, where aralkyl is as defined above.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyls are saturated, (i.e., containing single C—C bonds only) or partially unsaturated (i.e., containing one or more double bonds or triple bonds.) Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). A partially unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless stated otherwise specifically in the specification, the term “cycloalkyl” is meant to include cycloalkyl radicals that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Halo” or “halogen” refers to bromo, chloro, fluoro or iodo substituents.

“Haloalkyl” refers to an alkyl radical, as defined above, that is substituted by one or more halo radicals, as defined above.

“Haloalkoxy” refers to an alkoxy radical, as defined above, that is substituted by one or more halo radicals, as defined above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. The alkyl part of the fluoroalkyl radical are optionally substituted as defined above for an alkyl group.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic, or tetracyclic ring system, which include fused, spiro, or bridged ring systems. The heteroatoms in the heterocycloalkyl radical are optionally oxidized. One or more nitrogen atoms, if present, are optionally quaternized. The heterocycloalkyl radical is partially or fully saturated. In some embodiments, the heterocycloalkyl is attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Heteroaryl” refers to a radical derived from a 3- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above which are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{v}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O-heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, the compounds disclosed herein contain one or more asymmetric centers and thus give rise to enantiomers, diastereomers, and other stereoisomeric farms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

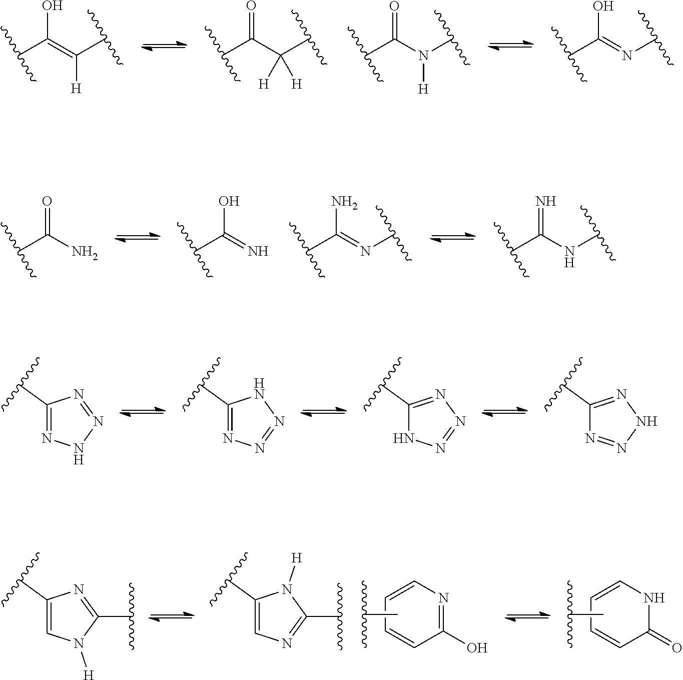

“Optional” or “optionally” means that a subsequently described event or circumstance may or may not occur and that the description includes instances when the event or circumstance occurs and instances in which it does not. For example, “optionally substituted aryl” means that the aryl radical are or are not substituted and that the description includes both substituted aryl radicals and aryl radicals having no substitution.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility, or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of prodrugs include any suitable derivatives of alcohol or amine functional groups in the active compounds and the like that are known to a skilled practitioner. Examples of any suitable derivatives include but are not limited to acetate, formate, and benzoate derivatives of alcohol or amine functional groups.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Compounds

In some embodiments is a compound of Formula (I):

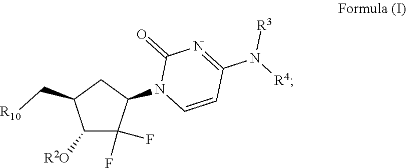

wherein:
R¹ is

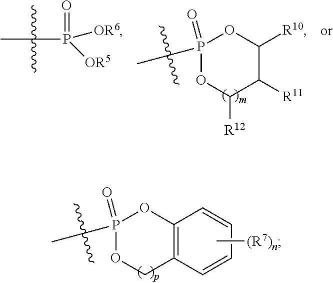

R² is —C(O)R⁸;
R³ is H, —C(O)R⁹, or —C(O)OR⁹;
R⁴ is H;
R⁵ is H, C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁶ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
each R⁷ is independently selected from halogen, C₁₋₈alkyl, C₁₋₈haloalkyl, and C₁₋₈alkoxy;
R⁸ is C₃₋₂₂alkyl, C₃₋₂₂alkenyl, C₃₋₂₂alkynyl, C₃₋₂₂haloalkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl; —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁹ is C₁₋₁₂alkyl;
R¹⁰ and R¹² are each independently H or C₁₋₁₂alkyl, or R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³,
R¹² is H or C₁₋₁₂alkyl;
each R¹³ is independently selected from C₁₋₁₂alkyl;
each R¹⁴ is independently selected from halogen, C₁₋₈alkyl, C₁₋₈haloalkyl, C₁₋₈alkoxy, and —C(O)R¹³;
m is 0 or 1,
n is 0, 1, 2, 3, or 4; and
p is 0 or 1.

In another embodiment is a compound of Formula (I), wherein R¹ is

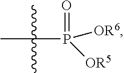

In another embodiment is a compound of Formula (I), wherein R¹ is

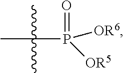

R⁵ is C₃₋₂₂alkyl, and R⁶ is C₃₋₂₂alkyl. In another embodiment is a compound of Formula (I), wherein R¹ is

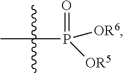

R⁵ is C₃₋₁₈alkyl, and R⁶ is C₃₋₁₈alkyl. In another embodiment is a compound of Formula (I), wherein R¹ is

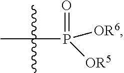

R⁵ is C₃₋₁₅alkyl, and R⁶ is C₃₋₁₅alkyl. In another embodiment is a compound of Formula (I), wherein R¹ is

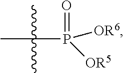

R⁵ is C₆₋₁₅alkyl, and R⁶ is C₆₋₁₅alkyl. In another embodiment is a compound of Formula (I), wherein R¹ is

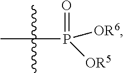

R⁵ is C₆₋₁₂alkyl, and R⁶ is C₆₋₁₂alkyl. In another embodiment is a compound of Formula (I), wherein R¹ is

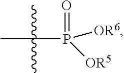

R⁵ is C₆₋₁₀alkyl, and R⁶ is C₆₋₁₀alkyl. In another embodiment is a compound of Formula (I), wherein R¹ is

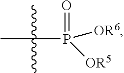

R⁵ is C₃₋₂₂alkenyl, and R⁶ is C₃₋₂₂alkenyl. In another embodiment is a compound of Formula (I), wherein R¹ is

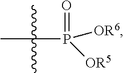

R⁵ is C₃₋₁₈alkenyl, and R⁶ is C₃₋₁₈alkenyl. In another embodiment is a compound of Formula (I), wherein R¹ is

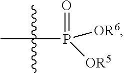

R⁵ is C₃₋₁₅alkenyl, and R⁶ is C₃₋₁₅alkenyl. In another embodiment is a compound of Formula (I), wherein R¹ is

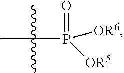

R⁵ is C₆₋₁₅alkenyl, and R⁶ is C₆₋₁₅alkenyl. In another embodiment is a compound of Formula (I), wherein R¹ is

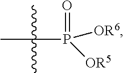

R⁵ is C₆₋₁₂alkenyl, and R⁶ is C₆₋₁₂alkenyl. In another embodiment is a compound of Formula (I), wherein R¹ is

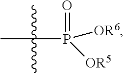

R⁵ is C₆₋₁₀alkenyl, and R⁶ is C₆₋₁₀alkenyl. In another embodiment is a compound of Formula (I), wherein R¹ is

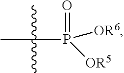

R⁵ is C₃₋₂₂alkynyl, and R⁶ is C₃₋₂₂alkynyl. In another embodiment is a compound of Formula (I), wherein R¹ is

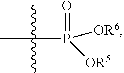

R⁵ is C₃₋₁₈alkynyl, and R⁶ is C₃₋₁₈alkynyl. In another embodiment is a compound of Formula (I), wherein R¹ is

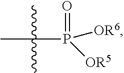

R⁵ is C₃₋₁₅alkynyl, and R⁶ is C₃₋₁₅alkynyl. In another embodiment is a compound of Formula (I), wherein R¹ is

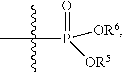

R⁵ is C₆₋₁₅alkynyl, and R⁶ is C₆₋₁₅alkynyl. In another embodiment is a compound of Formula (I), wherein R¹ is

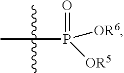

R⁵ is C₆₋₁₂alkynyl, and R⁶ is C₆₋₁₂alkynyl. In another embodiment is a compound of Formula (I), wherein R¹ is

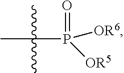

R⁵ is C₆₋₁₀alkynyl, and R⁶ is C₆₋₁₀alkynyl. In another embodiment is a compound of Formula (I), wherein R¹ is

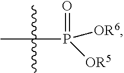

R⁵ is C₃₋₂₂haloalkyl, and R⁶ is C₃₋₂₂haloalkyl. In another embodiment is a compound of Formula (I), wherein R¹ is

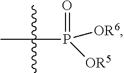

R⁵ is C₃₋₁₈haloalkyl, and R⁶ is C₃₋₁₈haloalkyl.

In another embodiment is a compound of Formula (I), wherein R¹ is

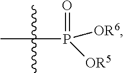

R⁵ is C₃₋₁₅haloalkyl, and R⁶ is C₃₋₁₅haloalkyl. In another embodiment is a compound of Formula (I), wherein R¹ is

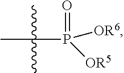

R⁵ is C₆₋₁₅haloalkyl, and R⁶ is C₆₋₁₅haloalkyl. In another embodiment is a compound of Formula (I), wherein R¹ is

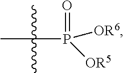

R⁵ is C₆₋₁₂haloalkyl, and R⁶ is C₆₋₁₂haloalkyl. In another embodiment is a compound of Formula (I), wherein R¹ is

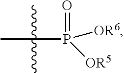

R⁵ is C₆₋₁₀haloalkyl, and R⁶ is C₆₋₁₀haloalkyl. In another embodiment is a compound of Formula (I), wherein R¹ is

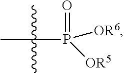

R⁵ is —C₁₋₄alkyl-OC(O)C₁₋₈alkyl, and R⁶ is —C₁₋₄alkyl-OC(O)C₁₋₈alkyl. In another embodiment is a compound of Formula (I), wherein R¹ is

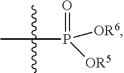

R⁵ is —C₁₋₂alkyl-OC(O)C₁₋₈alkyl, and R⁶ is —C₁₋₂alkyl-OC(O)C₁₋₈alkyl. In another embodiment is a compound of Formula (I), wherein R¹ is

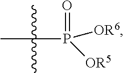

R⁵ is —CH₂OC(O)C₁₋₈alkyl, and R⁶ is —CH₂OC(O)C₁₋₈alkyl. In another embodiment is a compound of Formula (I), wherein R¹ is

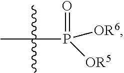

R⁵ is —C₁₋₄alkyl-OC(O)C₁₋₆alkyl, and R⁶ is —C₁₋₄alkyl-OC(O)C₁₋₆alkyl. In another embodiment is a compound of Formula (I), wherein R¹ is

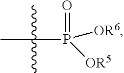

R⁵ is —C₁₋₂alkyl-OC(O)C₁₋₆alkyl, and R⁶ is —C₁₋₂alkyl-OC(O)C₁₋₆alkyl. In another embodiment is a compound of Formula (I), wherein R¹ is

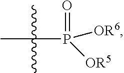

R⁵ is —CH₂OC(O)C₁₋₆alkyl, and R⁶ is —CH₂OC(O)C₁₋₆alkyl. In another embodiment is a compound of Formula (I), wherein R¹ is

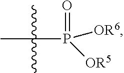

R⁵ is —C₁₋₄alkyl-OC(O)C₁₋₄alkyl, and R⁶ is —C₁₋₄alkyl-OC(O)C₁₋₄alkyl. In another embodiment is a compound of Formula (I), wherein R¹ is

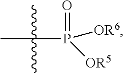

R⁵ is —C₁₋₂alkyl-OC(O)C₁₋₄alkyl, and R⁶ is —C₁₋₂alkyl-OC(O)C₁₋₄alkyl. In another embodiment is a compound of Formula (I), wherein R¹ is

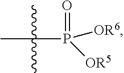

R⁵ is —CH₂OC(O)C₁₋₄alkyl, and R⁶ is —CH₂OC(O)C₁₋₄alkyl. In another embodiment is a compound of Formula (I), wherein R¹ is

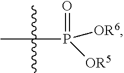

R⁵ is —C₁₋₄alkyl-OC(O)C(CH₃)₃, and R⁶ is —C₁₋₄alkyl-OC(O)C(CH₃)₃. In another embodiment is a compound of Formula (I), wherein R¹ is

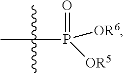

R⁵ is —C₁₋₂alkyl-OC(O)C(CH₃)₃, and R⁶ is —C₁₋₂alkyl-OC(O)C(CH₃)₃. In another embodiment is a compound of Formula (I), wherein R¹ is

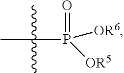

R⁵ is —CH₂OC(O)C(CH₃)₃, and R⁶ is —CH₂OC(O)C(CH₃)₃. In another embodiment is a compound of Formula (I), wherein R¹ is

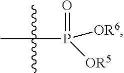

R⁵ is C₃₋₈cycloalkyl, and R⁶ is C₃₋₈cycloalkyl. In another embodiment is a compound of Formula (I), wherein R¹ is

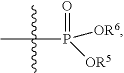

R⁵ is C₃₋₆cycloalkyl, and R⁶ is C₃₋₆cycloalkyl. In another embodiment is a compound of Formula (I), wherein R¹ is

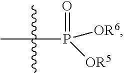

R⁵ is unsubstituted C₆₋₁₀aryl, and R⁶ is unsubstituted C₆₋₁₀aryl. In another embodiment is a compound of Formula (I), wherein R¹ is

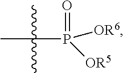

R⁵ is C₆₋₁₀aryl substituted with 1 or 2 R¹⁴, and R⁶ is C₆₋₁₀aryl substituted with 1 or 2 R¹⁴. In another embodiment is a compound of Formula (I), wherein R¹ is

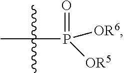

R⁵ is unsubstituted phenyl, and R⁶ is unsubstituted phenyl. In another embodiment is a compound of Formula (I), wherein R¹ is

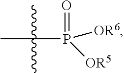

R⁵ is phenyl substituted with 1 or 2 R¹⁴, and R⁶ is phenyl substituted with 1 or 2 R¹⁴. In another embodiment is a compound of Formula (I), wherein R¹ is

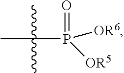

R⁵ is unsubstituted —C₁₋₈alkyl-C₆₋₁₀aryl, and R⁶ is unsubstituted —C₁₋₈alkyl-C₆₋₁₀aryl. In another embodiment is a compound of Formula (I), wherein R¹ is

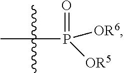

R⁵ is —C₁₋₈alkyl-C₆₋₁₀aryl substituted with 1 or 2 R¹⁴, and R⁶ is —C₁₋₈alkyl-C₆₋₁₀aryl substituted with 1 or 2 R¹⁴. In another embodiment is a compound of Formula (I), wherein R¹ is

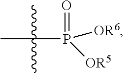

R⁵ is unsubstituted —CH₂-phenyl, and R⁶ is unsubstituted —CH₂-phenyl. In another embodiment is a compound of Formula (I), wherein R¹ is

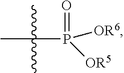

R⁵ is —CH₂-phenyl substituted with 1 or 2 R¹⁴, and R⁶ is —CH₂-phenyl substituted with 1 or 2 R¹⁴. In another embodiment is a compound of Formula (I), wherein R¹ is

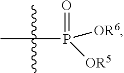

R⁵ is unsubstituted C₂₋₉heteroaryl, and R⁶ is unsubstituted C₂₋₉heteroaryl. In another embodiment is a compound of Formula (I), wherein R¹ is

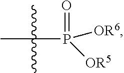

R⁵ is C₂₋₉heteroaryl substituted with 1 or 2 R¹⁴, and R⁶ is C₂₋₉heteroaryl substituted with 1 or 2 R¹⁴. In another embodiment is a compound of Formula (I), wherein R¹ is

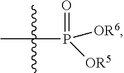

R⁵ is unsubstituted —C₁₋₈alkyl-C₂₋₉heteroaryl, and R⁶ is unsubstituted —C₁₋₈alkyl-C₂₋₉heteroaryl. In another embodiment is a compound of Formula (I), wherein R¹ is

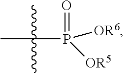

R⁵ is —C₁₋₈alkyl-C₂₋₉heteroaryl substituted with 1 or 2 R¹⁴, and R⁶ is —C₁₋₈alkyl-C₂₋₉heteroaryl substituted with 1 or 2 R¹⁴.

In another embodiment is a compound of Formula (I), wherein R¹ is

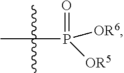

R⁵ is H, and R⁶ is C₃₋₂₂alkyl. In another embodiment is a compound of Formula (I), wherein R¹ is

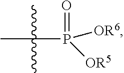

R⁵ is H, and R⁶ is C₃₋₁₈alkyl. In another embodiment is a compound of Formula (I), wherein R¹ is

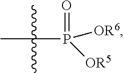

R⁵ is H, and R⁶ is C₆₋₁₅alkyl. In another embodiment is a compound of Formula (I), wherein R¹ is

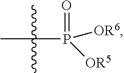

R⁵ is H, and R⁶ is C₆₋₁₀alkyl. In another embodiment is a compound of Formula (I), wherein R¹ is

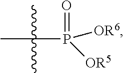

R⁵ is H, and R⁶ is C₃₋₂₂alkenyl. In another embodiment is a compound of Formula (I), wherein R¹ is

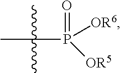

R⁵ is H, and R⁶ is C₃₋₁₈alkenyl. In another embodiment is a compound of Formula (I), wherein R¹ is

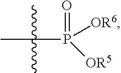

R⁵ is H, and R⁶ is C₆₋₁₅alkenyl. In another embodiment is a compound of Formula (I), wherein R¹ is

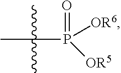

R⁵ is H, and R⁶ is C₆₋₁₀alkenyl. In another embodiment is a compound of Formula (I), wherein R¹ is

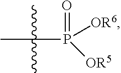

R⁵ is H, and R⁶ is C₃₋₂₂alkynyl. In another embodiment is a compound of Formula (I), wherein R¹ is

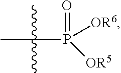

R⁵ is H, and R⁶ is C₃₋₁₈alkynyl. In another embodiment is a compound of Formula (I), wherein R¹ is

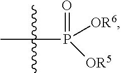

R⁵ is H, and R⁶ is C₆₋₁₅alkynyl. In another embodiment is a compound of Formula (I), wherein R¹ is

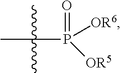

R⁵ is H, and R⁶ is C₆₋₁₀alkynyl. In another embodiment is a compound of Formula (I), wherein R¹ is

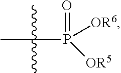

R⁵ is H, and R⁶ is C₃₋₂₂haloalkyl. In another embodiment is a compound of Formula (I), wherein R¹ is

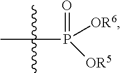

R⁵ is H, and R⁶ is C₃₋₁₈haloalkyl. In another embodiment is a compound of Formula (I), wherein R¹ is

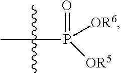

R⁵ is H, and R⁶ is C₆₋₁₅haloalkyl. In another embodiment is a compound of Formula (I), wherein R¹ is

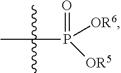

R⁵ is H, and R⁶ is C₆₋₁₀haloalkyl. In another embodiment is a compound of Formula (I), wherein R¹ is

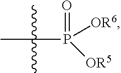

R⁵ is H, and R⁶ is —C₁₋₄alkyl-OC(O)C₁₋₈alkyl. In another embodiment is a compound of Formula (I), wherein R¹ is

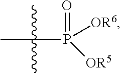

R⁵ is H, and R⁶ is —C₁₋₂alkyl-OC(O)C₁₋₈alkyl. In another embodiment is a compound of Formula (I), wherein R¹ is

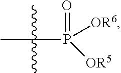

R⁵ is H, and R⁶ is —CH₂OC(O)C₁₋₈alkyl. In another embodiment is a compound of Formula (I), wherein R¹ is

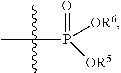

R⁵ is H, and R⁶ is —C₁₋₄alkyl-OC(O)C₁₋₄alkyl. In another embodiment is a compound of Formula (I), wherein R¹ is

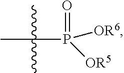

R⁵ is H, and R⁶ is —C₁₋₂alkyl-OC(O)C₁₋₄alkyl. In another embodiment is a compound of Formula (I), wherein R¹ is

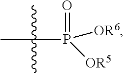

R⁵ is H, and R⁶ is —CH₂OC(O)C₁₋₄alkyl. In another embodiment is a compound of Formula (I), wherein R¹ is

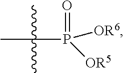

R⁵ is H, and R⁶ is —C₁₋₄alkyl-OC(O)C(CH₃)₃. In another embodiment is a compound of Formula (I), wherein R¹ is

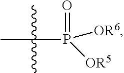

R⁵ is H, and R⁶ is —C₁₋₂alkyl-OC(O)C(CH₃)₃. In another embodiment is a compound of Formula (I), wherein R¹ is

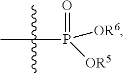

R⁵ is H, and R⁶ is —CH₂OC(O)C(CH₃)₃.

In another embodiment is a compound of Formula (I), wherein R¹ is

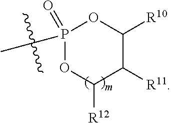

In another embodiment is a compound of Formula (I), wherein R¹ is

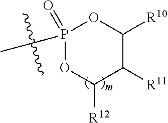

and m is 1. In another embodiment is a compound of Formula (I), wherein R¹ is

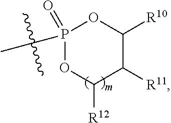

m is 1, and R¹⁰, R¹¹ and R¹² are each H. In another embodiment is a compound of Formula (I), wherein R¹ is

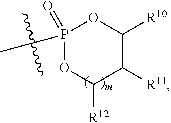

m is 1, and R¹⁰, R¹¹ and R¹² are each C₁₋₁₂alkyl. In another embodiment is a compound of Formula (I), wherein R¹ is

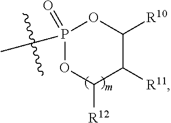

m is 1, and R¹⁰, R¹¹ and R¹² are each C₁₋₄alkyl. In another embodiment is a compound of Formula (I), wherein R¹ is

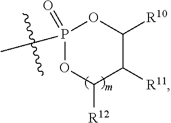

m is 1, and R¹⁰, R¹¹ and R¹² are each —CH₃. In another embodiment is a compound of Formula (I), wherein R¹ is

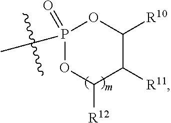

m is 1, R¹⁰ is C₁₋₁₂alkyl, and R¹¹ and R¹² are each H. In another embodiment is a compound of Formula (I), wherein R¹ is

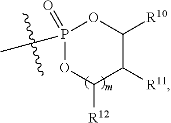

m is 1, R¹⁰ is C₁₋₄alkyl, and R¹¹ and R¹² are each H. In another embodiment is a compound of Formula (I), wherein R¹ is

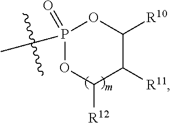

m is 1, R¹⁰ is —CH₃, and R¹¹ and R¹² are each H. In another embodiment is a compound of Formula (I), wherein R¹ is

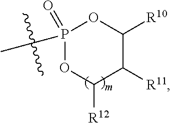

m is 1, R¹¹ is C₁₋₁₂alkyl, and R¹⁰ and R¹² are each H. In another embodiment is a compound of Formula (I), wherein R¹ is

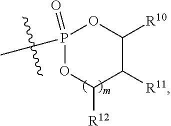

m is 1, R¹¹ is C₁₋₄alkyl, and R¹⁰ and R¹² are each H. In another embodiment is a compound of Formula (I), wherein

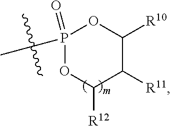

m is 1, R¹¹ is —CH₃, and R¹⁰ and R¹² are each H. In another embodiment is a compound of Formula (I), wherein R¹ is

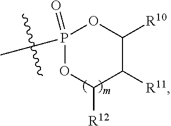

m is 1, R¹⁰ is H, and R¹¹ and R¹² are each C₁₋₁₂alkyl. In another embodiment is a compound of Formula (I), wherein R¹ is

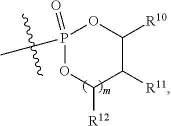

m is 1, R¹⁰ is H, and R¹¹ and R¹² are each C₁₋₄alkyl. In another embodiment is a compound of Formula (I), wherein R¹ is

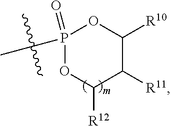

m is 1, R¹⁰ is H, and R¹¹ and R¹² are each —CH₃. In another embodiment is a compound of Formula (I), wherein R¹ is

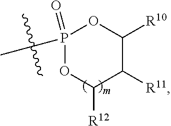

m is 1, R¹¹ is H, and R¹⁰ and R¹² are each C₁₋₁₂alkyl. In another embodiment is a compound of Formula (I), wherein R¹ is

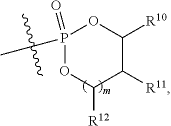

m is 1, R¹¹ is H, and R¹⁰ and R¹² are each C₁₋₄alkyl. In another embodiment is a compound of Formula (I), wherein R¹ is

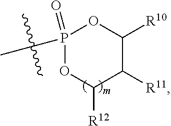

m is 1, R¹¹ is H, and R¹⁰ and R¹² are each —CH₃. In another embodiment is a compound of Formula (I), wherein R¹ is

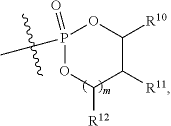

m is 1, R¹² is H, and R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In another embodiment is a compound of Formula (I), wherein R¹ is

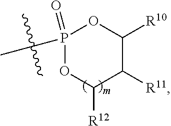

m is 1, R¹² is H, and R¹⁰ and R¹² form a 5- or 6-membered cycloalkyl ring optionally substituted with one or two R¹³. In another embodiment is a compound of Formula (I), wherein R¹ is

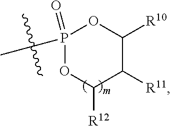

m is 1, R¹² is H, and R¹⁰ and R¹² form a 5- or 6-membered heterocycloalkyl ring optionally substituted with one or two R¹³. In another embodiment is a compound of Formula (I), wherein R¹ is

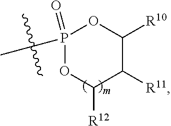

m is 1, R¹² is C₁₋₁₂alkyl, and R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In another embodiment is a compound of Formula (I), wherein R¹ is

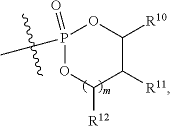

m is 1, R¹² is C₁₋₁₂alkyl, and R¹⁰ and R¹² form a 5- or 6-membered cycloalkyl ring optionally substituted with one or two R¹³. In another embodiment is a compound of Formula (I), wherein R¹ is

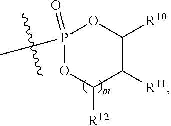

m is 1, R¹² is C₁₋₁₂alkyl, and R¹⁰ and R¹² form a 5- or 6-membered heterocycloalkyl ring optionally substituted with one or two R¹³. In another embodiment is a compound of Formula (I), wherein R¹ is

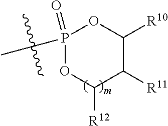

and m is 0. In another embodiment is a compound of Formula (I), wherein R¹ is

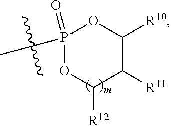

m is 0, and R¹⁰ and R¹² are each H. In another embodiment is a compound of Formula (I), wherein R¹ is

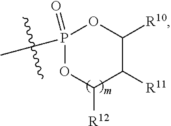

m is 0, and R¹⁰ and R¹¹ are each C₁₋₁₂alkyl. In another embodiment is a compound of Formula (I), wherein R¹ is

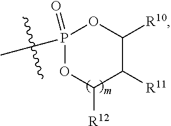

m is 0, and R¹⁰ and R¹² are each C₁₋₄alkyl. In another embodiment is a compound of Formula (I), wherein R¹ is

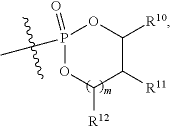

m is 0, and R¹⁰ and R¹² are each —CH₃. In another embodiment is a compound of Formula (I), wherein R¹ is

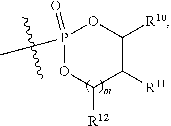

m is 0, R¹⁰ is H, and R¹¹ is C₁₋₁₂alkyl. In another embodiment is a compound of Formula (I), wherein R¹ is

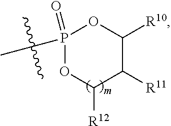

m is 0, R¹⁰ is H, and R¹¹ is C₁₋₄alkyl. In another embodiment is a compound of Formula (I), wherein R¹ is

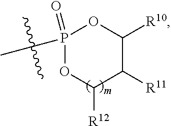

m is 0, R¹⁰ is H, and R¹¹ is —CH₃. In another embodiment is a compound of Formula (I), wherein R¹ is

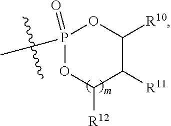

m is 0, R¹⁰ is C₁₋₁₂alkyl, and R¹¹ is H. In another embodiment is a compound of Formula (I), wherein R¹ is

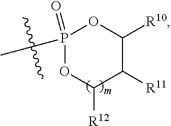

m is 0, R¹⁰ is C₁₋₄alkyl, and R¹¹ is H. In another embodiment is a compound of Formula (I), wherein R¹ is

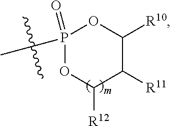

m is 0, R¹⁰ is —CH₃, and R¹¹ is H. In another embodiment is a compound of Formula (I), wherein R¹ is

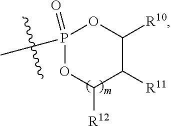

m is 0, and R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In another embodiment is a compound of Formula (I), wherein R¹ is

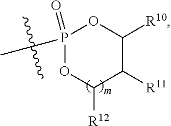

m is 0, and R¹⁰ and R¹² form a 5- or 6-membered cycloalkyl ring optionally substituted with one or two R¹³. In another embodiment is a compound of Formula (I), wherein R¹ is

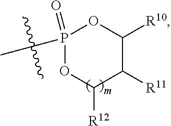

m is 0, and R¹⁰ and R¹² form a 5- or 6-membered heterocycloalkyl ring optionally substituted with one or two R¹³.

In another embodiment is a compound of Formula (I), wherein R¹ is

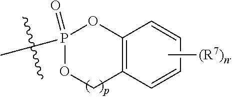

In another embodiment is a compound of Formula (I), or a pharmaceutically acceptable salt thereof, wherein R¹ is

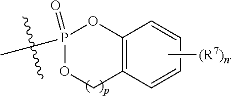

and p is 1. In another embodiment is a compound of Formula (I), wherein R¹ is

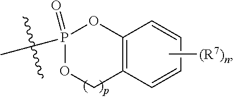

p is 1, and n is 0. In another embodiment is a compound of Formula (I), wherein R¹ is

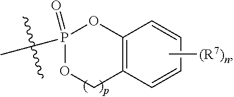

p is 1, and n is 1, 2, 3, or 4. In another embodiment is a compound of Formula (I), wherein R¹ is

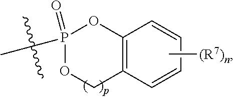

p is 1, and n is 1 or 2. In another embodiment is a compound of Formula (I), wherein R¹ is

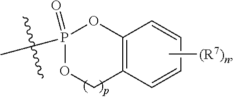

p is 1, and n is 1. In another embodiment is a compound of Formula (I), wherein R¹ is

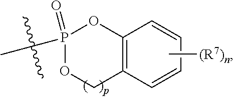

p is 1, n is 1, and R⁷ is halogen. In another embodiment is a compound of Formula (I), wherein R¹ is

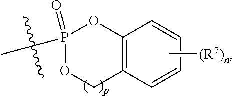

p is 1, n is 1, and R⁷ is C₁₋₈alkyl. In another embodiment is a compound of Formula (I), wherein R¹ is

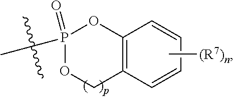

p is 1, n is 1, and R⁷ is C₁₋₈haloalkyl. In another embodiment is a compound of Formula (I), wherein R¹ is

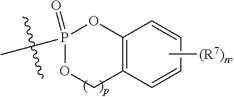

p is 1, n is 1, and R⁷ is C₁₋₈alkoxy. In another embodiment is a compound of Formula (I), wherein R¹ is

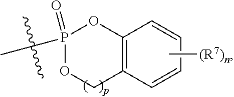

and p is 0. In another embodiment is a compound of Formula (I), wherein R¹ is

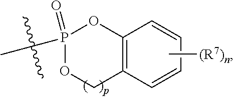

p is 0, and n is 0. In another embodiment is a compound of Formula (I), wherein R¹ is

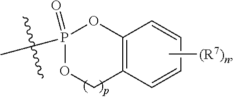

p is 0, and n is 1, 2, 3, or 4. In another embodiment is a compound of Formula (I), wherein R¹ is

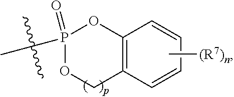

p is 0, and n is 1 or 2. In another embodiment is a compound of Formula (I), wherein R¹ is

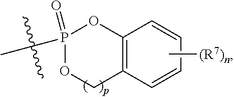

p is 0, and n is 1. In another embodiment is a compound of Formula (I), wherein R¹ is

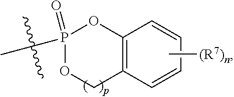

p is 0, n is 1, and R⁷ is halogen. In another embodiment is a compound of Formula (I), wherein R¹ is

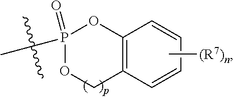

p is 0, n is 1, and R⁷ is C₁₋₈alkyl. In another embodiment is a compound of Formula (I), wherein R¹ is

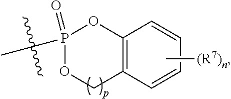

p is 0, n is 1, and R⁷ is C₁₋₈haloalkyl. In another embodiment is a compound of Formula (I), wherein R¹ is

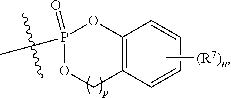

p is 0, n is 1, and R⁷ is C₁₋₈alkoxy.

In another embodiment is a compound of Formula (I), wherein R⁸ is C₃₋₂₂alkyl. In another embodiment is a compound of Formula (I), wherein R⁸ is C₃₋₁₈alkyl. In another embodiment is a compound of Formula (I), wherein R⁸ is C₃₋₁₂alkyl. In another embodiment is a compound of Formula (I), wherein R⁸ is C₆₋₁₂alkyl. In another embodiment is a compound of Formula (I), wherein R⁸ is C₆₋₁₀alkyl. In another embodiment is a compound of Formula (I), wherein R⁸ is C₈₋₁₀alkyl. In another embodiment is a compound of Formula (I), wherein R⁸ is —(CH₂)₂CH₃. In another embodiment is a compound of Formula (I), wherein R⁸ is —(CH₂)₃CH₃. In another embodiment is a compound of Formula (I), wherein R⁸ is —(CH₂)₄CH₃. In another embodiment is a compound of Formula (I), wherein R⁸ is —(CH₂)₅CH₃. In another embodiment is a compound of Formula (I), wherein R⁸ is —(CH₂)₆CH₃. In another embodiment is a compound of Formula (I), wherein R⁸ is —(CH₂)₇CH₃. In another embodiment is a compound of Formula (I), wherein R⁸ is —(CH₂)₈CH₃. In another embodiment is a compound of Formula (I), wherein R⁸ is —(CH₂)₉CH₃. In another embodiment is a compound of Formula (I), wherein R⁸ is —(CH₂)₁₀CH₃. In another embodiment is a compound of Formula (I), wherein R⁸ is —(CH₂)₁₁CH₃. In another embodiment is a compound of Formula (I), wherein R⁸ is —(CH₂)₁₂CH₃. In another embodiment is a compound of Formula (I), wherein R⁸ is —(CH₂)₁₃CH₃. In another embodiment is a compound of Formula (I), wherein R⁸ is —(CH₂)₁₄CH₃. In another embodiment is a compound of Formula (I), wherein R⁸ is —(CH₂)₁₅CH₃. In another embodiment is a compound of Formula (I), wherein R⁸ is —(CH₂)₁₆CH₃. In another embodiment is a compound of Formula (I), wherein R⁸ is —(CH₂)₁₇CH₃. In another embodiment is a compound of Formula (I), wherein R⁸ is C₃₋₂₂alkenyl. In another embodiment is a compound of Formula (I), wherein R⁸ is C₃₋₁₈alkenyl. In another embodiment is a compound of Formula (I), wherein R⁸ is C₃₋₁₂alkenyl. In another embodiment is a compound of Formula (I), wherein R⁸ is C₆₋₁₂alkenyl. In another embodiment is a compound of Formula (I), wherein R⁸ is C₆₋₁₀alkenyl. In another embodiment is a compound of Formula (I), wherein R⁸ is C₈₋₁₀alkenyl. In another embodiment is a compound of Formula (I), wherein R⁸ is C₃₋₂₂alkynyl. In another embodiment is a compound of Formula (I), wherein R⁸ is C₃₋₁₈alkynyl. In another embodiment is a compound of Formula (I), wherein R⁸ is C₃₋₁₂alkynyl. In another embodiment is a compound of Formula (I), wherein R⁸ is C₆₋₁₂alkynyl. In another embodiment is a compound of Formula (I), wherein R⁸ is C₆₋₁₀alkynyl. In another embodiment is a compound of Formula (I), wherein R⁸ is C₈₋₁₀alkynyl. In another embodiment is a compound of Formula (I), wherein R⁸ is C₃₋₂₂haloalkyl. In another embodiment is a compound of Formula (I), wherein R⁸ is C₃₋₁₈haloalkyl. In another embodiment is a compound of Formula (I), wherein R⁸ is C₃₋₁₂haloalkyl. In another embodiment is a compound of Formula (I), wherein R⁸ is C₆₋₁₂haloalkyl. In another embodiment is a compound of Formula (I), wherein R⁸ is C₆₋₁₀haloalkyl. In another embodiment is a compound of Formula (I), wherein R⁸ is C₈₋₁₀haloalkyl. In another embodiment is a compound of Formula (I), wherein R⁸ is C₃₋₈cycloalkyl. In another embodiment is a compound of Formula (I), wherein R⁸ is C₃₋₆cycloalkyl. In another embodiment is a compound of Formula (I), wherein R⁸ is C₆₋₁₀aryl optionally substituted with 1, 2, 3, or 4 R¹⁴. In another embodiment is a compound of Formula (I), wherein R⁸ is unsubstituted C₆₋₁₀aryl. In another embodiment is a compound of Formula (I), wherein R⁸ is C₆₋₁₀aryl substituted with 1 or 2 R¹⁴. In another embodiment is a compound of Formula (I), wherein R⁸ is phenyl optionally substituted with 1, 2, 3, or 4 R¹⁴. In another embodiment is a compound of Formula (I), wherein R⁸ is unsubstituted phenyl. In another embodiment is a compound of Formula (I), wherein R⁸ is phenyl substituted with 1 or 2 R¹⁴. In another embodiment is a compound of Formula (I), wherein R⁸ is —C₁₋₈alkyl-C₆₋₁₀aryl optionally substituted with 1, 2, 3, or 4 R¹⁴. In another embodiment is a compound of Formula (I), wherein R⁸ is unsubstituted —C₁₋₈alkyl-C₆₋₁₀aryl. In another embodiment is a compound of Formula (I), wherein R⁸ is —C₁₋₈alkyl-C₆₋₁₀aryl substituted with 1 or 2 R¹⁴. In another embodiment is a compound of Formula (I), wherein R⁸ is —CH₂-phenyl optionally substituted with 1, 2, 3, or 4 R¹⁴. In another embodiment is a compound of Formula (I), wherein R⁸ is unsubstituted —CH₂-phenyl. In another embodiment is a compound of Formula (I), wherein R⁸ is —CH₂-phenyl substituted with 1 or 2 R¹⁴. In another embodiment is a compound of Formula (I), wherein R⁸ is C₂₋₉heteroaryl optionally substituted with 1, 2, 3, or 4 R¹⁴. In another embodiment is a compound of Formula (I), wherein R⁸ is unsubstituted C₂₋₉heteroaryl. In another embodiment is a compound of Formula (I), wherein R⁸ is C₂₋₉heteroaryl substituted with 1 or 2 R¹⁴. In another embodiment is a compound of Formula (I), wherein R⁸ is —CH₂—C₂₋₉heteroaryl optionally substituted with 1, 2, 3, or 4 R¹⁴. In another embodiment is a compound of Formula (I), wherein R⁸ is unsubstituted —CH₂—C₂₋₉heteroaryl. In another embodiment is a compound of Formula (I), wherein R⁸ is —CH₂—C₂₋₉heteroaryl substituted with 1 or 2 R¹⁴.

In another embodiment is a compound of Formula (I), wherein R³ is H. In another embodiment is a compound of Formula (I), wherein R³ is —C(O)R⁹. In another embodiment is a compound of Formula (I), wherein R³ is —C(O)R⁹ and R⁹ is C₁₋₁₀alkyl. In another embodiment is a compound of Formula (I), wherein R³ is —C(O)R⁹ and R⁹ is C₁₋₆alkyl. In another embodiment is a compound of Formula (I), wherein R³ is —C(O)R⁹ and R⁹ is C₁₋₄alkyl. In another embodiment is a compound of Formula (I), wherein R³ is —C(O)R⁹ and R⁹ is —CH₃. In another embodiment is a compound of Formula (I), wherein R³ is —C(O)R⁹ and R⁹ is —CH₂CH₃. In another embodiment is a compound of Formula (I), wherein R³ is —C(O)OR⁹. In another embodiment is a compound of Formula (I), wherein R³ is —C(O)OR⁹ and R⁹ is C₁₋₁₀alkyl. In another embodiment is a compound of Formula (I), wherein R³ is —C(O)OR⁹ and R⁹ is C₁₋₆alkyl. In another embodiment is a compound of Formula (I), wherein R³ is —C(O)OR⁹ and R⁹ is C₁₋₄alkyl. In another embodiment is a compound of Formula (I), wherein R³ is —C(O)OR⁹ and R⁹ is —CH₃. In another embodiment is a compound of Formula (I), wherein R³ is —C(O)OR⁹ and R⁹ is —CH₂CH₃.

In some embodiments is a compound of Formula (I) having the structure of Formula (Ia):

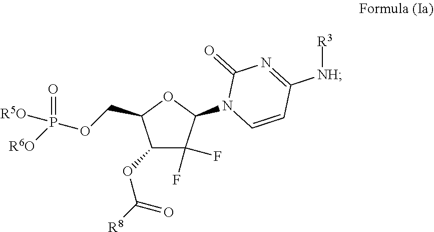

wherein:
R³ is H, —C(O)R⁹, or —C(O)OR⁹;
R⁵ is H, C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₃₋₈alkyl-C₂₋₉heteroaryl, wherein C₆₋₁₀aryl, —C₁₋₈alkyl-C₆₋₁₀aryl, C₂₋₉heteroaryl, or —C alkyl-C₂₋₉heteroaryl are optionally substituted with 1, 2, 3, or 4 R¹⁴;
R⁶ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁸ is C₃₋₂₂alkyl, C₃₋₂₂alkenyl, C₃₋₂₂alkynyl, C₃₋₂₂haloalkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁹ is C₁₋₁₂alkyl;
each R¹³ is independently selected from C₁₋₁₂alkyl; and
each R¹⁴ is independently selected from halogen, C₁₋₈alkyl, C₁₋₈haloalkyl, C₁₋₈alkoxy, and —C(O)R¹³.

In another embodiment is a compound of Formula (Ia), wherein R⁵ is C₃₋₂₂alkyl and R⁶ is C₃₋₂₂alkyl. In another embodiment is a compound of Formula (Ia), wherein R⁵ is C₃₋₁₈alkyl and R⁶ is C₃₋₁₈alkyl. In another embodiment is a compound of Formula (Ia), wherein R⁵ is C₃₋₁₅alkyl and R⁶ is C₃₋₁₅alkyl. In another embodiment is a compound of Formula (Ia), wherein R⁵ is C₆₋₁₅alkyl and R⁶ is C₆₋₁₅alkyl. In another embodiment is a compound of Formula (Ia), wherein R⁵ is C₆₋₁₂alkyl and R⁶ is C₆₋₁₂alkyl. In another embodiment is a compound of Formula (Ia), wherein R⁵ is C₆₋₁₀alkyl and R⁶ is C₆₋₁₀alkyl. In another embodiment is a compound of Formula (Ia), wherein R⁵ is C₃₋₂₂alkenyl and R⁶ is C₃₋₂₂alkenyl. In another embodiment is a compound of Formula (Ia), wherein R⁵ is C₃₋₁₈alkenyl and R⁶ is C₃₋₁₈alkenyl. In another embodiment is a compound of Formula (Ia), wherein R⁵ is C₃₋₁₅alkenyl and R⁶ is C₃₋₁₀alkenyl. In another embodiment is a compound of Formula (Ia), wherein R⁵ is C₆₋₁₅alkenyl and R⁶ is C₆₋₁₅alkenyl. In another embodiment is a compound of Formula (Ia), wherein R⁵ is C₆₋₁₂alkenyl and R⁶ is C₆₋₁₂alkenyl. In another embodiment is a compound of Formula (Ia), wherein R⁵ is C₆₋₁₀alkenyl and R⁶ is C₆₋₁₀alkenyl. In another embodiment is a compound of Formula (Ia), wherein R⁵ is C₃₋₂₂alkynyl and R⁶ is C₃₋₂₂alkynyl. In another embodiment is a compound of Formula (Ia), wherein R⁵ is C₃₋₁₈alkynyl and R⁶ is C₃₋₁₈alkynyl. In another embodiment is a compound of Formula (Ia), wherein R⁵ is C₃₋₁₈alkynyl and R⁶ is C₃₋₁₅alkynyl. In another embodiment is a compound of Formula (Ia), wherein R⁵ is C₆₋₁₅alkynyl and R⁶ is C₆₋₁₅alkynyl. In another embodiment is a compound of Formula (Ia), wherein R⁵ is C₆₋₁₂alkynyl and R⁶ is C₆₋₁₂alkynyl. In another embodiment is a compound of Formula (Ia), wherein R⁵ is C₆₋₁₀alkynyl and R⁶ is C₆₋₁₀alkynyl. In another embodiment is a compound of Formula (Ia), wherein R⁵ is C₃₋₂₂haloalkyl and R⁶ is C₃₋₂₂haloalkyl. In another embodiment is a compound of Formula (Ia), wherein R⁵ is C₃₋₁₈haloalkyl and R⁶ is C₃₋₁₈haloalkyl. In another embodiment is a compound of Formula (Ia), wherein R⁵ is C₃₋₁₅haloalkyl and R⁶ is C₃₋₁₅haloalkyl. In another embodiment is a compound of Formula (Ia), wherein R⁵ is C₆₋₁₅haloalkyl and R⁶ is C₆₋₁₅haloalkyl. In another embodiment is a compound of Formula (Ia), wherein R⁵ is C₆₋₁₂haloalkyl and R⁶ is C₆₋₁₂haloalkyl. In another embodiment is a compound of Formula (Ia), wherein R⁵ is C₆₋₁₀haloalkyl and R⁶ is C₆₋₁₀haloalkyl. In another embodiment is a compound of Formula (Ia), wherein R⁵ is —C₁₋₄alkyl-OC(O)C₁₋₈alkyl and R⁶ is —C₁₋₄alkyl-OC(O)C₁₋₈alkyl. In another embodiment is a compound of Formula (Ia), wherein R⁵ is —C₁₋₂alkyl-OC(O)C₁₋₈alkyl and R⁶ is —C₁₋₂alkyl-OC(O)C₁₋₈alkyl. In another embodiment is a compound of Formula (Ia), wherein R⁵ is —CH₂OC(O)C₁₋₈alkyl and R⁶ is —CH₂OC(O)C₁₋₈alkyl. In another embodiment is a compound of Formula (Ia), wherein R⁵ is —C₁₋₄alkyl-OC(O)C₁₋₆alkyl and R⁶ is —C₁₋₄alkyl-OC(O)C₁₋₆alkyl. In another embodiment is a compound of Formula (Ia), wherein R⁵ is —C₁₋₂alkyl-OC(O)C₁₋₆alkyl and R⁶ is —C₁₋₂alkyl-OC(O)C₁₋₆alkyl. In another embodiment is a compound of Formula (Ia), wherein R⁵ is —CH₂OC(O)C₁₋₆alkyl and R⁶ is —CH₂OC(O)C₁₋₆alkyl. In another embodiment is a compound of Formula (Ia), wherein R⁵ is —C₁₋₄alkyl-OC(O)C₁₋₄alkyl and R⁶ is —C₁₋₄alkyl-OC(O)C₁₋₄alkyl. In another embodiment is a compound of Formula (Ia), wherein R⁵ is —C₁₋₂alkyl-OC(O)C₁₋₄alkyl and R⁶ is —C₁₋₂alkyl-OC(O)C₁₋₄alkyl. In another embodiment is a compound of Formula (Ia), wherein R⁵ is —CH₂OC(O)C₁₋₄alkyl and R⁶ is —CH₂OC(O)C₁₋₄alkyl. In another embodiment is a compound of Formula (Ia), wherein R⁵ is —C₁₋₄alkyl-OC(O)C(CH₃)₃ and R⁶ is —C₁₋₄alkyl-OC(O)C(CH₃)₃. In another embodiment is a compound of Formula (Ia), wherein R⁵ is —C₁₋₂alkyl-OC(O)C(CH₃)₃ and R⁶ is —C₁₋₂alkyl-OC(O)C(CH₃)₃. In another embodiment is a compound of Formula (Ia), wherein R⁵ is —CH₂OC(O)C(CH₃)₃, and R⁶ is —CH₂OC(O)C(CH₃)₃. In another embodiment is a compound of Formula (Ia), wherein R⁵ is C₃₋₈cycloalkyl and R⁶ is C₃₋₈cycloalkyl. In another embodiment is a compound of Formula (Ia), wherein R⁵ is C₃₋₆cycloalkyl and R⁶ is C₃₋₆cycloalkyl. In another embodiment is a compound of Formula (Ia), wherein R⁵ is unsubstituted C₆₋₁₀aryl and R⁶ is unsubstituted C₆₋₁₀aryl. In another embodiment is a compound of Formula (Ia), wherein R⁵ is C₆₋₁₀aryl substituted with 1 or 2 R¹⁴, and R⁶ is C₆₋₁₀aryl substituted with 1 or 2 R¹⁴. In another embodiment is a compound of Formula (Ia), wherein R⁵ is unsubstituted phenyl, and R⁶ is unsubstituted phenyl. In another embodiment is a compound of Formula (Ia), wherein R⁵ is phenyl substituted with 1 or 2 R¹⁴, and R⁶ is phenyl substituted with 1 or 2 R¹⁴. In another embodiment is a compound of Formula (Ia), wherein R⁵ is unsubstituted —C₁₋₈alkyl-C₆₋₁₀aryl, and R⁶ is unsubstituted —C₁₋₈alkyl-C₆₋₁₀aryl. In another embodiment is a compound of Formula (Ia), wherein R⁵ is —C₁₋₈alkyl-C₆₋₁₀aryl substituted with 1 or 2 R¹⁴, and R⁶ is —C₁₋₈alkyl-C₆₋₁₀aryl substituted with 1 or 2 R¹⁴. In another embodiment is a compound of Formula (Ia), wherein R⁵ is unsubstituted —CH₂-phenyl, and R⁶ is unsubstituted —CH₂-phenyl. In another embodiment is a compound of Formula (Ia), wherein R⁵ is —CH₂-phenyl substituted with 1 or 2 R¹⁴, and R⁶ is —CH₂— phenyl substituted with 1 or 2 R¹⁴. In another embodiment is a compound of Formula (Ia), wherein R⁵ is unsubstituted C₂₋₉heteroaryl, and R⁶ is unsubstituted C₂₋₉heteroaryl. In another embodiment is a compound of Formula (Ia), wherein R⁵ is C₂₋₉heteroaryl substituted with 1 or 2 R¹⁴, and R⁶ is C₂₋₉heteroaryl substituted with 1 or 2 R¹⁴. In another embodiment is a compound of Formula (Ia), wherein R⁵ is unsubstituted —C₁₋₈alkyl-C₂₋₉heteroaryl, and R⁶ is unsubstituted —C₁₋₈alkyl-C₂₋₉heteroaryl. In another embodiment is a compound of Formula (Ia), wherein R⁵ is —C₁₋₈alkyl-C₂₋₉heteroaryl substituted with 1 or 2 R¹⁴, and R⁶ is —C₁₋₈alkyl-C₂₋₉heteroaryl substituted with 1 or 2 R¹⁴. In another embodiment is a compound of Formula (Ia), wherein R⁵ is H and R⁶ is C₃₋₂₂alkyl. In another embodiment is a compound of Formula (Ia), wherein R⁵ is H and R⁶ is C₃₋₁₈alkyl. In another embodiment is a compound of Formula (Ia), wherein R⁵ is H and R⁶ is C₆₋₁₅alkyl. In another embodiment is a compound of Formula (Ia), wherein R⁵ is H and R⁶ is C₆₋₁₀alkyl. In another embodiment is a compound of Formula (Ia), wherein R⁵ is H and R⁶ is C₃₋₂₂alkenyl. In another embodiment is a compound of Formula (Ia), wherein R⁵ is H and R⁶ is C₃₋₁₈alkenyl. In another embodiment is a compound of Formula (Ia), wherein R⁵ is H and R⁶ is C₆₋₁₅alkenyl. In another embodiment is a compound of Formula (Ia), wherein R⁵ is H and R⁶ is C₆₋₁₀alkenyl. In another embodiment is a compound of Formula (Ia), wherein R⁵ is H and R⁶ is C₃₋₂₂alkynyl. In another embodiment is a compound of Formula (Ia), wherein R⁵ is H and R⁶ is C₃₋₁₈alkynyl. In another embodiment is a compound of Formula (Ia), wherein R⁵ is H and R⁶ is C₆₋₁₅alkynyl. In another embodiment is a compound of Formula (Ia), wherein R⁵ is H and R⁶ is C₆₋₁₀alkynyl. In another embodiment is a compound of Formula (Ia), wherein R⁵ is H and R⁶ is C₃₋₂₂haloalkyl. In another embodiment is a compound of Formula (Ia), wherein R⁵ is H and R⁶ is C₃₋₁₈haloalkyl. In another embodiment is a compound of Formula (Ia), wherein R⁵ is H and R⁶ is C₆₋₁₅haloalkyl. In another embodiment is a compound of Formula (Ia), wherein R⁵ is H and R⁶ is C₆₋₁₀haloalkyl. In another embodiment is a compound of Formula (Ia), wherein R⁵ is H and R⁶ is —C₁₋₄alkyl-OC(O)C₁₋₈alkyl. In another embodiment is a compound of Formula (Ia), wherein R⁵ is H and R⁶ is —C₁₋₂alkyl-OC(O)C₁₋₈alkyl. In another embodiment is a compound of Formula (Ia), wherein R⁵ is H and R⁶ is —CH₂OC(O)C₁₋₈alkyl. In another embodiment is a compound of Formula (Ia), wherein R⁵ is H and R⁶ is —C₁₋₄alkyl-OC(O)C₁₋₄alkyl. In another embodiment is a compound of Formula (Ia), wherein R⁵ is H and R⁶ is —C₁₋₂alkyl-OC(O)C₁₋₄alkyl. In another embodiment is a compound of Formula (Ia), wherein R⁵ is H and R⁶ is —CH₂OC(O)C₁₋₄alkyl. In another embodiment is a compound of Formula (Ia), wherein R⁵ is H and R⁶ is —C₁₋₄alkyl-OC(O)C(CH₃)₃. In another embodiment is a compound of Formula (Ia), wherein R⁵ is H and R⁶ is —C₁₋₂alkyl-OC(O)C(CH₃)₃. In another embodiment is a compound of Formula (Ia), wherein R⁵ is H and R⁶ is —CH₂OC(O)C(CH₃)₃.

In another embodiment is a compound of Formula (Ia), wherein R⁸ is C₃₋₂₂alkyl. In another embodiment is a compound of Formula (Ia), wherein R⁸ is C₃₋₁₈alkyl. In another embodiment is a compound of Formula (Ia), wherein R⁸ is C₃₋₁₂alkyl. In another embodiment is a compound of Formula (Ia), wherein R⁸ is C₆₋₁₂alkyl. In another embodiment is a compound of Formula (Ia), wherein R⁸ is C₆₋₁₀alkyl. In another embodiment is a compound of Formula (Ia), wherein R⁸ is C₈₋₁₀alkyl. In another embodiment is a compound of Formula (Ia), wherein R⁸ is —(CH₂)₂CH₃. In another embodiment is a compound of Formula (Ia), wherein R⁸ is —(CH₂)₃CH₃. In another embodiment is a compound of Formula (Ia), wherein R⁸ is —(CH₂)₄CH₃. In another embodiment is a compound of Formula (Ia), wherein R⁸ is —(CH₂)₅CH₃. In another embodiment is a compound of Formula (Ia), wherein R⁸ is —(CH₂)₆CH₃. In another embodiment is a compound of Formula (Ia), wherein R⁸ is —(CH₂)₇CH₃. In another embodiment is a compound of Formula (Ia), wherein R⁸ is —(CH₂)₈CH₃. In another embodiment is a compound of Formula (Ia), wherein R⁸ is —(CH₂)₉CH₃. In another embodiment is a compound of Formula (Ia), wherein R⁸ is —(CH₂)₁₀CH₃. In another embodiment is a compound of Formula (Ia), wherein R⁸ is —(CH₂)₁₁CH₃. In another embodiment is a compound of Formula (Ia), wherein R⁸ is —(CH₂)₁₂CH₃. In another embodiment is a compound of Formula (Ia), wherein R⁸ is —(CH₂)₁₃CH₃. In another embodiment is a compound of Formula (Ia), wherein R⁸ is —(CH₂)₁₄CH₃. In another embodiment is a compound of Formula (Ia), wherein R⁸ is —(CH₂)₁₅CH₃. In another embodiment is a compound of Formula (Ia), wherein R⁸ is —(CH₂)₁₆CH₃. In another embodiment is a compound of Formula (Ia), wherein R⁸ is —(CH₂)₁₇CH₃. In another embodiment is a compound of Formula (Ia), wherein R⁸ is C₃₋₂₂alkenyl. In another embodiment is a compound of Formula (Ia), wherein R⁸ is C₃₋₁₈alkenyl. In another embodiment is a compound of Formula (Ia), wherein R⁸ is C₃₋₁₂alkenyl. In another embodiment is a compound of Formula (Ia), wherein R⁸ is C₆₋₁₂alkenyl. In another embodiment is a compound of Formula (Ia), wherein R⁸ is C₆₋₁₀alkenyl. In another embodiment is a compound of Formula (Ia), wherein R⁸ is C₈₋₁₀alkenyl. In another embodiment is a compound of Formula (Ia), wherein R⁸ is C₃₋₂₂alkynyl. In another embodiment is a compound of Formula (Ia), wherein R⁸ is C₃₋₁₈alkynyl. In another embodiment is a compound of Formula (Ia), wherein R⁸ is C₃₋₁₂alkynyl. In another embodiment is a compound of Formula (Ia), wherein R⁸ is C₆₋₁₂alkynyl. In another embodiment is a compound of Formula (Ia), wherein R⁸ is C₆₋₁₀alkynyl. In another embodiment is a compound of Formula (Ia), wherein R⁸ is C₈₋₁₀alkynyl. In another embodiment is a compound of Formula (Ia), wherein R⁸ is C₃₋₂₂haloalkyl. In another embodiment is a compound of Formula (Ia), wherein R⁸ is C₃₋₁₈haloalkyl. In another embodiment is a compound of Formula (Ia), wherein R⁸ is C₃₋₁₂haloalkyl. In another embodiment is a compound of Formula (Ia), wherein R⁸ is C₆₋₁₂haloalkyl. In another embodiment is a compound of Formula (Ia), wherein R⁸ is C₆₋₁₀haloalkyl. In another embodiment is a compound of Formula (Ia), wherein R⁸ is C₈₋₁₀haloalkyl. In another embodiment is a compound of Formula (Ia), wherein R⁸ is C₃₋₈cycloalkyl. In another embodiment is a compound of Formula (Ia), wherein R⁸ is C₃₋₆cycloalkyl. In another embodiment is a compound of Formula (Ia), wherein R⁸ is C₆₋₁₀aryl optionally substituted with 1, 2, 3, or 4 R¹⁴. In another embodiment is a compound of Formula (Ia), wherein R⁸ is unsubstituted C₆₋₁₀aryl. In another embodiment is a compound of Formula (Ia), wherein R⁸ is C₆₋₁₀aryl substituted with 1 or 2 R¹⁴. In another embodiment is a compound of Formula (Ia), wherein R⁸ is phenyl optionally substituted with 1, 2, 3, or 4 R¹⁴. In another embodiment is a compound of Formula (Ia), wherein R⁸ is unsubstituted phenyl. In another embodiment is a compound of Formula (Ia), wherein R⁸ is phenyl substituted with 1 or 2 R¹⁴. In another embodiment is a compound of Formula (Ia), wherein R⁸ is —C₁₋₈alkyl-C₆₋₁₀aryl optionally substituted with 1, 2, 3, or 4 R¹⁴. In another embodiment is a compound of Formula (Ia), wherein R⁸ is unsubstituted —C₁₋₈alkyl-C₆₋₁₀aryl. In another embodiment is a compound of Formula (Ia), wherein R⁸ is —C₁₋₈alkyl-C₆₋₁₀aryl substituted with 1 or 2 R¹⁴. In another embodiment is a compound of Formula (Ia), wherein R⁸ is —CH₂-phenyl optionally substituted with 1, 2, 3, or 4 R¹⁴. In another embodiment is a compound of Formula (Ia), wherein R⁸ is unsubstituted —CH₂-phenyl. In another embodiment is a compound of Formula (Ia), wherein R⁸ is —CH₂-phenyl substituted with 1 or 2 R¹⁴. In another embodiment is a compound of Formula (Ia), wherein R⁸ is C₂₋₉heteroaryl optionally substituted with 1, 2, 3, or 4 R¹⁴. In another embodiment is a compound of Formula (Ia), wherein R⁸ is unsubstituted C₂₋₉heteroaryl. In another embodiment is a compound of Formula (Ia), wherein R⁸ is C₂₋₉heteroaryl substituted with 1 or 2 R¹⁴. In another embodiment is a compound of Formula (Ia), wherein R⁸ is —CH₂—C₂₋₉heteroaryl optionally substituted with 1, 2, 3, or 4 R¹⁴. In another embodiment is a compound of Formula (Ia), wherein R⁸ is unsubstituted —CH₂—C₂₋₉heteroaryl. In another embodiment is a compound of Formula (Ia), wherein R⁸ is —CH₂—C₂₋₉heteroaryl substituted with 1 or 2 R¹⁴.

In another embodiment is a compound of Formula (Ia), wherein R³ is H, —C(O)R⁹, or —C(O)OR⁹. In another embodiment is a compound of Formula (Ia), wherein R³ is —C(O)R⁹. In another embodiment is a compound of Formula (Ia), wherein R³ is —C(O)R⁹ and R⁹ is C₁₋₁₀alkyl. In another embodiment is a compound of Formula (Ia), wherein R³ is —C(O)R⁹ and R⁹ is C₁₋₆alkyl. In another embodiment is a compound of Formula (Ia), wherein R³ is —C(O)R⁹ and R⁹ is C₁₋₄alkyl. In another embodiment is a compound of Formula (Ia), wherein R³ is —C(O)R⁹ and R⁹ is —CH₃. In another embodiment is a compound of Formula (Ia), wherein R³ is —C(O)R⁹ and R⁹ is —CH₂CH₃. In another embodiment is a compound of Formula (Ia), wherein R³ is —C(O)OR⁹. In another embodiment is a compound of Formula (Ia), wherein R³ is —C(O)OR⁹ and R⁹ is C₁₋₁₀alkyl. In another embodiment is a compound of Formula (Ia), wherein R³ is —C(O)OR⁹ and R⁹ is C₁₋₆alkyl. In another embodiment is a compound of Formula (Ia), wherein R³ is —C(O)OR⁹ and R⁹ is C₁₋₄alkyl. In another embodiment is a compound of Formula (Ia), wherein R³ is —C(O)OR⁹ and R⁹ is —CH₃. In another embodiment is a compound of Formula (Ia), wherein R³ is —C(O)OR⁹ and R⁹ is —CH₂CH₃.

In some embodiments is a compound of Formula (I) having the structure of Formula (Ib):

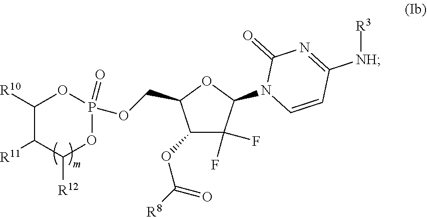

wherein:
R³ is H, —C(O)R⁹, or —C(O)OR⁹,
R⁸ is C₃₋₂₂alkyl, C₃₋₂₂alkenyl, C₃₋₂₂alkynyl, C₃₋₂₂haloalkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁹ is C₁₋₁₂alkyl;
R¹⁰ and R¹² are each independently H or C₁₋₁₂alkyl; or R¹⁰ and R¹² form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³;
R¹² is H or C₁₋₁₂alkyl;
each R¹³ is independently selected from C₁₋₁₂alkyl;
each R¹⁴ is independently selected from halogen, C₁₋₈alkyl, C₁₋₈haloalkyl, C₁₋₈alkoxy, and —C(O)R¹³; and
m is 0 or 1.

In another embodiment is a compound of Formula (Ib), wherein m is 1. In another embodiment is a compound of Formula (Ib), wherein m is 1 and R¹⁰, R¹¹ and R¹² are each H. In another embodiment is a compound of Formula (Ib), wherein m is 1 and R¹⁰, R¹¹ and R¹² are each C₁₋₁₂alkyl. In another embodiment is a compound of Formula (Ib), wherein m is 1 and R¹⁰, R¹¹ and R¹² are each C₁₋₄alkyl. In another embodiment is a compound of Formula (Ib), wherein m is 1 and R¹⁰, R¹¹ are R¹² each —CH₃. In another embodiment is a compound of Formula (Ib), wherein m is 1, R¹⁰ is C₁₋₁₂alkyl, and R¹¹ and R¹² are each H. In another embodiment is a compound of Formula (Ib), wherein m is 1, R¹⁰ is C₁₋₄alkyl, and R¹¹ and R¹² are each H. In another embodiment is a compound of Formula (Ib), wherein m is 1, R¹⁰ is —CH₃, and R¹¹ and R¹² are each H. In another embodiment is a compound of Formula (Ib), wherein m is 1, R¹¹ is C₁₋₁₂alkyl, and R¹⁰ and R¹² are each H. In another embodiment is a compound of Formula (Ib), wherein m is 1, R¹¹ is C₁₋₄alkyl, and R¹⁰ and R¹² are each H. In another embodiment is a compound of Formula (Ib), wherein m is 1, R¹¹ is —CH₃, and R¹⁰ and R¹² are each H. In another embodiment is a compound of Formula (Ib), wherein m is 1, R¹⁰ is H, and R¹¹ and R¹² are each C₁₋₁₂alkyl. In another embodiment is a compound of Formula (M), wherein m is 1, R¹⁰ is H, and R¹¹ and R¹² are each C₁₋₄alkyl. In another embodiment is a compound of Formula (Ib), wherein m is 1, R¹⁰ is H, and R¹¹ and R¹² are each —CH₃. In another embodiment is a compound of Formula (Ib), wherein m is 1, R¹¹ is H, and R¹⁰ and R¹² are each C₁₋₁₂alkyl. In another embodiment is a compound of Formula (Ib), wherein m is 1, R¹¹ is H, and R¹⁰ and R¹² are each C₁₋₄alkyl. In another embodiment is a compound of Formula (Ib), wherein m is 1, R¹¹ is H, and R¹⁰ and R¹² are each —CH₃. In another embodiment is a compound of Formula (Ib), wherein m is 1, R¹² is H, and R¹⁰ and R¹¹ form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In another embodiment is a compound of Formula (Ib), wherein m is 1, R¹² is H, and R¹⁰ and R¹¹ form a 5- or 6-membered cycloalkyl ring optionally substituted with one or two R¹³. In another embodiment is a compound of Formula (Ib), wherein m is 1, R¹² is H, and R¹⁰ and R¹¹ form a 5- or 6-membered heterocycloalkyl ring optionally substituted with one or two R^{H}. In another embodiment is a compound of Formula (Ib), wherein m is 1, R¹² is C₁₋₁₂alkyl, and R¹⁰ and R¹¹ form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In another embodiment is a compound of Formula (Ib), wherein m is 1, R¹² is C₁₋₁₂alkyl, and R¹⁰ and R¹¹ form a 5- or 6-membered cycloalkyl ring optionally substituted with one or two R¹³. In another embodiment is a compound of Formula (Ib), wherein m is 1, R¹² is C₁₋₁₂alkyl, and R¹⁰ and R¹¹ form a 5- or 6-membered heterocycloalkyl ring optionally substituted with one or two R¹³. In another embodiment is a compound of Formula (Ib), wherein m is 0. In another embodiment is a compound of Formula (Ib), wherein m is 0 and R¹⁰ and R¹¹ are each H. In another embodiment is a compound of Formula (Ib), wherein m is 0 and R¹⁰ and R¹¹ are each C₁₋₁₂alkyl. In another embodiment is a compound of Formula (Ib), wherein m is 0 and R¹⁰ and R¹¹ are each C₁₋₄alkyl. In another embodiment is a compound of Formula (Ib), wherein m is 0 and R¹⁰ and R¹¹ are each —CH₃. In another embodiment is a compound of Formula (Ib), wherein m is 0, R¹⁰ is H, and R¹¹ is C₁₋₁₂alkyl. In another embodiment is a compound of Formula (Ib), wherein m is 0, R¹⁰ is H, and R¹¹ is C₁₋₁₂alkyl. In another embodiment is a compound of Formula (Ib), wherein m is 0, R¹⁰ is H, and R¹¹ is —CH₃. In another embodiment is a compound of Formula (Ib), wherein m is 0, R¹⁰ is C₁₋₁₂alkyl, and R¹¹ is H. In another embodiment is a compound of Formula (Ib), wherein m is 0, R¹⁰ is C₁₋₄alkyl, and R¹¹ is H. In another embodiment is a compound of Formula (Ib), wherein m is 0, R¹⁰ is —CH₃, and R¹¹ is H. In another embodiment is a compound of Formula (Ib), wherein m is 0, and R¹⁰ and R¹¹ form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³. In another embodiment is a compound of Formula (Ib), wherein m is 0, and R¹⁰ and R¹¹ form a 5- or 6-membered cycloalkyl ring optionally substituted with one or two R¹³. In another embodiment is a compound of Formula (Ib), wherein m is 0, and R¹⁰ and R¹¹ form a 5- or 6-membered heterocycloalkyl ring optionally substituted with one or two R¹³.

In another embodiment is a compound of Formula (Ib), wherein R⁸ is C₃₋₂₂alkyl. In another embodiment is a compound of Formula (Ib), wherein R⁸ is C₃₋₁₈alkyl. In another embodiment is a compound of Formula (Ib), wherein R⁸ is C₃₋₁₂alkyl. In another embodiment is a compound of Formula (Ib), wherein R⁸ is C₆₋₁₂alkyl. In another embodiment is a compound of Formula (Ib), wherein R⁸ is C₆₋₁₀alkyl. In another embodiment is a compound of Formula (Ib), wherein R⁸ is C₈₋₁₀alkyl. In another embodiment is a compound of Formula (Ib), wherein R⁸ is —(CH₂)₂CH₃. In another embodiment is a compound of Formula (Ib), wherein R⁸ is —(CH₂)₃CH₃. In another embodiment is a compound of Formula (Ib), wherein R⁸ is —(CH₂)₄CH₃. In another embodiment is a compound of Formula (Ib), wherein R⁸ is —(CH₂)₅CH₃. In another embodiment is a compound of Formula (Ib), wherein R⁸ is —(CH₂)₆CH₃. In another embodiment is a compound of Formula (Ib), wherein R⁸ is —(CH₂)₇CH₃. In another embodiment is a compound of Formula (Ib), wherein R⁸ is —(CH₂)₈CH₃. In another embodiment is a compound of Formula (Ib), wherein R⁸ is —(CH₂)₉CH₃. In another embodiment is a compound of Formula (Ib), wherein R⁸ is —(CH₂)₁₀CH₃. In another embodiment is a compound of Formula (Ib), wherein R⁸ is —(CH₂)₁₁CH₃. In another embodiment is a compound of Formula (Ib), wherein R⁸ is —(CH₂)₁₂CH₃. In another embodiment is a compound of Formula (Ib), wherein R⁸ is —(CH₂)₁₃CH₃. In another embodiment is a compound of Formula (Ib), wherein R⁸ is —(CH₂)₁₄CH₃. In another embodiment is a compound of Formula (Ib), wherein R⁸ is —(CH₂)₁₅CH₃. In another embodiment is a compound of Formula (Ib), wherein R⁸ is —(CH₂)₁₆CH₃. In another embodiment is a compound of Formula (Ib), wherein R⁸ is —(CH₂)₁₇CH₃. In another embodiment is a compound of Formula (Ib), wherein R⁸ is C₃₋₂₂alkenyl. In another embodiment is a compound of Formula (Ib), wherein R⁸ is C₃₋₁₈alkenyl. In another embodiment is a compound of Formula (Ib), wherein R⁸ is C₃₋₁₂alkenyl. In another embodiment is a compound of Formula (Ib), wherein R⁸ is C₆₋₁₂alkenyl. In another embodiment is a compound of Formula (Ib), wherein R⁸ is C₆₋₁₀alkenyl. In another embodiment is a compound of Formula (Ib), wherein R⁸ is C₈₋₁₀alkenyl. In another embodiment is a compound of Formula (Ib), wherein R⁸ is C₃₋₂₂alkynyl. In another embodiment is a compound of Formula (Ib), wherein R⁸ is C₃₋₁₈alkynyl. In another embodiment is a compound of Formula (Ib), wherein R⁸ is C₃₋₁₂alkynyl. In another embodiment is a compound of Formula (Ib), wherein R⁸ is C₆₋₁₂alkynyl. In another embodiment is a compound of Formula (Ib), wherein R⁸ is C₆₋₁₂alkynyl. In another embodiment is a compound of Formula (Ib), wherein R⁸ is C₈₋₁₀alkynyl. In another embodiment is a compound of Formula (Ib), wherein R⁸ is C₃₋₂₂haloalkyl. In another embodiment is a compound of Formula (Ib), wherein R⁸ is C₃₋₁₈haloalkyl. In another embodiment is a compound of Formula (Ib), wherein R⁸ is C₃₋₁₂haloalkyl. In another embodiment is a compound of Formula (Ib), wherein R⁸ is C₆₋₁₂haloalkyl. In another embodiment is a compound of Formula (Ib), wherein R⁸ is C₆₋₁₀haloalkyl. In another embodiment is a compound of Formula (Ib), wherein R⁸ is C₈₋₁₀haloalkyl. In another embodiment is a compound of Formula (Ib), wherein R⁸ is C₃₋₈cycloalkyl. In another embodiment is a compound of Formula (Ib), wherein R⁸ is C₃₋₆cycloalkyl. In another embodiment is a compound of Formula (Ib), wherein R⁸ is C₆₋₁₀aryl optionally substituted with 1, 2, 3, or 4 R¹⁴. In another embodiment is a compound of Formula (Ib), wherein R⁸ is unsubstituted C₆₋₁₀aryl. In another embodiment is a compound of Formula (Ib), wherein R⁸ is C₆₋₁₀aryl substituted with 1 or 2 R¹⁴. In another embodiment is a compound of Formula (Ib), wherein R⁸ is phenyl optionally substituted with 1, 2, 3, or 4 R¹⁴. In another embodiment is a compound of Formula (Ib), wherein R⁸ is unsubstituted phenyl. In another embodiment is a compound of Formula (Ib), wherein R⁸ is phenyl substituted with 1 or 2 R¹⁴. In another embodiment is a compound of Formula (Ib), wherein R⁸ is —C₁₋₈alkyl-C₆₋₁₀aryl optionally substituted with 1, 2, 3, or 4 R¹⁴. In another embodiment is a compound of Formula (Ib), wherein R⁸ is unsubstituted —C₁₋₈alkyl-C₆₋₁₀aryl. In another embodiment is a compound of Formula (Ib), wherein R⁸ is —C₁₋₈alkyl-C₆₋₁₀aryl substituted with 1 or 2 R¹⁴. In another embodiment is a compound of Formula (Ib), wherein R⁸ is —CH₂-phenyl optionally substituted with 1, 2, 3, or 4 R¹⁴. In another embodiment is a compound of Formula (Ib), wherein R⁸ is unsubstituted —CH₂-phenyl. In another embodiment is a compound of Formula (Ib), wherein R⁸ is —CH₂-phenyl substituted with 1 or 2 R¹⁴. In another embodiment is a compound of Formula (Ib), wherein R⁸ is C₂₋₉heteroaryl optionally substituted with 1, 2, 3, or 4 R¹⁴. In another embodiment is a compound of Formula (Ib), wherein R⁸ is unsubstituted C₂₋₉heteroaryl. In another embodiment is a compound of Formula (Ib), wherein R⁸ is C₂₋₉heteroaryl substituted with 1 or 2 R¹⁴. In another embodiment is a compound of Formula (Ib), wherein R⁸ is —CH₂—C₂₋₉heteroaryl optionally substituted with 1, 2, 3, or 4 R¹⁴. In another embodiment is a compound of Formula (Ib), wherein R⁸ is unsubstituted —CH₂—C₂₋₉heteroaryl. In another embodiment is a compound of Formula (Ib), wherein R⁸ is —CH₂—C₂₋₉heteroaryl substituted with 1 or 2 R¹⁴.

In another embodiment is a compound of Formula (Ib), wherein R³ is H. In another embodiment is a compound of Formula (Ib), wherein R³ is —C(O)R⁹. In another embodiment is a compound of Formula (Ib), wherein R³ is —C(O)R⁹ and R⁹ is C₁₋₁₀alkyl. In another embodiment is a compound of Formula (Ib), wherein R³ is —C(O)R⁹ and R⁹ is C₁₋₆alkyl. In another embodiment is a compound of Formula (Ib), wherein R³ is —C(O)R⁹ and R⁹ is C₁₋₄alkyl. In another embodiment is a compound of Formula (Ib), wherein R³ is —C(O)R⁹ and R⁹ is —CH₃. In another embodiment is a compound of Formula (Ib), wherein R³ is —C(O)R⁹ and R⁹ is —CH₂CH₃. In another embodiment is a compound of Formula (Ib), wherein R³ is —C(O)OR⁹. In another embodiment is a compound of Formula (Ib), wherein R³ is —C(O)OR⁹ and R⁹ is C₁₋₃₀ alkyl. In another embodiment is a compound of Formula (Ib), wherein R³ is —C(O)OR⁹ and R⁹ is C₃₋₆ alkyl. In another embodiment is a compound of Formula (Ib), wherein R³ is —C(O)OR⁹ and R⁹ is C₃₋₄ alkyl. In another embodiment is a compound of Formula (Ib), wherein R³ is —C(O)OR⁹ and R⁹ is —CH₃. In another embodiment is a compound of Formula (Ib), wherein R³ is —C(O)OR⁹ and R⁹ is —CH₂CH₃.

In some embodiments is a compound of Formula (I) having the structure of Formula (Ic):

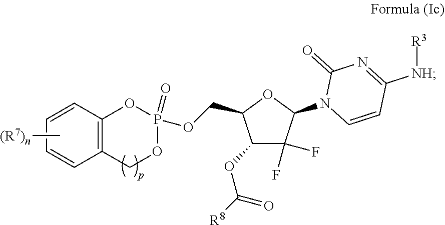

wherein:
R³ is H, —C(O)R⁹, or —C(O)OR⁹;
each R⁷ is independently selected from halogen, C₁₋₈alkyl, C₁₋₈haloalkyl, and C₁₋₈alkoxy;
R⁸ is C₃₋₂₂alkyl, C₃₋₂₂alkenyl, C₃₋₂₂alkynyl, C₃₋₂₂haloalkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁹ is C₃₋₁₂alkyl;
each R¹³ is independently selected from C₃₋₁₂alkyl;
each R¹⁴ is independently selected from halogen, C₁₋₈alkyl, C₁₋₈haloalkyl, C₁₋₈alkoxy, and —C(O)R¹³;
n is 0, 1, 2, 3, or 4; and
p is 0 or 1.

In another embodiment is a compound of Formula (Ic), or a pharmaceutically acceptable salt thereof, wherein p is 1. In another embodiment is a compound of Formula (Ic), wherein p is 1 and n is 0. In another embodiment is a compound of Formula (Ic), wherein p is 1 and n is 1, 2, 3, or 4. In another embodiment is a compound of Formula (Ic), wherein p is 1, n is 1, 2, 3, or 4, and each R⁷ is independently selected from C₁₋₈alkyl, C₁₋₈haloalkyl, and C₁₋₈alkoxy. In another embodiment is a compound of Formula (Ic), wherein p is 1 and n is 1 or 2. In another embodiment is a compound of Formula (Ic), wherein p is 1, n is 1 or 2, and each R⁷ is independently selected from C₁₋₈alkyl, C₁₋₈haloalkyl, and C₁₋₈alkoxy. In another embodiment is a compound of Formula (Ic), wherein p is 1, n is 2. In another embodiment is a compound of Formula (Ic), wherein p is 1, n is 2, and each R⁷ is independently selected from C₁₋₈alkyl, C₁₋₈haloalkyl, and C₁₋₈alkoxy. In another embodiment is a compound of Formula (Ic), wherein p is 1 and n is 1. In another embodiment is a compound of Formula (Ic), wherein p is 1, n is 1, and R⁷ is selected from C₁₋₈alkyl, C₁₋₈haloalkyl, and C₁₋₈alkoxy. In another embodiment is a compound of Formula (Ic), wherein p is 1, n is 1, and R⁷ is halogen. In another embodiment is a compound of Formula (Ic), wherein p is 1, n is 1, and R⁷ is C₁₋₈alkyl. In another embodiment is a compound of Formula (Ic), wherein p is 1, n is 1, and R⁷ is C₁₋₈haloalkyl. In another embodiment is a compound of Formula (Ic), wherein p is 1, n is 1, and R⁷ is C₁₋₈alkoxy. In another embodiment is a compound of Formula (Ic), wherein p is 0. In another embodiment is a compound of Formula (Ic), wherein p is 0 and n is 0. In another embodiment is a compound of Formula (Ic), wherein p is 0 and n is 1, 2, 3, or 4. In another embodiment is a compound of Formula (Ic), wherein p is 0, n is 1, 2, 3, or 4, and each R⁷ is independently selected from C₁₋₈alkyl, C₁₋₈haloalkyl, and C₁₋₈alkoxy. In another embodiment is a compound of Formula (Ic), wherein p is 0 and n is 1 or 2. In another embodiment is a compound of Formula (Ic), wherein p is 0, n is 1 or 2, and each R⁷ is independently selected from C₁₋₈alkyl, C₁₋₈haloalkyl, and C₁₋₈alkoxy. In another embodiment is a compound of Formula (Ic), wherein p is 0, n is 2. In another embodiment is a compound of Formula (Ic), wherein p is 0, n is 2, and each R⁷ is independently selected from C₁₋₈alkyl, C₁₋₈haloalkyl, and C₁₋₈alkoxy. In another embodiment is a compound of Formula (Ic), wherein p is 0 and n is 1. In another embodiment is a compound of Formula (Ic), wherein p is 0, n is 1, and R⁷ is selected from C₁₋₈alkyl, C₁₋₈haloalkyl, and C₁₋₈alkoxy. In another embodiment is a compound of Formula (Ic), wherein p is 0, n is 1, and R⁷ is halogen. In another embodiment is a compound of Formula (Ic), wherein p is 0, n is 1, and R⁷ is C₁₋₈alkyl. In another embodiment is a compound of Formula (Ic), wherein p is 0, n is 1, and R⁷ is C₁₋₈haloalkyl. In another embodiment is a compound of Formula (Ic), wherein p is 0, n is 1, and R⁷ is C₁₋₈alkoxy.

In another embodiment is a compound of Formula (Ic), wherein R⁸ is C₃₋₂₂alkyl. In another embodiment is a compound of Formula (Ic), wherein R⁸ is C₃₋₁₈alkyl. In another embodiment is a compound of Formula (Ic), wherein R⁸ is C₃₋₁₂alkyl. In another embodiment is a compound of Formula (Ic), wherein R⁸ is C₆₋₁₂alkyl. In another embodiment is a compound of Formula (Ic), wherein R⁸ is C₆₋₁₀alkyl. In another embodiment is a compound of Formula (Ic), wherein R⁸ is C₈₋₁₀alkyl. In another embodiment is a compound of Formula (Ic), wherein R⁸ is —(CH₂)₂CH₃. In another embodiment is a compound of Formula (Ic), wherein R⁸ is —(CH₂)₃CH₃. In another embodiment is a compound of Formula (Ic), wherein R⁸ is —(CH₂)₄CH₃. In another embodiment is a compound of Formula (Ic), wherein R⁸ is —(CH₂)₅CH₃. In another embodiment is a compound of Formula (Ic), wherein R⁸ is —(CH₂)₆CH₃. In another embodiment is a compound of Formula (Ic), wherein R⁸ is —(CH₂)₇CH₃. In another embodiment is a compound of Formula (Ic), wherein R⁸ is —(CH₂)₈CH₃. In another embodiment is a compound of Formula (Ic), wherein R⁸ is —(CH₂)₉CH₃. In another embodiment is a compound of Formula (Ic), wherein R⁸ is —(CH₂)₁₀CH₃. In another embodiment is a compound of Formula (Ic), wherein R⁸ is —(CH₂)₁₁CH₃. In another embodiment is a compound of Formula (Ic), wherein R⁸ is —(CH₂)₁₂CH₃. In another embodiment is a compound of Formula (Ic), wherein R⁸ is —(CH₂)₁₃CH₃. In another embodiment is a compound of Formula (Ic), wherein R⁸ is —(CH₂)₁₄CH₃. In another embodiment is a compound of Formula (Ic), wherein R⁸ is —(CH₂)₁₅CH₃. In another embodiment is a compound of Formula (Ic), wherein R⁸ is —(CH₂)₁₆CH₃. In another embodiment is a compound of Formula (Ic), wherein R⁸ is —(CH₂)₁₇CH₃. In another embodiment is a compound of Formula (Ic), wherein R⁸ is C₃₋₂₂alkenyl. In another embodiment is a compound of Formula (Ic), wherein R⁸ is C₃₋₁₈alkenyl. In another embodiment is a compound of Formula (Ic), wherein R⁸ is C₃₋₁₂alkenyl. In another embodiment is a compound of Formula (Ic), wherein R⁸ is C₆₋₁₂alkenyl. In another embodiment is a compound of Formula (Ic), wherein R⁸ is C₆₋₁₀alkenyl. In another embodiment is a compound of Formula (Ic), wherein R⁸ is C₈₋₁₀alkenyl. In another embodiment is a compound of Formula (Ic), wherein R⁸ is C₃₋₂₂alkynyl. In another embodiment is a compound of Formula (Ic), wherein R⁸ is C₃₋₁₈alkynyl. In another embodiment is a compound of Formula (Ic), wherein R⁸ is C₃₋₁₂alkynyl. In another embodiment is a compound of Formula (Ic), wherein R⁸ is C₆₋₁₂alkynyl. In another embodiment is a compound of Formula (Ic), wherein R⁸ is C₆₋₁₀alkynyl. In another embodiment is a compound of Formula (Ic), wherein R⁸ is C₈₋₁₀alkynyl. In another embodiment is a compound of Formula (Ic), wherein R⁸ is C₃₋₂₂haloalkyl. In another embodiment is a compound of Formula (Ic), wherein R⁸ is C₃₋₁₈haloalkyl. In another embodiment is a compound of Formula (Ic), wherein R⁸ is C₃₋₁₂haloalkyl. In another embodiment is a compound of Formula (Ic), wherein R⁸ is C₆₋₁₂haloalkyl. In another embodiment is a compound of Formula (Ic), wherein R⁸ is C₆₋₁₀haloalkyl. In another embodiment is a compound of Formula (Ic), wherein R⁸ is C₈₋₁₀haloalkyl. In another embodiment is a compound of Formula (Ic), wherein R⁸ is C₃₋₈cycloalkyl. In another embodiment is a compound of Formula (Ic), wherein R⁸ is C₃₋₆cycloalkyl. In another embodiment is a compound of Formula (Ic), wherein R⁸ is C₆₋₁₀aryl optionally substituted with 1, 2, 3, or 4 R¹⁴. In another embodiment is a compound of Formula (Ic), wherein R⁸ is unsubstituted C₆₋₁₀aryl. In another embodiment is a compound of Formula (Ic), wherein R⁸ is C₆₋₁₀aryl substituted with 1 or 2 R¹⁴. In another embodiment is a compound of Formula (Ic), wherein R⁸ is phenyl optionally substituted with 1, 2, 3, or 4 R¹⁴. In another embodiment is a compound of Formula (Ic), wherein R⁸ is unsubstituted phenyl. In another embodiment is a compound of Formula (Ic), wherein R⁸ is phenyl substituted with 1 or 2 R¹⁴. In another embodiment is a compound of Formula (Ic), wherein R⁸ is —C₁₋₈alkyl-C₆₋₁₀aryl optionally substituted with 1, 2, 3, or 4 R¹⁴. In another embodiment is a compound of Formula (Ic), wherein R⁸ is unsubstituted —C₁₋₈alkyl-C₆₋₁₀aryl. In another embodiment is a compound of Formula (Ic), wherein R⁸ is —C₁₋₈alkyl-C₆₋₁₀aryl substituted with 1 or 2 R¹⁴. In another embodiment is a compound of Formula (Ic), wherein R⁸ is —CH₂-phenyl optionally substituted with 1, 2, 3, or 4 R¹⁴. In another embodiment is a compound of Formula (Ic), wherein R⁸ is unsubstituted —CH₂-phenyl. In another embodiment is a compound of Formula (Ic), wherein R⁸ is —CH₂-phenyl substituted with 1 or 2 R¹⁴. In another embodiment is a compound of Formula (Ic), wherein R⁸ is C₂₋₉heteroaryl optionally substituted with 1, 2, 3, or 4 R¹⁴. In another embodiment is a compound of Formula (Ic), wherein R⁸ is unsubstituted C₂₋₉heteroaryl. In another embodiment is a compound of Formula (Ic), wherein R⁸ is C₂₋₉heteroaryl substituted with 1 or 2 R¹⁴. In another embodiment is a compound of Formula (Ic), wherein R⁸ is —CH₂—C₂₋₉heteroaryl optionally substituted with 1, 2, 3, or 4 R¹⁴. In another embodiment is a compound of Formula (Ic), wherein R⁸ is unsubstituted —CH₂—C₂₋₉heteroaryl. In another embodiment is a compound of Formula (Ic), wherein R⁸ is —CH₂—C₂₋₉heteroaryl substituted with 1 or 2 R¹⁴.

In another embodiment is a compound of Formula (Ic), wherein R³ is H. In another embodiment is a compound of Formula (Ic), wherein R³ is —C(O)R⁹. In another embodiment is a compound of Formula (Ic), wherein R³ is —C(O)R⁹ and R⁹ is C₁₋₁₀alkyl. In another embodiment is a compound of Formula (Ic), wherein R³ is —C(O)R⁹ and R⁹ is C₁₋₆alkyl. In another embodiment is a compound of Formula (Ic), wherein R³ is —C(O)R⁹ and R⁹ is C₁₋₄alkyl. In another embodiment is a compound of Formula (Ic), wherein R³ is —C(O)R⁹ and R⁹ is —CH₃. In another embodiment is a compound of Formula (Ic), wherein R³ is —C(O)R⁹ and R⁹ is —CH₂CH₃. In another embodiment is a compound of Formula (Ic), wherein R³ is —C(O)OR⁹. In another embodiment is a compound of Formula (Ic), wherein R³ is —C(O)OR⁹ and R⁹ is C₁₋₁₀alkyl. In another embodiment is a compound of Formula (Ic), wherein R³ is —C(O)OR⁹ and R⁹ is C₁₋₆alkyl. In another embodiment is a compound of Formula (Ic), or a pharmaceutically acceptable salt thereof, wherein R³ is —C(O)OR⁹ and R⁹ is C₁₋₄alkyl. In another embodiment is a compound of Formula (Ic), wherein R³ is —C(O)OR⁹ and R⁹ is —CH₃. In another embodiment is a compound of Formula (Ic), wherein R³ is —C(O)OR⁹ and R⁹ is —CH₂CH₃.

In another embodiment is a compound of Formula (II):

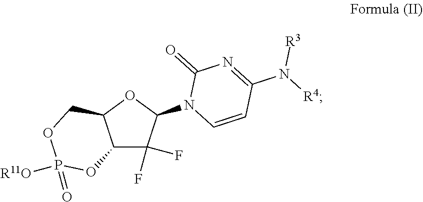

wherein:
R³ is H, —C(O)R⁹, or —C(O)OR⁹;
R⁴ is H;
R⁹ is C₁₋₈alkyl;
R¹¹ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹²;
each R¹² is independently selected from halogen, C₁₋₈alkyl, C₁₋₈haloalkyl, C₁₋₈alkoxy, and —C(O)R¹³; and
each R¹³ is independently selected from C₁₋₁₂alkyl.

In another embodiment is a compound of Formula (II), wherein R¹¹ is C₃₋₂₂alkyl. In another embodiment is a compound of Formula (II), wherein R¹¹ is C₃₋₁₈alkyl. In another embodiment is a compound of Formula (II), wherein R¹¹ is C₃₋₁₂alkyl. In another embodiment is a compound of Formula (II), wherein R¹¹ is C₆₋₁₂alkyl. In another embodiment is a compound of Formula (II), wherein is C₆₋₁₀alkyl. In another embodiment is a compound of Formula (II), wherein R¹¹ is C₈₋₁₀alkyl. In another embodiment is a compound of Formula (II), wherein R¹¹ is —(CH₂)₂CH₃. In another embodiment is a compound of Formula (II), wherein R¹¹ is —(CH₂)₃CH₃. In another embodiment is a compound of Formula (II), wherein R¹¹ is —(CH₂)₄CH₃. In another embodiment is a compound of Formula (II), wherein R¹¹ is —(CH₂)₅CH₃. In another embodiment is a compound of Formula (II), wherein R¹¹ is —(CH₂)₆CH₃. In another embodiment is a compound of Formula (II), wherein R¹¹ is —(CH₂)₇CH₃. In another embodiment is a compound of Formula (II), wherein R¹¹ is —(CH₂)₈CH₃. In another embodiment is a compound of Formula (II), wherein R¹¹ is —(CH₂)₉CH₃. In another embodiment is a compound of Formula (II), wherein R¹¹ is —(CH₂)₁₀CH₃. In another embodiment is a compound of Formula (II), wherein R¹¹ is —(CH₂)₁₁CH₃. In another embodiment is a compound of Formula (II), wherein R¹¹ is —(CH₂)₁₂CH₃. In another embodiment is a compound of Formula (II), wherein R¹¹ is —(CH₂)₁₃CH₃. In another embodiment is a compound of Formula (II), wherein R¹¹ is —(CH₂)₁₄CH₃. In another embodiment is a compound of Formula (II), wherein R¹¹ is —(CH₂)₁₅CH₃. In another embodiment is a compound of Formula (II), wherein R¹¹ is —(CH₂)₁₆CH₃. In another embodiment is a compound of Formula (II), wherein R¹¹ is —(CH₂)₁₇CH₃. In another embodiment is a compound of Formula (II), wherein R¹¹ is C₃₋₂₂alkenyl. In another embodiment is a compound of Formula (II), wherein R¹¹ is C₃₋₁₈alkenyl. In another embodiment is a compound of Formula (II), wherein R¹¹ is C₃₋₁₂alkenyl. In another embodiment is a compound of Formula (II), wherein R¹¹ is C₆₋₁₂alkenyl. In another embodiment is a compound of Formula (II), wherein R¹¹ is C₆₋₁₀alkenyl. In another embodiment is a compound of Formula (II), wherein R¹¹ is C₈₋₁₀alkenyl. In another embodiment is a compound of Formula (II), wherein R¹¹ is C₃₋₂₂alkynyl. In another embodiment is a compound of Formula (II), wherein R¹¹ is C₃₋₁₈alkynyl. In another embodiment is a compound of Formula (II), wherein R¹¹ is C₃₋₁₂alkynyl. In another embodiment is a compound of Formula (II), wherein R¹¹ is C₆₋₁₂alkynyl. In another embodiment is a compound of Formula (II), wherein R¹¹ is C₆₋₁₀alkynyl. In another embodiment is a compound of Formula (II), wherein R¹¹ is C₈₋₁₀alkynyl. In another embodiment is a compound of Formula (II), wherein R¹¹ is C₃₋₂₂haloalkyl. In another embodiment is a compound of Formula (II), wherein R¹¹ is C₃₋₁₈haloalkyl. In another embodiment is a compound of Formula (II), wherein R¹¹ is C₃₋₁₂haloalkyl. In another embodiment is a compound of Formula (II), wherein R¹¹ is C₆₋₁₂haloalkyl. In another embodiment is a compound of Formula (II), wherein R¹¹ is C₆₋₁₀haloalkyl. In another embodiment is a compound of Formula (II), wherein R¹¹ is C₈₋₁₀haloalkyl. In another embodiment is a compound of Formula (II), wherein R¹¹ is —C₁₋₄alkyl-OC(O)C₁₋₈alkyl. In another embodiment is a compound of Formula (II), wherein R¹¹ is —C₁₋₂alkyl-OC(O)C₁₋₈alkyl. In another embodiment is a compound of Formula (II), wherein R¹¹ is —CH₂OC(O)C₁₋₈alkyl. In another embodiment is a compound of Formula (II), wherein R¹¹ is —C₁₋₄alkyl-OC(O)C₁₋₆alkyl. In another embodiment is a compound of Formula (II), wherein R¹¹ is —C₁₋₂alkyl-OC(O)C₁₋₆alkyl. In another embodiment is a compound of Formula (II), wherein R¹¹ is —CH₂OC(O)C₁₋₆alkyl. In another embodiment is a compound of Formula (II), wherein R¹¹ is —C₁₋₄alkyl-OC(O)C₁₋₄alkyl. In another embodiment is a compound of Formula (II), wherein R¹¹ is —C₁₋₂alkyl-OC(O)C₁₋₄alkyl. In another embodiment is a compound of Formula (II), wherein R¹¹ is —CH₂OC(O)C₁₋₄alkyl. In another embodiment is a compound of Formula (II), wherein R¹¹ is —C₁₋₄alkyl-OC(O)C(CH₃)₃. In another embodiment is a compound of Formula (II), wherein R¹¹ is —C₁₋₂alkyl-OC(O)C(CH₃)₃. In another embodiment is a compound of Formula (II), wherein R¹¹ is —CH₂OC(O)C(CH₃)₃. In another embodiment is a compound of Formula (II), wherein R¹¹ is C₆₋₁₀aryl optionally substituted with 1, 2, 3, or 4 R¹². In another embodiment is a compound of Formula (II), wherein R¹¹ is unsubstituted C₆₋₁₀aryl. In another embodiment is a compound of Formula (II), wherein R¹¹ is C₆₋₁₀aryl substituted with 1, 2, 3, or 4 R¹². In another embodiment is a compound of Formula (II), wherein R¹¹ is C₆₋₁₀aryl substituted with 1, 2, or 3 R¹². In another embodiment is a compound of Formula (II), wherein R¹¹ is C₆₋₁₀aryl substituted with 1 or 2 R¹². In another embodiment is a compound of Formula (II), wherein R¹¹ is C₆₋₁₀aryl substituted with 1 or 2 R¹² and each R¹² is independently selected from halogen, C₁₋₈alkyl, C₁₋₈haloalkyl, and C₁₋₈alkoxy. In another embodiment is a compound of Formula (II), wherein R¹¹ is C₆₋₁₀aryl substituted with 1 or 2 R¹² and each R¹² is independently selected from C₁₋₈alkyl, C₁₋₈alkoxy, and —C(O)R¹³. In another embodiment is a compound of Formula (II), wherein R¹¹ is C₆₋₁₀aryl substituted with 1 or 2 R¹² and each R¹² is independently selected from C₁₋₈alkyl, C₁₋₈haloalkyl, and C₁₋₈alkoxy. In another embodiment is a compound of Formula (II), wherein R¹¹ is C₆₋₁₀aryl substituted with 1 or 2 R¹² and each R¹² is independently selected from C₁₋₈alkyl and C₁₋₈alkoxy. In another embodiment is a compound of Formula (II), wherein R¹¹ is C₆₋₁₀aryl substituted with 1 R¹². In another embodiment is a compound of Formula (II), wherein R¹¹ is C₆₋₁₀aryl substituted with 1 R¹² and R¹² is selected from halogen, C₁₋₈alkyl, C₁₋₈haloalkyl, and C₁₋₈alkoxy. In another embodiment is a compound of Formula (II), wherein R¹¹ is C₆₋₁₀aryl substituted with 1 R¹² and R¹² is selected from C₁₋₈alkyl, C₁₋₈alkoxy, and —C(O)R¹³. In another embodiment is a compound of Formula (II), wherein R¹¹ is C₆₋₁₀aryl substituted with 1 R¹² and R¹² is selected from C₁₋₈alkyl and C₁₋₈alkoxy. In another embodiment is a compound of Formula (II), wherein R¹¹ is C₆₋₁₀aryl substituted with 1 R¹² and R¹² is halogen. In another embodiment is a compound of Formula (II), wherein R¹¹ is C₆₋₁₀aryl substituted with 1 R¹² and R¹² is —F. In another embodiment is a compound of Formula (II), wherein R¹¹ is C₆₋₁₀aryl substituted with 1 R¹² and R¹² is —Cl. In another embodiment is a compound of Formula (II), wherein R¹¹ is C₆₋₁₀aryl substituted with 1 R¹² and R¹² is C₁₋₈alkyl. In another embodiment is a compound of Formula (II), wherein is C₆₋₁₀aryl substituted with 1 R¹² and R¹² is —CH₃. In another embodiment is a compound of Formula (II), wherein R¹¹ is C₆₋₁₀aryl substituted with 1 R¹² and R¹² is C₁₋₈haloalkyl. In another embodiment is a compound of Formula (II), wherein R¹¹ is C₆₋₁₀aryl substituted with 1 R¹² and R¹² is —CF₃. In another embodiment is a compound of Formula (II), wherein R¹¹ is C₆₋₁₀aryl substituted with 1 R¹² and R¹² is C₁₋₈alkoxy. In another embodiment is a compound of Formula (II), wherein R¹¹ is C₆₋₁₀aryl substituted with 1 R¹² and R¹² is —OCH₃. In another embodiment is a compound of Formula (II), wherein R¹¹ is C₆₋₁₀aryl substituted with 1 R¹² and R¹² is —C(O)R¹³. In another embodiment is a compound of Formula (II), wherein R¹¹ is phenyl optionally substituted with 1, 2, 3, or 4 R¹². In another embodiment is a compound of Formula (II), wherein R¹¹ is unsubstituted phenyl. In another embodiment is a compound of Formula (II), wherein R¹¹ is phenyl substituted with 1, 2, 3, or 4 R¹². In another embodiment is a compound of Formula (II), wherein R¹¹ is phenyl substituted with 1, 2, or 3 R¹². In another embodiment is a compound of Formula (II), wherein R¹¹ is phenyl substituted with 1 or 2 R¹². In another embodiment is a compound of Formula (II), wherein R¹¹ is phenyl substituted with 1 or 2 R¹² and each R¹² is independently selected from halogen, C₁₋₈alkyl, C₁₋₈haloalkyl, and C₁₋₈alkoxy. In another embodiment is a compound of Formula (II), wherein R¹¹ is phenyl substituted with 1 or 2 R¹² and each R¹² is independently selected from C₁₋₈alkoxy, and —C(O)R¹³. In another embodiment is a compound of Formula (II), wherein R¹¹ is phenyl substituted with 1 or 2 R¹² and each R¹² is independently selected from C₁₋₈alkyl, C₁₋₈haloalkyl, and C₁₋₈alkoxy. In another embodiment is a compound of Formula (II), wherein R¹¹ is phenyl substituted with 1 or 2 R¹² and each R¹² is independently selected from C₁₋₈alkyl and C₁₋₈alkoxy. In another embodiment is a compound of Formula (II), wherein R¹¹ is phenyl substituted with 1 R¹². In another embodiment is a compound of Formula (II), wherein is phenyl substituted with 1 R¹² and R¹² is selected from halogen, C₁₋₈alkyl, C₁₋₈haloalkyl, and C₁₋₈alkoxy. In another embodiment is a compound of Formula (II), wherein R¹¹ is phenyl substituted with 1 R¹² and R¹² is selected from C₁₋₈alkyl, C₁₋₈alkoxy, and —C(O)R¹³. In another embodiment is a compound of Formula (II), wherein R¹¹ is phenyl substituted with 1 R¹² and R¹² is selected from C₁₋₈alkyl and C₁₋₈alkoxy. In another embodiment is a compound of Formula (II), wherein R¹¹ is phenyl substituted with 1 R¹² and R¹² is halogen. In another embodiment is a compound of Formula (II), wherein R¹¹ is phenyl substituted with 1 R¹² and R¹² is —F. In another embodiment is a compound of Formula (II), wherein R¹¹ is phenyl substituted with 1 R¹² and R¹² is —Cl. In another embodiment is a compound of Formula (II), wherein R¹¹ is phenyl substituted with 1 R¹² and R¹² is C₁₋₈alkyl. In another embodiment is a compound of Formula (II), wherein R¹¹ is phenyl substituted with 1 R¹² and R¹² is —CH₃. In another embodiment is a compound of Formula (II), wherein R¹¹ is phenyl substituted with 1 R¹² and R¹² is C₁₋₈haloalkyl. In another embodiment is a compound of Formula (II), wherein R¹¹ is phenyl substituted with 1 R¹² and R¹² is —CF₃. In another embodiment is a compound of Formula (II), wherein R¹¹ is phenyl substituted with 1 R¹² and R¹² is C₁₋₈alkoxy. In another embodiment is a compound of Formula (II), wherein R¹¹ is phenyl substituted with 1 R¹² and R¹² is —OCH₃. In another embodiment is a compound of Formula (II), wherein R¹¹ is phenyl substituted with 1 R¹² and R′ is —C(O)R¹³. In another embodiment is a compound of Formula (II), wherein R¹¹ is —C₁₋₈alkyl-C₆₋₁₀aryl optionally substituted with 1, 2, 3, or 4 R¹². In another embodiment is a compound of Formula (II), wherein R¹¹ is unsubstituted —C₁₋₈alkyl-C₆₋₁₀aryl. In another embodiment is a compound of Formula (II), wherein R¹¹ is —C₁₋₈alkyl-C₆₋₁₀aryl substituted with 1, 2, 3, or 4 R¹. In another embodiment is a compound of Formula (II), wherein R¹¹ is —C₁₋₈alkyl-C₆₋₁₀aryl substituted with 1, 2, or 3 R¹². In another embodiment is a compound of Formula (II), wherein R¹¹ is —C₁₋₈alkyl-C₆₋₁₀aryl substituted with 1 or 2 R¹². In another embodiment is a compound of Formula (II), wherein R¹¹ is —C₁₋₈alkyl-C₆₋₁₀aryl substituted with 1 or 2 R¹² and each R¹² is independently selected from halogen, C₁₋₈alkyl, C₁₋₈haloalkyl, and C₁₋₈ alkoxy. In another embodiment is a compound of Formula (II), wherein R¹¹ is —C₁₋₈alkyl-C₆₋₁₀aryl substituted with 1 or 2 R¹² and each R¹² is independently selected from C₁₋₈alkyl, C₁₋₈alkoxy, and —C(O)R¹³. In another embodiment is a compound of Formula (II), wherein R¹¹ is —C₁₋₈alkyl-C₆₋₁₀aryl substituted with 1 or 2 R¹² and each R¹² is independently selected from C₁₋₈alkyl, C₁₋₈haloalkyl, and C₁₋₈alkoxy. In another embodiment is a compound of Formula (II), wherein R¹¹ is —C₁₋₈alkyl-C₆₋₁₀aryl substituted with 1 or 2 R¹² and each R¹² is independently selected from C₁₋₈alkyl and C₁₋₈alkoxy. In another embodiment is a compound of Formula (II), wherein R¹¹ is —C₁₋₈alkyl-C₆₋₁₀aryl substituted with 1 R¹². In another embodiment is a compound of Formula (II), wherein R¹¹ is —C₁₋₈alkyl-C₆₋₁₀aryl substituted with 1 R¹² and R¹² is selected from halogen, C₁₋₈alkyl, C₁₋₈haloalkyl, and C₁₋₈alkoxy. In another embodiment is a compound of Formula (II), wherein R¹¹ is —C₁₋₈alkyl-C₆₋₁₀aryl substituted with 1 R¹² and R¹² is selected from C₁₋₈alkyl, C₁₋₈alkoxy, and —C(O)R¹³. In another embodiment is a compound of Formula (II), wherein R¹¹ is —C₁₋₈alkyl-C₆₋₁₀aryl substituted with 1 R¹² and R¹² is selected from C₁₋₈alkyl and C₁₋₈alkoxy. In another embodiment is a compound of Formula (II), wherein R¹¹ is —C₁₋₈alkyl-C₆₋₁₀aryl substituted with 1 R¹² and R¹² is halogen. In another embodiment is a compound of Formula (II), wherein R¹¹ is —C₁₋₈alkyl-C₆₋₁₀aryl substituted with 1 R¹² and R¹² is —F. In another embodiment is a compound of Formula (II), wherein R¹¹ is —C₁₋₈alkyl-C₆₋₁₀aryl substituted with 1 R¹² and R¹² is —Cl. In another embodiment is a compound of Formula (II), wherein R¹¹ is —C₁₋₈alkyl-C₆₋₁₀aryl substituted with 1 R¹² and R¹² is C₁₋₈alkyl. In another embodiment is a compound of Formula (II), wherein R¹¹ is —C₁₋₈alkyl-C₆₋₁₀aryl substituted with 1 R¹² and R¹² is —CH₃. In another embodiment is a compound of Formula (II), wherein R¹¹ is —C₁₋₈alkyl-C₆₋₁₀aryl substituted with 1 R¹² and R¹² is C₁₋₈haloalkyl. In another embodiment is a compound of Formula (II), wherein R¹¹ is —C₁₋₈alkyl-C₆₋₁₀aryl substituted with 1 R¹² and R¹² is —CF₃. In another embodiment is a compound of Formula (II), wherein R¹¹ is —C₁₋₈alkyl-C₆₋₁₀aryl substituted with 1 R¹² and R¹² is C₁₋₈alkoxy. In another embodiment is a compound of Formula (II), wherein R¹¹ is —C₁₋₈alkyl-C₆₋₁₀aryl substituted with 1 R¹² and R¹² is —OCH₃. In another embodiment is a compound of Formula (II), wherein R¹¹ is —C₁₋₈alkyl-C₆₋₁₀aryl substituted with 1 R¹² and R¹² is —C(O)R¹³. In another embodiment is a compound of Formula (II), wherein R¹¹ is —CH₂-phenyl optionally substituted with 1, 2, 3, or 4 R¹². In another embodiment is a compound of Formula (II), wherein R¹¹ is unsubstituted —CH₂-phenyl. In another embodiment is a compound of Formula (II), wherein R¹¹ is —CH₂-phenyl substituted with 1, 2, 3, or 4 R¹². In another embodiment is a compound of Formula (II), wherein R¹¹ is —CH₂-phenyl substituted with 1, 2, or 3 R¹². In another embodiment is a compound of Formula (II), wherein R¹¹ is —CH₂-phenyl substituted with 1 or 2 R¹². In another embodiment is a compound of Formula (II), wherein R¹¹ is —CH₂-phenyl substituted with 1 or 2 R¹² and each R¹² is independently selected from halogen, C₁₋₈alkyl, C₁₋₈haloalkyl, and C₁₋₈alkoxy. In another embodiment is a compound of Formula (II), wherein R¹¹ is —CH₂-phenyl substituted with 1 or 2 R¹² and each R¹² is independently selected from C₁₋₈alkyl, C₁₋₈alkoxy, and —C(O)R¹³. In another embodiment is a compound of Formula (II), wherein R¹¹ is —CH₂-phenyl substituted with 1 or 2 R¹² and each R¹² is independently selected from C₁₋₈alkyl, C₁₋₈haloalkyl, and C₁₋₈alkoxy. In another embodiment is a compound of Formula (II), wherein R¹¹ is —CH₂-phenyl substituted with 1 or 2 R¹² and each R¹² is independently selected from C₁₋₈alkyl and C₁₋₈alkoxy. In another embodiment is a compound of Formula (II), wherein R¹¹ is —CH₂-phenyl substituted with 1 R¹². In another embodiment is a compound of Formula (II), wherein R¹¹ is —CH₂-phenyl substituted with 1 R¹² and R¹² is selected from halogen, C₁₋₈alkyl, C₁₋₈haloalkyl, and C₁₋₈alkoxy. In another embodiment is a compound of Formula (II), wherein R¹¹ is —CH₂-phenyl substituted with 1 R¹² and R¹² is selected from C₁₋₈alkyl, C₁₋₈alkoxy, and —C(O)R¹³. In another embodiment is a compound of Formula (II), wherein R¹¹ is —CH₂-phenyl substituted with 1 R¹² and R¹² is selected from C₁₋₈alkyl and C₁₋₈alkoxy. In another embodiment is a compound of Formula (II), wherein R¹¹ is —CH₂-phenyl substituted with 1 R¹² and R¹² is halogen. In another embodiment is a compound of Formula (II), wherein R¹¹ is —CH₂-phenyl with 1 R¹² and R¹² is —F. In another embodiment is a compound of Formula (II), wherein R¹¹ is —CH₂-phenyl substituted with 1 R¹² and R¹² is —Cl. In another embodiment is a compound of Formula (II), wherein R¹¹ is —CH₂-phenyl substituted with 1 R¹² and R¹² is C₁₋₈alkyl. In another embodiment is a compound of Formula (II), wherein R¹¹ is —CH₂-phenyl substituted with 1 R¹² and R¹² is —CH₃. In another embodiment is a compound of Formula (II), wherein R¹¹ is —CH₂-phenyl substituted with 1 R¹² and R¹² is C₁₋₈haloalkyl. In another embodiment is a compound of Formula (II), wherein R¹¹ is —CH₂-phenyl substituted with 1 R¹² and R¹² is —CF₃. In another embodiment is a compound of Formula (II), wherein R¹¹ is —CH₂-phenyl substituted with 1 R¹² and R¹² is C₁₋₈alkoxy. In another embodiment is a compound of Formula (II), wherein R¹¹ is —CH₂-phenyl substituted with 1 R¹² and R¹² is —OCH₃. In another embodiment is a compound of Formula (II), wherein R¹¹ is —CH₂-phenyl substituted with 1 R¹² and R¹² is —C(O)R¹³. In another embodiment is a compound of Formula (II), wherein R¹¹ is C₂₋₉heteroaryl optionally substituted with 1, 2, 3, or 4 R¹². In another embodiment is a compound of Formula (II), wherein R¹¹ is unsubstituted C₂₋₉heteroaryl. In another embodiment is a compound of Formula (II), wherein R¹¹ is C₂₋₉heteroaryl substituted with 1, 2, 3, or 4 R¹². In another embodiment is a compound of Formula (II), wherein R¹¹ is C₂₋₉heteroaryl substituted with 1, 2, or 3 R¹². In another embodiment is a compound of Formula (II), wherein R¹¹ is C₂₋₉heteroaryl substituted with 1 or 2 R¹². In another embodiment is a compound of Formula (II), wherein R¹¹ is C₂₋₉heteroaryl substituted with 1 or 2 R¹² and each R¹² is independently selected from halogen, C₁₋₈alkyl, C₁₋₈haloalkyl, and C₁₋₈alkoxy. In another embodiment is a compound of Formula (II), wherein R¹¹ is C₂₋₉heteroaryl substituted with 1 or 2 R¹² and each R¹² is independently selected from C₁₋₈alkyl, C₁₋₈alkoxy, and —C(O)R¹³. In another embodiment is a compound of Formula (II), wherein R¹¹ is C₂₋₉heteroaryl substituted with 1 or 2 R¹² and each R¹² is independently selected from C₁₋₈alkyl, C₁₋₈haloalkyl, and C₁₋₈alkoxy. In another embodiment is a compound of Formula (II), wherein R¹¹ is C₂₋₉heteroaryl substituted with 1 or 2 R¹² and each R¹² is independently selected from C₁₋₈alkyl and C₁₋₈alkoxy. In another embodiment is a compound of Formula (II), wherein R¹¹ is C₂₋₉heteroaryl substituted with 1 R¹². In another embodiment is a compound of Formula (II), wherein R¹¹ is C₂₋₉heteroaryl substituted with 1 R¹² and R¹² is selected from halogen, C₁₋₈alkyl, C₁₋₈haloalkyl, and C₁₋₈alkoxy. In another embodiment is a compound of Formula (II), wherein R¹¹ is C₂₋₉heteroaryl substituted with 1 R¹² and R¹² is selected from C₁₋₈alkyl, C₁₋₈alkoxy, and —C(O)R¹³. In another embodiment is a compound of Formula (II), wherein R¹¹ is C₂₋₉heteroaryl substituted with 1 R¹² and R¹² is selected from C₁₋₈alkyl and C₁₋₈alkoxy. In another embodiment is a compound of Formula (II), wherein R¹¹ is C₂₋₉heteroaryl substituted with 1 R¹² and R¹² is halogen. In another embodiment is a compound of Formula (II), wherein R¹¹ is C₂₋₉heteroaryl substituted with 1 R¹² and R¹² is —F. In another embodiment is a compound of Formula (II), wherein R¹¹ is C₂₋₉heteroaryl substituted with 1 R¹² and R¹² is —Cl. In another embodiment is a compound of Formula (II), wherein R¹¹ is C₂₋₉heteroaryl substituted with 1 R¹² and R¹² is C₁₋₈alkyl. In another embodiment is a compound of Formula (II), wherein R¹¹ is C₂₋₉heteroaryl substituted with 1 R¹² and R¹² is —CH₃. In another embodiment is a compound of Formula (II), wherein R¹¹ is C₂₋₉heteroaryl substituted with 1 R¹² and K is C₁₋₈haloalkyl. In another embodiment is a compound of Formula (II), wherein R¹¹ is C₂₋₉ heteroaryl substituted with 1 R¹² and R¹² is —CF₃. In another embodiment is a compound of Formula (II), wherein R¹¹ is C₂₋₉heteroaryl substituted with 1 R¹² and R¹² is C₁₋₈alkoxy. In another embodiment is a compound of Formula (II), wherein R¹¹ is C₂₋₉heteroaryl substituted with 1 R¹² and R¹² is —OCH₃. In another embodiment is a compound of Formula (II), wherein R¹¹ is C₂₋₉heteroaryl substituted with 1 R¹² and R¹² is —C(O)R¹³. In another embodiment is a compound of Formula (II), wherein R¹¹ is —C₁₋₈alkyl-C₂₋₉heteroaryl optionally substituted with 1, 2, 3, or 4 R¹². In another embodiment is a compound of Formula (II), wherein R¹¹ is unsubstituted —C₁₋₈alkyl-C₂₋₉heteroaryl. In another embodiment is a compound of Formula (II), wherein R¹¹ is —C₁₋₈alkyl-C₂₋₉heteroaryl substituted with 1, 2, 3, or 4 R¹². In another embodiment is a compound of Formula (II), wherein R¹¹ is —C₁₋₈alkyl-C₂₋₉heteroaryl substituted with 1, 2, or 3 R¹². In another embodiment is a compound of Formula (II), wherein R¹¹ is —C₁₋₈alkyl-C₂₋₉heteroaryl substituted with 1 or 2 R¹². In another embodiment is a compound of Formula (II), wherein R¹¹ is —C₁₋₈alkyl-C₂₋₉heteroaryl substituted with 1 or 2 R¹² and each R¹² is independently selected from halogen, C₁₋₈alkyl, C₁₋₈haloalkyl, and C₁₋₈alkoxy. In another embodiment is a compound of Formula (II), wherein R¹¹ is —C₁₋₈alkyl-C₂₋₉heteroaryl substituted with 1 or 2 R¹² and each R¹² is independently selected from C₁₋₈alkyl, C₁₋₈alkoxy, and —C(O)R¹³. In another embodiment is a compound of Formula (II), wherein R¹¹ is —C₁₋₈alkyl-C₂₋₉heteroaryl substituted with 1 or 2 R¹² and each R¹² is independently selected from C₁₋₈alkyl, C₁₋₈haloalkyl, and C₁₋₈alkoxy. In another embodiment is a compound of Formula (II), wherein R¹¹ is —C₁₋₈alkyl-C₂₋₉heteroaryl substituted with 1 or 2 R¹² and each R¹² is independently selected from C₁₋₈alkyl and C₁₋₈alkoxy. In another embodiment is a compound of Formula (II), wherein R¹¹ is —C₁₋₈alkyl-C₂₋₉heteroaryl substituted with 1 R¹². In another embodiment is a compound of Formula (II), wherein R¹¹ is —C₁₋₈alkyl-C₂₋₉heteroaryl substituted with 1 R¹² and R¹² is selected from halogen, C₁₋₈alkyl, C₁₋₈haloalkyl, and C₁₋₈alkoxy. In another embodiment is a compound of Formula (II), wherein R¹¹ is —C₁₋₈alkyl-C₂₋₉heteroaryl substituted with 1 R¹² and R¹² is selected from C₁₋₈alkyl, C₁₋₈alkoxy, and —C(O)R¹³. In another embodiment is a compound of Formula (II), wherein R¹¹ is —C₁₋₈alkyl-C₂₋₉heteroaryl substituted with 1 R¹² and R¹² is selected from C₁₋₈alkyl and C₁₋₈alkoxy. In another embodiment is a compound of Formula (II), wherein is —C₁₋₈alkyl-C₂₋₉heteroaryl substituted with 1 R¹² and R¹² is halogen. In another embodiment is a compound of Formula (II), wherein R¹¹ is —C₁₋₈alkyl-C₂₋₉heteroaryl substituted with 1 R¹² and R¹² is —F. In another embodiment is a compound of Formula wherein R¹¹ is —C₁₋₈alkyl-C₂₋₉heteroaryl substituted with 1 R¹² and R¹² is —Cl. In another embodiment is a compound of Formula (II), wherein R¹¹ is —C₁₋₈alkyl-C₂₋₉heteroaryl substituted with 1 R¹² and R¹² is C₁₋₈alkyl. In another embodiment is a compound of Formula (II), wherein R¹¹ is —C₁₋₈alkyl-C₂₋₉heteroaryl substituted with 1 R¹² and R¹² is —CH₃. In another embodiment is a compound of Formula (II), wherein R¹¹ is —C₁₋₈alkyl-C₂₋₉heteroaryl substituted with 1 R¹² and R¹² is C₁₋₈haloalkyl. In another embodiment is a compound of Formula (II), wherein R¹¹ is —C₁₋₈alkyl-C₂₋₉heteroaryl substituted with 1 R¹² and R¹² is —CF₃. In another embodiment is a compound of Formula (II), wherein R¹¹ is —C₁₋₈alkyl-C₂₋₉heteroaryl substituted with 1 R¹² and R¹² is C₁₋₈alkoxy. In another embodiment is a compound of Formula (II), wherein R¹¹ is —C₁₋₈alkyl-C₂₋₉heteroaryl substituted with 1 R¹² and R¹² is —OCH₃. In another embodiment is a compound of Formula (II), wherein is —C₁₋₈alkyl-C₂₋₉heteroaryl substituted with 1 R¹² and R¹² is —C(O)R¹³.

In another embodiment is a compound of Formula (II), wherein R³ is H, —C(O)R⁹, or —C(O)OR⁹. In another embodiment is a compound of Formula (II), wherein R³ is —C(O)R⁹. In another embodiment is a compound of Formula (II), wherein R³ is —C(O)R⁹ and R⁹ is C₁₋₁₀alkyl. In another embodiment is a compound of Formula (II), wherein R³ is —C(O)R⁹ and R⁹ is C₁₋₆alkyl. In another embodiment is a compound of Formula (II), wherein R³ is —C(O)R⁹ and R⁹ is C₁₋₄alkyl. In another embodiment is a compound of Formula (II), wherein R³ is —C(O)R⁹ and R⁹ is —CH₃. In another embodiment is a compound of Formula (II), wherein R³ is —C(O)R⁹ and R⁹ is —CH₂CH₃. In another embodiment is a compound of Formula (II), wherein R³ is —C(O)OR⁹. In another embodiment is a compound of Formula (II), wherein R³ is —C(O)OR⁹ and R⁹ is C₁₋₁₀alkyl. In another embodiment is a compound of Formula (II), wherein R³ is —C(O)OR⁹ and R⁹ is C₁₋₆alkyl. In another embodiment is a compound of Formula (II), wherein R³ is —C(O)OR⁹ and R⁹ is C₁₋₄alkyl. In another embodiment is a compound of Formula (II), wherein R³ is —C(O)OR⁹ and R⁹ is —CH₃. In another embodiment is a compound of Formula (II), wherein R³ is —C(O)OR⁹ and R⁹ is —CH₂CH₃.

Further embodiments provided herein include combinations of one or more of the particular embodiments set forth above.

In some embodiments is a compound selected from:

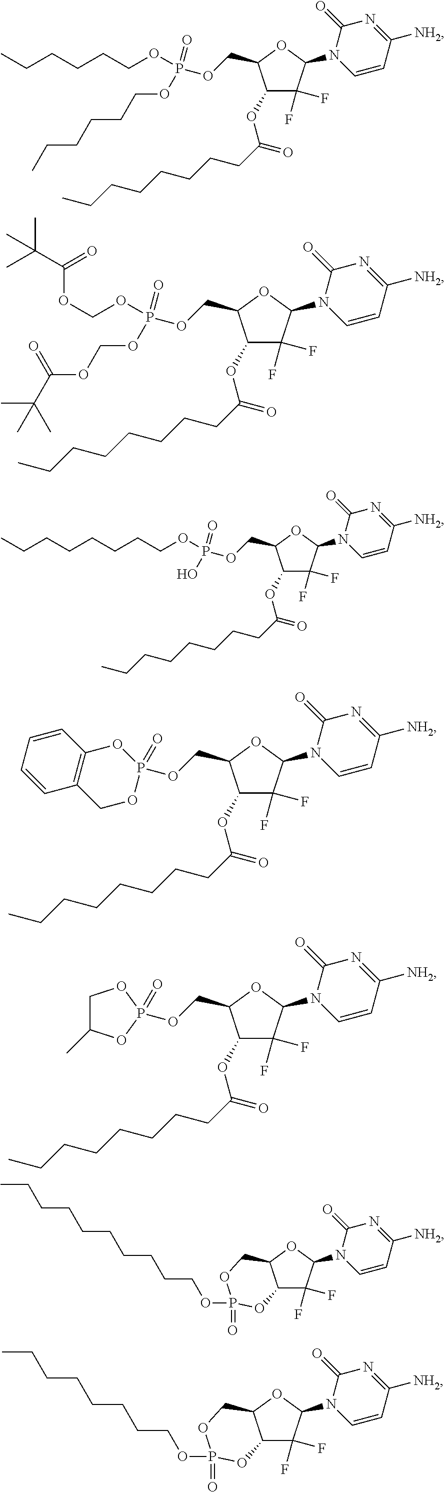

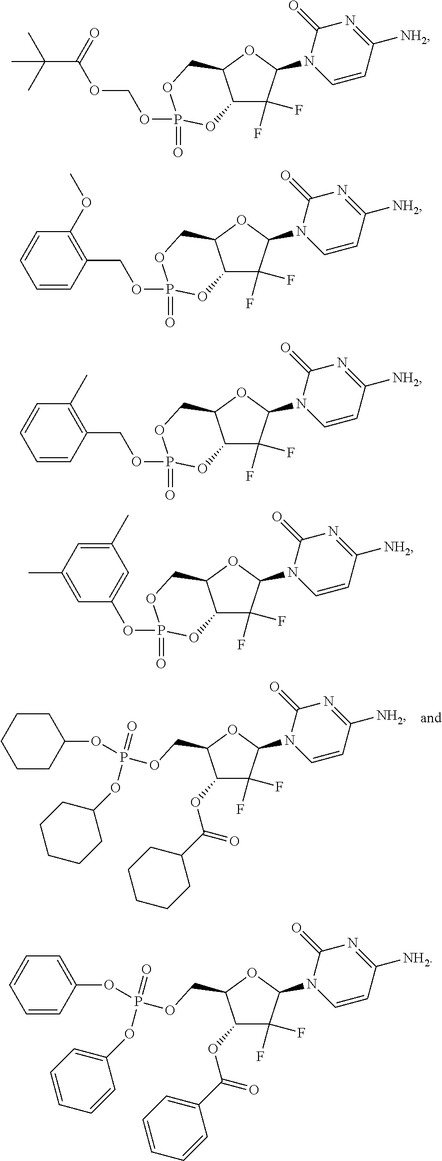

In some embodiments is a compound selected from:

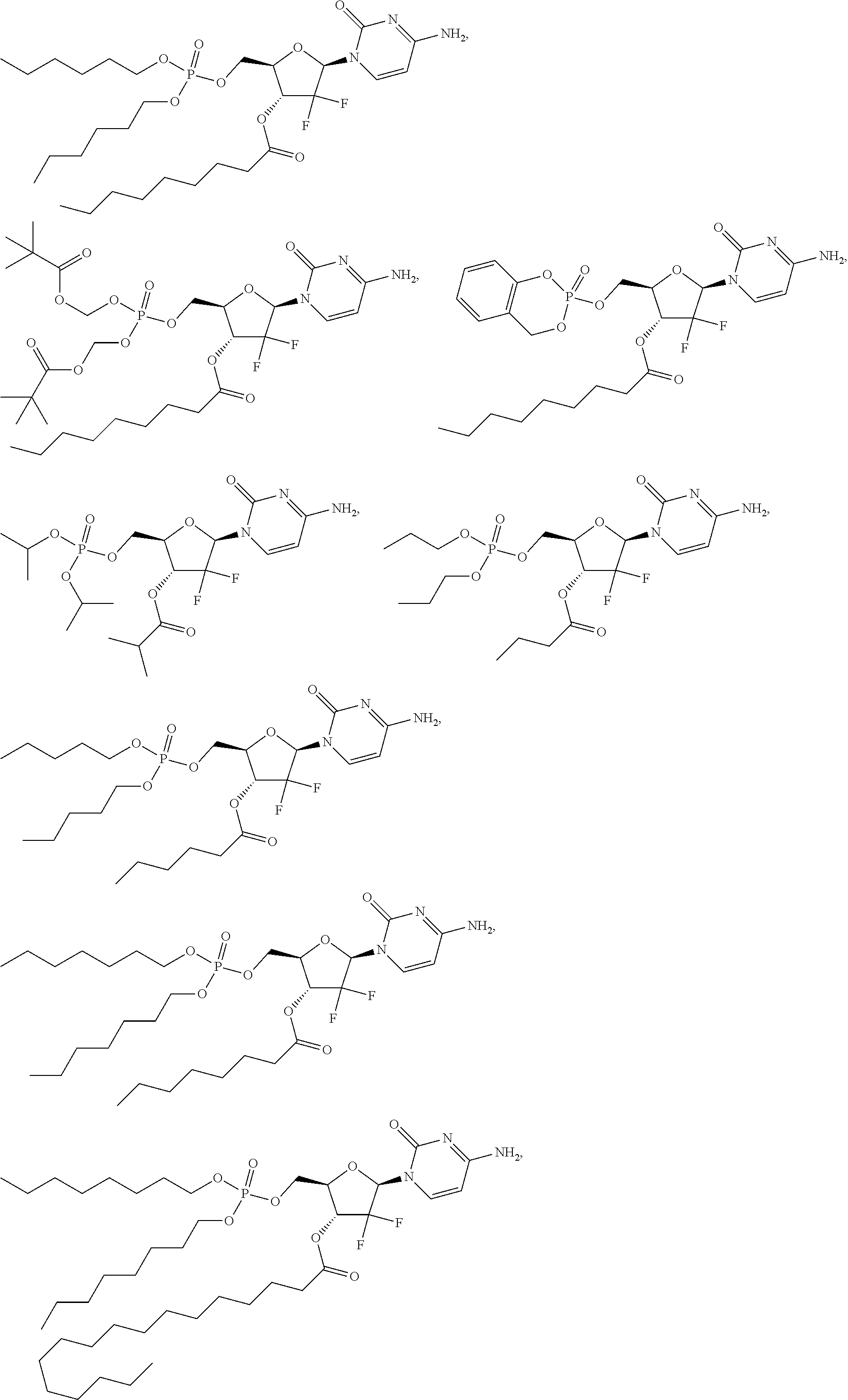

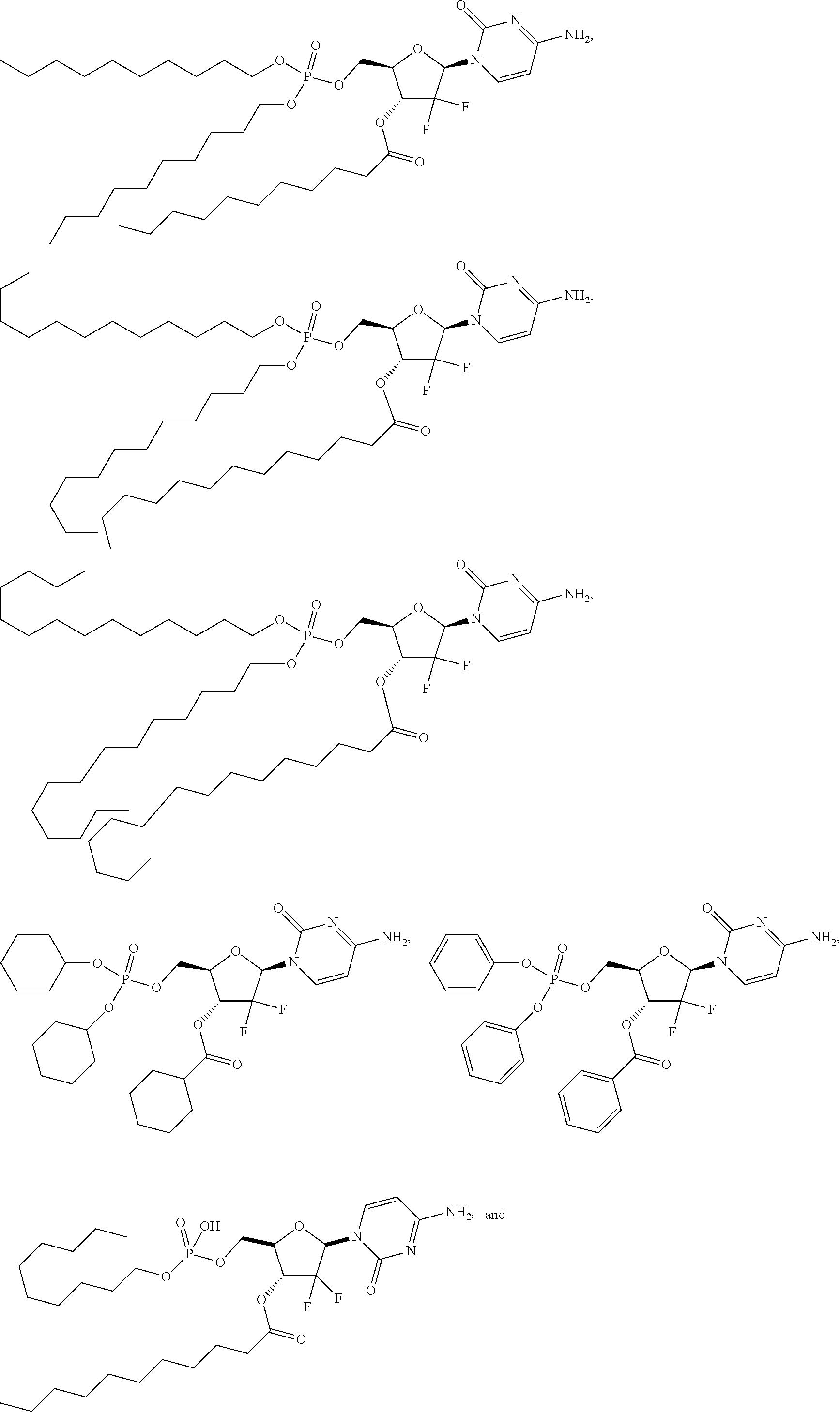

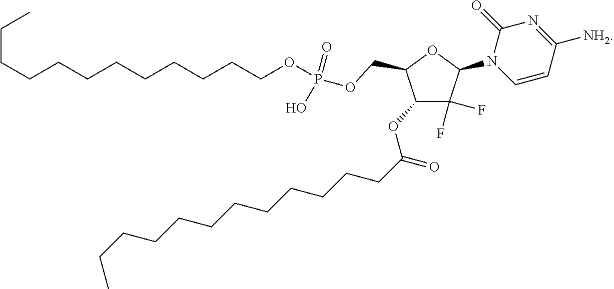 [SMILES: CCCCCCCCCCCCOP(=O)(O)OC[C@H]1O[C@@H](n2ccc(N)nc2=O)C(F)(F)[C@@H]1OC(=O)CCCCCCCCCCCC]

In some embodiments is a compound selected from:

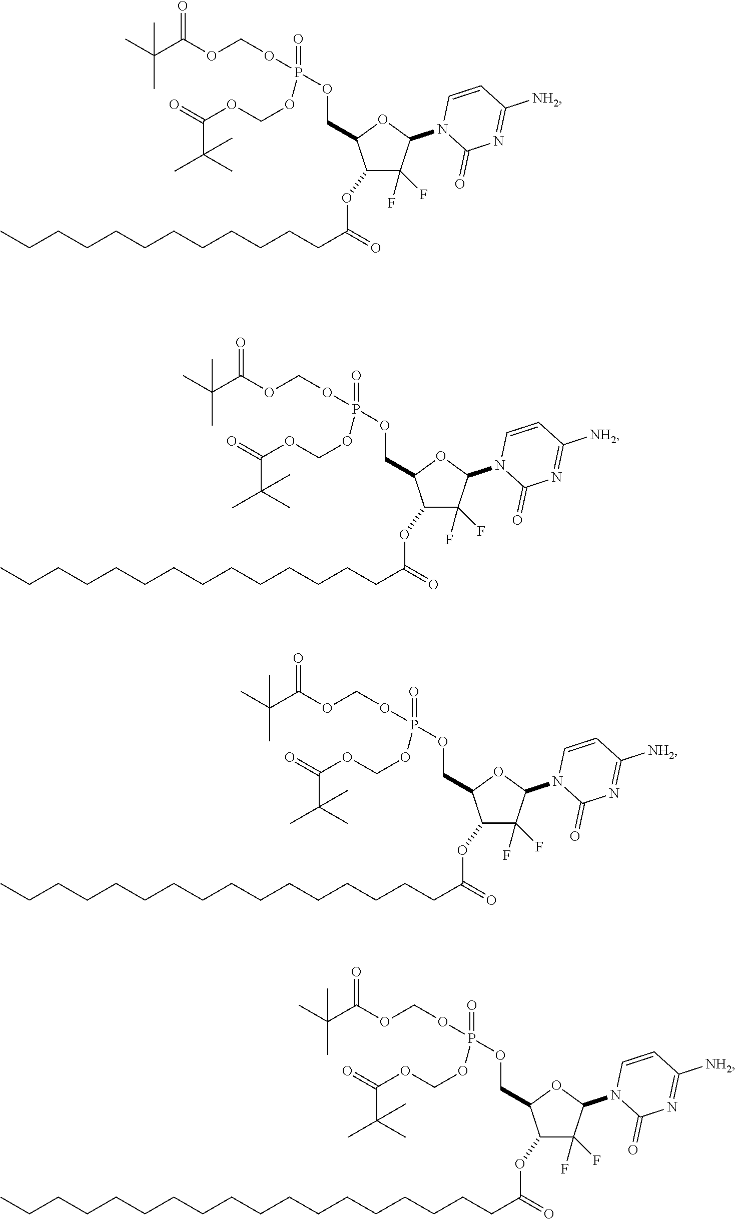 [SMILES: CCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F ; CCCCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F ; CCCCCCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F ; CCCCCCCCCCCCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F]

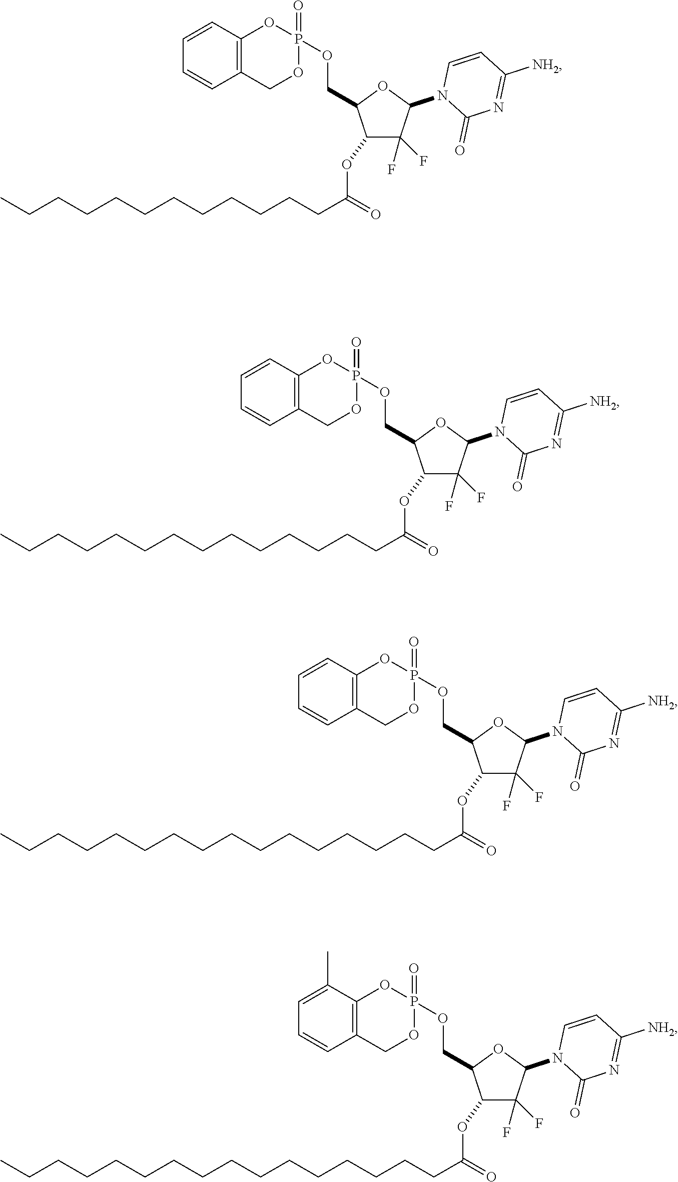 [SMILES: CCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCCCCCC(=O)O[C@@H]1[C@@H](COP2(=O)OCc3ccccc3O2)O[C@@H](n2ccc(N)nc2=O)C1(F)F ; CCCCCCCCCCCCCCCCC(=O)O[C@@H]1[C@@H](COP2(=O)OCc3cccc(C)c3O2)O[C@@H](n2ccc(N)nc2=O)C1(F)F]

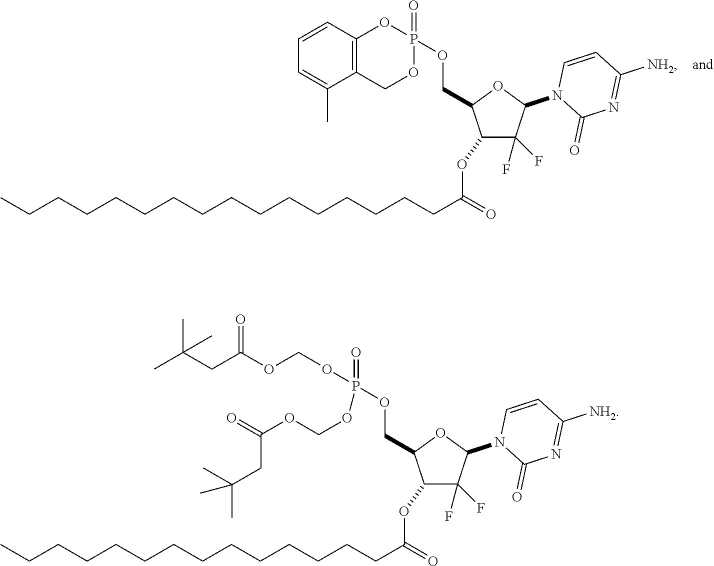 [SMILES: CCCCCCCCCCCCCCCCC(=O)O[C@@H]1[C@@H](COP2(=O)OCc3c(C)cccc3O2)O[C@@H](n2ccc(N)nc2=O)C1(F)F ; CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]

In some embodiments is any one of the compounds from the following table:

| Compound | | |
|---|---|---|
| No. | Structure | Chemical Name |
| 1 | 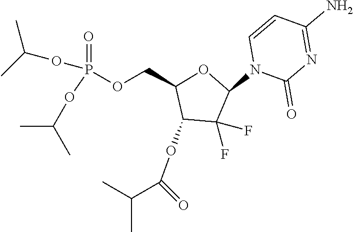 [SMILES: CC(C)OP(=O)(OC[C@H]1O[C@@H](n2ccc(N)nc2=O)C(F)(F)[C@@H]1OC(=O)C(C)C)OC(C)C] | (2R,3R,5R)-5-(4-amino- 2-oxopyrimidin-1(2H)- yl)-2-(((diisopropoxy- phosphoryl)oxy)methyl)- 4,4-difluorotetrahydro- furan-3-yl isobutyrate |
| 2 | 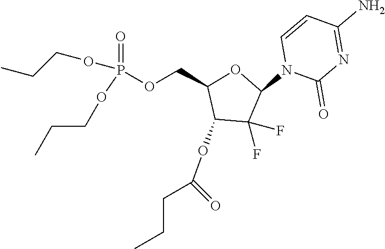 [SMILES: CCCOP(=O)(OCCC)OC[C@H]1O[C@@H](n2ccc(N)nc2=O)C(F)(F)[C@@H]1OC(=O)CCC] | (2R,3R,5R)-5-(4-amino- 2-oxopyrimidin-1(2H)- yl)-2-(((dipropoxy- phosphoryl)oxy)methyl)- 4,4-difluorotetrahydro- furan-3-yl butyrate |
| 3 | 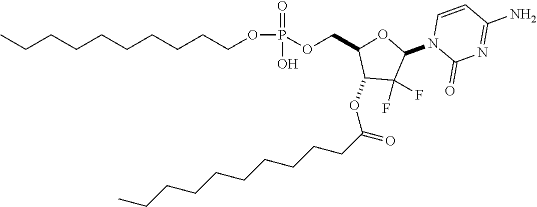 [SMILES: CCCCCCCCCCOP(=O)(O)OC[C@H]1O[C@@H](n2ccc(N)nc2=O)C(F)(F)[C@@H]1OC(=O)CCCCCCCCCC] | (2R,3R,5R)-5-(4-amino- 2-oxopyrimidin-1(2H)- yl)-2-((((decyloxy)- (hydroxy)phosphoryl)- oxy)methyl)-4,4-difluoro- tetrahydrofuran-3-yl undecanoate |
| 4 | 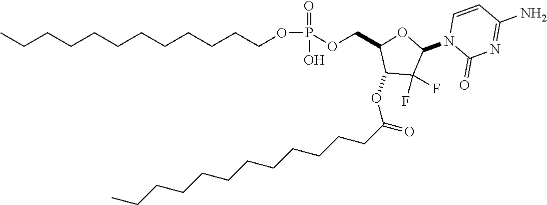 [SMILES: CCCCCCCCCCCCOP(=O)(O)OC[C@H]1O[C@@H](n2ccc(N)nc2=O)C(F)(F)[C@@H]1OC(=O)CCCCCCCCCCCC] | (2R,3R,5R)-5-(4-amino- 2-oxopyrimidin-1(2H)- yl)-2-((((dodecyloxy)- (hydroxy)phosphoryl)- oxy)methyl)-4,4-difluoro- tetrahydrofuran-3-yl tridecanoate |
| 5 | 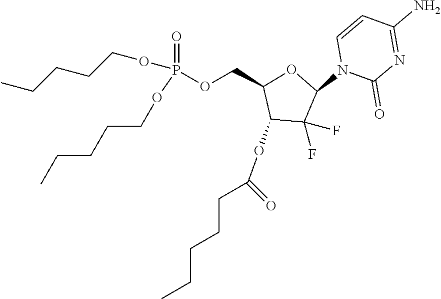 [SMILES: CCCCCOP(=O)(OCCCCC)OC[C@H]1O[C@@H](n2ccc(N)nc2=O)C(F)(F)[C@@H]1OC(=O)CCCCC] | (2R,3R,5R)-5-(4-amino- 2-oxopyrimidin-1(2H)- yl)-2-(((bis(pentyloxy)- phosphoryl)oxy)methyl)- 4,4-difluorotetrahydro- furan-3-yl hexanoate |
| 6 | 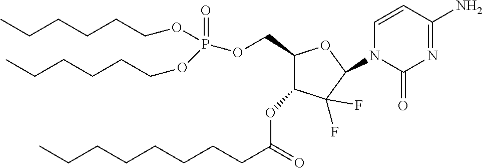 [SMILES: CCCCCCCCC(=O)O[C@@H]1[C@@H](COP(=O)(OCCCCCC)OCCCCCC)O[C@@H](n2ccc(N)nc2=O)C1(F)F] | (2R,3R,5R)-5-(4-amino- 2-oxopyrimidin-1(2H)- yl)-2-(((bis(hexyloxy)- phosphoryl)oxy)methyl)- 4,4-difluorotetrahydro- furan-3-yl nonanoate |
| 7 | 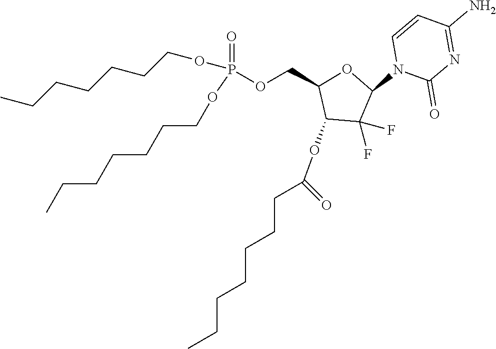 [SMILES: CCCCCCCOP(=O)(OCCCCCCC)OC[C@H]1O[C@@H](n2ccc(N)nc2=O)C(F)(F)[C@@H]1OC(=O)CCCCCCC] | (2R,3R,5R)-5-(4-amino- 2-oxopyrimidin-1(2H)- yl)-2-(((bis(heptyloxy)- phosphoryl)oxy)methyl)- 4,4-difluorotetrahydro- furan-3-yl octanoate |
| 8 | 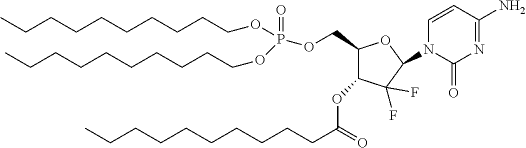 [SMILES: CCCCCCCCCCOP(=O)(OCCCCCCCCCC)OC[C@H]1O[C@@H](n2ccc(N)nc2=O)C(F)(F)[C@@H]1OC(=O)CCCCCCCCCC] | (2R,3R,5R)-5-(4-amino- 2-oxopyrimidin-1(2H)- yl)-2-(((bis(decyloxy)- phosphoryl)oxy)methyl)- 4,4-difluorotetrahydro- furan-3-yl undecanoate |
| 9 | 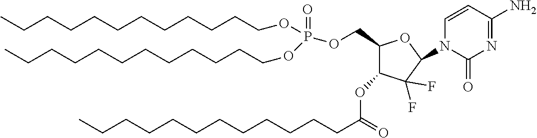 [SMILES: CCCCCCCCCCCCOP(=O)(OCCCCCCCCCCCC)OC[C@H]1O[C@@H](n2ccc(N)nc2=O)C(F)(F)[C@@H]1OC(=O)CCCCCCCCCCCC] | (2R,3R,5R)-5-(4-amino- 2-oxopyrimidin-1(2H)- yl)-2-(((bis(dodecyloxy)- phosphoryl)oxy)methyl)- 4,4-difluorotetrahydro- furan-3-yl tridecanoate |
| 10 | 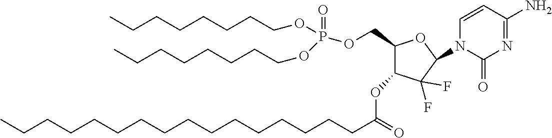 [SMILES: CCCCCCCCCCCCCCCCC(=O)O[C@@H]1[C@@H](COP(=O)(OCCCCCCCC)OCCCCCCCC)O[C@@H](n2ccc(N)nc2=O)C1(F)F] | (2R,3R,5R)-5-(4-amino- 2-oxopyrimidin-1(2H)- yl)-2-(((bis(octyloxy)- phosphoryl)oxy)methyl)- 4,4-difluorotetrahydro- furan-3-yl heptadecanoate |
| 11 | 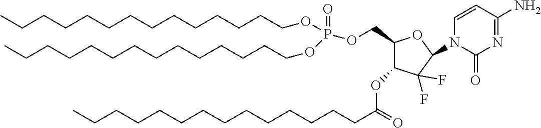 [SMILES: CCCCCCCCCCCCCCOP(=O)(OCCCCCCCCCCCCCC)OC[C@H]1O[C@@H](n2ccc(N)nc2=O)C(F)(F)[C@@H]1OC(=O)CCCCCCCCCCCCCC] | (2R,3R,5R)-5-(4-amino- 2-oxopyrimidin-1(2H)- yl)-2-(((bis(tetradecyloxy)- phosphoryl)oxy)methyl)- 4,4-difluorotetrahydro- furan-3-yl pentadecanoate |
| 12 | 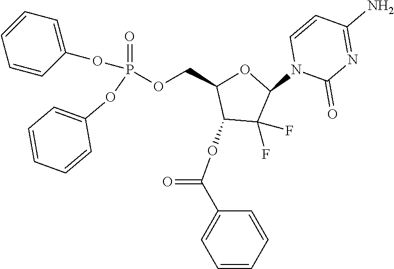 [SMILES: Nc1ccn([C@@H]2O[C@H](COP(=O)(Oc3ccccc3)Oc3ccccc3)[C@@H](OC(=O)c3ccccc3)C2(F)F)c(=O)n1] | (2R,3R,5R)-5-(4-amino- 2-oxopyrimidin-1(2H)- yl)-2-(((diphenoxyphos- phoryl)oxy)methyl)-4,4- difluorotetrahydrofuran- 3-yl benzoate |
| 13 | 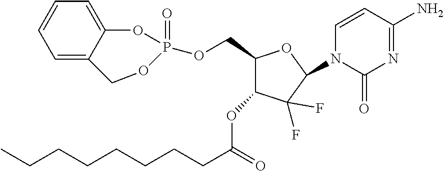 [SMILES: CCCCCCCCC(=O)O[C@@H]1[C@@H](COP2(=O)OCc3ccccc3O2)O[C@@H](n2ccc(N)nc2=O)C1(F)F] | (2R,3R,5R)-5-(4-amino- 2-oxopyrimidin-1(2H)- yl)-4,4-difluoro-2-(((2- oxido-4H-benzo[d][1,3,2]- dioxaphosphinin-2-yl)- oxy)methyl)tetrahydro- furan-3-yl nonanoate |
| 14 | 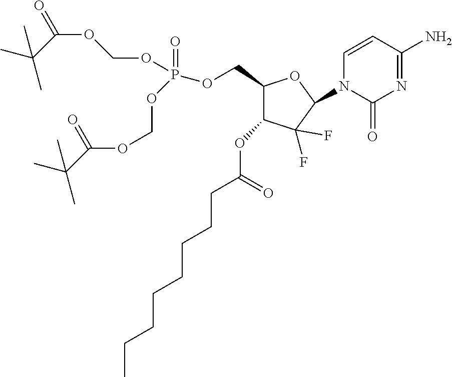 [SMILES: CCCCCCCCC(=O)O[C@@H]1[C@@H](COP(=O)(OCOC(=O)C(C)(C)C)OCOC(=O)C(C)(C)C)O[C@@H](n2ccc(N)nc2=O)C1(F)F] | [symbol] |
| 15 | 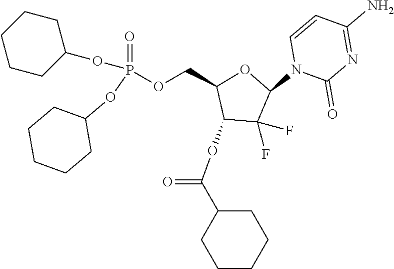 [SMILES: Nc1ccn([C@@H]2O[C@H](COP(=O)(OC3CCCCC3)OC3CCCCC3)[C@@H](OC(=O)C3CCCCC3)C2(F)F)c(=O)n1] | (2R,3R,5R)-5-(4-amino- 2-oxopyrimidin-1(2H)- yl)-2-((bis(cyclohexyl- oxy)phosphoryl)oxy)- methyl)-4,4-difluoro- tetrahydrofuran-3-yl cyclohexanecarboxylate |
| 16 | 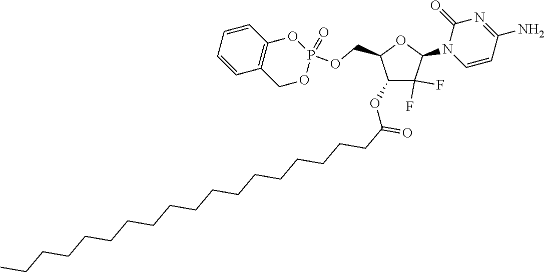 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F] | (2R,3R,5R)-5-(4-amino- 2-oxopyrimidin-1(2H)- yl)-4,4-difluoro-2-(((2- oxido-4H-benzo[d]- [1,3,2]dioxaphosphinin- 2-yl)oxy)methyl)- tetrahydrofuran-3-yl nonadecanoate |
| 17 | 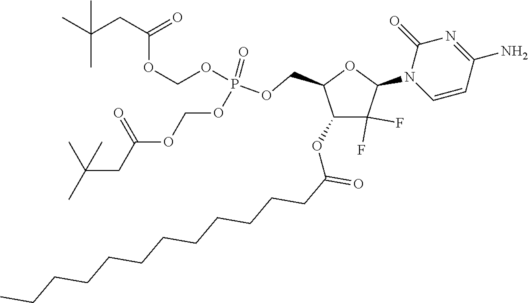 [SMILES: CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F] | [symbol] |
| 18 | 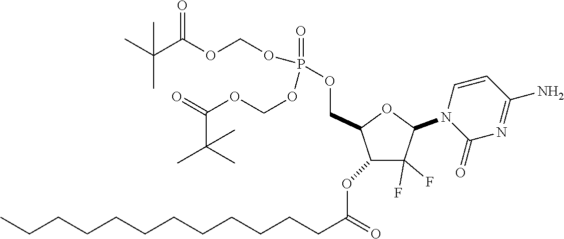 | [symbol] |
| 19 | 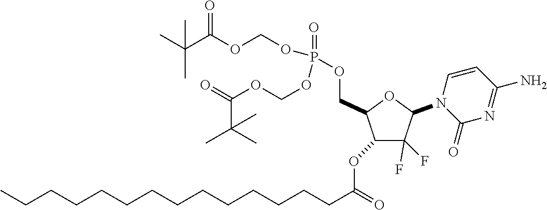 | [symbol] |
| 20 | 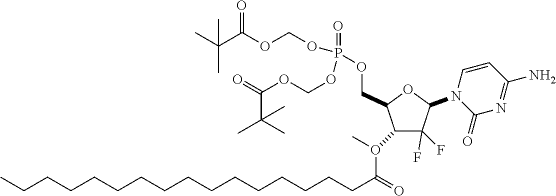 | [symbol] |
| 21 | 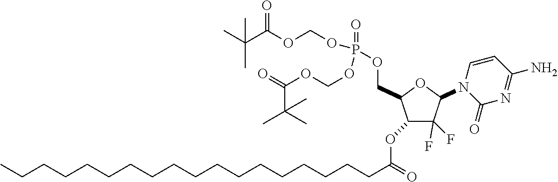 | [symbol] |
| 22 | 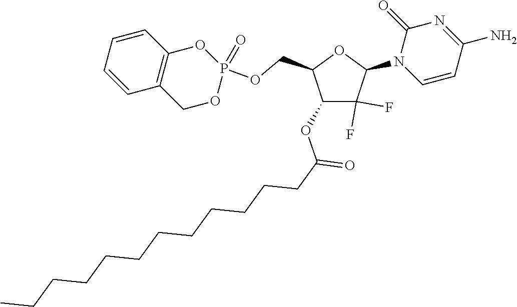 [SMILES: CCCCCCCCCCCCC(=O)O[C@@H]1[C@@H](COP2(=O)OCc3ccccc3O2)O[C@@H](n2ccc(N)nc2=O)C1(F)F] | (2R,3R,5R)-5-(4-amino- 2-oxopyrimidin-1(2H)- yl)-4,4-difluoro-2-(((2- oxido-4H-benzo[d]- [1,3,2]dioxaphosphinin- 2-yl)oxy)methyl)- tetrahydrofuran-3-yl tridecanoate |
| 23 | 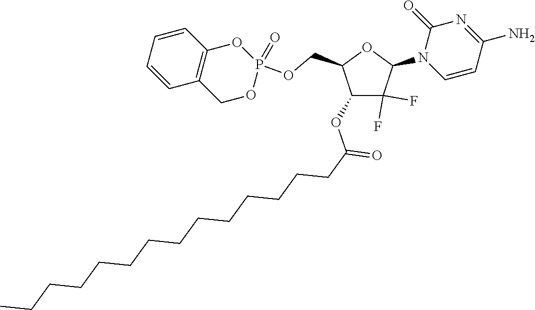 [SMILES: CCCCCCCCCCCCCCC(=O)O[C@@H]1[C@@H](COP2(=O)OCc3ccccc3O2)O[C@@H](n2ccc(N)nc2=O)C1(F)F] | (2R,3R,5R)-5-(4-amino- 2-oxopyrimidin-1(2H)- yl)-4,4-difluoro-2-(((2- oxido-4H-benzo[d]- [1,3,2]dioxaphosphinin- 2-yl)oxy)methyl)- tetrahydrofuran-3-yl pentadecanoate |
| 24 | 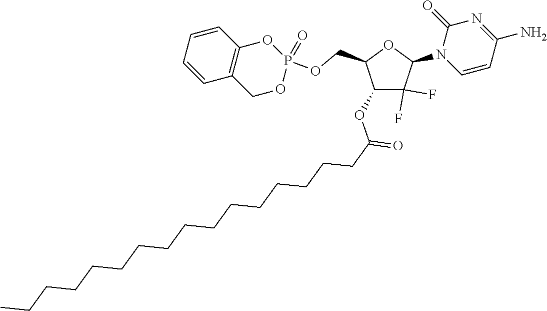 [SMILES: CCCCCCCCCCCCCCCCC(=O)O[C@@H]1[C@@H](COP2(=O)OCc3ccccc3O2)O[C@@H](n2ccc(N)nc2=O)C1(F)F] | (2R,3R,5R)-5-(4-amino- 2-oxopyrimidin-1(2H)- yl)-4,4-difluoro-2-(((2- oxido-4H-benzo[d]- [1,3,2]dioxaphosphinin- 2-yl)oxy)methyl)- tetrahydrofuran-3-yl heptadecanoate |
| 25 | 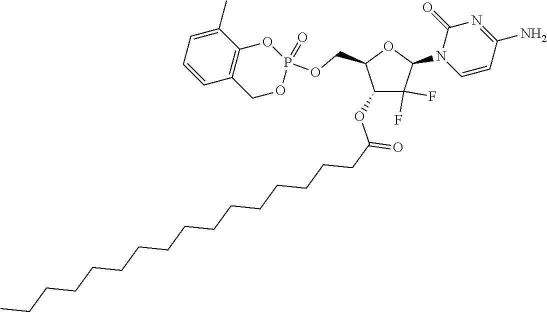 [SMILES: CCCCCCCCCCCCCCCCC(=O)O[C@@H]1[C@@H](COP2(=O)OCc3cccc(C)c3O2)O[C@@H](n2ccc(N)nc2=O)C1(F)F] | (2R,3R,5R)-5-(4-amino- 2-oxopyrimidin-1(2H)- yl)-4,4-difluoro-2-(((8- methyl-2-oxido-4H- benzo[d][1,3,2]dioxa- phosphinin-2-yl)oxy)- methyl)tetrahydrofuran- 3-yl heptadecanoate |
| 26 | 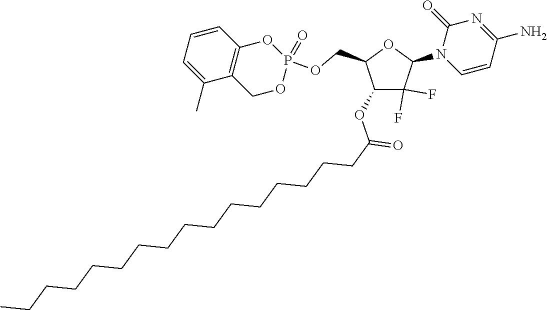 [SMILES: CCCCCCCCCCCCCCCCC(=O)O[C@@H]1[C@@H](COP2(=O)OCc3c(C)cccc3O2)O[C@@H](n2ccc(N)nc2=O)C1(F)F] | (2R,3R,5R)-5-(4-amino- 2-oxopyrimidin-1(2H)- yl)-4,4-difluoro-2-(((5- methyl-2-oxido-4H- benzo[d][1,3,2]dioxa- phosphinin-2-yl)oxy)- methyl)tetrahydrofuran- 3-yl heptadecanoate |
| 27 | 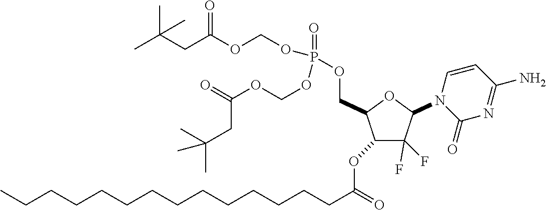 | [symbol] |

[symbol]

indicates data missing or illegible when filed

### Preparation of Compounds

The compounds used in the reactions described herein are made according to organic synthesis techniques, starting from commercially available chemicals and/or from compounds described in the chemical literature. “Commercially available chemicals” are obtained from standard commercial sources including, but not limited to, Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C.). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, compounds described herein are synthesized as described in PCT/US18/44389, which is hereby incorporated by reference in its entirety.

### Prodrugs

In some embodiments, compounds described herein are prodrugs. A “prodrug” refers to an agent that is converted into the parent drug in vivo. Prodrugs are often useful because, in some situations, they are easier to administer than the parent drug. In some embodiments, the prodrug is a substrate for a transporter. In some embodiments, the prodrug also has improved solubility in pharmaceutical compositions over the parent drug. In some embodiments, the design of a prodrug increases the effective water solubility. In some embodiments, the design of a prodrug decreases the effective water solubility. An example, without limitation, of a prodrug is a compound described herein, which is administered as an ester (the “prodrug”) but then is metabolically hydrolyzed to provide the active entity. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the parent compound is incorporated into an acyloxyalkyl ester, alkoxycarbonyloxyalkyl ester, aryl ester, phosphate ester, sugar ester, ether, and the like.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or MeOH. In addition, the compounds provided herein exist in unsolvated as well as solvated forms. In general, the solvated forms are considered equivalent to the unsolvated forms for the purposes of the compounds and methods provided herein.

### Pharmaceutically Acceptable Carrier

In some embodiments, the composition described herein also comprise a pharmaceutically acceptable carrier. In some embodiments, the pharmaceutically acceptable carrier is a protein. The term “protein’ as used herein refers to polypeptides or polymers comprising of amino acids of any length (including full length or fragments). These polypeptides or polymers are linear or branched, comprise modified amino acids, and/or are interrupted by non-amino acids. The term also encompasses an amino acid polymer that has been modified by natural means or by chemical modification. Examples of chemical modifications include, but are not limited to, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or synthesized by recombinant DNA techniques). In some embodiments, the protein is naturally occurring. In some embodiments, the protein is obtained or derived from a natural source. In some embodiments, the protein is synthetically prepared.

Examples of suitable pharmaceutically acceptable carriers include proteins normally found in blood or plasma, such as albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the pharmaceutically acceptable carrier is a non-blood protein. Examples of non-blood protein include but are not limited to casein, C.-lactalbumin, and B-lactoglobulin.

In some embodiments, the pharmaceutically acceptable carrier is albumin. In some embodiments, the albumin is human serum albumin (HSA). Human serum albumin is the most abundant protein in human blood and is a highly soluble globular protein that consists of 585 amino acids and has a molecular weight of 66.5 kDa. Other albumins suitable for use include, but are not limited to, bovine serum albumin.

In some non-limiting embodiments, the composition described herein further comprises one or more albumin stabilizers. In some embodiments, the albumin stabilizer is N-acetyl tryptophan, octanoate, cholesterol, or a combination thereof.

In some embodiments, the molar ratio of the compound to pharmaceutically acceptable carrier is from about 1:1 to about 40:1. In some embodiments, the molar ratio of the compound to pharmaceutically acceptable carrier is from about 1:1 to about 20:1. In some embodiments, the molar ratio of the compound to pharmaceutically acceptable carrier is from about 2:1 to about 12:1.

In some embodiments, the molar ratio of the compound to pharmaceutically acceptable carrier is about 40:1. In some embodiments, the molar ratio of the compound to pharmaceutically acceptable carrier is about 35:1. In some embodiments, the molar ratio of the compound to pharmaceutically acceptable carrier is about 30:1. In some embodiments, the molar ratio of the compound to pharmaceutically acceptable carrier is about 25:1. In some embodiments, the molar ratio of the compound to pharmaceutically acceptable carrier is about 20:1. In some embodiments, the molar ratio of the compound to pharmaceutically acceptable carrier is about 19:1. In some embodiments, the molar ratio of the compound to pharmaceutically acceptable carrier is about 18:1. In some embodiments, the molar ratio of the compound to pharmaceutically acceptable carrier is about 17:1. In some embodiments, the molar ratio of the compound to pharmaceutically acceptable carrier is about 16:1. In some embodiments, the molar ratio of the compound to pharmaceutically acceptable carrier is about 15:1. In some embodiments, the molar ratio of the compound to pharmaceutically acceptable carrier is about 14:1. In some embodiments, the molar ratio of the compound to pharmaceutically acceptable carrier is about 13:1. In some embodiments, the molar ratio of the compound to pharmaceutically acceptable carrier is about 12:1. In some embodiments, the molar ratio of the compound to pharmaceutically acceptable carrier is about 11:1. In some embodiments, the molar ratio of the compound to pharmaceutically acceptable carrier is about 10:1. In some embodiments, the molar ratio of the compound to pharmaceutically acceptable carrier is about 9:1. In some embodiments, the molar ratio of the compound to pharmaceutically acceptable carrier is about 8:1. In some embodiments, the molar ratio of the compound to pharmaceutically acceptable carrier is about 7:1. In some embodiments, the molar ratio of the compound to pharmaceutically acceptable carrier is about 6:1. In some embodiments, the molar ratio of the compound to pharmaceutically acceptable carrier is about 5:1. In some embodiments, the molar ratio of the compound to pharmaceutically acceptable carrier is about 4:1. In some embodiments, the molar ratio of the compound to pharmaceutically acceptable carrier is about 3:1. In some embodiments, the molar ratio of the compound to pharmaceutically acceptable carrier is about 2:1. In some embodiments, the molar ratio of the compound to pharmaceutically acceptable carrier is about 1:1.

### Nanoparticles

Described herein in one aspect is a composition comprising nanoparticles comprising any one of the compounds described herein, such as a compound of Formula (I) or Formula (II); and a pharmaceutically acceptable carrier.

In some embodiments, the nanoparticles have an average diameter of about 1000 nm or less for a predetermined amount of time after nanoparticle formation. In some embodiments, the nanoparticles have an average diameter of about 950 nm or less for a predetermined amount of time after nanoparticle formation. In some embodiments, the nanoparticles have an average diameter of about 900 nm or less for a predetermined amount of time after nanoparticle formation. In some embodiments, the nanoparticles have an average diameter of about 850 nm or less for a predetermined amount of time after nanoparticle formation. In some embodiments, the nanoparticles have an average diameter of about 800 nm or less for a predetermined amount of time after nanoparticle formation. In some embodiments, the nanoparticles have an average diameter of about 750 nm or less for a predetermined amount of time after nanoparticle formation. In some embodiments, the nanoparticles have an average diameter of about 700 nm or less for a predetermined amount of time after nanoparticle formation. In some embodiments, the nanoparticles have an average diameter of about 650 nm or less for a predetermined amount of time after nanoparticle formation. In some embodiments, the nanoparticles have an average diameter of about 600 nm or less for a predetermined amount of time after nanoparticle formation. In some embodiments, the nanoparticles have an average diameter of about 550 nm or less for a predetermined amount of time after nanoparticle formation. In some embodiments, the nanoparticles have an average diameter of about 500 nm or less for a predetermined amount of time after nanoparticle formation. In some embodiments, the nanoparticles have an average diameter of about 450 nm or less for a predetermined amount of time after nanoparticle formation. In some embodiments, the nanoparticles have an average diameter of about 400 nm or less for a predetermined amount of time after nanoparticle formation. In some embodiments, the nanoparticles have an average diameter of about 350 nm or less for a predetermined amount of time after nanoparticle formation. In some embodiments, the nanoparticles have an average diameter of about 300 nm or less for a predetermined amount of time after nanoparticle formation. In some embodiments, the nanoparticles have an average diameter of about 250 nm or less for a predetermined amount of time after nanoparticle formation. In some embodiments, the nanoparticles have an average diameter of about 240 nm or less for a predetermined amount of time after nanoparticle formation. In some embodiments, the nanoparticles have an average diameter of about 230 nm or less for a predetermined amount of time after nanoparticle formation. In some embodiments, the nanoparticles have an average diameter of about 220 nm or less for a predetermined amount of time after nanoparticle formation. In some embodiments, the nanoparticles have an average diameter of about 210 nm or less for a predetermined amount of time after nanoparticle formation. In some embodiments, the nanoparticles have an average diameter of about 200 nm or less for a predetermined amount of time after nanoparticle formation. In some embodiments, the nanoparticles have an average diameter of about 190 nm or less for a predetermined amount of time after nanoparticle formation. In some embodiments, the nanoparticles have an average diameter of about 180 nm or less for a predetermined amount of time after nanoparticle formation. In some embodiments, the nanoparticles have an average diameter of about 170 nm or less for a predetermined amount of time after nanoparticle formation. In some embodiments, the nanoparticles have an average diameter of about 160 nm or less for a predetermined amount of time after nanoparticle formation. In some embodiments, the nanoparticles have an average diameter of about 150 nm or less for a predetermined amount of time after nanoparticle formation. In some embodiments, the nanoparticles have an average diameter of about 140 nm or less for a predetermined amount of time after nanoparticle formation. In some embodiments, the nanoparticles have an average diameter of about 130 nm or less for a predetermined amount of time after nanoparticle formation. In some embodiments, the nanoparticles have an average diameter of about 120 nm or less for a predetermined amount of time after nanoparticle formation. In some embodiments, the nanoparticles have an average diameter of about 110 nm or less for a predetermined amount of time after nanoparticle formation. In some embodiments, the nanoparticles have an average diameter of about 100 nm or less for a predetermined amount of time after nanoparticle formation. In some embodiments, the nanoparticles have an average diameter of about 90 nm or less for a predetermined amount of time after nanoparticle formation. In some embodiments, the nanoparticles have an average diameter of about 80 nm or less for a predetermined amount of time after nanoparticle formation. In some embodiments, the nanoparticles have an average diameter of about 70 nm or less for a predetermined amount of time after nanoparticle formation. In some embodiments, the nanoparticles have an average diameter of about 60 nm or less for a predetermined amount of time after nanoparticle formation. In some embodiments, the nanoparticles have an average diameter of about 50 nm or less for a predetermined amount of time after nanoparticle formation. In some embodiments, the nanoparticles have an average diameter of about 40 nm or less for a predetermined amount of time after nanoparticle formation. In some embodiments, the nanoparticles have an average diameter of about 30 nm or less for a predetermined amount of time after nanoparticle formation. In some embodiments, the nanoparticles have an average diameter of about 20 nm or less for a predetermined amount of time after nanoparticle formation. In some embodiments, the nanoparticles have an average diameter of about 10 nm or less for a predetermined amount of time after nanoparticle formation.

In some embodiments, the nanoparticles have an average diameter of about 10 nm or greater for a predetermined amount of time after nanoparticle formation. In some embodiments, the nanoparticles have an average diameter of about 20 nm or greater for a predetermined amount of time after nanoparticle formation. In some embodiments, the nanoparticles have an average diameter of about 30 nm or greater for a predetermined amount of time after nanoparticle formation. In some embodiments, the nanoparticles have an average diameter of about 40 nm or greater for a predetermined amount of time after nanoparticle formation. In some embodiments, the nanoparticles have an average diameter of about 50 nm or greater for a predetermined amount of time after nanoparticle formation. In some embodiments, the nanoparticles have an average diameter of about 60 nm or greater for a predetermined amount of time after nanoparticle formation. In some embodiments, the nanoparticles have an average diameter of about 70 nm or greater for a predetermined amount of time after nanoparticle formation. In some embodiments, the nanoparticles have an average diameter of about 80 nm or greater for a predetermined amount of time after nanoparticle formation. In some embodiments, the nanoparticles have an average diameter of about 90 nm or greater for a predetermined amount of time after nanoparticle formation. In some embodiments, the nanoparticles have an average diameter of about 100 nm or greater for a predetermined amount of time after nanoparticle formation. In some embodiments, the nanoparticles have an average diameter of about 110 nm or greater for a predetermined amount of time after nanoparticle formation. In some embodiments, the nanoparticles have an average diameter of about 120 nm or greater for a predetermined amount of time after nanoparticle formation. In some embodiments, the nanoparticles have an average diameter of about 130 nm or greater for a predetermined amount of time after nanoparticle formation. In some embodiments, the nanoparticles have an average diameter of about 140 nm or greater for a predetermined amount of time after nanoparticle formation. In some embodiments, the nanoparticles have an average diameter of about 150 nm or greater for a predetermined amount of time after nanoparticle formation. In some embodiments, the nanoparticles have an average diameter of about 160 nm or greater for a predetermined amount of time after nanoparticle formation. In some embodiments, the nanoparticles have an average diameter of about 170 nm or greater for a predetermined amount of time after nanoparticle formation. In some embodiments, the nanoparticles have an average diameter of about 180 nm or greater for a predetermined amount of time after nanoparticle formation. In some embodiments, the nanoparticles have an average diameter of about 190 nm or greater for a predetermined amount of time after nanoparticle formation. In some embodiments, the nanoparticles have an average diameter of about 200 nm or greater for a predetermined amount of time after nanoparticle formation. In some embodiments, the nanoparticles have an average diameter of about 210 nm or greater for a predetermined amount of time after nanoparticle formation. In some embodiments, the nanoparticles have an average diameter of about 220 nm or greater for a predetermined amount of time after nanoparticle formation. In some embodiments, the nanoparticles have an average diameter of about 230 nm or greater for a predetermined amount of time after nanoparticle formation. In some embodiments, the nanoparticles have an average diameter of about 240 nm or greater for a predetermined amount of time after nanoparticle formation. In some embodiments, the nanoparticles have an average diameter of about 250 nm or greater for a predetermined amount of time after nanoparticle formation. In some embodiments, the nanoparticles have an average diameter of about 300 nm or greater for a predetermined amount of time after nanoparticle formation. In some embodiments, the nanoparticles have an average diameter of about 350 nm or greater for a predetermined amount of time after nanoparticle formation. In some embodiments, the nanoparticles have an average diameter of about 400 nm or greater for a predetermined amount of time after nanoparticle formation. In some embodiments, the nanoparticles have an average diameter of about 450 nm or greater for a predetermined amount of time after nanoparticle formation. In some embodiments, the nanoparticles have an average diameter of about 500 nm or greater for a predetermined amount of time after nanoparticle formation. In some embodiments, the nanoparticles have an average diameter of about 550 nm or greater for a predetermined amount of time after nanoparticle formation. In some embodiments, the nanoparticles have an average diameter of about 600 nm or greater for a predetermined amount of time after nanoparticle formation. In some embodiments, the nanoparticles have an average diameter of about 650 nm or greater for a predetermined amount of time after nanoparticle formation. In some embodiments, the nanoparticles have an average diameter of about 700 nm or greater for a predetermined amount of time after nanoparticle formation. In some embodiments, the nanoparticles have an average diameter of about 750 nm or greater for a predetermined amount of time after nanoparticle formation. In some embodiments, the nanoparticles have an average diameter of about 800 nm or greater for a predetermined amount of time after nanoparticle formation. In some embodiments, the nanoparticles have an average diameter of about 850 nm or greater for a predetermined amount of time after nanoparticle formation. In some embodiments, the nanoparticles have an average diameter of about 900 nm or greater for a predetermined amount of time after nanoparticle formation. In some embodiments, the nanoparticles have an average diameter of about 950 nm or greater for a predetermined amount of time after nanoparticle formation

In some embodiments, the nanoparticles have an average diameter of from about 10 nm to about 1000 nm for a predetermined amount of time after nanoparticle formation. In some embodiments, the nanoparticles have an average diameter of from about 10 nm to about 950 nm for a predetermined amount of time after nanoparticle formation. In some embodiments, the nanoparticles have an average diameter of from about 10 nm to about 900 nm for a predetermined amount of time after nanoparticle formation. In some embodiments, the nanoparticles have an average diameter of from about 10 nm to about 850 nm for a predetermined amount of time after nanoparticle formation. In some embodiments, the nanoparticles have an average diameter of from about 10 nm to about 800 nm for a predetermined amount of time after nanoparticle formation. In some embodiments, the nanoparticles have an average diameter of from about 10 nm to about 750 nm for a predetermined amount of time after nanoparticle formation. In some embodiments, the nanoparticles have an average diameter of from about 10 nm to about 700 nm for a predetermined amount of time after nanoparticle formation. In some embodiments, the nanoparticles have an average diameter of from about 10 nm to about 650 nm for a predetermined amount of time after nanoparticle formation. In some embodiments, the nanoparticles have an average diameter of from about 10 nm to about 600 nm for a predetermined amount of time after nanoparticle formation. In some embodiments, the nanoparticles have an average diameter of from about 10 nm to about 550 nm for a predetermined amount of time after nanoparticle formation for a predetermined amount of time after nanoparticle formation. In some embodiments, the nanoparticles have an average diameter of from about 10 nm to about 500 nm for a predetermined amount of time after nanoparticle formation. In some embodiments, the nanoparticles have an average diameter of from about 10 nm to about 450 nm for a predetermined amount of time after nanoparticle formation. In some embodiments, the nanoparticles have an average diameter of from about 10 nm to about 400 nm for a predetermined amount of time after nanoparticle formation. In some embodiments, the nanoparticles have an average diameter of from about 10 nm to about 350 nm for a predetermined amount of time after nanoparticle formation. In some embodiments, the nanoparticles have an average diameter of from about 10 nm to about 300 nm for a predetermined amount of time after nanoparticle formation. In some embodiments, the nanoparticles have an average diameter of from about 10 nm to about 250 nm for a predetermined amount of time after nanoparticle formation. In some embodiments, the nanoparticles have an average diameter of from about 10 nm to about 240 nm for a predetermined amount of time after nanoparticle formation. In some embodiments, the nanoparticles have an average diameter of from about 10 nm to about 230 nm for a predetermined amount of time after nanoparticle formation. In some embodiments, the nanoparticles have an average diameter of from about 10 nm to about 220 nm for a predetermined amount of time after nanoparticle formation. In some embodiments, the nanoparticles have an average diameter of from about 10 nm to about 210 nm for a predetermined amount of time after nanoparticle formation. In some embodiments, the nanoparticles have an average diameter of from about 10 nm to about 200 nm for a predetermined amount of time after nanoparticle formation. In some embodiments, the nanoparticles have an average diameter of from about 10 nm to about 190 nm for a predetermined amount of time after nanoparticle formation. In some embodiments, the nanoparticles have an average diameter of from about 10 nm to about 180 nm for a predetermined amount of time after nanoparticle formation. In some embodiments, the nanoparticles have an average diameter of from about 10 nm to about 170 nm for a predetermined amount of time after nanoparticle formation. In some embodiments, the nanoparticles have an average diameter of from about 10 nm to about 160 nm for a predetermined amount of time after nanoparticle formation. In some embodiments, the nanoparticles have an average diameter of from about 10 nm to about 150 nm for a predetermined amount of time after nanoparticle formation. In some embodiments, the nanoparticles have an average diameter of from about 10 nm to about 140 nm for a predetermined amount of time after nanoparticle formation. In some embodiments, the nanoparticles have an average diameter of from about 10 nm to about 130 nm for a predetermined amount of time after nanoparticle formation. In some embodiments, the nanoparticles have an average diameter of from about 10 nm to about 120 nm for a predetermined amount of time after nanoparticle formation. In some embodiments, the nanoparticles have an average diameter of from about 10 nm to about 110 nm for a predetermined amount of time after nanoparticle formation. In some embodiments, the nanoparticles have an average diameter of from about 10 nm to about 100 nm for a predetermined amount of time after nanoparticle formation. In some embodiments, the nanoparticles have an average diameter of from about 10 nm to about 90 nm for a predetermined amount of time after nanoparticle formation. In some embodiments, the nanoparticles have an average diameter of from about 10 nm to about 80 nm for a predetermined amount of time after nanoparticle formation. In some embodiments, the nanoparticles have an average diameter of from about 10 nm to about 70 nm for a predetermined amount of time after nanoparticle formation. In some embodiments, the nanoparticles have an average diameter of from about 10 nm to about 60 nm for a predetermined amount of time after nanoparticle formation. In some embodiments, the nanoparticles have an average diameter of from about 10 nm to about 50 nm for a predetermined amount of time after nanoparticle formation. In some embodiments, the nanoparticles have an average diameter of from about 10 nm to about 40 nm for a predetermined amount of time after nanoparticle formation. In some embodiments, the nanoparticles have an average diameter of from about 10 nm to about 30 nm for a predetermined amount of time after nanoparticle formation. In some embodiments, the nanoparticles have an average diameter of from about 10 nm to about 20 nm for a predetermined amount of time after nanoparticle formation.

In some embodiments, the nanoparticles have an average diameter of about 10 nm for a predetermined amount of time after nanoparticle formation. In some embodiments, the nanoparticles have an average diameter of about 20 nm for a predetermined amount of time after nanoparticle formation. In some embodiments, the nanoparticles have an average diameter of about 30 nm for a predetermined amount of time after nanoparticle formation. In some embodiments, the nanoparticles have an average diameter of about 40 nm for a predetermined amount of time after nanoparticle formation. In some embodiments, the nanoparticles have an average diameter of about 50 nm for a predetermined amount of time after nanoparticle formation. In some embodiments, the nanoparticles have an average diameter of about 60 nm for a predetermined amount of time after nanoparticle formation. In some embodiments, the nanoparticles have an average diameter of about 70 nm for a predetermined amount of time after nanoparticle formation. In some embodiments, the nanoparticles have an average diameter of about 80 nm for a predetermined amount of time after nanoparticle formation. In some embodiments, the nanoparticles have an average diameter of about 90 nm for a predetermined amount of time after nanoparticle formation. In some embodiments, the nanoparticles have an average diameter of about 100 nm for a predetermined amount of time after nanoparticle formation. In some embodiments, the nanoparticles have an average diameter of about 110 nm for a predetermined amount of time after nanoparticle formation. In some embodiments, the nanoparticles have an average diameter of about 120 nm for a predetermined amount of time after nanoparticle formation. In some embodiments, the nanoparticles have an average diameter of about 130 nm for a predetermined amount of time after nanoparticle formation. In some embodiments, the nanoparticles have an average diameter of about 140 nm for a predetermined amount of time after nanoparticle formation. In some embodiments, the nanoparticles have an average diameter of about 150 nm for a predetermined amount of time after nanoparticle formation. In some embodiments, the nanoparticles have an average diameter of about 160 nm for a predetermined amount of time after nanoparticle formation. In some embodiments, the nanoparticles have an average diameter of about 170 nm for a predetermined amount of time after nanoparticle formation. In some embodiments, the nanoparticles have an average diameter of about 180 nm for a predetermined amount of time after nanoparticle formation. In some embodiments, the nanoparticles have an average diameter of about 190 nm for a predetermined amount of time after nanoparticle formation. In some embodiments, the nanoparticles have an average diameter of about 200 nm for a predetermined amount of time after nanoparticle formation. In some embodiments, the nanoparticles have an average diameter of about 210 nm for a predetermined amount of time after nanoparticle formation. In some embodiments, the nanoparticles have an average diameter of about 220 nm for a predetermined amount of time after nanoparticle formation. In some embodiments, the nanoparticles have an average diameter of about 230 nm for a predetermined amount of time after nanoparticle formation. In some embodiments, the nanoparticles have an average diameter of about 240 nm for a predetermined amount of time after nanoparticle formation. In some embodiments, the nanoparticles have an average diameter of about 250 nm for a predetermined amount of time after nanoparticle formation. In some embodiments, the nanoparticles have an average diameter of about 300 nm for a predetermined amount of time after nanoparticle formation. In some embodiments, the nanoparticles have an average diameter of about 350 nm for a predetermined amount of time after nanoparticle formation. In some embodiments, the nanoparticles have an average diameter of about 400 nm for a predetermined amount of time after nanoparticle formation. In some embodiments, the nanoparticles have an average diameter of about 450 nm for a predetermined amount of time after nanoparticle formation. In some embodiments, the nanoparticles have an average diameter of about 500 nm for a predetermined amount of time after nanoparticle formation. In some embodiments, the nanoparticles have an average diameter of about 550 nm for a predetermined amount of time after nanoparticle formation. In some embodiments, the nanoparticles have an average diameter of about 600 nm for a predetermined amount of time after nanoparticle formation. In some embodiments, the nanoparticles have an average diameter of about 650 nm for a predetermined amount of time after nanoparticle formation. In some embodiments, the nanoparticles have an average diameter of about 700 nm for a predetermined amount of time after nanoparticle formation. In some embodiments, the nanoparticles have an average diameter of about 750 nm for a predetermined amount of time after nanoparticle formation. In some embodiments, the nanoparticles have an average diameter of about 800 nm for a predetermined amount of time after nanoparticle formation. In some embodiments, the nanoparticles have an average diameter of about 850 nm for a predetermined amount of time after nanoparticle formation. In some embodiments, the nanoparticles have an average diameter of about 900 nm for a predetermined amount of time after nanoparticle formation. In some embodiments, the nanoparticles have an average diameter of about 950 nm for a predetermined amount of time after nanoparticle formation. In some embodiments, the nanoparticles have an average diameter of about 1000 nm for a predetermined amount of time after nanoparticle formation.

In some embodiments, the predetermined amount of time is at least about 15 minutes. In some embodiments, the predetermined amount of time is at least about 30 minutes. In some embodiments, the predetermined amount of time is at least about 45 minutes. In some embodiments, the predetermined amount of time is at least about 1 hour. In some embodiments, the predetermined amount of time is at least about 2 hours. In some embodiments, the predetermined amount of time is at least about 3 hours. In some embodiments, the predetermined amount of time is at least about 4 hours. In some embodiments, the predetermined amount of time is at least about 5 hours. In some embodiments, the predetermined amount of time is at least about 6 hours. In some embodiments, the predetermined amount of time is at least about 7 hours. In some embodiments, the predetermined amount of time is at least about 8 hours. In some embodiments, the predetermined amount of time is at least about 9 hours. In some embodiments, the predetermined amount of time is at least about 10 hours. In some embodiments, the predetermined amount of time is at least about 11 hours. In some embodiments, the predetermined amount of time is at least about 12 hours. In some embodiments, the predetermined amount of time is at least about 1 day. In some embodiments, the predetermined amount of time is at least about 2 days. In some embodiments, the predetermined amount of time is at least about 3 days. In some embodiments, the predetermined amount of time is at least about 4 days. In some embodiments, the predetermined amount of time is at least about 5 days. In some embodiments, the predetermined amount of time is at least about 6 days. In some embodiments, the predetermined amount of time is at least about 7 days. In some embodiments, the predetermined amount of time is at least about 14 days. In some embodiments, the predetermined amount of time is at least about 21 days. In some embodiments, the predetermined amount of time is at least about 30 days.

In some embodiments, the predetermined amount of time is from about 15 minutes to about 30 days. In some embodiments, the predetermined amount of time is about 30 minutes to about 30 days. In some embodiments, the predetermined amount of time is from about 45 minutes to about 30 days. In some embodiments, the predetermined amount of time is from about 1 hour to about 30 days. In some embodiments, the predetermined amount of time is from about 2 hours to about 30 days. In some embodiments, the predetermined amount of time is from about 3 hours to about 30 days. In some embodiments, the predetermined amount of time is from about 4 hours to about 30 days. In some embodiments, the predetermined amount of time is from about 5 hours to about 30 days. In some embodiments, the predetermined amount of time is from about 6 hours to about 30 days. In some embodiments, the predetermined amount of time is from about 7 hours to about 30 days. In some embodiments, the predetermined amount of time is from about 8 hours to about 30 days. In some embodiments, the predetermined amount of time is from about 9 hours to about 30 days. In some embodiments, the predetermined amount of time is from about 10 hours to about 30 days. In some embodiments, the predetermined amount of time is from about 11 hours to about 30 days. In some embodiments, the predetermined amount of time is from about 12 hours to about 30 days. In some embodiments, the predetermined amount of time is from about 1 day to about 30 days. In some embodiments, the predetermined amount of time is from about 2 days to about 30 days. In some embodiments, the predetermined amount of time is from about 3 days to about 30 days. In some embodiments, the predetermined amount of time is from about 4 days to about 30 days. In some embodiments, the predetermined amount of time is from about 5 days to about 30 days. In some embodiments, the predetermined amount of time is from about 6 days to about 30 days. In some embodiments, the predetermined amount of time is from about 7 days to about 30 days. In some embodiments, the predetermined amount of time is from about 14 days to about 30 days. In some embodiments, the predetermined amount of time is from about 21 days to about 30 days.

In some embodiments, the nanoparticles have an average diameter of about 1000 nm or less for at least about 15 minutes after nanoparticle formation. In some embodiments, the nanoparticles have an average diameter of about 950 nm or less for at least about 15 minutes after nanoparticle formation. In some embodiments, the nanoparticles have an average diameter of about 900 nm or less for at least about 15 minutes after nanoparticle formation. In some embodiments, the nanoparticles have an average diameter of about 850 nm or less for at least about 15 minutes after nanoparticle formation. In some embodiments, the nanoparticles have an average diameter of about 800 nm or less for at least about 15 minutes after nanoparticle formation. In some embodiments, the nanoparticles have an average diameter of about 750 nm or less for at least about 15 minutes after nanoparticle formation. In some embodiments, the nanoparticles have an average diameter of about 700 nm or less for at least about 15 minutes after nanoparticle formation. In some embodiments, the nanoparticles have an average diameter of about 650 nm or less for at least about 15 minutes after nanoparticle formation. In some embodiments, the nanoparticles have an average diameter of about 600 nm or less for at least about 15 minutes after nanoparticle formation. In some embodiments, the nanoparticles have an average diameter of about 550 nm or less for at least about 15 minutes after nanoparticle formation. In some embodiments, the nanoparticles have an average diameter of about 500 nm or less for at least about 15 minutes after nanoparticle formation. In some embodiments, the nanoparticles have an average diameter of about 450 nm or less for at least about 15 minutes after nanoparticle formation. In some embodiments, the nanoparticles have an average diameter of about 400 nm or less for at least about 15 minutes after nanoparticle formation. In some embodiments, the nanoparticles have an average diameter of about 350 nm or less for at least about 15 minutes after nanoparticle formation. In some embodiments, the nanoparticles have an average diameter of about 300 nm or less for at least about 15 minutes after nanoparticle formation. In some embodiments, the nanoparticles have an average diameter of about 250 nm or less for at least about 15 minutes after nanoparticle formation. In some embodiments, the nanoparticles have an average diameter of about 240 nm or less for at least about 15 minutes after nanoparticle formation. In some embodiments, the nanoparticles have an average diameter of about 230 nm or less for at least about 15 minutes after nanoparticle formation. In some embodiments, the nanoparticles have an average diameter of about 220 nm or less for at least about 15 minutes after nanoparticle formation. In some embodiments, the nanoparticles have an average diameter of about 210 nm or less for at least about 15 minutes after nanoparticle formation. In some embodiments, the nanoparticles have an average diameter of about 200 nm or less for at least about 15 minutes after nanoparticle formation. In some embodiments, the nanoparticles have an average diameter of about 190 nm or less for at least about 15 minutes after nanoparticle formation. In some embodiments, the nanoparticles have an average diameter of about 180 nm or less for at least about 15 minutes after nanoparticle formation. In some embodiments, the nanoparticles have an average diameter of about 170 nm or less for at least about 15 minutes after nanoparticle formation. In some embodiments, the nanoparticles have an average diameter of about 160 nm or less for at least about 15 minutes after nanoparticle formation. In some embodiments, the nanoparticles have an average diameter of about 150 nm or less for at least about 15 minutes after nanoparticle formation. In some embodiments, the nanoparticles have an average diameter of about 140 nm or less for at least about 15 minutes after nanoparticle formation. In some embodiments, the nanoparticles have an average diameter of about 130 nm or less for at least about 15 minutes after nanoparticle formation. In some embodiments, the nanoparticles have an average diameter of about 120 nm or less for at least about 15 minutes after nanoparticle formation. In some embodiments, the nanoparticles have an average diameter of about 110 nm or less for at least about 15 minutes after nanoparticle formation. In some embodiments, the nanoparticles have an average diameter of about 100 nm or less for at least about 15 minutes after nanoparticle formation. In some embodiments, the nanoparticles have an average diameter of about 90 nm or less for at least about 15 minutes after nanoparticle formation. In some embodiments, the nanoparticles have an average diameter of about 80 nm or less for at least about 15 minutes after nanoparticle formation. In some embodiments, the nanoparticles have an average diameter of about 70 nm or less for at least about 15 minutes after nanoparticle formation. In some embodiments, the nanoparticles have an average diameter of about 60 nm or less for at least about 15 minutes after nanoparticle formation. In some embodiments, the nanoparticles have an average diameter of about 50 nm or less for at least about 15 minutes after nanoparticle formation. In some embodiments, the nanoparticles have an average diameter of about 40 nm or less for at least about 15 minutes after nanoparticle formation. In some embodiments, the nanoparticles have an average diameter of about 30 nm or less for at least about 15 minutes after nanoparticle formation. In some embodiments, the nanoparticles have an average diameter of about 20 nm or less for at least about 15 minutes after nanoparticle formation. In some embodiments, the nanoparticles have an average diameter of about 10 nm or less for at least about 15 minutes after nanoparticle formation.

In some embodiments, the nanoparticles have an average diameter of about 10 nm or greater for at least about 15 minutes after nanoparticle formation. In some embodiments, the nanoparticles have an average diameter of about 20 nm or greater for at least about 15 minutes after nanoparticle formation. In some embodiments, the nanoparticles have an average diameter of about 30 nm or greater for at least about 15 minutes after nanoparticle formation. In some embodiments, the nanoparticles have an average diameter of about 40 nm or greater for at least about 15 minutes after nanoparticle formation. In some embodiments, the nanoparticles have an average diameter of about 50 nm or greater for at least about 15 minutes after nanoparticle formation. In some embodiments, the nanoparticles have an average diameter of about 60 nm or greater for at least about 15 minutes after nanoparticle formation. In some embodiments, the nanoparticles have an average diameter of about 70 nm or greater for at least about 15 minutes after nanoparticle formation. In some embodiments, the nanoparticles have an average diameter of about 80 nm or greater for at least about 15 minutes after nanoparticle formation. In some embodiments, the nanoparticles have an average diameter of about 90 nm or greater for at least about 15 minutes after nanoparticle formation. In some embodiments, the nanoparticles have an average diameter of about 100 nm or greater for at least about 15 minutes after nanoparticle formation. In some embodiments, the nanoparticles have an average diameter of about 110 nm or greater for at least about 15 minutes after nanoparticle formation. In some embodiments, the nanoparticles have an average diameter of about 120 nm or greater for at least about 15 minutes after nanoparticle formation. In some embodiments, the nanoparticles have an average diameter of about 130 nm or greater for at least about 15 minutes after nanoparticle formation. In some embodiments, the nanoparticles have an average diameter of about 140 nm or greater for at least about 15 minutes after nanoparticle formation. In some embodiments, the nanoparticles have an average diameter of about 150 nm or greater for at least about 15 minutes after nanoparticle formation. In some embodiments, the nanoparticles have an average diameter of about 160 nm or greater for at least about 15 minutes after nanoparticle formation. In some embodiments, the nanoparticles have an average diameter of about 170 nm or greater for at least about 15 minutes after nanoparticle formation. In some embodiments, the nanoparticles have an average diameter of about 180 nm or greater for at least about 15 minutes after nanoparticle formation. In some embodiments, the nanoparticles have an average diameter of about 190 nm or greater for at least about 15 minutes after nanoparticle formation. In some embodiments, the nanoparticles have an average diameter of about 200 nm or greater for at least about 15 minutes after nanoparticle formation. In some embodiments, the nanoparticles have an average diameter of about 210 nm or greater for at least about 15 minutes after nanoparticle formation. In some embodiments, the nanoparticles have an average diameter of about 220 nm or greater for at least about 15 minutes after nanoparticle formation. In some embodiments, the nanoparticles have an average diameter of about 230 nm or greater for at least about 15 minutes after nanoparticle formation. In some embodiments, the nanoparticles have an average diameter of about 240 nm or greater for at least about 15 minutes after nanoparticle formation. In some embodiments, the nanoparticles have an average diameter of about 250 nm or greater for at least about 15 minutes after nanoparticle formation. In some embodiments, the nanoparticles have an average diameter of about 300 nm or greater for at least about 15 minutes after nanoparticle formation. In some embodiments, the nanoparticles have an average diameter of about 350 nm or greater for at least about 15 minutes after nanoparticle formation. In some embodiments, the nanoparticles have an average diameter of about 400 nm or greater for at least about 15 minutes after nanoparticle formation. In some embodiments, the nanoparticles have an average diameter of about 450 nm or greater for at least about 15 minutes after nanoparticle formation. In some embodiments, the nanoparticles have an average diameter of about 500 nm or greater for at least about 15 minutes after nanoparticle formation. In some embodiments, the nanoparticles have an average diameter of about 550 nm or greater for at least about 15 minutes after nanoparticle formation. In some embodiments, the nanoparticles have an average diameter of about 600 nm or greater for at least about 15 minutes after nanoparticle formation. In some embodiments, the nanoparticles have an average diameter of about 650 nm or greater for at least about 15 minutes after nanoparticle formation. In some embodiments, the nanoparticles have an average diameter of about 700 nm or greater for at least about 15 minutes after nanoparticle formation. In some embodiments, the nanoparticles have an average diameter of about 750 nm or greater for at least about 15 minutes after nanoparticle formation. In some embodiments, the nanoparticles have an average diameter of about 800 nm or greater for at least about 15 minutes after nanoparticle formation. In some embodiments, the nanoparticles have an average diameter of about 850 nm or greater for at least about 15 minutes after nanoparticle formation. In some embodiments, the nanoparticles have an average diameter of about 900 nm or greater for at least about 15 minutes after nanoparticle formation. In some embodiments, the nanoparticles have an average diameter of about 950 nm or greater for at least about 15 minutes after nanoparticle formation

In some embodiments, the nanoparticles have an average diameter of from about 10 nm to about 1000 nm for at least about 15 minutes after nanoparticle formation. In some embodiments, the nanoparticles have an average diameter of from about 10 nm to about 950 nm for at least about 15 minutes after nanoparticle formation. In some embodiments, the nanoparticles have an average diameter of from about 10 nm to about 900 nm for at least about 15 minutes after nanoparticle formation. In some embodiments, the nanoparticles have an average diameter of from about 10 nm to about 850 nm for at least about 15 minutes after nanoparticle formation. In some embodiments, the nanoparticles have an average diameter of from about 10 nm to about 800 nm for at least about 15 minutes after nanoparticle formation. In some embodiments, the nanoparticles have an average diameter of from about 10 nm to about 750 nm for at least about 15 minutes after nanoparticle formation. In some embodiments, the nanoparticles have an average diameter of from about 10 nm to about 700 nm for at least about 15 minutes after nanoparticle formation. In some embodiments, the nanoparticles have an average diameter of from about 10 nm to about 650 nm for at least about 15 minutes after nanoparticle formation. In some embodiments, the nanoparticles have an average diameter of from about 10 nm to about 600 nm for at least about 15 minutes after nanoparticle formation. In some embodiments, the nanoparticles have an average diameter of from about 10 nm to about 550 nm for at least about 15 minutes after nanoparticle formation for at least about 15 minutes after nanoparticle formation. In some embodiments, the nanoparticles have an average diameter of from about 10 nm to about 500 nm for at least about 15 minutes after nanoparticle formation. In some embodiments, the nanoparticles have an average diameter of from about 10 nm to about 450 nm for at least about 15 minutes after nanoparticle formation. In some embodiments, the nanoparticles have an average diameter of from about 10 nm to about 400 nm for at least about 15 minutes after nanoparticle formation. In some embodiments, the nanoparticles have an average diameter of from about 10 nm to about 350 nm for at least about 15 minutes after nanoparticle formation. In some embodiments, the nanoparticles have an average diameter of from about 10 nm to about 300 nm for at least about 15 minutes after nanoparticle formation. In some embodiments, the nanoparticles have an average diameter of from about 10 nm to about 250 nm for at least about 15 minutes after nanoparticle formation. In some embodiments, the nanoparticles have an average diameter of from about 10 nm to about 240 nm for at least about 15 minutes after nanoparticle formation. In some embodiments, the nanoparticles have an average diameter of from about 10 nm to about 230 nm for at least about 15 minutes after nanoparticle formation. In some embodiments, the nanoparticles have an average diameter of from about 10 nm to about 220 nm for at least about 15 minutes after nanoparticle formation. In some embodiments, the nanoparticles have an average diameter of from about 10 nm to about 210 nm for at least about 15 minutes after nanoparticle formation. In some embodiments, the nanoparticles have an average diameter of from about 10 nm to about 200 nm for at least about 15 minutes after nanoparticle formation. In some embodiments, the nanoparticles have an average diameter of from about 10 nm to about 190 nm for at least about 15 minutes after nanoparticle formation. In some embodiments, the nanoparticles have an average diameter of from about 10 nm to about 180 nm for at least about 15 minutes after nanoparticle formation. In some embodiments, the nanoparticles have an average diameter of from about 10 nm to about 170 nm for at least about 15 minutes after nanoparticle formation. In some embodiments, the nanoparticles have an average diameter of from about 10 nm to about 160 nm for at least about 15 minutes after nanoparticle formation. In some embodiments, the nanoparticles have an average diameter of from about 10 nm to about 150 nm for at least about 15 minutes after nanoparticle formation. In some embodiments, the nanoparticles have an average diameter of from about 10 nm to about 140 nm for at least about 15 minutes after nanoparticle formation. In some embodiments, the nanoparticles have an average diameter of from about 10 nm to about 130 nm for at least about 15 minutes after nanoparticle formation. In some embodiments, the nanoparticles have an average diameter of from about 10 nm to about 120 nm for at least about 15 minutes after nanoparticle formation. In some embodiments, the nanoparticles have an average diameter of from about 10 nm to about 110 nm for at least about 15 minutes after nanoparticle formation. In some embodiments, the nanoparticles have an average diameter of from about 10 nm to about 100 nm for at least about 15 minutes after nanoparticle formation. In some embodiments, the nanoparticles have an average diameter of from about 10 nm to about 90 nm for at least about 15 minutes after nanoparticle formation. In some embodiments, the nanoparticles have an average diameter of from about 10 nm to about 80 nm for at least about 15 minutes after nanoparticle formation. In some embodiments, the nanoparticles have an average diameter of from about 10 nm to about 70 nm for at least about 15 minutes after nanoparticle formation. In some embodiments, the nanoparticles have an average diameter of from about 10 nm to about 60 nm for at least about 15 minutes after nanoparticle formation. In some embodiments, the nanoparticles have an average diameter of from about 10 nm to about 50 nm for at least about 15 minutes after nanoparticle formation. In some embodiments, the nanoparticles have an average diameter of from about 10 nm to about 40 nm for at least about 15 minutes after nanoparticle formation. In some embodiments, the nanoparticles have an average diameter of from about 10 nm to about 30 nm for at least about 15 minutes after nanoparticle formation. In some embodiments, the nanoparticles have an average diameter of from about 10 nm to about 20 nm for at least about 15 minutes after nanoparticle formation.

In some embodiments, the nanoparticles have an average diameter of about 10 nm for at least about 15 minutes after nanoparticle formation. In some embodiments, the nanoparticles have an average diameter of about 20 nm for at least about 15 minutes after nanoparticle formation. In some embodiments, the nanoparticles have an average diameter of about 30 nm for at least about 15 minutes after nanoparticle formation. In some embodiments, the nanoparticles have an average diameter of about 40 nm for at least about 15 minutes after nanoparticle formation. In some embodiments, the nanoparticles have an average diameter of about 50 nm for at least about 15 minutes after nanoparticle formation. In some embodiments, the nanoparticles have an average diameter of about 60 nm for at least about 15 minutes after nanoparticle formation. In some embodiments, the nanoparticles have an average diameter of about 70 nm for at least about 15 minutes after nanoparticle formation. In some embodiments, the nanoparticles have an average diameter of about 80 nm for at least about 15 minutes after nanoparticle formation. In some embodiments, the nanoparticles have an average diameter of about 90 nm for at least about 15 minutes after nanoparticle formation. In some embodiments, the nanoparticles have an average diameter of about 100 nm for at least about 15 minutes after nanoparticle formation. In some embodiments, the nanoparticles have an average diameter of about 110 nm for at least about 15 minutes after nanoparticle formation. In some embodiments, the nanoparticles have an average diameter of about 120 nm for at least about 15 minutes after nanoparticle formation. In some embodiments, the nanoparticles have an average diameter of about 130 nm for at least about 15 minutes after nanoparticle formation. In some embodiments, the nanoparticles have an average diameter of about 140 nm for at least about 15 minutes after nanoparticle formation. In some embodiments, the nanoparticles have an average diameter of about 150 nm for at least about 15 minutes after nanoparticle formation. In some embodiments, the nanoparticles have an average diameter of about 160 nm for at least about 15 minutes after nanoparticle formation. In some embodiments, the nanoparticles have an average diameter of about 170 nm for at least about 15 minutes after nanoparticle formation. In some embodiments, the nanoparticles have an average diameter of about 180 nm for at least about 15 minutes after nanoparticle formation. In some embodiments, the nanoparticles have an average diameter of about 190 nm for at least about 15 minutes after nanoparticle formation. In some embodiments, the nanoparticles have an average diameter of about 200 nm for at least about 15 minutes after nanoparticle formation. In some embodiments, the nanoparticles have an average diameter of about 210 nm for at least about 15 minutes after nanoparticle formation. In some embodiments, the nanoparticles have an average diameter of about 220 nm for at least about 15 minutes after nanoparticle formation. In some embodiments, the nanoparticles have an average diameter of about 230 nm for at least about 15 minutes after nanoparticle formation. In some embodiments, the nanoparticles have an average diameter of about 240 nm for at least about 15 minutes after nanoparticle formation. In some embodiments, the nanoparticles have an average diameter of about 250 nm for at least about 15 minutes after nanoparticle formation. In some embodiments, the nanoparticles have an average diameter of about 300 nm for at least about 15 minutes after nanoparticle formation. In some embodiments, the nanoparticles have an average diameter of about 350 nm for at least about 15 minutes after nanoparticle formation. In some embodiments, the nanoparticles have an average diameter of about 400 nm for at least about 15 minutes after nanoparticle formation. In some embodiments, the nanoparticles have an average diameter of about 450 nm for at least about 15 minutes after nanoparticle formation. In some embodiments, the nanoparticles have an average diameter of about 500 nm for at least about 15 minutes after nanoparticle formation. In some embodiments, the nanoparticles have an average diameter of about 550 nm for at least about 15 minutes after nanoparticle formation. In some embodiments, the nanoparticles have an average diameter of about 600 nm for at least about 15 minutes after nanoparticle formation. In some embodiments, the nanoparticles have an average diameter of about 650 nm for at least about 15 minutes after nanoparticle formation. In some embodiments, the nanoparticles have an average diameter of about 700 nm for at least about 15 minutes after nanoparticle formation. In some embodiments, the nanoparticles have an average diameter of about 750 nm for at least about 15 minutes after nanoparticle formation. In some embodiments, the nanoparticles have an average diameter of about 800 nm for at least about 15 minutes after nanoparticle formation. In some embodiments, the nanoparticles have an average diameter of about 850 nm for at least about 15 minutes after nanoparticle formation. In some embodiments, the nanoparticles have an average diameter of about 900 nm for at least about 15 minutes after nanoparticle formation. In some embodiments, the nanoparticles have an average diameter of about 950 nm for at least about 15 minutes after nanoparticle formation. In some embodiments, the nanoparticles have an average diameter of about 1000 nm for at least about 15 minutes after nanoparticle formation.

In some embodiments, the nanoparticles have an average diameter of about 1000 nm or less for at least about 4 hours after nanoparticle formation. In some embodiments, the nanoparticles have an average diameter of about 950 nm or less for at least about 4 hours after nanoparticle formation. In some embodiments, the nanoparticles have an average diameter of about 900 nm or less for at least about 4 hours after nanoparticle formation. In some embodiments, the nanoparticles have an average diameter of about 850 nm or less for at least about 4 hours after nanoparticle formation. In some embodiments, the nanoparticles have an average diameter of about 800 nm or less for at least about 4 hours after nanoparticle formation. In some embodiments, the nanoparticles have an average diameter of about 750 nm or less for at least about 4 hours after nanoparticle formation. In some embodiments, the nanoparticles have an average diameter of about 700 nm or less for at least about 4 hours after nanoparticle formation. In some embodiments, the nanoparticles have an average diameter of about 650 nm or less for at least about 4 hours after nanoparticle formation. In some embodiments, the nanoparticles have an average diameter of about 600 nm or less for at least about 4 hours after nanoparticle formation. In some embodiments, the nanoparticles have an average diameter of about 550 nm or less for at least about 4 hours after nanoparticle formation. In some embodiments, the nanoparticles have an average diameter of about 500 nm or less for at least about 4 hours after nanoparticle formation. In some embodiments, the nanoparticles have an average diameter of about 450 nm or less for at least about 4 hours after nanoparticle formation. In some embodiments, the nanoparticles have an average diameter of about 400 nm or less for at least about 4 hours after nanoparticle formation. In some embodiments, the nanoparticles have an average diameter of about 350 nm or less for at least about 4 hours after nanoparticle formation. In some embodiments, the nanoparticles have an average diameter of about 300 nm or less for at least about 4 hours after nanoparticle formation. In some embodiments, the nanoparticles have an average diameter of about 250 nm or less for at least about 4 hours after nanoparticle formation. In some embodiments, the nanoparticles have an average diameter of about 240 nm or less for at least about 4 hours after nanoparticle formation. In some embodiments, the nanoparticles have an average diameter of about 230 nm or less for at least about 4 hours after nanoparticle formation. In some embodiments, the nanoparticles have an average diameter of about 220 nm or less for at least about 4 hours after nanoparticle formation. In some embodiments, the nanoparticles have an average diameter of about 210 nm or less for at least about 4 hours after nanoparticle formation. In some embodiments, the nanoparticles have an average diameter of about 200 nm or less for at least about 4 hours after nanoparticle formation. In some embodiments, the nanoparticles have an average diameter of about 190 nm or less for at least about 4 hours after nanoparticle formation. In some embodiments, the nanoparticles have an average diameter of about 180 nm or less for at least about 4 hours after nanoparticle formation. In some embodiments, the nanoparticles have an average diameter of about 170 nm or less for at least about 4 hours after nanoparticle formation. In some embodiments, the nanoparticles have an average diameter of about 160 nm or less for at least about 4 hours after nanoparticle formation. In some embodiments, the nanoparticles have an average diameter of about 150 nm or less for at least about 4 hours after nanoparticle formation. In some embodiments, the nanoparticles have an average diameter of about 140 nm or less for at least about 4 hours after nanoparticle formation. In some embodiments, the nanoparticles have an average diameter of about 130 nm or less for at least about 4 hours after nanoparticle formation. In some embodiments, the nanoparticles have an average diameter of about 120 nm or less for at least about 4 hours after nanoparticle formation. In some embodiments, the nanoparticles have an average diameter of about 110 nm or less for at least about 4 hours after nanoparticle formation. In some embodiments, the nanoparticles have an average diameter of about 100 nm or less for at least about 4 hours after nanoparticle formation. In some embodiments, the nanoparticles have an average diameter of about 90 nm or less for at least about 4 hours after nanoparticle formation. In some embodiments, the nanoparticles have an average diameter of about 80 nm or less for at least about 4 hours after nanoparticle formation. In some embodiments, the nanoparticles have an average diameter of about 70 nm or less for at least about 4 hours after nanoparticle formation. In some embodiments, the nanoparticles have an average diameter of about 60 nm or less for at least about 4 hours after nanoparticle formation. In some embodiments, the nanoparticles have an average diameter of about 50 nm or less for at least about 4 hours after nanoparticle formation. In some embodiments, the nanoparticles have an average diameter of about 40 nm or less for at least about 4 hours after nanoparticle formation. In some embodiments, the nanoparticles have an average diameter of about 30 nm or less for at least about 4 hours after nanoparticle formation. In some embodiments, the nanoparticles have an average diameter of about 20 nm or less for at least about 4 hours after nanoparticle formation. In some embodiments, the nanoparticles have an average diameter of about 10 nm or less for at least about 4 hours after nanoparticle formation.

In some embodiments, the nanoparticles have an average diameter of about 10 nm or greater for at least about 4 hours after nanoparticle formation. In some embodiments, the nanoparticles have an average diameter of about 20 nm or greater for at least about 4 hours after nanoparticle formation. In some embodiments, the nanoparticles have an average diameter of about 30 nm or greater for at least about 4 hours after nanoparticle formation. In some embodiments, the nanoparticles have an average diameter of about 40 nm or greater for at least about 4 hours after nanoparticle formation. In some embodiments, the nanoparticles have an average diameter of about 50 nm or greater for at least about 4 hours after nanoparticle formation. In some embodiments, the nanoparticles have an average diameter of about 60 nm or greater for at least about 4 hours after nanoparticle formation. In some embodiments, the nanoparticles have an average diameter of about 70 nm or greater for at least about 4 hours after nanoparticle formation. In some embodiments, the nanoparticles have an average diameter of about 80 nm or greater for at least about 4 hours after nanoparticle formation. In some embodiments, the nanoparticles have an average diameter of about 90 nm or greater for at least about 4 hours after nanoparticle formation. In some embodiments, the nanoparticles have an average diameter of about 100 nm or greater for at least about 4 hours after nanoparticle formation. In some embodiments, the nanoparticles have an average diameter of about 110 nm or greater for at least about 4 hours after nanoparticle formation. In some embodiments, the nanoparticles have an average diameter of about 120 nm or greater for at least about 4 hours after nanoparticle formation. In some embodiments, the nanoparticles have an average diameter of about 130 nm or greater for at least about 4 hours after nanoparticle formation. In some embodiments, the nanoparticles have an average diameter of about 140 nm or greater for at least about 4 hours after nanoparticle formation. In some embodiments, the nanoparticles have an average diameter of about 150 nm or greater for at least about 4 hours after nanoparticle formation. In some embodiments, the nanoparticles have an average diameter of about 160 nm or greater for at least about 4 hours after nanoparticle formation. In some embodiments, the nanoparticles have an average diameter of about 170 nm or greater for at least about 4 hours after nanoparticle formation. In some embodiments, the nanoparticles have an average diameter of about 180 nm or greater for at least about 4 hours after nanoparticle formation. In some embodiments, the nanoparticles have an average diameter of about 190 nm or greater for at least about 4 hours after nanoparticle formation. In some embodiments, the nanoparticles have an average diameter of about 200 nm or greater for at least about 4 hours after nanoparticle formation. In some embodiments, the nanoparticles have an average diameter of about 210 nm or greater for at least about 4 hours after nanoparticle formation. In some embodiments, the nanoparticles have an average diameter of about 220 nm or greater for at least about 4 hours after nanoparticle formation. In some embodiments, the nanoparticles have an average diameter of about 230 nm or greater for at least about 4 hours after nanoparticle formation. In some embodiments, the nanoparticles have an average diameter of about 240 nm or greater for at least about 4 hours after nanoparticle formation. In some embodiments, the nanoparticles have an average diameter of about 250 nm or greater for at least about 4 hours after nanoparticle formation. In some embodiments, the nanoparticles have an average diameter of about 300 nm or greater for at least about 4 hours after nanoparticle formation. In some embodiments, the nanoparticles have an average diameter of about 350 nm or greater for at least about 4 hours after nanoparticle formation. In some embodiments, the nanoparticles have an average diameter of about 400 nm or greater for at least about 4 hours after nanoparticle formation. In some embodiments, the nanoparticles have an average diameter of about 450 nm or greater for at least about 4 hours after nanoparticle formation. In some embodiments, the nanoparticles have an average diameter of about 500 nm or greater for at least about 4 hours after nanoparticle formation. In some embodiments, the nanoparticles have an average diameter of about 550 nm or greater for at least about 4 hours after nanoparticle formation. In some embodiments, the nanoparticles have an average diameter of about 600 nm or greater for at least about 4 hours after nanoparticle formation. In some embodiments, the nanoparticles have an average diameter of about 650 nm or greater for at least about 4 hours after nanoparticle formation. In some embodiments, the nanoparticles have an average diameter of about 700 nm or greater for at least about 4 hours after nanoparticle formation. In some embodiments, the nanoparticles have an average diameter of about 750 nm or greater for at least about 4 hours after nanoparticle formation. In some embodiments, the nanoparticles have an average diameter of about 800 nm or greater for at least about 4 hours after nanoparticle formation. In some embodiments, the nanoparticles have an average diameter of about 850 nm or greater for at least about 4 hours after nanoparticle formation. In some embodiments, the nanoparticles have an average diameter of about 900 nm or greater for at least about 4 hours after nanoparticle formation. In some embodiments, the nanoparticles have an average diameter of about 950 nm or greater for at least about 4 hours after nanoparticle formation

In some embodiments, the nanoparticles have an average diameter of from about 10 nm to about 1000 nm for at least about 4 hours after nanoparticle formation. In some embodiments, the nanoparticles have an average diameter of from about 10 nm to about 950 nm for at least about 4 hours after nanoparticle formation. In some embodiments, the nanoparticles have an average diameter of from about 10 nm to about 900 nm for at least about 4 hours after nanoparticle formation. In some embodiments, the nanoparticles have an average diameter of from about 10 nm to about 850 nm for at least about 4 hours after nanoparticle formation. In some embodiments, the nanoparticles have an average diameter of from about 10 nm to about 800 nm for at least about 4 hours after nanoparticle formation. In some embodiments, the nanoparticles have an average diameter of from about 10 nm to about 750 nm for at least about 4 hours after nanoparticle formation. In some embodiments, the nanoparticles have an average diameter of from about 10 nm to about 700 nm for at least about 4 hours after nanoparticle formation. In some embodiments, the nanoparticles have an average diameter of from about 10 nm to about 650 nm for at least about 4 hours after nanoparticle formation. In some embodiments, the nanoparticles have an average diameter of from about 10 nm to about 600 nm for at least about 4 hours after nanoparticle formation. In some embodiments, the nanoparticles have an average diameter of from about 10 nm to about 550 nm for at least about 4 hours after nanoparticle formation for at least about 4 hours after nanoparticle formation. In some embodiments, the nanoparticles have an average diameter of from about 10 nm to about 500 nm for at least about 4 hours after nanoparticle formation. In some embodiments, the nanoparticles have an average diameter of from about 10 nm to about 450 nm for at least about 4 hours after nanoparticle formation. In some embodiments, the nanoparticles have an average diameter of from about 10 nm to about 400 nm for at least about 4 hours after nanoparticle formation. In some embodiments, the nanoparticles have an average diameter of from about 10 nm to about 350 nm for at least about 4 hours after nanoparticle formation. In some embodiments, the nanoparticles have an average diameter of from about 10 nm to about 300 nm for at least about 4 hours after nanoparticle formation. In some embodiments, the nanoparticles have an average diameter of from about 10 nm to about 250 nm for at least about 4 hours after nanoparticle formation. In some embodiments, the nanoparticles have an average diameter of from about 10 nm to about 240 nm for at least about 4 hours after nanoparticle formation. In some embodiments, the nanoparticles have an average diameter of from about 10 nm to about 230 nm for at least about 4 hours after nanoparticle formation. In some embodiments, the nanoparticles have an average diameter of from about 10 nm to about 220 nm for at least about 4 hours after nanoparticle formation. In some embodiments, the nanoparticles have an average diameter of from about 10 nm to about 210 nm for at least about 4 hours after nanoparticle formation. In some embodiments, the nanoparticles have an average diameter of from about 10 nm to about 200 nm for at least about 4 hours after nanoparticle formation. In some embodiments, the nanoparticles have an average diameter of from about 10 nm to about 190 nm for at least about 4 hours after nanoparticle formation. In some embodiments, the nanoparticles have an average diameter of from about 10 nm to about 180 nm for at least about 4 hours after nanoparticle formation. In some embodiments, the nanoparticles have an average diameter of from about 10 nm to about 170 nm for at least about 4 hours after nanoparticle formation. In some embodiments, the nanoparticles have an average diameter of from about 10 nm to about 160 nm for at least about 4 hours after nanoparticle formation. In some embodiments, the nanoparticles have an average diameter of from about 10 nm to about 150 nm for at least about 4 hours after nanoparticle formation. In some embodiments, the nanoparticles have an average diameter of from about 10 nm to about 140 nm for at least about 4 hours after nanoparticle formation. In some embodiments, the nanoparticles have an average diameter of from about 10 nm to about 130 nm for at least about 4 hours after nanoparticle formation. In some embodiments, the nanoparticles have an average diameter of from about 10 nm to about 120 nm for at least about 4 hours after nanoparticle formation. In some embodiments, the nanoparticles have an average diameter of from about 10 nm to about 110 nm for at least about 4 hours after nanoparticle formation. In some embodiments, the nanoparticles have an average diameter of from about 10 nm to about 100 nm for at least about 4 hours after nanoparticle formation. In some embodiments, the nanoparticles have an average diameter of from about 10 nm to about 90 nm for at least about 4 hours after nanoparticle formation. In some embodiments, the nanoparticles have an average diameter of from about 10 nm to about 80 nm for at least about 4 hours after nanoparticle formation. In some embodiments, the nanoparticles have an average diameter of from about 10 nm to about 70 nm for at least about 4 hours after nanoparticle formation. In some embodiments, the nanoparticles have an average diameter of from about 10 nm to about 60 nm for at least about 4 hours after nanoparticle formation. In some embodiments, the nanoparticles have an average diameter of from about 10 nm to about 50 nm for at least about 4 hours after nanoparticle formation. In some embodiments, the nanoparticles have an average diameter of from about 10 nm to about 40 nm for at least about 4 hours after nanoparticle formation. In some embodiments, the nanoparticles have an average diameter of from about 10 nm to about 30 nm for at least about 4 hours after nanoparticle formation. In some embodiments, the nanoparticles have an average diameter of from about 10 nm to about 20 nm for at least about 4 hours after nanoparticle formation.

In some embodiments, the nanoparticles have an average diameter of about 10 nm for at least about 4 hours after nanoparticle formation. In some embodiments, the nanoparticles have an average diameter of about 20 nm for at least about 4 hours after nanoparticle formation. In some embodiments, the nanoparticles have an average diameter of about 30 nm for at least about 4 hours after nanoparticle formation. In some embodiments, the nanoparticles have an average diameter of about 40 nm for at least about 4 hours after nanoparticle formation. In some embodiments, the nanoparticles have an average diameter of about 50 nm for at least about 4 hours after nanoparticle formation. In some embodiments, the nanoparticles have an average diameter of about 60 nm for at least about 4 hours after nanoparticle formation. In some embodiments, the nanoparticles have an average diameter of about 70 nm for at least about 4 hours after nanoparticle formation. In some embodiments, the nanoparticles have an average diameter of about 80 nm for at least about 4 hours after nanoparticle formation. In some embodiments, the nanoparticles have an average diameter of about 90 nm for at least about 4 hours after nanoparticle formation. In some embodiments, the nanoparticles have an average diameter of about 100 nm for at least about 4 hours after nanoparticle formation. In some embodiments, the nanoparticles have an average diameter of about 110 nm for at least about 4 hours after nanoparticle formation. In some embodiments, the nanoparticles have an average diameter of about 120 nm for at least about 4 hours after nanoparticle formation. In some embodiments, the nanoparticles have an average diameter of about 130 nm for at least about 4 hours after nanoparticle formation. In some embodiments, the nanoparticles have an average diameter of about 140 nm for at least about 4 hours after nanoparticle formation. In some embodiments, the nanoparticles have an average diameter of about 150 nm for at least about 4 hours after nanoparticle formation. In some embodiments, the nanoparticles have an average diameter of about 160 nm for at least about 4 hours after nanoparticle formation. In some embodiments, the nanoparticles have an average diameter of about 170 nm for at least about 4 hours after nanoparticle formation. In some embodiments, the nanoparticles have an average diameter of about 180 nm for at least about 4 hours after nanoparticle formation. In some embodiments, the nanoparticles have an average diameter of about 190 nm for at least about 4 hours after nanoparticle formation. In some embodiments, the nanoparticles have an average diameter of about 200 nm for at least about 4 hours after nanoparticle formation. In some embodiments, the nanoparticles have an average diameter of about 210 nm for at least about 4 hours after nanoparticle formation. In some embodiments, the nanoparticles have an average diameter of about 220 nm for at least about 4 hours after nanoparticle formation. In some embodiments, the nanoparticles have an average diameter of about 230 nm for at least about 4 hours after nanoparticle formation. In some embodiments, the nanoparticles have an average diameter of about 240 nm for at least about 4 hours after nanoparticle formation. In some embodiments, the nanoparticles have an average diameter of about 250 nm for at least about 4 hours after nanoparticle formation. In some embodiments, the nanoparticles have an average diameter of about 300 nm for at least about 4 hours after nanoparticle formation. In some embodiments, the nanoparticles have an average diameter of about 350 nm for at least about 4 hours after nanoparticle formation. In some embodiments, the nanoparticles have an average diameter of about 400 nm for at least about 4 hours after nanoparticle formation. In some embodiments, the nanoparticles have an average diameter of about 450 nm for at least about 4 hours after nanoparticle formation. In some embodiments, the nanoparticles have an average diameter of about 500 nm for at least about 4 hours after nanoparticle formation. In some embodiments, the nanoparticles have an average diameter of about 550 nm for at least about 4 hours after nanoparticle formation. In some embodiments, the nanoparticles have an average diameter of about 600 nm for at least about 4 hours after nanoparticle formation. In some embodiments, the nanoparticles have an average diameter of about 650 nm for at least about 4 hours after nanoparticle formation. In some embodiments, the nanoparticles have an average diameter of about 700 nm for at least about 4 hours after nanoparticle formation. In some embodiments, the nanoparticles have an average diameter of about 750 nm for at least about 4 hours after nanoparticle formation. In some embodiments, the nanoparticles have an average diameter of about 800 nm for at least about 4 hours after nanoparticle formation. In some embodiments, the nanoparticles have an average diameter of about 850 nm for at least about 4 hours after nanoparticle formation. In some embodiments, the nanoparticles have an average diameter of about 900 nm for at least about 4 hours after nanoparticle formation. In some embodiments, the nanoparticles have an average diameter of about 950 nm for at least about 4 hours after nanoparticle formation. In some embodiments, the nanoparticles have an average diameter of about 1000 nm for at least about 4 hours after nanoparticle formation.

In some embodiments, the nanoparticles have an average diameter of from about 10 nm to about 1000 nm. In some embodiments, the nanoparticles have an average diameter of from about 10 nm to about 950 nm. In some embodiments, the nanoparticles have an average diameter of from about 10 nm to about 900 nm. In some embodiments, the nanoparticles have an average diameter of from about 10 nm to about 850 nm. In some embodiments, the nanoparticles have an average diameter of from about 10 nm to about 800 nm. In some embodiments, the nanoparticles have an average diameter of from about 10 nm to about 750 nm. In some embodiments, the nanoparticles have an average diameter of from about 10 nm to about 700 nm. In some embodiments, the nanoparticles have an average diameter of from about 10 nm to about 650 nm. In some embodiments, the nanoparticles have an average diameter of from about 10 nm to about 600 nm. In some embodiments, the nanoparticles have an average diameter of from about 10 nm to about 550 nm. In some embodiments, the nanoparticles have an average diameter of from about 10 nm to about 500 nm. In some embodiments, the nanoparticles have an average diameter of from about 10 nm to about 450 nm. In some embodiments, the nanoparticles have an average diameter of from about 10 nm to about 400 nm. In some embodiments, the nanoparticles have an average diameter of from about 10 nm to about 350 nm. In some embodiments, the nanoparticles have an average diameter of from about 10 nm to about 300 nm. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm. In some embodiments, the nanoparticles have an average diameter of from about 10 nm to about 190 nm. In some embodiments, the nanoparticles have an average diameter of from about 10 nm to about 180 nm. In some embodiments, the nanoparticles have an average diameter of from about 10 nm to about 170 nm. In some embodiments, the nanoparticles have an average diameter of from about 10 nm to about 160 nm. In some embodiments, the nanoparticles have an average diameter of from about 10 nm to about 150 nm. In some embodiments, the nanoparticles have an average diameter of from about 10 nm to about 140 nm. In some embodiments, the nanoparticles have an average diameter of from about 10 nm to about 130 nm. In some embodiments, the nanoparticles have an average diameter of from about 10 nm to about 120 nm. In some embodiments, the nanoparticles have an average diameter of from about 10 nm to about 110 nm. In some embodiments, the nanoparticles have an average diameter of from about 10 nm to about 100 nm. In some embodiments, the nanoparticles have an average diameter of from about 10 nm to about 90 nm. In some embodiments, the nanoparticles have an average diameter of from about 10 nm to about 80 nm. In some embodiments, the nanoparticles have an average diameter of from about 10 nm to about 70 nm. In some embodiments, the nanoparticles have an average diameter of from about 10 nm to about 60 nm. In some embodiments, the nanoparticles have an average diameter of from about 10 nm to about 50 nm. In some embodiments, the nanoparticles have an average diameter of from about 10 nm to about 40 nm. In some embodiments, the nanoparticles have an average diameter of from about 10 nm to about 30 nm. In some embodiments, the nanoparticles have an average diameter of from about 10 nm to about 20 nm.

In some embodiments, the nanoparticles have an average diameter of from about 20 nm to about 1000 nm. In some embodiments, the nanoparticles have an average diameter of from about 20 nm to about 950 nm. In some embodiments, the nanoparticles have an average diameter of from about 20 nm to about 900 nm. In some embodiments, the nanoparticles have an average diameter of from about 20 nm to about 850 nm. In some embodiments, the nanoparticles have an average diameter of from about 20 nm to about 800 nm. In some embodiments, the nanoparticles have an average diameter of from about 20 nm to about 750 nm. In some embodiments, the nanoparticles have an average diameter of from about 20 nm to about 700 nm. In some embodiments, the nanoparticles have an average diameter of from about 20 nm to about 650 nm. In some embodiments, the nanoparticles have an average diameter of from about 20 nm to about 600 nm. In some embodiments, the nanoparticles have an average diameter of from about 20 nm to about 550 nm. In some embodiments, the nanoparticles have an average diameter of from about 20 nm to about 500 nm. In some embodiments, the nanoparticles have an average diameter of from about 20 nm to about 450 nm. In some embodiments, the nanoparticles have an average diameter of from about 20 nm to about 400 nm. In some embodiments, the nanoparticles have an average diameter of from about 20 nm to about 350 nm. In some embodiments, the nanoparticles have an average diameter of from about 20 nm to about 300 nm. In some embodiments, the nanoparticles have an average diameter of from about 20 nm to about 250 nm. In some embodiments, the nanoparticles have an average diameter of from about 20 nm to about 240 nm. In some embodiments, the nanoparticles have an average diameter of from about 20 nm to about 230 nm. In some embodiments, the nanoparticles have an average diameter of from about 20 nm to about 220 nm. In some embodiments, the nanoparticles have an average diameter of from about 20 nm to about 210 nm. In some embodiments, the nanoparticles have an average diameter of from about 20 nm to about 200 nm. In some embodiments, the nanoparticles have an average diameter of from about 20 nm to about 190 nm. In some embodiments, the nanoparticles have an average diameter of from about 20 nm to about 180 nm. In some embodiments, the nanoparticles have an average diameter of from about 20 nm to about 170 nm. In some embodiments, the nanoparticles have an average diameter of from about 20 nm to about 160 nm. In some embodiments, the nanoparticles have an average diameter of from about 20 nm to about 150 nm. In some embodiments, the nanoparticles have an average diameter of from about 20 nm to about 140 nm. In some embodiments, the nanoparticles have an average diameter of from about 20 nm to about 130 nm. In some embodiments, the nanoparticles have an average diameter of from about 20 nm to about 120 nm. In some embodiments, the nanoparticles have an average diameter of from about 20 nm to about 110 nm. In some embodiments, the nanoparticles have an average diameter of from about 20 nm to about 100 nm. In some embodiments, the nanoparticles have an average diameter of from about 20 nm to about 90 nm. In some embodiments, the nanoparticles have an average diameter of from about 20 nm to about 80 nm. In some embodiments, the nanoparticles have an average diameter of from about 20 nm to about 70 nm. In some embodiments, the nanoparticles have an average diameter of from about 20 nm to about 60 nm. In some embodiments, the nanoparticles have an average diameter of from about 20 nm to about 50 nm. In some embodiments, the nanoparticles have an average diameter of from about 20 nm to about 40 nm. In some embodiments, the nanoparticles have an average diameter of from about 20 nm to about 30 nm.

In some embodiments, the nanoparticles have an average diameter of from about 30 nm to about 1000 nm. In some embodiments, the nanoparticles have an average diameter of from about 30 nm to about 950 nm. In some embodiments, the nanoparticles have an average diameter of from about 30 nm to about 900 nm. In some embodiments, the nanoparticles have an average diameter of from about 30 nm to about 850 nm. In some embodiments, the nanoparticles have an average diameter of from about 30 nm to about 800 nm. In some embodiments, the nanoparticles have an average diameter of from about 30 nm to about 750 nm. In some embodiments, the nanoparticles have an average diameter of from about 30 nm to about 700 nm. In some embodiments, the nanoparticles have an average diameter of from about 30 nm to about 650 nm. In some embodiments, the nanoparticles have an average diameter of from about 30 nm to about 600 nm. In some embodiments, the nanoparticles have an average diameter of from about 30 nm to about 550 nm. In some embodiments, the nanoparticles have an average diameter of from about 30 nm to about 500 nm. In some embodiments, the nanoparticles have an average diameter of from about 30 nm to about 450 nm. In some embodiments, the nanoparticles have an average diameter of from about 30 nm to about 400 nm. In some embodiments, the nanoparticles have an average diameter of from about 30 nm to about 350 nm. In some embodiments, the nanoparticles have an average diameter of from about 30 nm to about 300 nm. In some embodiments, the nanoparticles have an average diameter of from about 30 nm to about 250 nm. In some embodiments, the nanoparticles have an average diameter of from about 30 nm to about 240 nm. In some embodiments, the nanoparticles have an average diameter of from about 30 nm to about 230 nm. In some embodiments, the nanoparticles have an average diameter of from about 30 nm to about 220 nm. In some embodiments, the nanoparticles have an average diameter of from about 30 nm to about 210 nm. In some embodiments, the nanoparticles have an average diameter of from about 30 nm to about 200 nm. In some embodiments, the nanoparticles have an average diameter of from about 30 nm to about 190 nm. In some embodiments, the nanoparticles have an average diameter of from about 30 nm to about 180 nm. In some embodiments, the nanoparticles have an average diameter of from about 30 nm to about 170 nm. In some embodiments, the nanoparticles have an average diameter of from about 30 nm to about 160 nm. In some embodiments, the nanoparticles have an average diameter of from about 30 nm to about 150 nm. In some embodiments, the nanoparticles have an average diameter of from about 30 nm to about 140 nm. In some embodiments, the nanoparticles have an average diameter of from about 30 nm to about 130 nm. In some embodiments, the nanoparticles have an average diameter of from about 30 nm to about 120 nm. In some embodiments, the nanoparticles have an average diameter of from about 30 nm to about 110 nm. In some embodiments, the nanoparticles have an average diameter of from about 30 nm to about 100 nm. In some embodiments, the nanoparticles have an average diameter of from about 30 nm to about 90 nm. In some embodiments, the nanoparticles have an average diameter of from about 30 nm to about 80 nm. In some embodiments, the nanoparticles have an average diameter of from about 30 nm to about 70 nm. In some embodiments, the nanoparticles have an average diameter of from about 30 nm to about 60 nm. In some embodiments, the nanoparticles have an average diameter of from about 30 nm to about 50 nm. In some embodiments, the nanoparticles have an average diameter of from about 30 nm to about 40 nm. In some embodiments, the nanoparticles have an average diameter of from about 30 nm to about 40 nm.

In some embodiments, the nanoparticles have an average diameter of from about 40 nm to about 1000 nm. In some embodiments, the nanoparticles have an average diameter of from about 40 nm to about 950 nm. In some embodiments, the nanoparticles have an average diameter of from about 40 nm to about 900 nm. In some embodiments, the nanoparticles have an average diameter of from about 40 nm to about 850 nm. In some embodiments, the nanoparticles have an average diameter of from about 40 nm to about 800 nm. In some embodiments, the nanoparticles have an average diameter of from about 40 nm to about 750 nm. In some embodiments, the nanoparticles have an average diameter of from about 40 nm to about 700 nm. In some embodiments, the nanoparticles have an average diameter of from about 40 nm to about 650 nm. In some embodiments, the nanoparticles have an average diameter of from about 40 nm to about 600 nm. In some embodiments, the nanoparticles have an average diameter of from about 40 nm to about 550 nm. In some embodiments, the nanoparticles have an average diameter of from about 40 nm to about 500 nm. In some embodiments, the nanoparticles have an average diameter of from about 40 nm to about 450 nm. In some embodiments, the nanoparticles have an average diameter of from about 40 nm to about 400 nm. In some embodiments, the nanoparticles have an average diameter of from about 40 nm to about 350 nm. In some embodiments, the nanoparticles have an average diameter of from about 40 nm to about 300 nm. In some embodiments, the nanoparticles have an average diameter of from about 40 nm to about 250 nm. In some embodiments, the nanoparticles have an average diameter of from about 40 nm to about 240 nm. In some embodiments, the nanoparticles have an average diameter of from about 40 nm to about 230 nm. In some embodiments, the nanoparticles have an average diameter of from about 40 nm to about 220 nm. In some embodiments, the nanoparticles have an average diameter of from about 40 nm to about 210 nm. In some embodiments, the nanoparticles have an average diameter of from about 40 nm to about 200 nm. In some embodiments, the nanoparticles have an average diameter of from about 40 nm to about 190 nm. In some embodiments, the nanoparticles have an average diameter of from about 40 nm to about 180 nm. In some embodiments, the nanoparticles have an average diameter of from about 40 nm to about 170 nm. In some embodiments, the nanoparticles have an average diameter of from about 40 nm to about 160 nm. In some embodiments, the nanoparticles have an average diameter of from about 40 nm to about 150 nm. In some embodiments, the nanoparticles have an average diameter of from about 40 nm to about 140 nm. In some embodiments, the nanoparticles have an average diameter of from about 40 nm to about 130 nm. In some embodiments, the nanoparticles have an average diameter of from about 40 nm to about 120 nm. In some embodiments, the nanoparticles have an average diameter of from about 40 nm to about 110 nm. In some embodiments, the nanoparticles have an average diameter of from about 40 nm to about 100 nm. In some embodiments, the nanoparticles have an average diameter of from about 40 nm to about 90 nm. In some embodiments, the nanoparticles have an average diameter of from about 40 nm to about 80 nm. In some embodiments, the nanoparticles have an average diameter of from about 40 nm to about 70 nm. In some embodiments, the nanoparticles have an average diameter of from about 40 nm to about 60 nm. In some embodiments, the nanoparticles have an average diameter of from about 40 nm to about 50 nm.

In some embodiments, the nanoparticles have an average diameter of from about 50 nm to about 1000 nm. In some embodiments, the nanoparticles have an average diameter of from about 50 nm to about 950 nm. In some embodiments, the nanoparticles have an average diameter of from about 50 nm to about 900 nm. In some embodiments, the nanoparticles have an average diameter of from about 50 nm to about 850 nm. In some embodiments, the nanoparticles have an average diameter of from about 50 nm to about 800 nm. In some embodiments, the nanoparticles have an average diameter of from about 50 nm to about 750 nm. In some embodiments, the nanoparticles have an average diameter of from about 50 nm to about 700 nm. In some embodiments, the nanoparticles have an average diameter of from about 50 nm to about 650 nm. In some embodiments, the nanoparticles have an average diameter of from about 50 nm to about 600 nm. In some embodiments, the nanoparticles have an average diameter of from about 50 nm to about 550 nm. In some embodiments, the nanoparticles have an average diameter of from about 50 nm to about 500 nm. In some embodiments, the nanoparticles have an average diameter of from about 50 nm to about 450 nm. In some embodiments, the nanoparticles have an average diameter of from about 50 nm to about 400 nm. In some embodiments, the nanoparticles have an average diameter of from about 50 nm to about 350 nm. In some embodiments, the nanoparticles have an average diameter of from about 50 nm to about 300 nm. In some embodiments, the nanoparticles have an average diameter of from about 50 nm to about 250 nm. In some embodiments, the nanoparticles have an average diameter of from about 50 nm to about 240 nm. In some embodiments, the nanoparticles have an average diameter of from about 50 nm to about 230 nm. In some embodiments, the nanoparticles have an average diameter of from about 50 nm to about 220 nm. In some embodiments, the nanoparticles have an average diameter of from about 50 nm to about 210 nm. In some embodiments, the nanoparticles have an average diameter of from about 50 nm to about 200 nm. In some embodiments, the nanoparticles have an average diameter of from about 50 nm to about 190 nm. In some embodiments, the nanoparticles have an average diameter of from about 50 nm to about 180 nm. In some embodiments, the nanoparticles have an average diameter of from about 50 nm to about 170 nm. In some embodiments, the nanoparticles have an average diameter of from about 50 nm to about 160 nm. In some embodiments, the nanoparticles have an average diameter of from about 50 nm to about 150 nm. In some embodiments, the nanoparticles have an average diameter of from about 50 nm to about 140 nm. In some embodiments, the nanoparticles have an average diameter of from about 50 nm to about 130 nm. In some embodiments, the nanoparticles have an average diameter of from about 50 nm to about 120 nm. In some embodiments, the nanoparticles have an average diameter of from about 50 nm to about 110 nm. In some embodiments, the nanoparticles have an average diameter of from about 50 nm to about 100 nm. In some embodiments, the nanoparticles have an average diameter of from about 50 nm to about 90 nm. In some embodiments, the nanoparticles have an average diameter of from about 50 nm to about 80 nm. In some embodiments, the nanoparticles have an average diameter of from about 50 nm to about 70 nm. In some embodiments, the nanoparticles have an average diameter of from about 50 nm to about 60 nm.

In some embodiments, the nanoparticles have an average diameter of about 10 nm. In some embodiments, the nanoparticles have an average diameter of about 20 nm. In some embodiments, the nanoparticles have an average diameter of about 30 nm. In some embodiments, the nanoparticles have an average diameter of about 40 nm. In some embodiments, the nanoparticles have an average diameter of about 50 nm. In some embodiments, the nanoparticles have an average diameter of about 60 nm. In some embodiments, the nanoparticles have an average diameter of about 70 nm. In some embodiments, the nanoparticles have an average diameter of about 80 nm. In some embodiments, the nanoparticles have an average diameter of about 90 nm. In some embodiments, the nanoparticles have an average diameter of about 100 nm. In some embodiments, the nanoparticles have an average diameter of about 110 nm. In some embodiments, the nanoparticles have an average diameter of about 120 nm. In some embodiments, the nanoparticles have an average diameter of about 130 nm. In some embodiments, the nanoparticles have an average diameter of about 140 nm. In some embodiments, the nanoparticles have an average diameter of about 150 nm. In some embodiments, the nanoparticles have an average diameter of about 160 nm. In some embodiments, the nanoparticles have an average diameter of about 170 nm. In some embodiments, the nanoparticles have an average diameter of about 180 nm. In some embodiments, the nanoparticles have an average diameter of about 190 nm. In some embodiments, the nanoparticles have an average diameter of about 200 nm. In some embodiments, the nanoparticles have an average diameter of about 210 nm. In some embodiments, the nanoparticles have an average diameter of about 220 nm. In some embodiments, the nanoparticles have an average diameter of about 230 nm. In some embodiments, the nanoparticles have an average diameter of about 240 nm. In some embodiments, the nanoparticles have an average diameter of about 250 nm. In some embodiments, the nanoparticles have an average diameter of about 300 nm. In some embodiments, the nanoparticles have an average diameter of about 350 nm. In some embodiments, the nanoparticles have an average diameter of about 400 nm. In some embodiments, the nanoparticles have an average diameter of about 450 nm. In some embodiments, the nanoparticles have an average diameter of about 500 nm. In some embodiments, the nanoparticles have an average diameter of about 550 nm. In some embodiments, the nanoparticles have an average diameter of about 600 nm. In some embodiments, the nanoparticles have an average diameter of about 650 nm. In some embodiments, the nanoparticles have an average diameter of about 700 nm. In some embodiments, the nanoparticles have an average diameter of about 750 nm. In some embodiments, the nanoparticles have an average diameter of about 800 nm. In some embodiments, the nanoparticles have an average diameter of about 850 nm. In some embodiments, the nanoparticles have an average diameter of about 900 nm. In some embodiments, the nanoparticles have an average diameter of about 950 nm. In some embodiments, the nanoparticles have an average diameter of about 1000 nm.

In some embodiments, the composition is sterile filterable. In some embodiments, the nanoparticles have an average diameter of about 250 nm or less. In some embodiments, the nanoparticles have an average diameter of about 240 nm or less. In some embodiments, the nanoparticles have an average diameter of about 230 nm or less. In some embodiments, the nanoparticles have an average diameter of about 220 nm or less. In some embodiments, the nanoparticles have an average diameter of about 210 nm or less. In some embodiments, the nanoparticles have an average diameter of about 200 nm or less. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm.

In some embodiments, the nanoparticles are suspended, dissolved, or emulsified in a liquid. In some embodiments, the nanoparticles are suspended in a liquid. In some embodiments, the nanoparticles are dissolved in a liquid. In some embodiments, the nanoparticles are emulsified in a liquid.

In some embodiments, the nanoparticles are cross-linked using glutaraldehyde, glucose, or UV irradiation.

### Dehydrated Composition

In some embodiments, the composition is dehydrated. In some embodiments, the composition is a lyophilized composition. In some embodiments, the dehydrated composition comprises less than about 10%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water. In some embodiments, the dehydrated composition comprises less than about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water.

In some embodiments, when the composition is dehydrated composition, such as a lyophilized composition, the composition comprises from about 0.1% to about 99% by weight of the compound. In some embodiments, the composition comprises from about 0.1% to about 75% by weight of the compound. In some embodiments, the composition comprises from about 0.1% to about 50% by weight of the compound. In some embodiments, the composition comprises from about 0.1% to about 25% by weight of the compound. In some embodiments, the composition comprises from about 0.1% to about 20% by weight of the compound. In some embodiments, the composition comprises from about 0.1% to about 15% by weight of the compound. In some embodiments, the composition comprises from about 0.1% to about 10% by weight of the compound.

In some embodiments, when the composition is dehydrated composition, such as a lyophilized composition, the composition comprises from about 0.5% to about 99% by weight of the compound. In some embodiments, the composition comprises from about 0.5% to about 75% by weight of the compound. In some embodiments, the composition comprises from about 0.5% to about 50% by weight of the compound. In some embodiments, the composition comprises from about 0.5% to about 25% by weight of the compound. In some embodiments, the composition comprises from about 0.5% to about 20% by weight of the compound. In some embodiments, the composition comprises from about 0.5% to about 15% by weight of the compound. In some embodiments, the composition comprises from about 0.5% to about 10% by weight of the compound.

In some embodiments, when the composition is dehydrated composition, such as a lyophilized composition, the composition comprises from about 0.9% to about 24% by weight of the compound. In some embodiments, the composition comprises from about 1.8% to about 16% by weight of the compound.

In some embodiments, when the composition is dehydrated composition, such as a lyophilized composition, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by weight of the compound. In some embodiments, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the compound. In some embodiments, the composition comprises about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, or about 24% by weight of the compound. In some embodiments, the composition comprises about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, or about 16% by weight of the compound.

In some embodiments, when the composition is dehydrated composition, such as a lyophilized composition, the composition comprises from about 50% to about 99% by weight of the pharmaceutically acceptable carrier. In some embodiments, the composition comprises from about 55% to about 99% by weight of the pharmaceutically acceptable carrier. In some embodiments, the composition comprises from about 60% to about 99% by weight of the pharmaceutically acceptable carrier. In some embodiments, the composition comprises from about 65% to about 99% by weight of the pharmaceutically acceptable carrier. In some embodiments, the composition comprises from about 70% to about 99% by weight of the pharmaceutically acceptable carrier. In some embodiments, the composition comprises from about 75% to about 99% by weight of the pharmaceutically acceptable carrier. In some embodiments, the composition comprises from about 80% to about 99% by weight of the pharmaceutically acceptable carrier. In some embodiments, the composition comprises from about 85% to about 99% by weight of the pharmaceutically acceptable carrier. In some embodiments, the composition comprises from about 90% to about 99% by weight of the pharmaceutically acceptable carrier.

In some embodiments, when the composition is dehydrated composition, such as a lyophilized composition, the composition comprises from about 76% to about 99% by weight of the pharmaceutically acceptable carrier. In some embodiments, the composition comprises from about 84% to about 98% by weight of the pharmaceutically acceptable carrier.

In some embodiments, when the composition is dehydrated composition, such as a lyophilized composition, the composition comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier.

### Reconstitution

In some embodiments, the composition is reconstituted with an appropriate biocompatible liquid to provide a reconstituted composition. In some embodiments, appropriate biocompatible liquid is a buffered solution. Examples of suitable buffered solutions include, but are not limited to, buffered solutions of amino acids, buffered solutions of proteins, buffered solutions of sugars, buffered solutions of vitamins, buffered solutions of synthetic polymers, buffered solutions of salts (such as buffered saline or buffered aqueous media), any similar buffered solutions, or any suitable combination thereof. In some embodiments, the appropriate biocompatible liquid is a solution comprising dextrose. In some embodiments, the appropriate biocompatible liquid is a solution comprising one or more salts. In some embodiments, the appropriate biocompatible liquid is a solution suitable for intravenous use. Examples of solutions that are suitable for intravenous use, include, but are not limited to, balanced solutions, which are different solutions with different electrolyte compositions that are close to plasma composition. Such electrolyte compositions comprise crystalloids or colloids. Examples of suitable appropriate biocompatible liquids include, but are not limited to, sterile water, saline, phosphate-buffered saline, 5% dextrose in water solution, Ringer's solution, or Ringer's lactate solution. In some embodiments, the appropriate biocompatible liquid is sterile water, saline, phosphate-buffered saline, 5% dextrose in water solution, Ringer's solution, or Ringer's lactate solution. In some embodiments, the appropriate biocompatible liquid is sterile water. In some embodiments, the appropriate biocompatible liquid is saline. In some embodiments, the appropriate biocompatible liquid is phosphate-buffered saline. In some embodiments, the appropriate biocompatible liquid is 5% dextrose in water solution. In some embodiments, the appropriate biocompatible liquid is Ringer's solution. In some embodiments, the appropriate biocompatible liquid is Ringer's lactate solution. In some embodiments, the appropriate biocompatible liquid is a balanced solution, or a solution with an electrolyte composition that resembles plasma.

In some embodiments, the nanoparticles have an average diameter of from about 10 nm to about 1000 nm after reconstitution. In some embodiments, the nanoparticles have an average diameter of from about 10 nm to about 950 nm after reconstitution. In some embodiments, the nanoparticles have an average diameter of from about 10 nm to about 900 nm after reconstitution. In some embodiments, the nanoparticles have an average diameter of from about 10 nm to about 850 nm after reconstitution. In some embodiments, the nanoparticles have an average diameter of from about 10 nm to about 800 nm after reconstitution. In some embodiments, the nanoparticles have an average diameter of from about 10 nm to about 750 nm after reconstitution. In some embodiments, the nanoparticles have an average diameter of from about 10 nm to about 700 nm after reconstitution. In some embodiments, the nanoparticles have an average diameter of from about 10 nm to about 650 nm after reconstitution. In some embodiments, the nanoparticles have an average diameter of from about 10 nm to about 600 nm after reconstitution. In some embodiments, the nanoparticles have an average diameter of from about 10 nm to about 550 nm after reconstitution. In some embodiments, the nanoparticles have an average diameter of from about 10 nm to about 500 nm after reconstitution. In some embodiments, the nanoparticles have an average diameter of from about 10 nm to about 450 nm after reconstitution. In some embodiments, the nanoparticles have an average diameter of from about 10 nm to about 400 nm after reconstitution. In some embodiments, the nanoparticles have an average diameter of from about 10 nm to about 350 nm after reconstitution. In some embodiments, the nanoparticles have an average diameter of from about 10 nm to about 300 nm after reconstitution. In some embodiments, the nanoparticles have an average diameter of from about 10 nm to about 250 nm after reconstitution. In some embodiments, the nanoparticles have an average diameter of from about 10 nm to about 240 nm after reconstitution. In some embodiments, the nanoparticles have an average diameter of from about 10 nm to about 230 nm after reconstitution. In some embodiments, the nanoparticles have an average diameter of from about 10 nm to about 220 nm after reconstitution. In some embodiments, the nanoparticles have an average diameter of from about 10 nm to about 210 nm after reconstitution. In some embodiments, the nanoparticles have an average diameter of from about 10 nm to about 200 nm after reconstitution. In some embodiments, the nanoparticles have an average diameter of from about 10 nm to about 190 nm after reconstitution. In some embodiments, the nanoparticles have an average diameter of from about 10 nm to about 180 nm after reconstitution. In some embodiments, the nanoparticles have an average diameter of from about 10 nm to about 170 nm after reconstitution. In some embodiments, the nanoparticles have an average diameter of from about 10 nm to about 160 nm after reconstitution. In some embodiments, the nanoparticles have an average diameter of from about 10 nm to about 150 nm after reconstitution. In some embodiments, the nanoparticles have an average diameter of from about 10 nm to about 140 nm after reconstitution. In some embodiments, the nanoparticles have an average diameter of from about 10 nm to about 130 nm after reconstitution. In some embodiments, the nanoparticles have an average diameter of from about 10 nm to about 120 nm after reconstitution. In some embodiments, the nanoparticles have an average diameter of from about 10 nm to about 110 nm after reconstitution. In some embodiments, the nanoparticles have an average diameter of from about 10 nm to about 100 nm after reconstitution. In some embodiments, the nanoparticles have an average diameter of from about 10 nm to about 90 nm after reconstitution. In some embodiments, the nanoparticles have an average diameter of from about 10 nm to about 80 nm after reconstitution. In some embodiments, the nanoparticles have an average diameter of from about 10 nm to about 70 nm after reconstitution. In some embodiments, the nanoparticles have an average diameter of from about 10 nm to about 60 nm after reconstitution. In some embodiments, the nanoparticles have an average diameter of from about 10 nm to about 50 nm after reconstitution. In some embodiments, the nanoparticles have an average diameter of from about 10 nm to about 40 nm after reconstitution. In some embodiments, the nanoparticles have an average diameter of from about 10 nm to about 30 nm after reconstitution. In some embodiments, the nanoparticles have an average diameter of from about 10 nm to about 20 nm after reconstitution.

In some embodiments, the nanoparticles have an average diameter of from about 20 nm to about 1000 nm after reconstitution. In some embodiments, the nanoparticles have an average diameter of from about 20 nm to about 950 nm after reconstitution. In some embodiments, the nanoparticles have an average diameter of from about 20 nm to about 900 nm after reconstitution. In some embodiments, the nanoparticles have an average diameter of from about 20 nm to about 850 nm after reconstitution. In some embodiments, the nanoparticles have an average diameter of from about 20 nm to about 800 nm after reconstitution. In some embodiments, the nanoparticles have an average diameter of from about 20 nm to about 750 nm after reconstitution. In some embodiments, the nanoparticles have an average diameter of from about 20 nm to about 700 nm after reconstitution. In some embodiments, the nanoparticles have an average diameter of from about 20 nm to about 650 nm after reconstitution. In some embodiments, the nanoparticles have an average diameter of from about 20 nm to about 600 nm after reconstitution. In some embodiments, the nanoparticles have an average diameter of from about 20 nm to about 550 nm after reconstitution. In some embodiments, the nanoparticles have an average diameter of from about 20 nm to about 500 nm after reconstitution. In some embodiments, the nanoparticles have an average diameter of from about 20 nm to about 450 nm after reconstitution. In some embodiments, the nanoparticles have an average diameter of from about 20 nm to about 400 nm after reconstitution. In some embodiments, the nanoparticles have an average diameter of from about 20 nm to about 350 nm after reconstitution. In some embodiments, the nanoparticles have an average diameter of from about 20 nm to about 300 nm after reconstitution. In some embodiments, the nanoparticles have an average diameter of from about 20 nm to about 250 nm after reconstitution. In some embodiments, the nanoparticles have an average diameter of from about 20 nm to about 240 nm after reconstitution. In some embodiments, the nanoparticles have an average diameter of from about 20 nm to about 230 nm after reconstitution. In some embodiments, the nanoparticles have an average diameter of from about 20 nm to about 220 nm after reconstitution. In some embodiments, the nanoparticles have an average diameter of from about 20 nm to about 210 nm after reconstitution. In some embodiments, the nanoparticles have an average diameter of from about 20 nm to about 200 nm after reconstitution. In some embodiments, the nanoparticles have an average diameter of from about 20 nm to about 190 nm after reconstitution. In some embodiments, the nanoparticles have an average diameter of from about 20 nm to about 180 nm after reconstitution. In some embodiments, the nanoparticles have an average diameter of from about 20 nm to about 170 nm after reconstitution. In some embodiments, the nanoparticles have an average diameter of from about 20 nm to about 160 nm after reconstitution. In some embodiments, the nanoparticles have an average diameter of from about 20 nm to about 150 nm after reconstitution. In some embodiments, the nanoparticles have an average diameter of from about 20 nm to about 140 nm after reconstitution. In some embodiments, the nanoparticles have an average diameter of from about 20 nm to about 130 nm after reconstitution. In some embodiments, the nanoparticles have an average diameter of from about 20 nm to about 120 nm after reconstitution. In some embodiments, the nanoparticles have an average diameter of from about 20 nm to about 110 nm after reconstitution. In some embodiments, the nanoparticles have an average diameter of from about 20 nm to about 100 nm after reconstitution. In some embodiments, the nanoparticles have an average diameter of from about 20 nm to about 90 nm after reconstitution. In some embodiments, the nanoparticles have an average diameter of from about 20 nm to about 80 nm after reconstitution. In some embodiments, the nanoparticles have an average diameter of from about 20 nm to about 70 nm after reconstitution. In some embodiments, the nanoparticles have an average diameter of from about 20 nm to about 60 nm after reconstitution. In some embodiments, the nanoparticles have an average diameter of from about 20 nm to about 50 nm after reconstitution. In some embodiments, the nanoparticles have an average diameter of from about 20 nm to about 40 nm after reconstitution. In some embodiments, the nanoparticles have an average diameter of from about 20 nm to about 30 nm after reconstitution.

In some embodiments, the nanoparticles have an average diameter of from about 30 nm to about 1000 nm after reconstitution. In some embodiments, the nanoparticles have an average diameter of from about 30 nm to about 950 nm after reconstitution. In some embodiments, the nanoparticles have an average diameter of from about 30 nm to about 900 nm after reconstitution. In some embodiments, the nanoparticles have an average diameter of from about 30 nm to about 850 nm after reconstitution. In some embodiments, the nanoparticles have an average diameter of from about 30 nm to about 800 nm after reconstitution. In some embodiments, the nanoparticles have an average diameter of from about 30 nm to about 750 nm after reconstitution. In some embodiments, the nanoparticles have an average diameter of from about 30 nm to about 700 nm after reconstitution. In some embodiments, the nanoparticles have an average diameter of from about 30 nm to about 650 nm after reconstitution. In some embodiments, the nanoparticles have an average diameter of from about 30 nm to about 600 nm after reconstitution. In some embodiments, the nanoparticles have an average diameter of from about 30 nm to about 550 nm after reconstitution. In some embodiments, the nanoparticles have an average diameter of from about 30 nm to about 500 nm. In some embodiments, the nanoparticles have an average diameter of from about 30 nm to about 450 nm after reconstitution. In some embodiments, the nanoparticles have an average diameter of from about 30 nm to about 400 nm after reconstitution. In some embodiments, the nanoparticles have an average diameter of from about 30 nm to about 350 nm after reconstitution. In some embodiments, the nanoparticles have an average diameter of from about 30 nm to about 300 nm after reconstitution. In some embodiments, the nanoparticles have an average diameter of from about 30 nm to about 250 nm after reconstitution. In some embodiments, the nanoparticles have an average diameter of from about 30 nm to about 240 nm after reconstitution. In some embodiments, the nanoparticles have an average diameter of from about 30 nm to about 230 nm after reconstitution. In some embodiments, the nanoparticles have an average diameter of from about 30 nm to about 220 nm after reconstitution. In some embodiments, the nanoparticles have an average diameter of from about 30 nm to about 210 nm after reconstitution. In some embodiments, the nanoparticles have an average diameter of from about 30 nm to about 200 nm after reconstitution. In some embodiments, the nanoparticles have an average diameter of from about 30 nm to about 190 nm after reconstitution. In some embodiments, the nanoparticles have an average diameter of from about 30 nm to about 180 nm after reconstitution. In some embodiments, the nanoparticles have an average diameter of from about 30 nm to about 170 nm after reconstitution. In some embodiments, the nanoparticles have an average diameter of from about 30 nm to about 160 nm after reconstitution. In some embodiments, the nanoparticles have an average diameter of from about 30 nm to about 150 nm after reconstitution. In some embodiments, the nanoparticles have an average diameter of from about 30 nm to about 140 nm after reconstitution. In some embodiments, the nanoparticles have an average diameter of from about 30 nm to about 130 nm after reconstitution. In some embodiments, the nanoparticles have an average diameter of from about 30 nm to about 120 nm after reconstitution. In some embodiments, the nanoparticles have an average diameter of from about 30 nm to about 110 nm after reconstitution. In some embodiments, the nanoparticles have an average diameter of from about 30 nm to about 100 nm after reconstitution. In some embodiments, the nanoparticles have an average diameter of from about 30 nm to about 90 nm after reconstitution. In some embodiments, the nanoparticles have an average diameter of from about 30 nm to about 80 nm after reconstitution. In some embodiments, the nanoparticles have an average diameter of from about 30 nm to about 70 nm after reconstitution. In some embodiments, the nanoparticles have an average diameter of from about 30 nm to about 60 nm after reconstitution. In some embodiments, the nanoparticles have an average diameter of from about 30 nm to about 50 nm after reconstitution. In some embodiments, the nanoparticles have an average diameter of from about 30 nm to about 40 nm after reconstitution.

In some embodiments, the nanoparticles have an average diameter of from about 40 nm to about 1000 nm after reconstitution. In some embodiments, the nanoparticles have an average diameter of from about 40 nm to about 950 nm after reconstitution. In some embodiments, the nanoparticles have an average diameter of from about 40 nm to about 900 nm after reconstitution. In some embodiments, the nanoparticles have an average diameter of from about 40 nm to about 850 nm after reconstitution. In some embodiments, the nanoparticles have an average diameter of from about 40 nm to about 800 nm after reconstitution. In some embodiments, the nanoparticles have an average diameter of from about 40 nm to about 750 nm after reconstitution. In some embodiments, the nanoparticles have an average diameter of from about 40 nm to about 700 nm after reconstitution. In some embodiments, the nanoparticles have an average diameter of from about 40 nm to about 650 nm after reconstitution. In some embodiments, the nanoparticles have an average diameter of from about 40 nm to about 600 nm after reconstitution. In some embodiments, the nanoparticles have an average diameter of from about 40 nm to about 550 nm after reconstitution. In some embodiments, the nanoparticles have an average diameter of from about 40 nm to about 500 nm after reconstitution. In some embodiments, the nanoparticles have an average diameter of from about 40 nm to about 450 nm after reconstitution. In some embodiments, the nanoparticles have an average diameter of from about 40 nm to about 400 nm after reconstitution. In some embodiments, the nanoparticles have an average diameter of from about 40 nm to about 350 nm after reconstitution. In some embodiments, the nanoparticles have an average diameter of from about 40 nm to about 300 nm. In some embodiments, the nanoparticles have an average diameter of from about 40 nm to about 250 nm after reconstitution. In some embodiments, the nanoparticles have an average diameter of from about 40 nm to about 240 nm after reconstitution. In some embodiments, the nanoparticles have an average diameter of from about 40 nm to about 230 nm after reconstitution. In some embodiments, the nanoparticles have an average diameter of from about 40 nm to about 220 nm after reconstitution. In some embodiments, the nanoparticles have an average diameter of from about 40 nm to about 210 nm after reconstitution. In some embodiments, the nanoparticles have an average diameter of from about 40 nm to about 200 nm after reconstitution. In some embodiments, the nanoparticles have an average diameter of from about 40 nm to about 190 nm after reconstitution. In some embodiments, the nanoparticles have an average diameter of from about 40 nm to about 180 nm after reconstitution. In some embodiments, the nanoparticles have an average diameter of from about 40 nm to about 170 nm after reconstitution. In some embodiments, the nanoparticles have an average diameter of from about 40 nm to about 160 nm after reconstitution. In some embodiments, the nanoparticles have an average diameter of from about 40 nm to about 150 nm after reconstitution. In some embodiments, the nanoparticles have an average diameter of from about 40 nm to about 140 nm after reconstitution. In some embodiments, the nanoparticles have an average diameter of from about 40 nm to about 130 nm after reconstitution. In some embodiments, the nanoparticles have an average diameter of from about 40 nm to about 120 nm after reconstitution. In some embodiments, the nanoparticles have an average diameter of from about 40 nm to about 110 nm after reconstitution. In some embodiments, the nanoparticles have an average diameter of from about 40 nm to about 100 nm after reconstitution. In some embodiments, the nanoparticles have an average diameter of from about 40 nm to about 90 nm after reconstitution. In some embodiments, the nanoparticles have an average diameter of from about 40 nm to about 80 nm after reconstitution. In some embodiments, the nanoparticles have an average diameter of from about 40 nm to about 70 nm after reconstitution. In some embodiments, the nanoparticles have an average diameter of from about 40 nm to about 60 nm after reconstitution. In some embodiments, the nanoparticles have an average diameter of from about 40 nm to about 50 nm after reconstitution.

In some embodiments, the nanoparticles have an average diameter of from about 50 nm to about 1000 nm after reconstitution. In some embodiments, the nanoparticles have an average diameter of from about 50 nm to about 950 nm after reconstitution. In some embodiments, the nanoparticles have an average diameter of from about 50 nm to about 900 nm after reconstitution. In some embodiments, the nanoparticles have an average diameter of from about 50 nm to about 850 nm after reconstitution. In some embodiments, the nanoparticles have an average diameter of from about 50 nm to about 800 nm after reconstitution. In some embodiments, the nanoparticles have an average diameter of from about 50 nm to about 750 nm after reconstitution. In some embodiments, the nanoparticles have an average diameter of from about 50 nm to about 700 nm after reconstitution. In some embodiments, the nanoparticles have an average diameter of from about 50 nm to about 650 nm after reconstitution. In some embodiments, the nanoparticles have an average diameter of from about 50 nm to about 600 nm after reconstitution. In some embodiments, the nanoparticles have an average diameter of from about 50 nm to about 550 nm after reconstitution. In some embodiments, the nanoparticles have an average diameter of from about 50 nm to about 500 nm after reconstitution. In some embodiments, the nanoparticles have an average diameter of from about 50 nm to about 450 nm after reconstitution. In some embodiments, the nanoparticles have an average diameter of from about 50 nm to about 400 nm after reconstitution. In some embodiments, the nanoparticles have an average diameter of from about 50 nm to about 350 nm after reconstitution. In some embodiments, the nanoparticles have an average diameter of from about 50 nm to about 300 nm after reconstitution. In some embodiments, the nanoparticles have an average diameter of from about 50 nm to about 250 nm after reconstitution. In some embodiments, the nanoparticles have an average diameter of from about 50 nm to about 240 nm after reconstitution. In some embodiments, the nanoparticles have an average diameter of from about 50 nm to about 230 nm after reconstitution. In some embodiments, the nanoparticles have an average diameter of from about 50 nm to about 220 nm after reconstitution. In some embodiments, the nanoparticles have an average diameter of from about 50 nm to about 210 nm after reconstitution. In some embodiments, the nanoparticles have an average diameter of from about 50 nm to about 200 nm after reconstitution. In some embodiments, the nanoparticles have an average diameter of from about 50 nm to about 190 nm after reconstitution. In some embodiments, the nanoparticles have an average diameter of from about 50 nm to about 180 nm after reconstitution. In some embodiments, the nanoparticles have an average diameter of from about 50 nm to about 170 nm after reconstitution. In some embodiments, the nanoparticles have an average diameter of from about 50 nm to about 160 nm after reconstitution. In some embodiments, the nanoparticles have an average diameter of from about 50 nm to about 150 nm after reconstitution. In some embodiments, the nanoparticles have an average diameter of from about 50 nm to about 140 nm after reconstitution. In some embodiments, the nanoparticles have an average diameter of from about 50 nm to about 130 nm after reconstitution. In some embodiments, the nanoparticles have an average diameter of from about 50 nm to about 120 nm after reconstitution. In some embodiments, the nanoparticles have an average diameter of from about 50 nm to about 110 nm after reconstitution. In some embodiments, the nanoparticles have an average diameter of from about 50 nm to about 100 nm after reconstitution. In some embodiments, the nanoparticles have an average diameter of from about 50 nm to about 90 nm after reconstitution. In some embodiments, the nanoparticles have an average diameter of from about 50 nm to about 80 nm after reconstitution. In some embodiments, the nanoparticles have an average diameter of from about 50 nm to about 70 nm after reconstitution. In some embodiments, the nanoparticles have an average diameter of from about 50 nm to about 60 nm after reconstitution.

In some embodiments, the nanoparticles have an average diameter of about 10 nm after reconstitution. In some embodiments, the nanoparticles have an average diameter of about 20 nm after reconstitution. In some embodiments, the nanoparticles have an average diameter of about 30 nm after reconstitution. In some embodiments, the nanoparticles have an average diameter of about 40 nm after reconstitution. In some embodiments, the nanoparticles have an average diameter of about 50 nm after reconstitution. In some embodiments, the nanoparticles have an average diameter of about 60 nm after reconstitution. In some embodiments, the nanoparticles have an average diameter of about 70 nm after reconstitution. In some embodiments, the nanoparticles have an average diameter of about 80 nm after reconstitution. In some embodiments, the nanoparticles have an average diameter of about 90 nm after reconstitution. In some embodiments, the nanoparticles have an average diameter of about 100 nm after reconstitution. In some embodiments, the nanoparticles have an average diameter of about 110 nm after reconstitution. In some embodiments, the nanoparticles have an average diameter of about 120 nm after reconstitution. In some embodiments, the nanoparticles have an average diameter of about 130 nm after reconstitution. In some embodiments, the nanoparticles have an average diameter of about 140 nm after reconstitution. In some embodiments, the nanoparticles have an average diameter of about 150 nm after reconstitution. In some embodiments, the nanoparticles have an average diameter of about 160 nm after reconstitution. In some embodiments, the nanoparticles have an average diameter of about 170 nm after reconstitution. In some embodiments, the nanoparticles have an average diameter of about 180 nm. In some embodiments, the nanoparticles have an average diameter of about 190 nm after reconstitution. In some embodiments, the nanoparticles have an average diameter of about 200 nm after reconstitution. In some embodiments, the nanoparticles have an average diameter of about 210 nm after reconstitution. In some embodiments, the nanoparticles have an average diameter of about 220 nm after reconstitution. In some embodiments, the nanoparticles have an average diameter of about 230 nm after reconstitution. In some embodiments, the nanoparticles have an average diameter of about 240 nm after reconstitution. In some embodiments, the nanoparticles have an average diameter of about 250 nm after reconstitution. In some embodiments, the nanoparticles have an average diameter of about 300 nm after reconstitution. In some embodiments, the nanoparticles have an average diameter of about 350 nm after reconstitution. In some embodiments, the nanoparticles have an average diameter of about 400 nm after reconstitution. In some embodiments, the nanoparticles have an average diameter of about 450 nm after reconstitution. In some embodiments, the nanoparticles have an average diameter of about 500 nm after reconstitution. In some embodiments, the nanoparticles have an average diameter of about 550 nm after reconstitution. In some embodiments, the nanoparticles have an average diameter of about 600 nm after reconstitution. In some embodiments, the nanoparticles have an average diameter of about 650 nm after reconstitution. In some embodiments, the nanoparticles have an average diameter of about 700 nm after reconstitution. In some embodiments, the nanoparticles have an average diameter of about 750 nm after reconstitution. In some embodiments, the nanoparticles have an average diameter of about 800 nm. In some embodiments, the nanoparticles have an average diameter of about 850 nm after reconstitution. In some embodiments, the nanoparticles have an average diameter of about 900 nm after reconstitution. In some embodiments, the nanoparticles have an average diameter of about 950 nm after reconstitution. In some embodiments, the nanoparticles have an average diameter of about 1000 nm after reconstitution.

### Preparation of Nanoparticles

Provided in another aspect is a process of preparing any one of the compositions comprising the nanoparticles described herein, comprising:
a) dissolving a compound of Formula (I) or Formula (II) in a volatile solvent to form a solution comprising a dissolved compound of Formula (I) or Formula (II);
b) adding the solution comprising the dissolved compound of Formula (I) or Formula (II) to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the any one of the compositions described herein.

In some embodiments, the adding the solution comprising the dissolved compound of Formula (I) or Formula (II) to a pharmaceutically acceptable carrier in an aqueous solution from step b) further comprises mixing to form an emulsion. In some embodiments, the mixing is performed with a homogenizer. In some embodiments, the volatile solvent is a chlorinated solvent, alcohol, ketone, ester, ether, acetonitrile, or any combination thereof. In some embodiments, volatile solvent is a chlorinated solvent. Examples of chlorinated solvents include, but are not limited to, chloroform, dichloromethane, and 1,2, dichloroethane. In some embodiments, volatile solvent is an alcohol. Examples of alcohols, include but are not limited to, methanol, ethanol, butanol (such as t-butyl and n-butyl alcohol), and propanol (such as iso-propyl alcohol). In some embodiments, volatile solvent is a ketone. An example of a ketone includes, but is not limited to, acetone. In some embodiments, volatile solvent is an ester. An example of an ester includes, but is not limited to ethyl acetate. In some embodiments, volatile solvent is an ether. In some embodiments, the volatile solvent is acetonitrile. In some embodiments, the volatile solvent is mixture of a chlorinated solvent with an alcohol.

In some embodiments, the volatile solvent is chloroform, ethanol, butanol, methanol, propanol, or a combination thereof. In some embodiments, volatile solvent is a mixture of chloroform and ethanol. In some embodiments, the volatile solvent is methanol. In some embodiments, the volatile solvent is a mixture of chloroform and methanol. In some embodiments, the volatile solvent is butanol, such as t-butanol or n-butanol. In some embodiments, the volatile solvent is a mixture of chloroform and butanol. In some embodiments, the volatile solvent is acetone. In some embodiments, the volatile solvent is acetonitrile. In some embodiments, the volatile solvent is dichloromethane. In some embodiments, the volatile solvent is 1,2 dichloroethane. In some embodiments the volatile solvent is ethyl acetate. In some embodiments, the volatile solvent is isopropyl alcohol. In some embodiments, the volatile solvent is chloroform. In some embodiments, the volatile solvent is ethanol. In some embodiments, the volatile solvent is a combination of ethanol and chloroform.

In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the appropriate amount of cycles is about 1, about 2, about 3, about 4, about 5, about 6, about 7, about 8, about 9, or about 10 cycles.

In some embodiments, the evaporation is accomplished with suitable equipment known for this purpose. Such suitable equipment include, but not limited to, rotary evaporators, falling film evaporators, wiped film evaporators, spray driers, and the like that can be operated in batch mode or in continuous operation. In some embodiments, the evaporation is accomplished with a rotary evaporator. In some embodiments, the evaporation is under reduced pressure.

### Administration

In some embodiments, the composition is suitable for injection. In some embodiments, the composition is suitable for parenteral administration. Examples of parenteral administration include but are not limited to subcutaneous injections, intravenous, or intramuscular injections or infusion techniques. In some embodiments, the composition is suitable for intravenous administration.

In some embodiments, the composition is administered intraperitoneally, intraarterially, intrapulmonarily, orally, by inhalation, intravesicularly, intramuscularly, intratracheally, subcutaneously, intraocularly, intratumorally, intrathecally, or transdermally. In some embodiments, the composition is administered intravenously. In some embodiments, the composition is administered intraarterially. In some embodiments, the composition is administered intrapulmonarily. In some embodiments, the composition is administered orally. In some embodiments, the composition is administered by inhalation. In some embodiments, the composition is administered intravesicularly. In some embodiments, the composition is administered intramuscularly. In some embodiments, the composition is administered intratracheally. In some embodiments, the composition is administered subcutaneously. In some embodiments, the composition is administered intraocularly. In some embodiments, the composition is administered intrathecally. In some embodiments, the composition is administered transdermally.

### Methods

Also provided herein in another aspect is a method of treating a disease in a subject in need thereof comprising administering any one of the compositions described herein.

In some embodiments, disease is cancer. Examples of cancers, include but not limited to solid tumors (e.g., tumors of the lung, breast, colon, prostate, bladder, rectum, brain or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, multiple myeloma, mesothelioma, childhood solid tumors such as brain neuroblastoma, retinoblastoma, Wilms' tumor, bone cancer and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma and retinoblastoma. In some embodiments, the cancer is breast cancer, ovarian cancer, non-small cell lung cancer, pancreatic cancer, or bladder cancer.

In some embodiments, the disease is caused by an infection. In some embodiments, the infection is viral. Examples of viral infection include, but are not limited to, picornaviruses (poliovirus, coxsackievirus, hepatitis A virus, echovirus, human rhinovirus, cardioviruses (e.g. mengovirus and encephalomyocarditis virus) and foot-and-mouth disease virus); immunodeficiency virus (e.g., HIV-1, HIV-2 and related viruses including FIV-1 and SIV-1); hepatitis B virus (HBV); papillomavirus; Epstein-Barr virus (EBV); T-cell leukemia virus, e.g., HTLV-I, HTLV-II and related viruses, including bovine leukemia virus (BLV) and simian T-cell leukemia virus (STLV-I); hepatitis C virus (HCV); cytomegalovirus (CMV); influenza virus; herpes simplex virus (HSV). In some embodiments, the viral infection is human immunodeficiency virus (HIV), hepatitis B virus (HBV), hepatitis C virus. (HCV), Epstein-Barr virus (EBV), cytomegalovirus (CMV), or herpes simplex virus (HSV).

In some embodiments, the compound is an anticancer agent. In some embodiments, the compound is an antiviral agent.

Also disclosed herein is a method of delivering a compound of Formula (I) or Formula (II) to a subject in need thereof comprising administering any one of the compositions described herein.

Disclosed compositions are administered to patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors, with the appropriate dosage ultimately being at the discretion of the attendant physician. For treating diseases noted above, a contemplated composition disclosed herein is administered orally, subcutaneously, topically, parenterally, by inhalation spray, or rectally in dosage unit formulations containing conventional non-toxic pharmaceutically acceptable carriers, adjuvants and vehicles. Parenteral administration include subcutaneous injections, intravenous, or intramuscular injections or infusion techniques.

The following examples are provided merely as illustrative of various embodiments and shall not be construed to limit the invention in any way.

### EXAMPLES

### List of Abbreviations

As used above, and throughout the description of the invention, the following abbreviations, unless otherwise indicated, shall be understood to have the following meanings:

ACN acetonitrile

Bn benzyl

BOC or Boc tert-butyl carbamate

DCC N,N′-dicyclohexylcarbodiimide

DCM dichloromethane

DIPEA N,N-diisopropylethylamine

DMAP 4-(N,N-dimethylamino)pyridine

DMF dimethylformamide

DMA N,N-dimethylacetamide

DMSO dimethylsulfoxide

equiv equivalent(s)

EDCI 1-(3-dimethyl aminopropyl)-3-ethylcarbodiimide

Et ethyl

EtOH ethanol

EtOAc ethyl acetate

HF hydrofluoric acid

HMDS bis(trimethylsilyl)amine

HPLC high performance liquid chromatography

Me methyl

MeOH methanol

MMTr 4-methoxytrityl

MMTrCl 4-methoxytrityl chloride

MS mass spectroscopy

NMM N-methylmorpholine

NMR nuclear magnetic resonance

TBHP tert-butyl hydroperoxide

TEA triethylamine

TFA trifluoroacetic acid

THF tetrahydrofuran

TLC thin layer chromatography

TBDMSCl tert-butyldimethylsilyl chloride

TMSCl trimethylsilyl chloride

TMSOTf trimethylsilyl trifluoromethanesulfonate

Examples of No Albumin Nanoparticles Produced with Unmodified Gemcitabine

Example 1

This example demonstrates the inability of unmodified Gemcitabine to form a nanoparticle with albumin. 39.2 mL of a human albumin solution (1.47% w/v) was prepared diluting from 25% human albumin U.S.P. solution with water. Gemcitabine (22 mg) was dissolved in 800 μL ethanol. The organic solvent solution was added dropwise to the albumin solution while homogenizing for 5 minutes at 5000 rpm (IKA Ultra-Turrax T 18 rotor-stator, S 18N-19G dispersing element) which was transferred into a high-pressure homogenizer (Avestin, Emulsiflex-C5) for 2 minutes at high pressure (12,000 psi to 20,000 psi) while cooling (4° to 10° C.). The resulting solution was transferred into a rotary evaporator (Buchi, Switzerland), where the ethanol was removed at 40° C. under reduced pressure (approximately 25 mm Hg) for 4 to 8 minutes. The solution was then sterile filtered, and the average particle size (Zₐᵥ, Malvern Nano-S) was determined to be less than 20 nm, with over 99.9% of particles the same as the input 4 nm diameter human albumin.

### Example 2

This example demonstrates the inability of unmodified Gemcitabine to form a nanoparticle with albumin. 39.2 mL of a human albumin solution (1.47% w/v) was prepared diluting from a 25% human albumin U.S.P. solution using chloroform saturated water. Gemcitabine (34 mg) was dissolved in 800 μL chloroform/methanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 to 8 minutes. The suspension was then sterile filtered, and the average particle size (Zₐᵥ, Malvern Nano-S) determined to be less than 20 nm, with over 99.9% of particles the same as the input 4 nm diameter human albumin.

### Example 3

This example demonstrates the inability of unmodified Gemcitabine to form a nanoparticle with albumin. 39.2 mL of a human albumin solution (1.47% w/v) was prepared diluting from a 25% human albumin U.S.P. solution using chloroform saturated water. Gemcitabine (17 mg) was dissolved in 800 μL chloroform/methanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 to 8 minutes. The suspension was then sterile filtered, and the average particle size (Zₐᵥ, Malvern Nano-S) determined to be less than 20 nm, with over 99.9% of particles the same as the input 4 nm diameter human albumin.

Examples of No Albumin Nanoparticles Produced with Some Gemcitabine Monophosphate Prodrugs

### Example 4

This example demonstrates the inability of the gemcitabine monophosphate prodrug Compound 1 to form a stable albumin nanoparticle. 39.2 mL of a human albumin solution (1.47% w/v) was prepared diluting from a 25% human albumin U.S.P. solution using chloroform saturated water. Compound 1 (43 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) determined to be less than 20 nm, with over 99.9% of particles the same as the input 4 nm diameter human albumin.

### Example 5

This example demonstrates the inability of the gemcitabine monophosphate prodrug Compound 2 to form a stable albumin nanoparticle. 39.2 mL of a human albumin solution (1.47% w/v) was prepared diluting from a 25% human albumin U.S.P. solution using chloroform saturated water. Compound 2 (43 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) determined to be less than 20 nm, with over 99.9% of particles the same as the input 4 nm diameter human albumin.

### Example 6

This example demonstrates the inability of the gemcitabine monophosphate prodrug Compound 3 to form a stable albumin nanoparticle. 39.2 mL of a human albumin solution (1.47% w/v) was prepared diluting from a 25% human albumin U.S.P. solution using chloroform saturated water. Compound 3 (45 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) determined to be less than 20 nm, with over 99.9% of particles the same as the input 4 nm diameter human albumin.

### Example 7

This example demonstrates the inability of the gemcitabine monophosphate prodrug Compound 4 to form a stable albumin nanoparticle. 19.6 mL of a human albumin solution (1.47% w/v) was prepared diluting from a 25% human albumin U.S.P. solution using chloroform saturated water. Compound 4 (24 mg) was dissolved in 400 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 5 minutes. The suspension was then sterile filtered, and the average particle size (Zₐᵥ, Malvern Nano-S) determined to be 26 nm, with 2 distinct subpopulations: over 99.9% of particles by volume at 4 nm and a small subpopulation of 0.1% of particles by volume with an average diameter of 85 nm.

### Exemplary Nanoparticle Compositions Containing Gemcitabine Monophosphate Prodrugs

### Example 8

This example demonstrates the preparation of a nanoparticle pharmaceutical composition comprising Compound 5 and albumin. 39.2 mL of a human albumin solution (1.47% w/v) was prepared diluting from a 25% human albumin U.S.P. solution using chloroform saturated water. Compound 5 (39 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 162 nm initially, 182 nm after 15 minutes, and 393 nm after 24 hours at room temperature.

### Example 9

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (44 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 95 nm initially, 106 nm after 15 minutes, and 211 nm after 18 hours at room temperature.

### Example 10

This example demonstrates the preparation of a nanoparticle pharmaceutical composition comprising Compound 7 and albumin. 39.2 mL of a human albumin solution (1.47% w/v) was prepared diluting from a 25% human albumin U.S.P. solution using chloroform saturated water. Compound 7 (57 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 8 minutes. The suspension was then sterile filtered, and the average particle size (Zₐᵥ, Malvern Nano-S) was determined to be 68 nm initially, 76 nm after 15 minutes, and 177 nm after 24 hours at room temperature.

### Example 11

This example demonstrates the preparation of a nanoparticle pharmaceutical composition comprising Compound 8 and albumin. 39.2 mL of a human albumin solution (1.47% w/v) was prepared diluting from a 25% human albumin U.S.P. solution using chloroform saturated water. Compound 8 (54 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 69 nm initially, 69 nm after 15 minutes, and 69 nm after 24 hours at room temperature.

### Example 12

This example demonstrates the preparation of a nanoparticle pharmaceutical composition comprising Compound 9 and albumin. 39.2 mL of a human albumin solution (1.47% w/v) was prepared diluting from a 25% human albumin U.S.P. solution using chloroform saturated water. Compound 9 (59 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 64 nm initially, 64 nm after 15 minutes, and 65 nm after 24 hours at room temperature.

### Example 13

This example demonstrates the preparation of a nanoparticle pharmaceutical composition comprising Compound 10 and albumin. 39.2 mL of a human albumin solution (1.47% w/v) was prepared diluting from a 25% human albumin U.S.P. solution using chloroform saturated water. Compound 10 (57 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 65 nm initially, 65 nm after 15 minutes, and 69 nm after 24 hours at room temperature.

### Example 14

This example demonstrates the preparation of a nanoparticle pharmaceutical composition comprising Compound 11 and albumin. 39.2 mL of a human albumin solution (1.47% w/v) was prepared diluting from a 25% human albumin U.S.P. solution using chloroform saturated water. Compound 11 (66 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 73 nm initially, 75 nm after 15 minutes, and 104 nm after 24 hours at room temperature.

### Example 15

This example demonstrates the preparation of a nanoparticle pharmaceutical composition comprising Compound 8 and albumin. 39.6 mL of a human albumin solution (1.47% w/v) was prepared diluting from a 25% human albumin U.S.P. solution using chloroform saturated water. Compound 8 (53 mg) was dissolved in 400 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 121 nm after 15 minutes, and 120 nm after 22 hours at room temperature.

### Example 16

This example demonstrates the preparation of a nanoparticle pharmaceutical composition comprising Compound 8 and albumin. 38.4 mL of a human albumin solution (1.47% w/v) was prepared diluting from a 25% human albumin U.S.P. solution using chloroform saturated water. Compound 8 (55 mg) was dissolved in 1600 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 45 nm initially, 44 nm after 15 minutes, and 46 nm after 24 hours at room temperature.

### Example 17

This example demonstrates the preparation of a nanoparticle pharmaceutical composition comprising Compound 7 and albumin. 38.4 mL of a human albumin solution (1.47% w/v) was prepared diluting from a 25% human albumin U.S.P. solution using chloroform saturated water. Compound 7 (46 mg) was dissolved in 1600 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 61 nm initially, 71 nm after 15 minutes, and 175 nm after 24 hours at room temperature.

### Example 18

This example demonstrates the preparation of a nanoparticle pharmaceutical composition comprising Compound 12 and albumin. 39.6 mL of a human albumin solution (1.47% w/v) was prepared diluting from a 25% human albumin U.S.P. solution using chloroform saturated water. Compound 12 (41 mg) was dissolved in 400 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then filtered at 0.45 and the average particle size (Zₐᵥ, Malvern Nano-S) was determined to be 188 nm initially, 202 nm after 15 minutes, and 353 nm after 24 hours at room temperature.

### Example 19

This example demonstrates the preparation of a nanoparticle pharmaceutical composition comprising Compound 12 and albumin. 38.4 mL of a human albumin solution (1.47% w/v) was prepared diluting from a 25% human albumin U.S.P. solution using chloroform saturated water. Compound 12 (41 mg) was dissolved in 1600 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then filtered at 0.45 μm, and the average particle size (Zₐᵥ, Malvern Nano-S) was determined to be 205 nm initially, 246 nm after 3 hours, and 291 nm after 24 hours at room temperature.

### Example 20

This example demonstrates the preparation of a nanoparticle pharmaceutical composition comprising Compound 13 and albumin. 49.0 mL of a human albumin solution (1.47% w/v) was prepared diluting from a 25% human albumin U.S.P. solution using chloroform saturated water. Compound 13 (49 mg) was dissolved in 1000 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 3 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 8 minutes. The suspension was then sterile filtered, and the average particle size (Zₐᵥ, Malvern Nano-S) was determined to be 84 nm initially, 89 nm after 15 minutes, and 87 nm after 4 hours at room temperature.

### Example 21

This example demonstrates the preparation of a nanoparticle pharmaceutical composition comprising Compound 14 and albumin. 39.2 mL of a human albumin solution (1.47% w/v) was prepared diluting from a 25% human albumin U.S.P. solution using chloroform saturated water. Compound 14 (49 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 117 nm initially, 134 nm after 15 minutes, and 257 nm after 24 hours at room temperature.

### Example 22

This example demonstrates the preparation of a nanoparticle pharmaceutical composition comprising Compound 15 and albumin. 39.2 mL of a human albumin solution (1.47% w/v) was prepared diluting from a 25% human albumin U.S.P. solution using chloroform saturated water. Compound 15 (42 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 165 nm initially, 191 nm after 15 minutes, and 241 nm after 24 hours at room temperature.

### Example 23

This example demonstrates the preparation of a nanoparticle pharmaceutical composition comprising Compound 12 and albumin. 39.2 mL of a human albumin solution (1.47% w/v) was prepared diluting from a 25% human albumin U.S.P. solution using chloroform saturated water. Compound 12 (52 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 155 nm initially, 165 nm after 15 minutes, and 224 nm after 4 hours at room temperature.

### Example 24

This example demonstrates the preparation of a nanoparticle pharmaceutical composition comprising Compound 18 and albumin. 19.6 mL of a human albumin solution (1.47% w/v) was prepared diluting from a 25% human albumin U.S.P. solution using chloroform saturated water. Compound 18 (26 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 68 nm initially, 89 nm after 120 minutes, and 139 nm after 24 hours at room temperature.

### Example 25

This example demonstrates the preparation of a nanoparticle pharmaceutical composition comprising Compound 19 and albumin. 19.6 mL of a human albumin solution (1.47% w/v) was prepared diluting from a 25% human albumin U.S.P. solution using chloroform saturated water. Compound 19 (28 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 63 nm initially, 68 nm after 120 minutes, and 85 nm after 24 hours at room temperature.

### Example 26

This example demonstrates the preparation of a nanoparticle pharmaceutical composition comprising Compound 20 and albumin. 49.0 mL of a human albumin solution (1.47% w/v) was prepared diluting from a 25% human albumin U.S.P. solution using chloroform saturated water. Compound 20 (70 mg) was dissolved in 1000 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 8 minutes. The suspension was then sterile filtered, and the average particle size (Zₐᵥ, Malvern Nano-S) was determined to be 52 nm initially, 53 nm after 120 minutes, and 62 nm after 24 hours at room temperature.

### Example 27

This example demonstrates the preparation of a nanoparticle pharmaceutical composition comprising Compound 21 and albumin. 49.0 mL of a human albumin solution (1.47% w/v) was prepared diluting from a 25% human albumin U.S.P. solution using chloroform saturated water. Compound 21 (73 mg) was dissolved in 1000 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 8 minutes. The suspension was then sterile filtered, and the average particle size (Zₐᵥ, Malvern Nano-S) was determined to be 52 nm initially, 51 nm after 120 minutes, and 54 nm after 24 hours at room temperature.

### Example 28

This example demonstrates the preparation of a nanoparticle pharmaceutical composition comprising Compound 22 and albumin. 19.6 mL of a human albumin solution (1.47% w/v) was prepared diluting from a 25% human albumin U.S.P. solution using chloroform saturated water. Compound 22 (22 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 90 nm initially, 110 nm after 60 minutes, and 130 nm after 4 hours at room temperature.

### Example 29

This example demonstrates the preparation of a nanoparticle pharmaceutical composition comprising Compound 23 and albumin. 19.6 mL of a human albumin solution (1.47% w/v) was prepared diluting from a 25% human albumin U.S.P. solution using chloroform saturated water. Compound 23 (23 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 67 nm initially, 91 nm after 60 minutes, and 109 nm after 4 hours at room temperature.

### Example 30

This example demonstrates the preparation of a nanoparticle pharmaceutical composition comprising Compound 24 and albumin. 19.6 mL of a human albumin solution (1.47% w/v) was prepared diluting from a 25% human albumin U.S.P. solution using chloroform saturated water. Compound 24 (23 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 65 nm initially, 72 nm after 4 hours, and 80 nm after 24 hours at room temperature.

### Example 31

This example demonstrates the preparation of a nanoparticle pharmaceutical composition comprising Compound 16 and albumin. 39.2 mL of a human albumin solution (1.47% w/v) was prepared diluting from a 25% human albumin U.S.P. solution using chloroform saturated water. Compound 16 (49 mg) was dissolved in 800 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 56 nm initially, 57 nm after 160 minutes, and 73 nm after 21 hours at room temperature.

### Example 32

This example demonstrates the preparation of a nanoparticle pharmaceutical composition comprising Compound 25 and albumin. 19.6 mL of a human albumin solution (1.47% w/v) was prepared diluting from a 25% human albumin U.S.P. solution using chloroform saturated water. Compound 25 (24 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 55 nm initially, 60 nm after 4 hours, and 72 nm after 5 days at room temperature.

### Example 33

This example demonstrates the preparation of a nanoparticle pharmaceutical composition comprising Compound 26 and albumin. 19.6 mL of a human albumin solution (1.47% w/v) was prepared diluting from a 25% human albumin U.S.P. solution using chloroform saturated water. Compound 26 (25 mg) was dissolved in 400 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 82 nm initially, 78 nm after 4 hours, and 102 nm after 3 days at room temperature.

### Example 34

This example demonstrates the preparation of a nanoparticle pharmaceutical composition comprising Compound 17 and albumin. 39.2 mL of a human albumin solution (1.47% w/v) was prepared diluting from a 25% human albumin U.S.P. solution using chloroform saturated water. Compound 17 (54 mg) was dissolved in 800 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 66 nm initially, 81 nm after 160 minutes, and 120 nm after 24 hours at room temperature.

### Example 35

This example demonstrates the preparation of a nanoparticle pharmaceutical composition comprising Compound 27 and albumin. 39.2 mL of a human albumin solution (1.47% w/v) was prepared diluting from a 25% human albumin U.S.P. solution using chloroform saturated water. Compound 27 (56 mg) was dissolved in 800 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 64 nm initially, 65 nm after 120 minutes, and 77 nm after 24 hours at room temperature.

### Exemplary Nanoparticle Compositions Containing Gemcitabine Monophosphate Prodrugs at Different Molecular Ratios

### Example 36

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (6 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 45 nm initially, 65 nm after 15 minutes, and 178 nm after 19 hours at room temperature.

### Example 37

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (17 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 57 nm initially, 79 nm after 15 minutes, and 204 nm after 19 hours at room temperature.

### Example 38

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (34 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 76 nm initially, 94 nm after 15 minutes, and 153 nm after 4 hours at room temperature.

### Example 39

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (56 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 79 nm initially, 97 nm after 15 minutes, and 221 nm after 24 hours at room temperature.

### Example 40

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (84 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 75 nm initially, 97 nm after 15 minutes, and 215 nm after 24 hours at room temperature.

### Example 41

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (112 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 93 nm initially, 111 nm after 15 minutes, and 147 nm after 2 hours at room temperature.

### Example 42

This example demonstrates the preparation of a nanoparticle pharmaceutical composition comprising Compound 6 and albumin. 39.2 mL of a human albumin solution (1.47% w/v) was prepared diluting from a 25% human albumin U.S.P. solution using chloroform saturated water. Compound 6 (56 mg) was dissolved in 800 μL chloroform/t-butyl alcohol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 72 nm initially, 92 nm after 15 minutes, and 117 nm after 60 minutes at room temperature.

### Exemplary Nanoparticle Compositions Upon Lyophilization and Rehydration

### Example 43

This example demonstrates the lyophilization and rehydration into each of: water, 5% dextrose water, and 0.9% saline for a nanoparticle pharmaceutical composition comprising Compound 8 and albumin. Immediately after sterile filtration, the nanoparticle suspension from Example 11 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 81 nm initially, 82 nm after 15 minutes, and 82 nm after 2 hours at room temperature. Upon hydration into 5% dextrose water, the average particle size (Zₐᵥ, Malvern Nano-S) was determined to be 93 nm initially, 94 nm after 15 minutes, and 91 nm after 2 hours at room temperature. Upon hydration into 0.9% saline, the average particle size (Zₐᵥ, Malvern Nano-S) was determined to be 79 nm initially, 81 nm after 15 minutes, and 79 nm after 2 hours at room temperature.

### Example 44

This example demonstrates the lyophilization and rehydration into each of: water, 5% dextrose water, and 0.9% saline for a nanoparticle pharmaceutical composition comprising Compound 10 and albumin. Immediately after sterile filtration, the nanoparticle suspension from Example 13 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 84 nm initially, 84 nm after 15 minutes, and 82 nm after 90 minutes at room temperature. Upon hydration into 5% dextrose water, the average particle size (Zₐᵥ, Malvern Nano-S) was determined to be 95 nm initially, 94 nm after 15 minutes, and 91 nm after 90 minutes at room temperature. Upon hydration into 0.9% saline, the average particle size (Zₐᵥ, Malvern Nano-S) was determined to be 83 nm initially, 87 nm after 15 minutes, and 97 nm after 90 minutes at room temperature.

### Example 45

This example demonstrates the lyophilization and rehydration into each of: water, 5% dextrose water, and 0.9% saline for a nanoparticle pharmaceutical composition comprising Compound 20 and albumin. Immediately after sterile filtration, the nanoparticle suspension from Example 26 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 74 nm initially, 71 nm after 30 minutes, and 69 nm after 2 hours at room temperature. Upon hydration into 5% dextrose water, the average particle size (Zₐᵥ, Malvern Nano-S) was determined to be 87 nm initially, 85 nm after 30 minutes, and 83 nm after 2 hours at room temperature. Upon hydration into 0.9% saline, the average particle size (Zₐᵥ, Malvern Nano-S) was determined to be 83 nm initially, 84 nm after 30 minutes, and 88 nm after 2 hours at room temperature.

### Example 46

This example demonstrates the lyophilization and rehydration into each of: water, 5% dextrose water, and 0.9% saline for a nanoparticle pharmaceutical composition comprising Compound 24 and albumin. Immediately after sterile filtration, a nanoparticle suspension prepared using the same method as Example 30 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 80 nm initially, 81 nm after 30 minutes, and 81 nm after 2 hours at room temperature. Upon hydration into 5% dextrose water, the average particle size (Zₐᵥ, Malvern Nano-S) was determined to be 90 nm initially, 90 nm after 30 minutes, and 90 nm after 2 hours at room temperature. Upon hydration into 0.9% saline, the average particle size (Zₐᵥ, Malvern Nano-S) was determined to be 98 nm initially, 102 nm after 30 minutes, and 107 nm after 2 hours at room temperature.

### Example 47

This example demonstrates the lyophilization and rehydration into each of: water, 5% dextrose water, and 0.9% saline for a nanoparticle pharmaceutical composition comprising Compound 16 and albumin. Immediately after sterile filtration, the nanoparticle suspension from Example 31 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 60 nm initially, 57 nm after 30 minutes, and 58 nm after 2 hours at room temperature. Upon hydration into 5% dextrose water, the average particle size (Zₐᵥ, Malvern Nano-S) was determined to be 67 nm initially, 65 nm after 30 minutes, and 65 nm after 2 hours at room temperature. Upon hydration into 0.9% saline, the average particle size (Zₐᵥ, Malvern Nano-S) was determined to be 61 nm initially, 64 nm after 30 minutes, and 68 nm after 2 hours at room temperature.

### Example 48

This example demonstrates the lyophilization and rehydration into each of: water, 5% dextrose water, and saline for a nanoparticle pharmaceutical composition comprising Compound 27 and albumin. Immediately after sterile filtration, the nanoparticle suspension from Example 35 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 87 nm initially, 86 nm after 30 minutes, and 87 nm after 2 hours at room temperature. Upon hydration into 5% dextrose water, the average particle size (Zₐᵥ, Malvern Nano-S) was determined to be 106 nm initially, 103 nm after 30 minutes, and 104 nm after 2 hours at room temperature. Upon hydration into 0.9% saline, the average particle size (Zₐᵥ, Malvern Nano-S) was determined to be 107 nm initially, 114 nm after 30 minutes, and 121 nm after 2 hours at room temperature.

### Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants (J) are reported in Hertz. For proton spectra the solvent peak was used as the reference peak.

### Example 49: Synthesis of (2R,3R,5R)-5-(4-amino-2-oxopyrimidin-1(2H)-yl)-4,4-difluoro-2-(((2-oxido-4H-benzo[d][1,3,2]dioxaphosphinin-2-yl)oxy)methyl)tetrahydrofuran-3-yl Nonadecanoate (Compound 16)

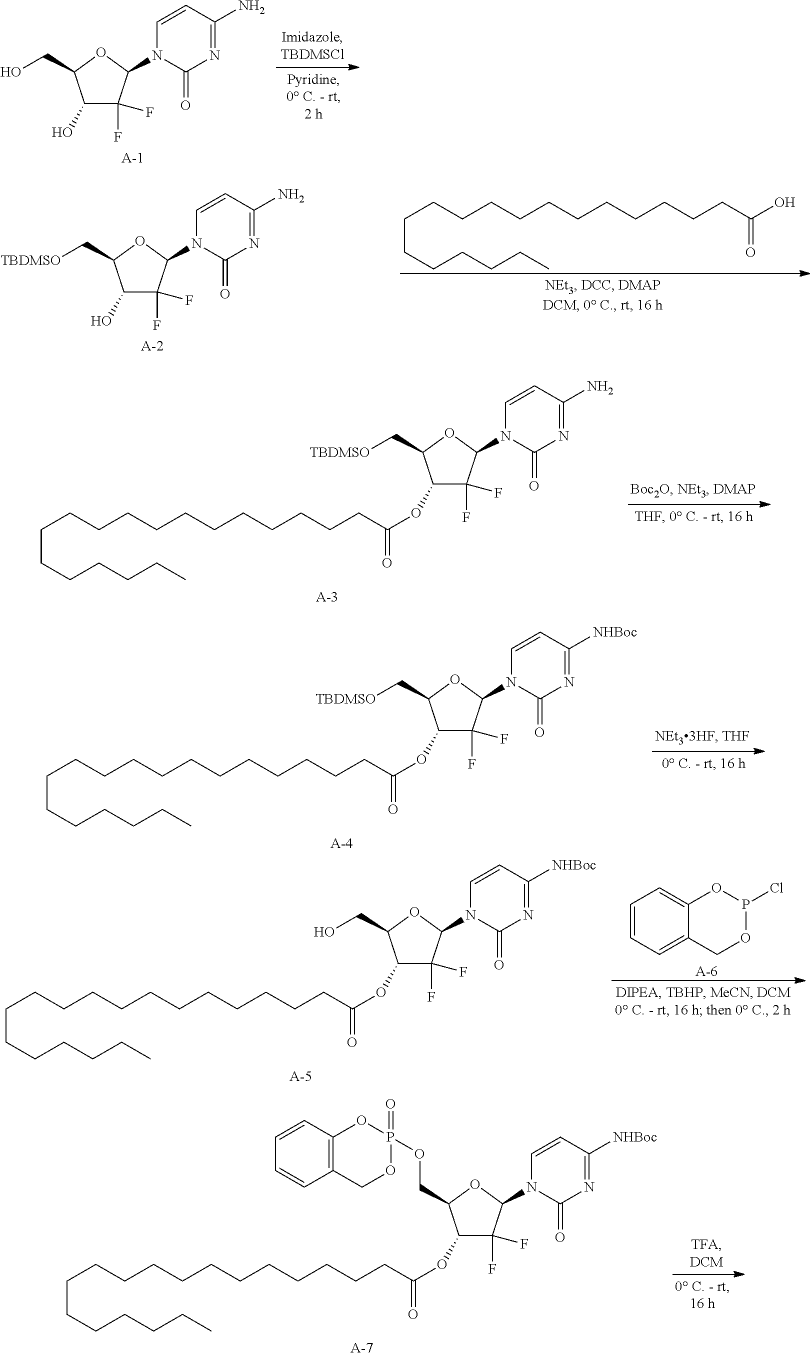

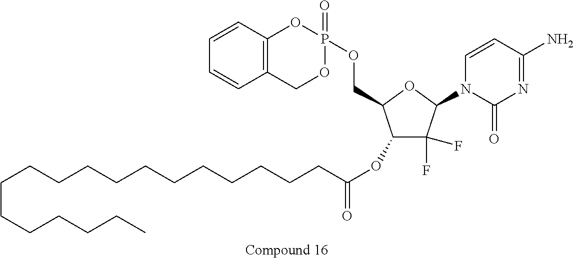 [SMILES: CCCCCCCCCCCCCCCCCCC(=O)O[C@@H]1[C@@H](COP2(=O)OCc3ccccc3O2)O[C@@H](n2ccc(N)nc2=O)C1(F)F ; "Compound 16"]

### Synthesis of 2-chloro-4H-benzo[d][1,3,2]dioxaphosphinine (A-6)

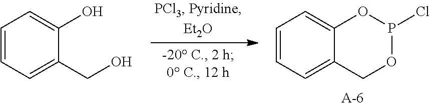

To a solution of 2-(hydroxymethyl)phenol (5 g, 40.0 mmol) in dry diethyl ether (150 mL), freshly distilled PCl₃ (3.8 mL, 0.44 mmol) was added over a period of 15 min at −20° C., and then dry pyridine (10.6 mL, 120.0 mmol) in diethyl ether (50 mL) was added dropwise over a period of 2 h at −20° C. and stirred at rt for 2 h, then the reaction mixture was stored at 0° C. for 12 h. The reaction mixture was filtered under inert atmosphere and the filtrate was concentrated under reduced pressure to afford 2-chloro-4H-benzo[d][1,3,2]dioxaphosphinine (A-6) (6 g) as a yellow oil which was used without further purification.

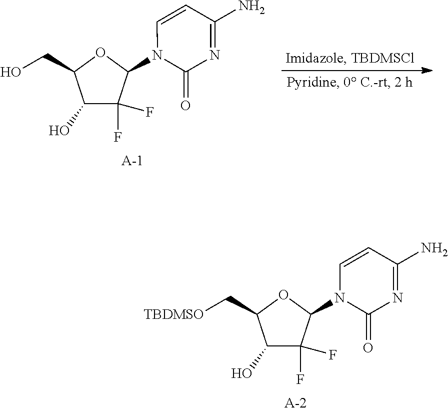

To (2R,3S,5R)-5-(6-amino-9H-purin-9-yl)-2-(hydroxymethyl)tetrahydrofuran-3-ol (A-1) (5 g, 18.99 mmol) in pyridine (50 mL, 10 vol.) at rt was added imidazole (3.87 g, 56.99 mmol), TBDMS-Cl (4.27 g, 28.49 mmol) and stirred at rt for 2 h. The solvent was evaporated, the residue was taken in water (100 mL) and extracted with ethyl acetate (4×50 mL). The combined organic layer was washed with water (50 mL), brine (50 mL), dried over anhydrous Na₂SO₄, filtered and evaporated. The crude residue was purified by column chromatography (SiO₂, 100-200 mesh) to afford 4-amino-((2R,4R,5R)-5-(((tert-butyl dimethylsilyl)oxy)methyl)-3,3-difluoro-4-hydroxytetrahydrofuran-2-yl)pyrimidin-2(1H)-one (A-2) (4 g, 56%) as a white solid.

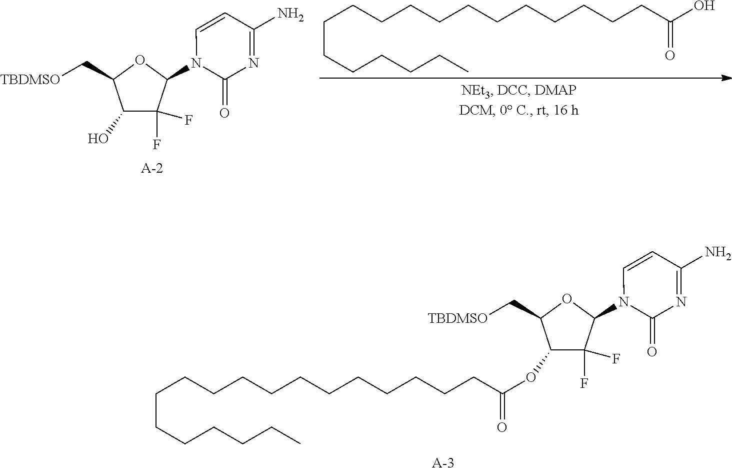

To a solution of (4-amino-1-((2R,4R,5R)-5-(((tert-butyldimethylsilyl)oxy)methyl)-3,3-difluoro-4-hydroxytetrahydrofuran-2-yl)pyrimidin-2(1H)-one (A-2) (5.0 g, 13.2 mmol) in 1,4-dioxane (200 mL, 20 vol.) at 0° C. was added nonadecanoic acid (11.8 g, 39.7 mmol), NEt₃ (9.3 mL, 66.3 mmol), DCC (8.2 g, 39.7 mmol) and DMAP (0.16 g, 0.13 mmol) and stirred at rt for 16 h. The reaction mixture was poured into cold water (150 mL) and extracted with EtOAc (4×10 mL). The combined organic layer was washed with brine (100 mL), dried over anhydrous Na₂SO₄, filtered and concentrated under reduced pressure. The crude residue was purified by column chromatography (SiO₂, 100-200 mesh) to afford (2R,3R,5R)-5-(4-amino-2-oxopyrimidin-1(2H)-yl)-2-(((tert-butyldimethylsilyl)oxy)methyl)-4,4-difluorotetrahydrofuran-3-yl nonadecanoate (A-3) (5.0 g, 57%) as a white solid.

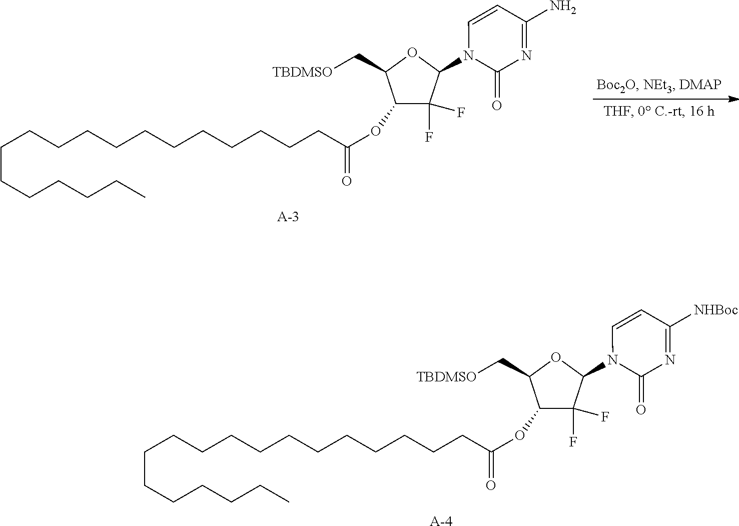 [SMILES: CCCCCCCCCCCCCCCCCCC(=O)O[C@@H]1[C@@H](CO[Si](C)(C)C(C)(C)C)O[C@@H](n2ccc(N)nc2=O)C1(F)F ; "A-3" ; CCCCCCCCCCCCCCCCCCC(=O)O[C@@H]1[C@@H](CO[Si](C)(C)C(C)(C)C)O[C@@H](n2ccc(NC(=O)OC(C)(C)C)nc2=O)C1(F)F ; "A-4"]

To a solution of (2R,3R,5R)-5-(4-amino-2-oxopyrimidin-1(2H)-yl)-2-(((tert-butyldimethylsilyl)oxy)methyl)-4,4-difluorotetrahydrofuran-3-yl nonadecanoate (A-3) (5 g, 7.9 mmol) in 1,4-dioxane (20 mL) at 0° C. was added NEt₃ (3.18 mL, 22.8 mmol), DMAP (0.1 g, 0.79 mmol) followed by Boc₂O (2.5 mL, 11.85 mmol) and stirred at rt for 16 h. The reaction mixture was diluted with water (100 mL) and extracted with ethyl acetate (4×70 mL). The combined organic layer was washed with brine (50 mL), dried over anhydrous Na₂SO₄, filtered and concentrated under reduced pressure. The crude residue was purified by column chromatography (SiO₂, 100-200 mesh) to afford (2R,3R,5R)-5-(4-((tert-butoxycarbonyl)amino)-2-oxopyrimidin-1(2H)-yl)-2-(((tert-butyldimethylsilyl)oxy)methyl)-4,4-difluorotetrahydrofuran-3-yl nonadecanoate (A-4) (3.2 g, 55%) as a white solid.

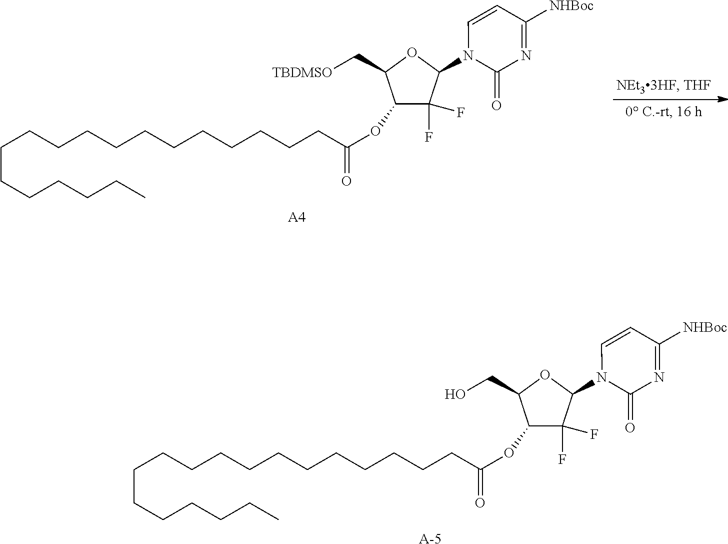 [SMILES: CCCCCCCCCCCCCCCCCCC(=O)O[C@@H]1[C@@H](CO[Si](C)(C)C(C)(C)C)O[C@@H](n2ccc(NC(=O)OC(C)(C)C)nc2=O)C1(F)F ; "A4" ; CCCCCCCCCCCCCCCCCCC(=O)O[C@@H]1[C@@H](CO)O[C@@H](n2ccc(NC(=O)OC(C)(C)C)nc2=O)C1(F)F ; "A-5"]

To a solution of (2R,3R,5R)-5-(4-((tert-butoxycarbonyl)amino)-2-oxopyrimidin-1(2H)-yl)-2-(((tert-butyldimethylsilyl)oxy)methyl)-4,4-difluorotetrahydrofuran-3-yl nonadecanoate (A-4) (3.2 g, 4.22 mmol) in THF (60 mL) at 0° C. was added NEt₃.3HF (3.5 g, 21.1 mmol) dropwise over a period of 30 min. The reaction mixture was slowly warm to room temperature and stirred for 12 h. The solvent was evaporated, the crude was dissolved in EtOAc (100 mL), washed with water (2×50 mL), brine (50 mL), dried over anhydrous Na₂SO₄, filtered and concentrated under reduced pressure to give a crude compound. The crude compound was purified by column chromatography by using 100-200 mesh silica gel to afford (2R,3R,5R)-5-(4-((tert-butoxycarbonyl)amino)-2-oxopyrimidin-1(2H)-yl)-4,4-difluoro-2-(hydroxymethyl)tetrahydrofuran-3-yl nonadecanoate (A-5) (1.6 g, 59%) as an oil.

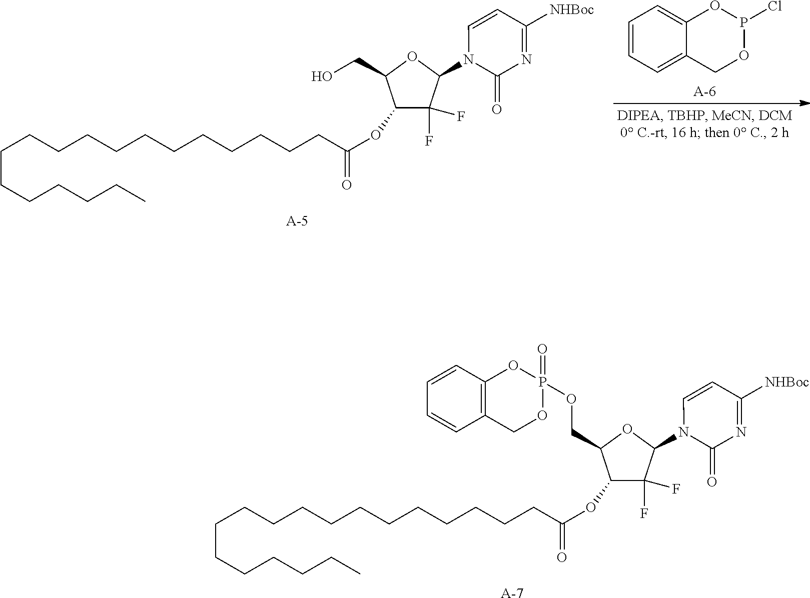

To a solution of (2R,3R,5R)-5-(4-((tert-butoxycarbonyl)amino)-2-oxopyrimidin-1(2H)-yl)-4,4-difluoro-2-(hydroxymethyl)tetrahydrofuran-3-yl nonadecanoate (A-5) (1.8 g, 2.79 mmol) in dry acetonitrile (40 mL) was added DIPEA (3 mL, 16.79 mmol) followed by the dropwise addition over a period of 15 min at 0° C. of a solution of 2-chloro-4H-benzo[d][1,3,2]dioxaphosphinine (A-6) (1.35 g, 6.97 mmol) in dry DCM (10 mL). The reaction mixture was stirred at rt for 16 h. To the reaction mixture was added 5M TBHP (1.24 mL, 13.95 mmol) at 0° C. and the mixture was stirred for 2 h. The solvent was evaporated, the residue was dissolved in EtOAc (50 mL) and washed with water (2×50 mL) and brine (25 mL). The organic layer was dried over anhydrous Na₂SO₄, filtered and evaporated under reduced pressure. The crude compound was purified by column chromatography (SiO₂, 100-200 mesh) to afford (2R,3R,5R)-5-(4-((tert-butoxycarbonyl)amino)-2-oxopyrimidin-1(2H)-yl)-4,4-difluoro-2-4(2-oxido-4H-benzo[d][1,3,2]dioxaphosphinin-2-yl)oxy)methyl)tetrahydrofuran-3-yl nonadecanoate (A-7) (750 mg, 33%) as a colorless liquid.

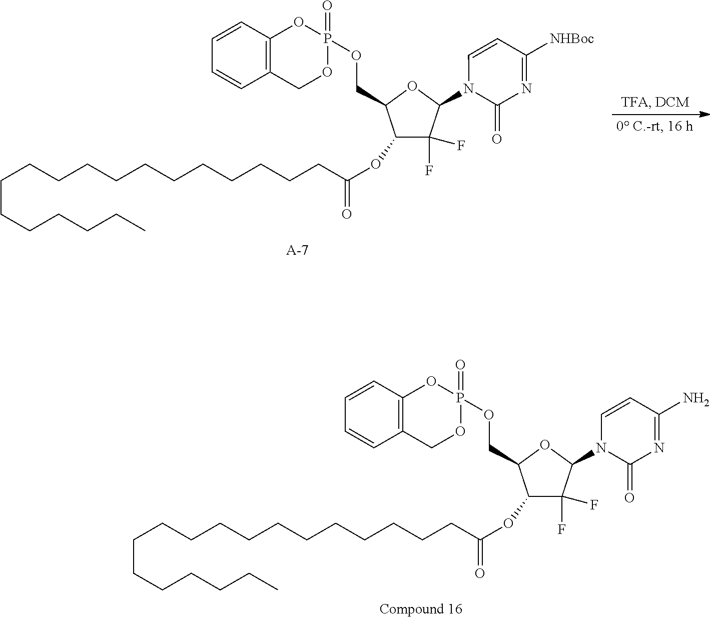 [SMILES: CCCCCCCCCCCCCCCCCCC(=O)O[C@@H]1[C@@H](COP2(=O)OCc3ccccc3O2)O[C@@H](n2ccc(NC(=O)OC(C)(C)C)nc2=O)C1(F)F ; "A-7" ; CCCCCCCCCCCCCCCCCCC(=O)O[C@@H]1[C@@H](COP2(=O)OCc3ccccc3O2)O[C@@H](n2ccc(N)nc2=O)C1(F)F ; "Compound 16"]

To a solution of (2R,3R,5R)-5-(4-((tert-butoxycarbonyl)amino)-2-oxopyrimidin-1(2H)-yl)-4,4-difluoro-2-(((2-oxido-4H-benzo[d][1,3,2]dioxaphosphinin-2-yl)oxy)methyl)tetrahydrofuran-3-yl nonadecanoate (A-7) (0.75 g, 0.9 mmol) in dry DCM (20 mL) at 0° C. was added TFA (0.35 mL, 4.6 mmol) dropwise over a period of 15 min. The reaction mixture was stirred at room temperature for 16 h. The solvent was evaporate under reduced pressure and the residue was dissolved in EtOAc (50 mL) and washed with saturated NaHCO₃ (2×25 mL). The combined extracts was dried over anhydrous Na₂SO₄, filtered and evaporated under reduced pressure. The crude compound was purified by column chromatography (SiO₂, 100-200 mesh) to afford (2R,3R,5R)-5-(4-amino-2-oxopyrimidin-1(2H)-yl)-4,4-difluoro-2-(((2-oxido-4H-benzo[d][1,3,2]dioxaphosphinin-2-yl)oxy)methyl)tetrahydrofuran-3-yl nonadecanoate (Compound 16) (0.41 g, 62%) as a white solid. MS(ESI) m/z 712.40 [M+H]⁺; ¹HNMR (400 MHz, DMSO-d₆) δ 7.5-7.1 (m, 7H), 6.22 (br s, 1H), 5.75 (dd, J=7.6, 1.2 Hz, 1H), 5.58-5.3 (m, 3H), 4.52-4.37 (m, 3H), 2.39 (q, J=7.2 Hz, 2H), 1.52 (t, J=6.4 Hz, 2H), 1.3-1.15 (m, 30H), 0.85 (t, J=7.2 Hz, 3H).

### Example 50: Synthesis of Compound 17

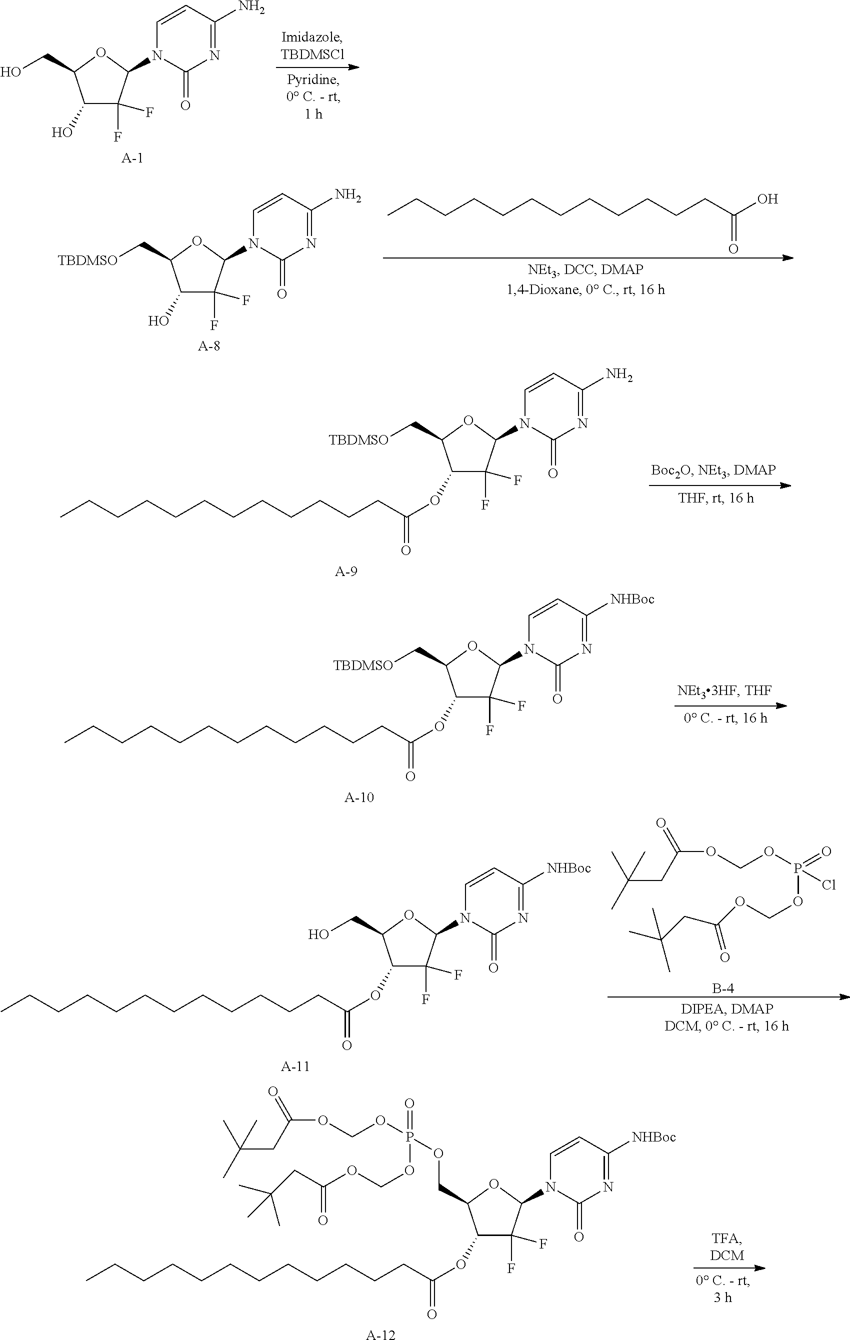

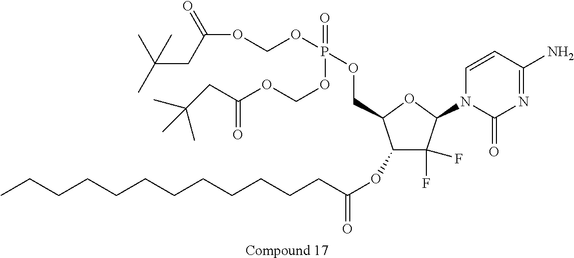 [SMILES: CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F ; "Compound 17"]

### Synthesis of ((chlorophosphoryl)bis(oxy))bis(methylene) bis(3,3-dimethylbutanoate) (B-4)

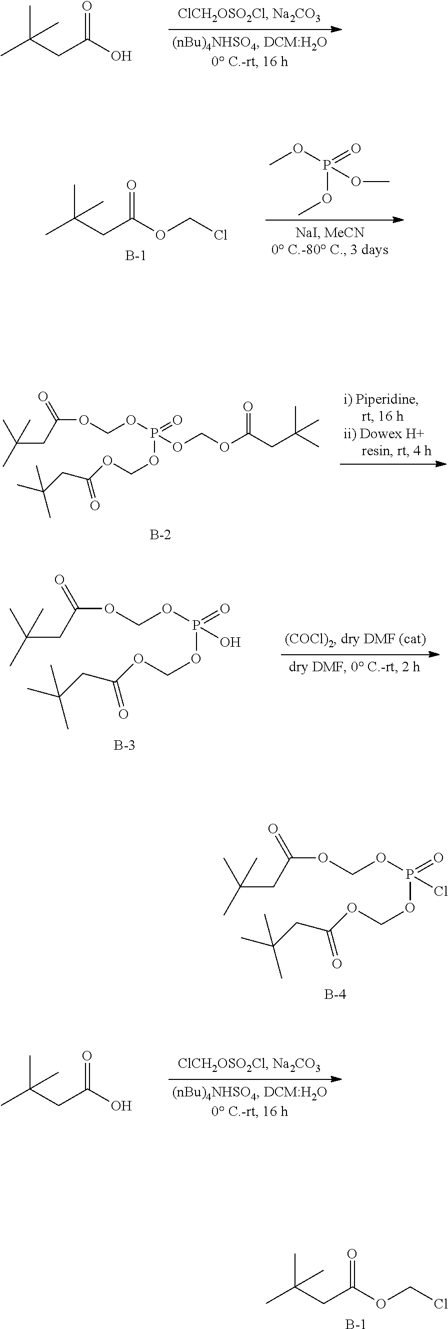

To a stirred solution of 3,3-dimethylbutanoic acid (25 g, 215.52 mmol) in DCM:water (1:1, 2 lit) was added chloromethyl chlorosulphate (42.6 g, 258.62 mmol), NaHCO₃ (72.4 g, 862.02 mmol) and tetrabutylammonium hydrogen sulfate (7.3 g, 21.55 mmol). The reaction mixture was stirred at rt for 16 h. The organic layer was separated, dried over anhydrous Na₂SO₄, and evaporated to afford (B-1) (32 g, 91%) as a yellow liquid.

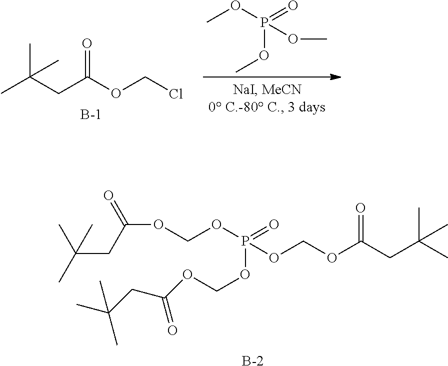

To a stirred solution of trimethyl phosphate (5 g, 35.46 mmol) in acetonitrile (50 mL, 10 vol.) was added chloromethyl 3,3-dimethylbutanoate (B-1) (23.2 g, 141.84 mmol) and NaI (15.8 g, 106.38 mmol) and stirred at 80° C. for 3 days. The reaction mixture was concentrated under reduced pressure. The residue was diluted with water and extracted with EtOAc (2×80 mL), The organic layer was separated, dried over Na₂SO₄, and evaporated. The crude compound was purified by column chromatography (SiO₂, 100-200 mesh) to afford (B-2) (10 g, 58%) as a yellow oil.

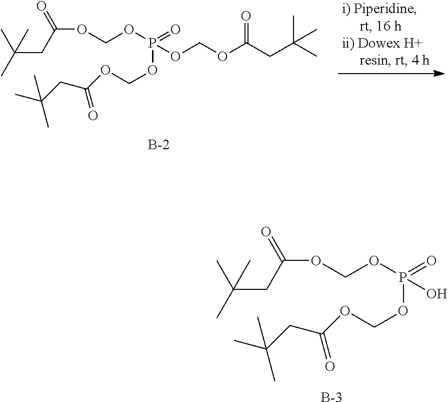

To ((oxo-15-phosphanetriyl)tris(oxy))tris(methylene) tris(3,3-dimethylbutanoate) (B-2) (5 g, 10.37 mmol) was added piperidine (35 mL, 7 vol.) at rt and the reaction mixture was stirred at rt for 16 h. The reaction mixture was concentrated under vacuum. The residue was diluted with water (30 mL, 6 vol.) and acidified with DOWEX (pH˜2) at 0° C. and stirred at rt for 2 h. The resin was removed by filtration and the filtrate were passed through a pad of resin column and evaporated under reduced pressure up to dryness to afford ((hydroxyphosphoryl)bis(oxy))bis(methylene) bis(3,3-dimethylbutanoate) (B-3) (2.1 g, 57%) as a pale yellow liquid.

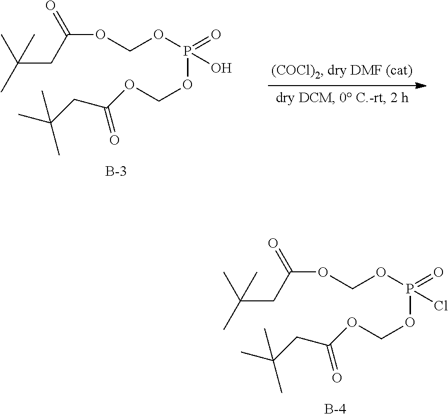

To a stirred solution of ((hydroxyphosphoryl)bis(oxy))bis(methylene) bis(3,3-dimethylbutanoate) (B-3) (4 g, 12.27 mmol) in dry DCM (40 mL, 10 vol.) and dry DMF (cat. 2 drops) was added oxalylchloride (4.2 mL, 49.08 mmol) slowly dropwise over a period of 10 min at 0° C. The reaction mixture was then stirred at rt for 2 h. The reaction mixture was concentrated under reduced pressure (up to consistent weight) under argon to afford ((chlorophosphoryl)bis(oxy))bis(methylene)bis(3,3-dimethylbutanoate) (B-4) (4.5 g, quantitative) as a yellow oil which was used without further purification.

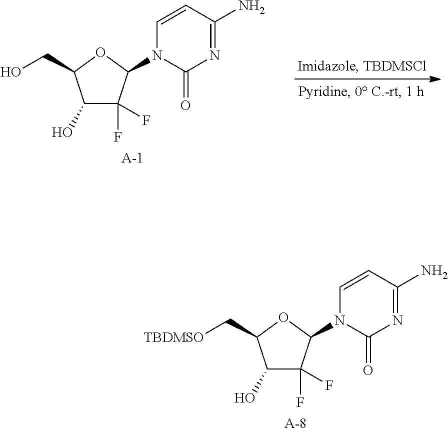

To (2R,3S,5R)-5-(6-amino-9H-purin-9-yl)-2-(hydroxymethyl)tetrahydrofuran-3-ol (A-1) (5 g, 18.99 mmol) in pyridine (50 mL, 10 vol.) at rt was added imidazole (3.87 g, 56.99 mmol) and TBDMS-Cl (4.27 g, 28.49 mmol). The reaction mixture was stirred at rt for 1 h. The solvent was evaporated and the residue was taken in water (100 mL) and extracted with ethyl acetate (4×50 mL). The combined organic layer was washed with water (50 mL), brine (50 mL), dried over anhydrous Na₂SO₄, filtered and evaporated. The crude residue was purified by column chromatography (SiO2, 100-200 mesh) to afford 4-amino-1-((2R,4R,5R)-5-(((tert-butyl dimethyl silyl)oxy)methyl)-3,3-difluoro-4-hydroxytetrahydrofuran-2-yl)pyrimidin-2(1H)-one (A-8) (4 g, 56%) as a white solid.

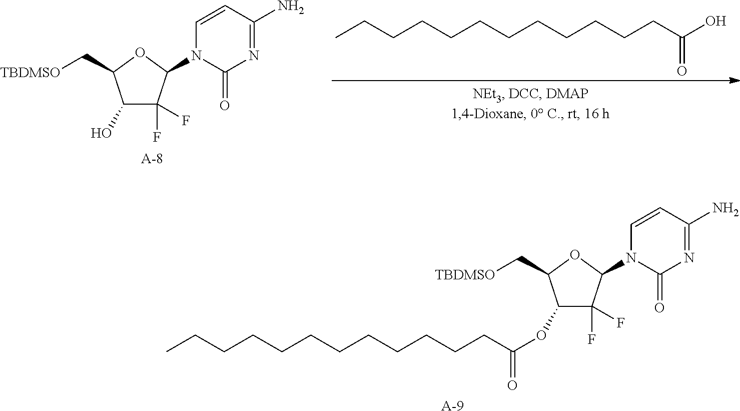

To a solution of (4-amino-1-((2R,4R,5R)-5-(((tert-butyldimethylsilyl)oxy)methyl)-3,3-difluoro-4-hydroxytetrahydrofuran-2-yl)pyrimidin-2(1H)-one (A-8) (9 g, 23.87 mmol) in 1,4-Dioxane (90 mL, 10 vol.) at rt was added tridecanoic acid (15.6 g, 71.618 mmol), Et₃N (16.6 mL, 119.36 mmol), DCC (14.7 g, 71.62 mmol) and DMAP (291 mg, 2.387 mmol) and stirred at rt for 16 h. After completion of the reaction by TLC, the reaction mixture was poured into cold water (100 mL) and extracted with EtOAc (2×500 mL). The combined organic layer was washed with water (500 mL), brine (250 mL), dried over anhydrous Na₂SO₄, filtered and concentrated under reduced pressure. The crude material was purified by column chromatography (SiO₂, 100-200 mesh) to afford (2R,3R,5R)-5-(4-amino-2-oxopyrimidin-1(2H)-yl)-2-(((tert-butyldimethylsilyl)oxy)methyl)-4,4-difluorotetrahydrofuran-3-yl tridecanoate (A-9) (4 g, 29%) as a white solid.

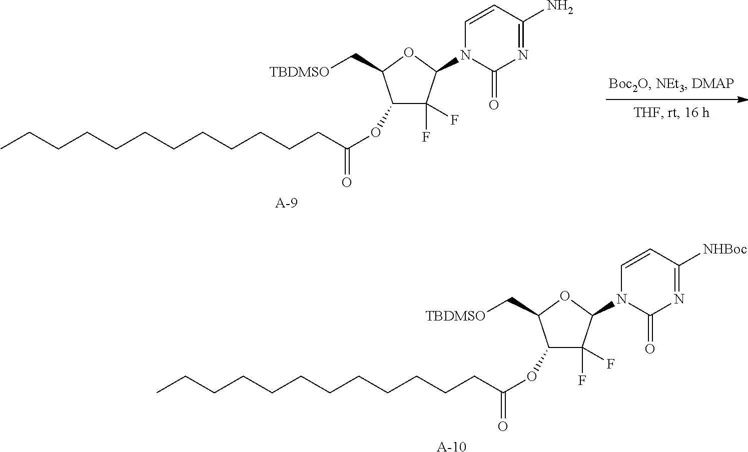 [SMILES: CCCCCCCCCCCCC(=O)O[C@@H]1[C@@H](CO[Si](C)(C)C(C)(C)C)O[C@@H](n2ccc(N)nc2=O)C1(F)F ; "A-9" ; CCCCCCCCCCCCC(=O)O[C@@H]1[C@@H](CO[Si](C)(C)C(C)(C)C)O[C@@H](n2ccc(NC(=O)OC(C)(C)C)nc2=O)C1(F)F ; "A-10"]

To a stirred solution of (2R,3R,5R)-5-(4-amino-2-oxopyrimidin-1(2H)-yl)-2-(((tert-butyldimethylsilyl)oxy)methyl)-4,4-difluorotetrahydrofuran-3-yl tridecanoate (A-9) (4 g, 6.98 mmol) in THF (40 mL) at rt was added Et₃N (2.9 mL, 20.94 mmol), DMAP (85 mg, 0.698 mmol) followed by Boc₂O (2.4 mL, 10.47 mmol). The reaction mixture was stirred at rt for 16 h. The reaction was quenched with water (200 mL) and extracted with ethyl acetate (2×100 mL). The combined organic layer was washed with water (80 mL), brine (80 mL), dried over anhydrous Na₂SO₄, filtered and concentrated under reduced pressure. The residue was purified by column chromatography (SiO₂, 100-200 mesh) to afford (2R,3R,5R)-5-(4-((tert-butoxycarbonyl)amino)-2-oxopyrimidin-1(2H)-yl)-2-(((tert-butyl dimethylsilyl)oxy)methyl)-4,4-difluorotetrahydrofuran-3-yl tridecanoate (A-10) (3.8 g, 80%) as an off-white semisolid.

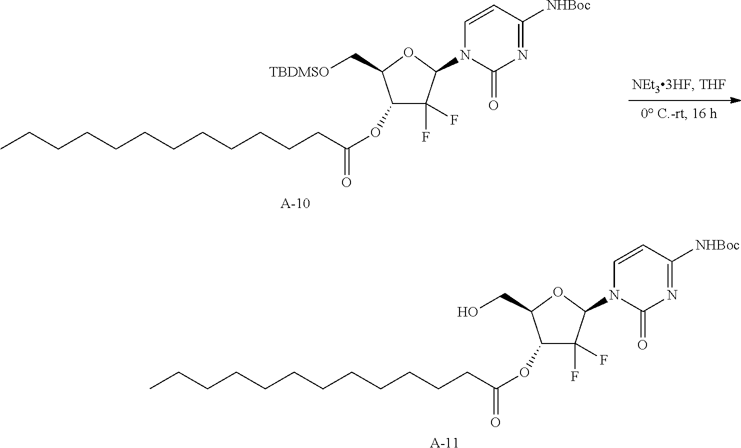 [SMILES: CCCCCCCCCCCCC(=O)O[C@@H]1[C@@H](CO[Si](C)(C)C(C)(C)C)O[C@@H](n2ccc(NC(=O)OC(C)(C)C)nc2=O)C1(F)F ; "A-10" ; CCCCCCCCCCCCC(=O)O[C@@H]1[C@@H](CO)O[C@@H](n2ccc(NC(=O)OC(C)(C)C)nc2=O)C1(F)F ; "A-11"]

To a solution of (2R,3R,5R)-5-(4-((tert-butoxycarbonyl)amino)-2-oxopyrimidin-1(2H)-yl)-2-(((tert-butyl dimethylsilyl)oxy)methyl)-4,4-difluorotetrahydrofuran-3-yl tridecanoate (A-10) (15.6 g, 23.18 mmol) in THF (160 mL, 10 vol.) was added NEt₃.3HF (18.6 g, 115.89 mmol) dropwise over 30 min at 0° C. The reaction mixture was slowly warm to room temperature and stirred for 16 h at rt. The solvent was evaporated. The residue was dissolved in EtOAc (400 mL), washed with water (2×100 mL), brine (100 mL), dried over anhydrous Na₂SO₄, filtered and evaporated under reduced pressure to give a crude compound. The crude compound was purified by column chromatography using 100-200 mesh silica gel to afford (2R,3R,5R)-5-(4-((tert-butoxycarbonyl)amino)-2-oxopyrimidin-1(2H)-yl)-4,4-difluoro-2-(hydroxymethyl) tetrahydrofuran-3-yl tridecanoate (A-11) (10.58 g, 81%) as a light brown liquid.

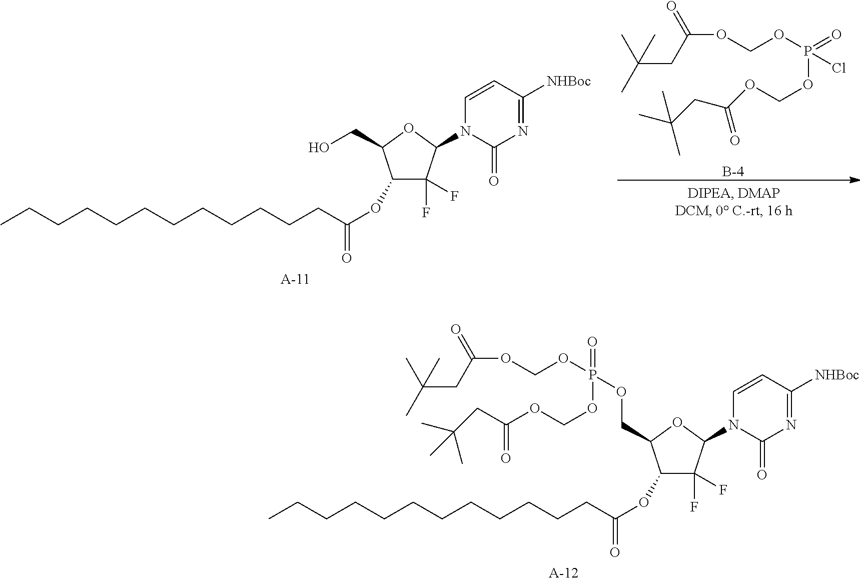

To a solution of ((chlorophosphoryl)bis(oxy))bis(methylene)bis(3,3-dimethylbutanoate) (B-4) (2.3 g, 6.261 mmol) in DCM (15 mL) was added a mixture of (2R,3R,5R)-5-(4-((tert-butoxycarbonyl)amino)-2-oxopyrimidin-1 (2H)-yl)-4,4-difluoro-2-(hydroxymethyl) tetrahydrofuran-3-yl tridecanoate (A-11) (700 mg, 1.252 mmol), DIPEA (1.3 mL, 7.513 mmol) and DMAP (15 mg, 0.125 mmol) in DCM (7 mL) slowly dropwise over a period of 10 min at 0° C. The reaction mixture was stirred at rt for 16 h. The reaction mixture was quenched with water and extracted with EtOAc (2×50 mL). The organic layer was dried over anhydrous Na₂SO₄, filtered and evaporated under reduced pressure. The crude compound was purified by column chromatography (SiO₂, 100-200 mesh) to afford (A-12) (200 mg, 19%) as a brown liquid.

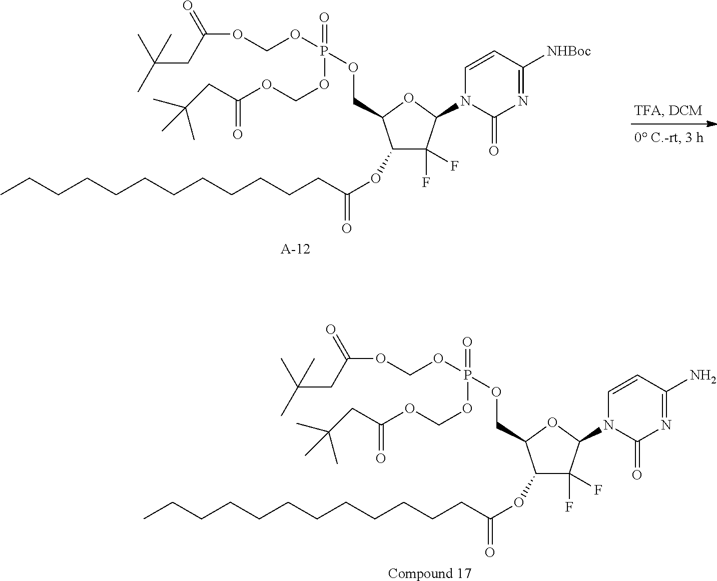 [SMILES: CCCCCCCCCCCCC(=O)O[C@@H]1[C@@H](COP(=O)(OCOC(=O)CC(C)(C)C)OCOC(=O)CC(C)(C)C)O[C@@H](n2ccc(NC(=O)OC(C)(C)C)nc2=O)C1(F)F ; "A-12" ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F ; "Compound 17"]

To a solution of (A-12) (900 mg, 1.005 mmol) in DCM (9 mL) was added TFA (1.8 mL, 2 vol.) dropwise over a period of 10 min at 0° C. The reaction mixture was stirred at room temperature for 3 h. The reaction mixture was concentrated under reduced pressure. The residue was dissolved in EtOAc (100 mL) and washed with saturated NaHCO₃ (2×50 mL). The organic layer was dried over anhydrous Na₂SO₄, filtered and evaporated under reduced pressure. The crude compound was purified by column chromatography (SiO₂, 100-200 mesh) to afford (Compound 17) (0.265 g, 33%) as a colorless semisolid. MS (ESI) m/z 796.46 [M+H]⁺; ¹H NMR (400 MHz, DMSO-d₆) δ 7.57 (d, J=7.6 Hz, 1H), 7.50 (d, J=16.4 Hz, 2H), 6.25 (br s, 1H), 5.81 (d, J=7.6 Hz, 1H), 5.62 (d, J=4.4 Hz, 4H), 5.39-5.62 (br s, 1H), 4.35 (d, J=5.2 Hz, 3H), 2.44 (t, J=8 Hz, 2H), 2.26 (s, 4H), 1.57-1.52 (m, 2H), 1.23 (br m, 18H), 0.98-0.95 (m, 18H), 0.85 (t, J=6.4 Hz, 3H).

### Example 51: Synthesis of Compound 27

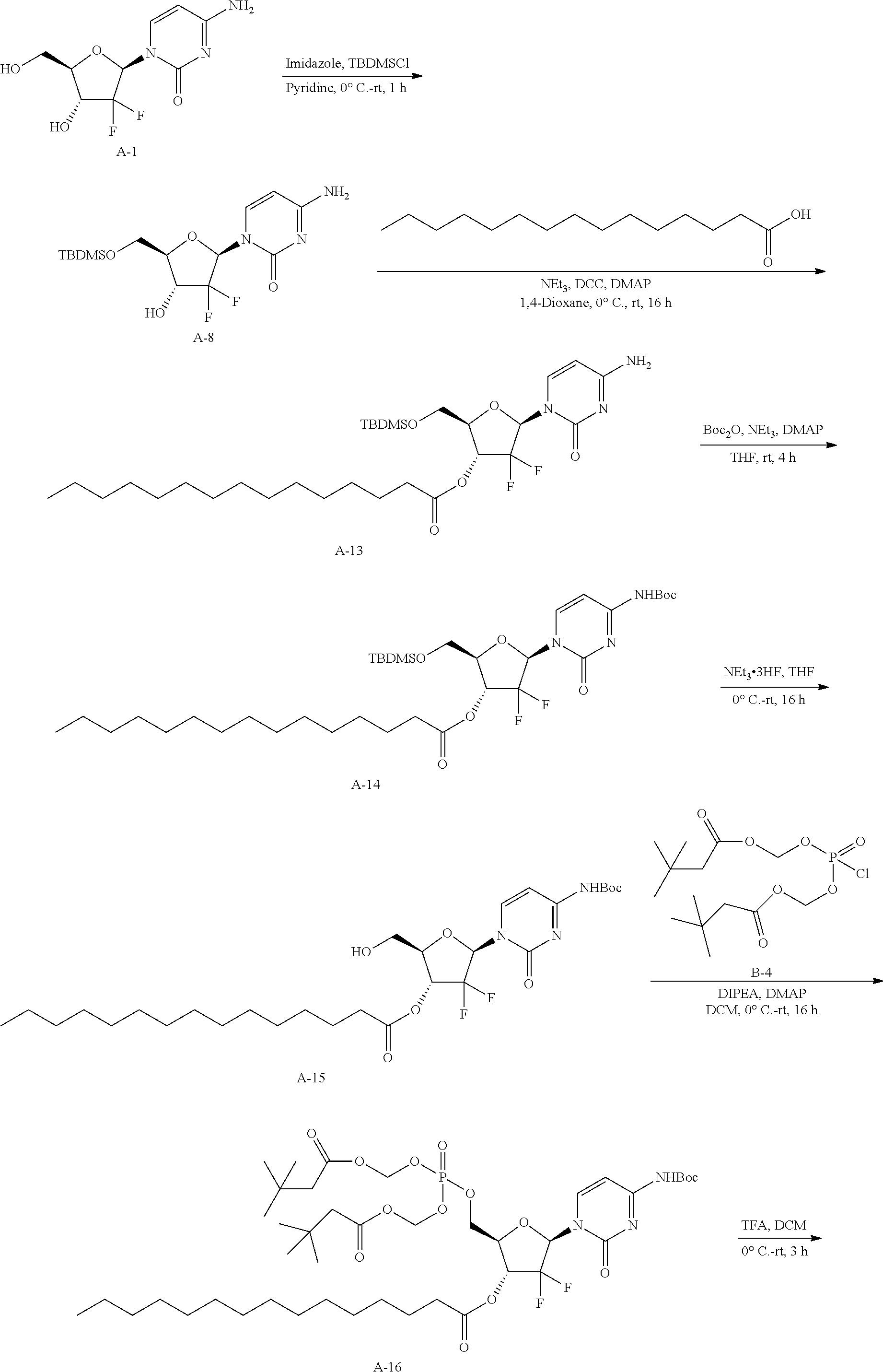

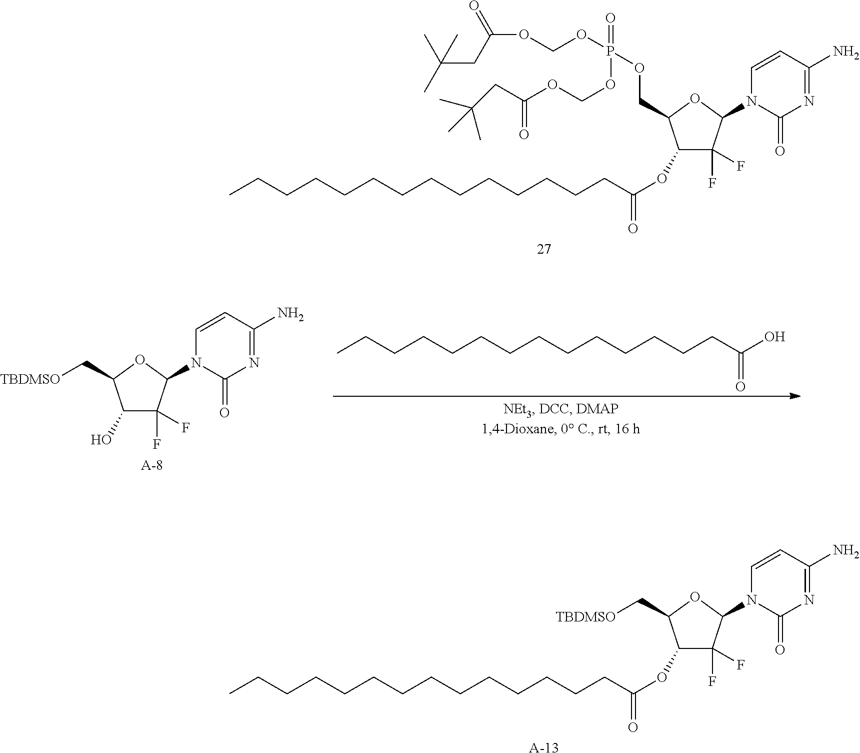

To a solution of (4-amino-1-((2R,4R,5R)-5-(((tert-butyldimethylsilyl)oxy)methyl)-3,3-difluoro-4-hydroxytetrahydrofuran-2-yl)pyrimidin-2(1H)-one (A-8) (16.8 g, 44.562 mmol) in 1,4-dioxane (168 mL, 10 vol.) at rt was added pentadecanoic acid (32.4 g, 133.68 mmol), Et₃N (30.9 mL, 222.8 mmol), DCC (27.5 g, 133.68 mmol) and DMAP (543 mg, 4.456 mmol) and stirred at rt for 16 h. The reaction mixture was poured into cold water (100 mL) and extracted with EtOAc (2×800 mL). The combined organic layer was washed with water (500 mL), brine (500 mL), dried over anhydrous Na₂SO₄, filtered and concentrated under reduced pressure. The crude material was purified by column chromatography (SiO₂, 100-200 mesh) to afford (2R,3R,5R)-5-(4-amino-2-oxopyrimidin-1(2H)-yl)-2-(((tert-butyldimethylsilyl)oxy)methyl)-4,4-difluorotetrahydrofuran-3-yl pentadecanoate (A-13) (8.5 g, 31%) as a white solid.

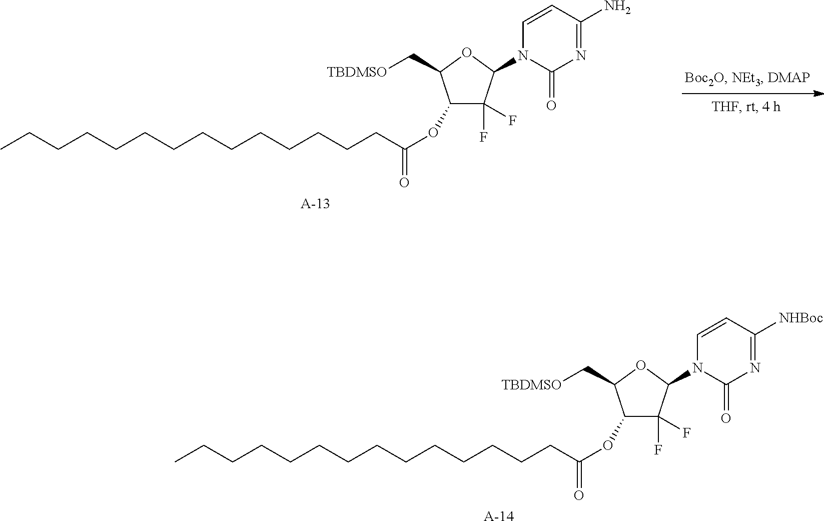 [SMILES: CCCCCCCCCCCCCCC(=O)O[C@@H]1[C@@H](CO[Si](C)(C)C(C)(C)C)O[C@@H](n2ccc(N)nc2=O)C1(F)F ; "A-13" ; CCCCCCCCCCCCCCC(=O)O[C@@H]1[C@@H](CO[Si](C)(C)C(C)(C)C)O[C@@H](n2ccc(NC(=O)OC(C)(C)C)nc2=O)C1(F)F ; "A-14"]

To a stirred solution of (2R,3R,5R)-5-(4-amino-2-oxopyrimidin-1(2H)-yl)-2-(((tert-butyldimethylsilyl)oxy)methyl)-4,4-difluorotetrahydrofuran-3-yl pentadecanoate (A-13) (12.2 g, 20.29 mmol) in THF (120 mL, 10 vol.) at rt was added Et₃N (8.4 mL, 60.89 mmol), DMAP (247 mg, 2.03 mmol) followed by Boc₂O (6.93 mL, 30.45 mmol). The reaction mixture was stirred at rt for 4 h. The reaction was quenched with water (500 mL) and extracted with ethyl acetate (2×600 mL). The combined organic layer was washed with water (300 mL), brine (300 mL), dried over anhydrous Na₂SO₄, filtered and concentrated under reduced pressure. The crude residue was purified by column chromatography (SiO₂, 100-200 mesh) to afford (2R,3R,5R)-5-(4-((tert-butoxycarbonyl)amino)-2-oxopyrimidin-1(2H)-yl)-2-(((tert-butyl dimethyl silyl)oxy)methyl)-4,4-difluorotetrahydrofuran-3-yl pentadecanoate (A-14) (7.2 g, 50%) as an off-white semisolid.

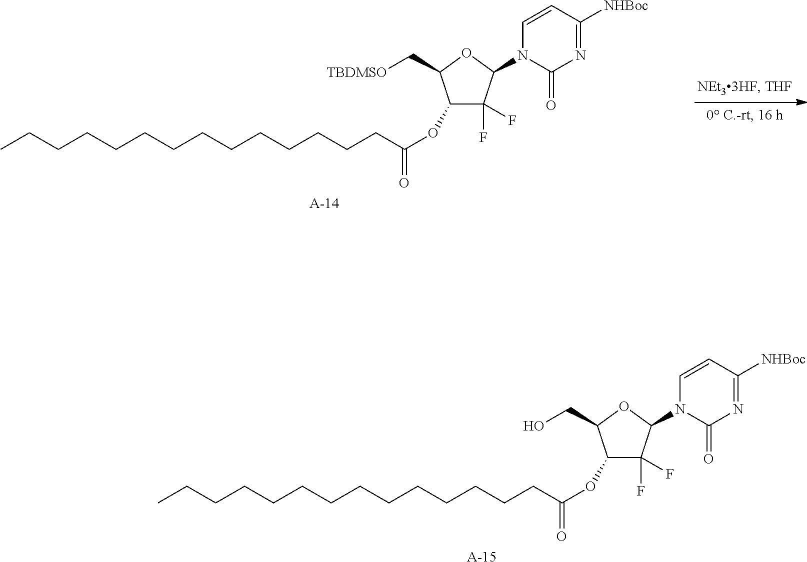 [SMILES: CCCCCCCCCCCCCCC(=O)O[C@@H]1[C@@H](CO[Si](C)(C)C(C)(C)C)O[C@@H](n2ccc(NC(=O)OC(C)(C)C)nc2=O)C1(F)F ; "A-14" ; CCCCCCCCCCCCCCC(=O)O[C@@H]1[C@@H](CO)O[C@@H](n2ccc(NC(=O)OC(C)(C)C)nc2=O)C1(F)F ; "A-15"]

To a solution of (2R,3R,5R)-5-(4-((tert-butoxycarbonyl)amino)-2-oxopyrimidin-1(2H)-yl)-2-(((tert-butyl dimethyl silyl)oxy)methyl)-4,4-difluorotetrahydrofuran-3-yl pentadecanoate (A-14) (7.2 g, 10.27 mmol) in THF (70 mL) was added NEt₃.3HF (8.2 g, 51.355 mmol) dropwise over a period of 30 min at 0° C. The reaction mixture was slowly warmed to room temperature and stirred for 12 h at rt. The solvent was then evaporated. The residue was dissolved in EtOAc (500 mL), washed with water (2×150 mL), brine (150 mL), dried over anhydrous Na₂SO₄, filtered, and evaporated under reduced pressure. The crude compound was purified by column chromatography (SiO₂, 100-200 mesh) to afford (2R,3R,5R)-5-(4-((tert-butoxycarbonyl)amino)-2-oxopyrimidin-1(2H)-yl)-4,4-difluoro-2-(hydroxymethyl) tetrahydrofuran-3-yl pentadecanoate (A-15) (4.7 g, 78%) as a light brown liquid.

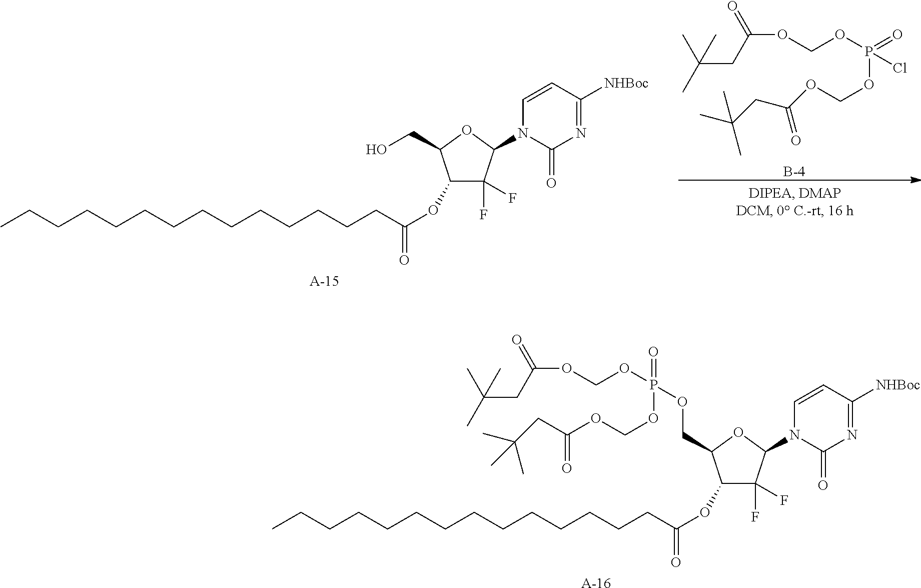

To a solution of ((chlorophosphoryl)bis(oxy))bis(methylene) bis(3,3-dimethylbutanoate) (B-4) (1.58 g, 4.251 mmol) in DCM (10 mL) was added a mixture of (2R,3R,5R)-5-(4-((tert-butoxycarbonyl)amino)-2-oxopyrimidin-1(2H)-yl)-4,4-difluoro-2-(hydroxymethyl) tetrahydrofuran-3-yl pentadecanoate (A-15) (500 mg, 0.85 mmol), DIPEA (0.8 mL, 5.10 mmol) and DMAP (10 mg, 0.085 mmol) in DCM (5 mL) slowly dropwise over a period of 10 min at 0° C. The reaction mixture was stirred at rt for 16 h. The reaction mixture was quenched with water and extracted with EtOAc (2×50 mL). The organic layer was dried over anhydrous Na₂SO₄, filtered, and evaporated under reduced pressure. The crude compound was purified by column chromatography (SiO₂, 100-200 mesh) to afford Compound (A-16) (180 mg, 23%) as a brown liquid.

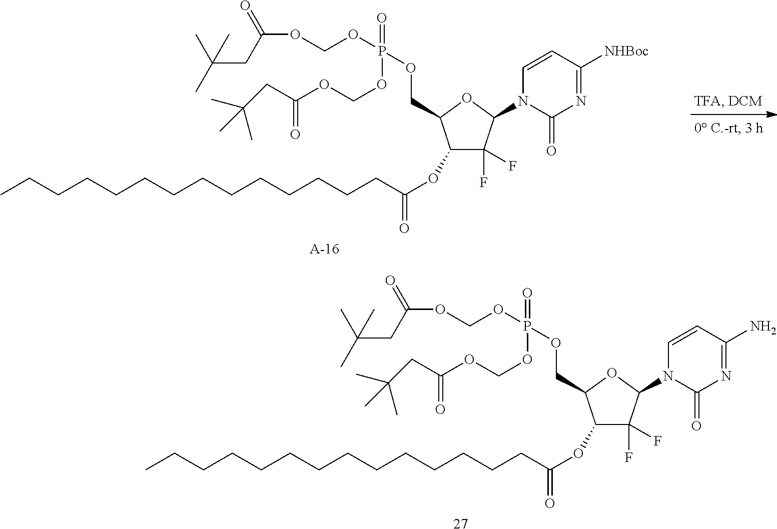 [SMILES: CCCCCCCCCCCCCCC(=O)O[C@@H]1[C@@H](COP(=O)(OCOC(=O)CC(C)(C)C)OCOC(=O)CC(C)(C)C)O[C@@H](n2ccc(NC(=O)OC(C)(C)C)nc2=O)C1(F)F ; "A-16" ; CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F ; "27"]

To a solution of Compound (A-16) (360 mg, 0.39 mmol) in DCM (7 mL) was added TFA (0.7 mL, 2 vol.) dropwise over a period of 5 min at 0° C. The reaction mixture was stirred at room temperature for 3 h. The reaction mixture was concentrated under reduced pressure. The residue was dissolved in EtOAc (100 mL) and washed with saturated NaHCO₃ (2×50 mL). The organic layer was dried over anhydrous Na₂SO₄, filtered, and evaporated under reduced pressure. The crude compound was purified by column chromatography (SiO₂, 100-200 mesh) to afford Compound 27 (0.17 g, 53%) as a colorless semisolid. MS (ESI) m/z 824.50 [M+H]⁺; ¹H NMR (400 MHz, DMSO-d₆) δ 7.57 (d, J=7.6 Hz, 1H), 7.48 (d, J=14.8 Hz, 2H), 6.22 (br s, 1H), 6.25 (br s, 1H), 5.61-5.57 (m, 4H), 5.39 (br s, 1H), 4.36 (d, J=5.2 Hz, 3H), 2.43 (t, J=7.2 Hz, 2H), 2.26 (s, 4H), 1.57-1.54 (m, 2H), 1.23 (br s, 22H), 0.98-0.92 (m, 18H), 0.83 (t, J=6.8 Hz, 3H).

### Example 52: Cellular Pharmacology

Compounds are tested for their ability to impair cancer cell proliferation and/or induce cell death. For cellular proliferation studies, cultured cells are treated with the test compound for 24-120 hours. After compound treatment, cell proliferation is assessed by using methods including, but not limited to, Cell-Titer-Glo® (Promega), Alamar Blue, LIVE/DEAD® (ThermoFisher), BrdU incorporation, and live-cell imaging. The cancer lines used include, but are not limited to BxPC-3 (pancreatic cancer). Gemcitabine Hydrochloride serves as a control for activity.

### Example 52A: Cellular Proliferation Assay in BxPC-3 Cells

BxPC-3 (pancreatic adenocarcinoma) cell line was purchased from the American Type Culture Collection (Catalog #CRL-1687) and grown in RPMI-1640 medium (e.g. Corning #10-040-CV) with 10% Heat Inactivated Fetal Calf Serum at 37° C. and 5% CO2 (as recommended by the ATCC).

Cultures were grown in 175 mm² plates to 80% confluence, and cells were trypsinized to a single-cell suspension. Cells were then resuspended in growth medium to a density of 25,00 cells/ml. They were then plated into 96-well assay plates (Corning #3917) in a volume of 100 ul/well (2,500 cells/well). Cells were allowed to adhere to plates for 24 h at 37° C. and 5% CO₂). Compounds were then added to the wells using an 11-fold serial dilution scheme (over 9 dilutions, generally ranging from 30 μM-30 pM), and the cells were incubated for an additional 120 hours. After 120 h, 90 ul of Cell-Titer Glo reagent (Promega #G7572) was added, and the plates were read using a luminescence counter (e.g., BioTek Synergy HTX at 100 ms read time).

Potency determination were performed by 4-parameter fit of the dose vs. luminescence data using XLFit software (IDBS) with a one site dose response model (Model 205; fit=(A+((B−A)/(1+((C/x)\hat{ }D)))). The EC₅₀ was generally expressed as the inflection point (C parameter) for the fit when the upper and lower portions of the response curve were well-defined. In cases where full inhibition of cell growth was not observed at the highest concentration used, EC₅₀ was reported as the concentration that resulted in 50% loss of Cell-Titer-Glo signal (compared to untreated control). Potency of compounds BxPC-3 cells is shown in following table:

| Compound | EC₅₀ |
|---|---|
| Gemcitabine Hydrochloride | +++ |
| Compound 16 | ++++ |
| Compound 17 | +++ |
| Compound 27 | ++++ |

EC₅₀: + = 1 μM to 25 μM; ++ = 10 nM to 1 μM; +++ = 1 nM to 10 nM; ++++ = <1 nM

### Example 53: In Vivo Xenograft Efficacy

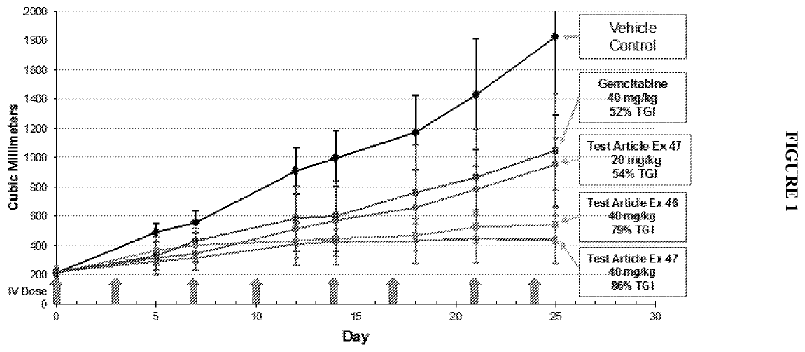

Nanoparticle compositions were tested in an in vivo xenograft efficacy model in tumor-bearing mice. Athymic Nude-Foxn1nu mice were implanted subcutaneously with a human patient-derived xenograft (PDX) derived from a human pancreatic adenocarcinoma tumor (CTG-0687, Champions Oncology). Tumors were allowed grow to 200 cubic mm before commencement of treatment (day 0). Lyophilized nanoparticle formulations of Compound 24 and Compound 16 (lyophilized nanoparticle compositions described in Example 46 and Example 47 respectively) were rehydrated in sterile 0.9% NaCl in water immediately prior to dosing. Mice were then dosed intravenously with various nanoparticle formulations or Gemcitabine (dissolved directly in 0.9% NaCl) twice weekly for 4 weeks. Tumor volume was assessed by caliper measurement using the following formula: Tumor volume=width²×length×0.52. Tumor growth inhibition (TGI) at day 25 was assessed using the following formula: % TGI=100×([Volume at day 25−Volume at day 0] for treatment group)/([Volume at day 25−Volume at day 0] for vehicle control group). As shown in FIG. 1, the nanoparticle formulation (denoted Test Article Ex 46) of Compound 24, and the nanoparticle formulation (denoted Test Article Ex 47) of Compound 16 both exhibited superior efficacy to Gemcitabine at 40 mg/kg dosing.

What is claimed is:

1. A composition comprising nanoparticles, wherein the nanoparticles comprise a compound of Formula (I): 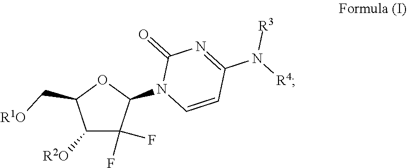
wherein:
R¹ is 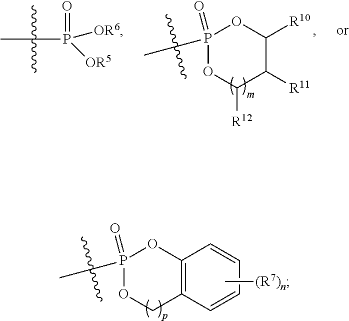
R² is —C(O)R⁸;
R³ is H, —C(O)R⁹, or —C(O)OR⁹;
R⁴ is H;
R⁵ is H, C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀ aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁶ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀ aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
each R⁷ is independently selected from halogen, C₁₋₈alkyl, C₁₋₈haloalkyl, and C₁₋₈alkoxy;
R⁸ is C₃₋₂₂alkyl, C₃₋₂₂alkenyl, C₃₋₂₂alkynyl, C₃₋₂₂haloalkyl, C₃₋₈cycloalkyl, C₆₋₁₀aryl, —C₁₋₈alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹⁴;
R⁹ is C₁₋₁₂alkyl,
R¹⁰ and R¹¹ are each independently H or C₁₋₁₂alkyl, or R¹⁰ and R¹¹ form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³;
R¹² is H or C₁₋₁₂alkyl;
each R¹³ is independently selected from C₁₋₁₂alkyl;
each R¹⁴ is independently selected from halogen, C₁₋₈alkyl, C₁₋₈haloalkyl, C₁₋₈alkoxy, and —C(O)R¹³;
m is 0 or 1,
n is 0, 1, 2, 3, or 4; and
p is 0 or 1; and
a pharmaceutically acceptable carrier; wherein the pharmaceutically acceptable carrier comprises albumin.

2. The composition of claim 1, wherein R¹ is

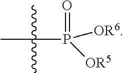

3. The composition of claim 1 or claim 2, wherein R⁵ is C₃₋₁₂alkyl.

4. The composition of any one of claims 1-3, wherein R⁵ is C₆₋₁₀alkyl.

5. The composition of claim 1 or claim 2, wherein R⁵ is —C₁₋₄alkyl-OC(O)C₁₋₈alkyl.

6. The composition of claim 5, wherein R⁵ is —C₁₋₂alkyl-OC(O)C₁₋₆alkyl.

7. The composition of claim 6, wherein R⁵ is —CH₂—OC(O)C(CH₃)₃.

8. The composition of claim 1 or claim 2, wherein R⁵ is H.

9. The composition of any one of claims 1-8, wherein R⁶ is C₃₋₁₂alkyl.

10. The composition of any one of claims 1-9, wherein R⁶ is C₆₋₁₀alkyl.

11. The composition of any one of claims 1-8, wherein R⁶ is —C₁₋₄alkyl-OC(O)C₁₋₈alkyl.

12. The composition of claim 11, wherein R⁶ is —C₁₋₂alkyl-OC(O)C₁₋₆alkyl.

13. The composition of claim 12, wherein R⁶ is —CH₂—OC(O)C(CH₃)₃.

14. The composition of claim 1, wherein R¹ is

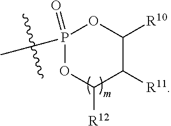

15. The composition of claim 14, wherein m is 0.

16. The composition of claim 14, wherein m is 1.

17. The composition of any one of claims 14-16, wherein R¹⁰ is H.

18. The composition of any one of claims 14-16, wherein R¹⁰ is C₁₋₁₂alkyl.

19. The composition of any one of claims 14-18, wherein R¹¹ is H.

20. The composition of any one of claims 14-16, wherein R¹⁰ and R¹¹ form a 5- or 6-membered cycloalkyl ring or a 5- or 6-membered heterocycloalkyl ring, wherein the 5- or 6-membered cycloalkyl ring or the 5- or 6-membered heterocycloalkyl ring are optionally substituted with one or two R¹³.

21. The claim of claim 1, wherein R¹ is

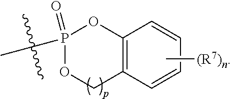

22. The composition of claim 21, wherein each R⁷ is independently selected from C₁₋₈alkyl, C₁₋₈haloalkyl, and C₁₋₈alkoxy.

23. The composition of claim 22, wherein each R⁷ is independently selected from C₁₋₈alkyl.

24. The composition of any one of claims 21-23, wherein n is 1 or 2.

25. The composition of claim 21, wherein n is 0.

26. The composition of any one of claims 21-25, wherein p is 0.

27. The composition of any one of claims 21-25, wherein p is 1.

28. The composition of any one of claims 1-27, wherein R⁸ is C₃₋₁₅alkyl.

29. The composition of any one of claims 1-28, wherein R⁸ is C₆₋₁₂alkyl.

30. The composition of any one of claims 1-29, wherein R⁸ is —(CH₂)₇CH₃.

31. A composition comprising nanoparticles, wherein the nanoparticles comprise a compound of Formula (II): 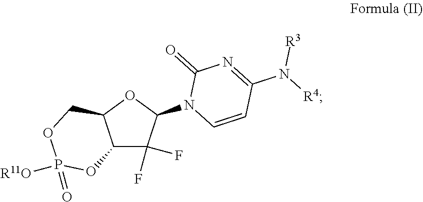
wherein:
R³ is H, —C(O)R⁹, or —C(O)OR⁹;
R⁴ is H;
R⁹ is C₁₋₈alkyl;
R¹¹ is C₃₋₂₂alkyl, C₃₋₂₂alkenyl, C₃₋₂₂alkynyl, C₃₋₂₂haloalkyl, —C₁₋₄alkyl-OC(O)C₁₋₈alkyl, C₆₋₁₀aryl, —C₁₋₈ alkyl-C₆₋₁₀aryl, C₂₋₉heteroaryl, or —C₁₋₈alkyl-C₂₋₉heteroaryl, wherein C₆₋₁₀aryl, —C₁₋₈alkyl-C₆₋₁₀aryl, C₂₋₉heteroaryl, or —C₁₋₈alkyl-C₂₋₉heteroaryl are optionally substituted with 1, 2, 3, or 4 R¹²;
each R¹² is independently selected from halogen, C₁₋₈alkyl, C₁₋₈haloalkyl, C₁₋₈alkoxy, and —C(O)R¹³; and
each R¹³ is independently selected from C₁₋₁₂alkyl; and
a pharmaceutically acceptable carrier; wherein the pharmaceutically acceptable carrier comprises albumin.

32. The composition of claim 31, wherein R¹¹ is C₃₋₁₅alkyl.

33. The composition of claim 31 or claim 32, wherein R¹¹ is C₆₋₁₂alkyl.

34. The composition of any one of claims 31-33, wherein R¹¹ is C₈₋₁₀alkyl.

35. The composition of claim 31, wherein R¹¹ is —C₁₋₄alkyl-OC(O)C₁₋₈alkyl.

36. The composition of claim 35, wherein R¹¹ is —C₁₋₂alkyl-OC(O)C₁₋₆alkyl.

37. The composition of claim 36, wherein R¹¹ is —CH₂—OC(O)C(CH₃)₃.

38. The composition of claim 31, wherein R¹¹ is C₆₋₁₀aryl optionally substituted with 1, 2, 3, or 4 R¹².

39. The composition of claim 38, wherein R¹¹ is phenyl optionally substituted with 1, 2, or 3 R¹².

40. The composition of claim 31, wherein R¹¹ is phenyl optionally substituted with 1, 2, or 3 R¹², and each R¹² is independently selected from C₁₋₈alkyl, C₁₋₈alkoxy, and —C(O)R¹³.

41. The composition of claim 40, wherein R¹¹ is phenyl optionally substituted with 1 or 2 R¹², and each R¹² is independently selected from C₁₋₈alkyl and C₁₋₈alkoxy.

42. The composition of claim 41, wherein R¹¹ is —C₁₋₈alkyl-C₆₋₁₀aryl optionally substituted with 1, 2, 3, or 4 R¹².

43. The composition of claim 42, wherein R¹¹ is —CH₂-phenyl optionally substituted with 1, 2, or 3 R¹².

44. The composition of claim 43, wherein R¹¹ is —CH₂-phenyl optionally substituted with 1, 2, or 3 R¹², and each R¹² is independently selected from C₁₋₈alkyl, C₁₋₈alkoxy, and —C(O)R¹³.

45. The composition of claim 44, wherein R¹¹ is —CH₂-phenyl optionally substituted with 1 or 2 R¹², and each R¹² is independently selected from C₁₋₈alkyl and C₁₋₈alkoxy.

46. The composition of any one of claims 1-45, wherein R³ is H.

47. The composition of any one of claims 1-45, wherein R³ is —C(O)R⁹.

48. The composition of any one of claims 1-45, wherein R³ is —C(O)OR⁹.

49. A composition comprising nanoparticles, wherein the nanoparticles comprise a compound selected from: 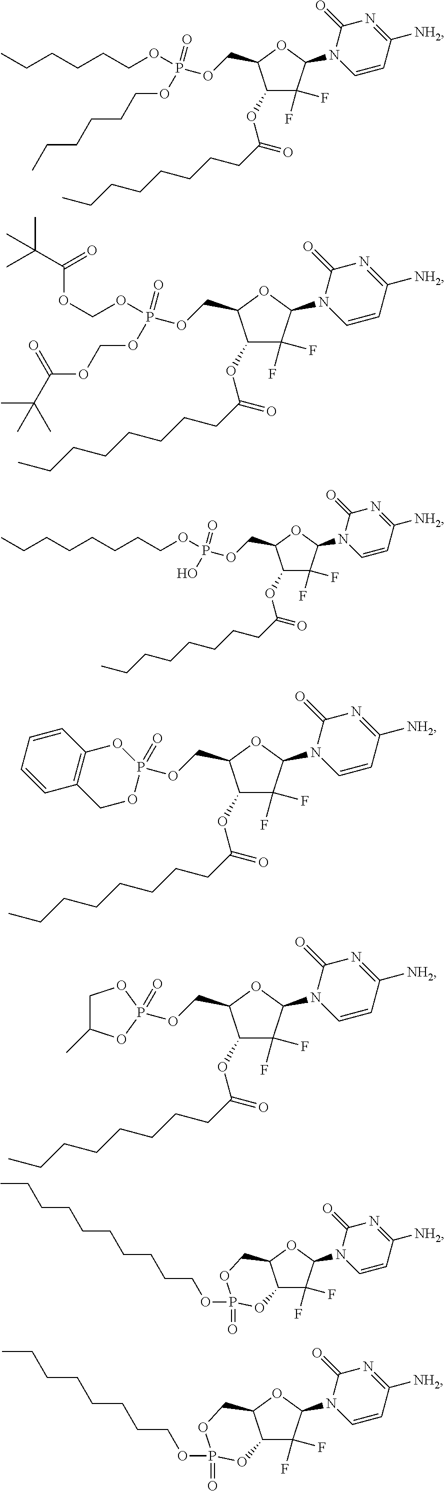 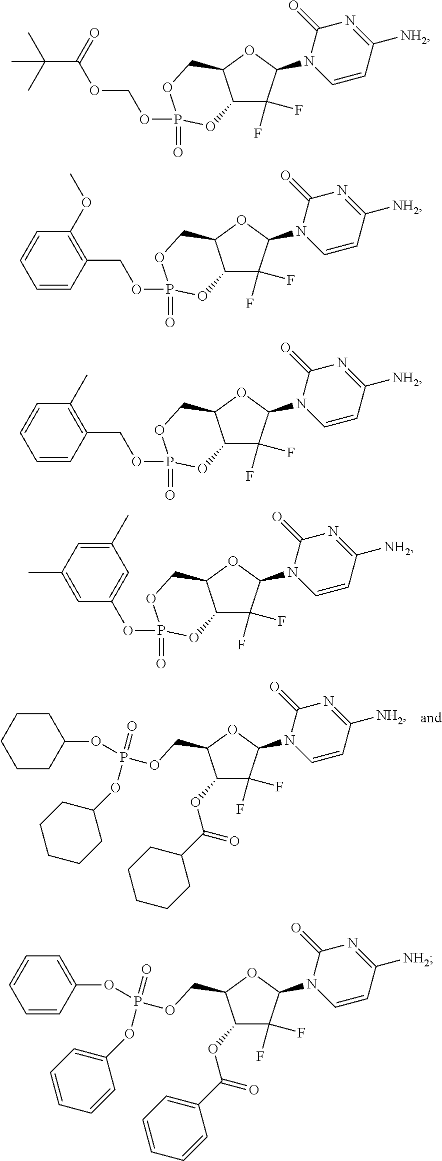
and
a pharmaceutically acceptable carrier; wherein the pharmaceutically acceptable carrier comprises albumin.

50. A composition comprising nanoparticles, wherein the nanoparticles comprise a compound selected from: 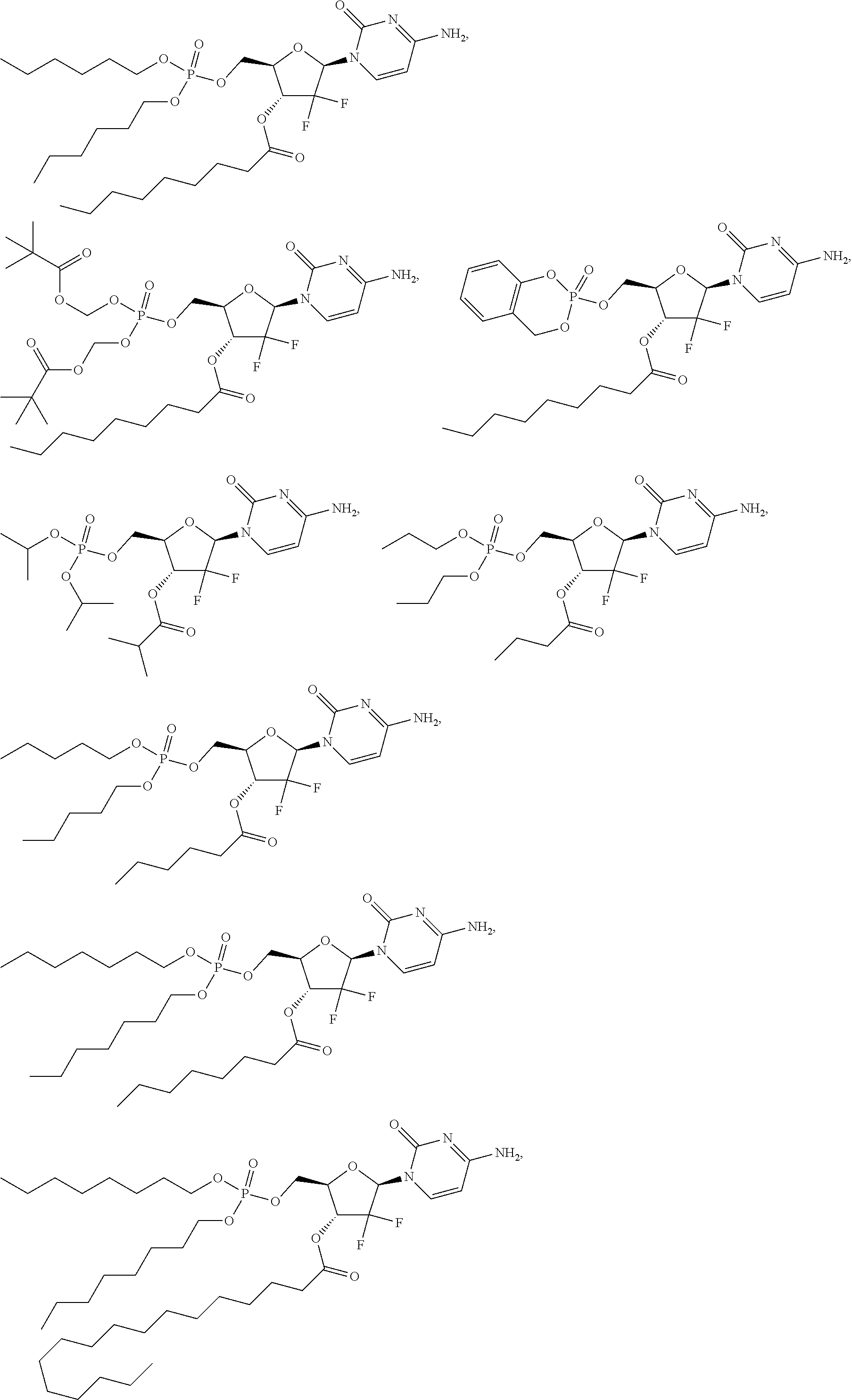 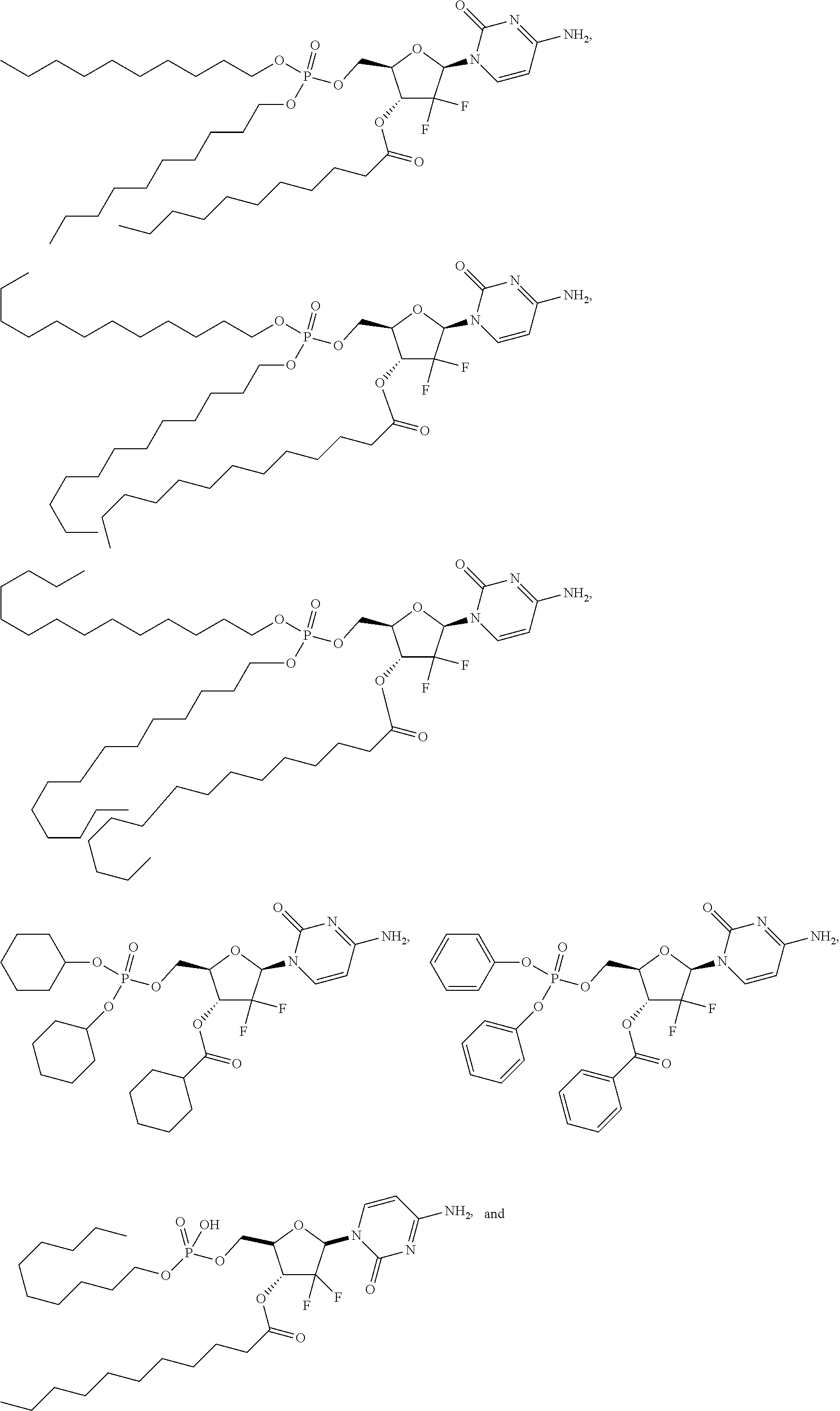 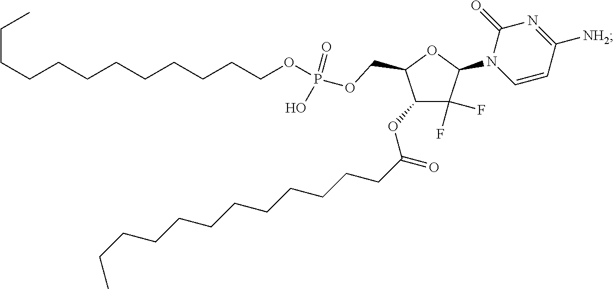 [SMILES: CCCCCCCCCCCCOP(=O)(O)OC[C@H]1O[C@@H](n2ccc(N)nc2=O)C(F)(F)[C@@H]1OC(=O)CCCCCCCCCCCC]
and
a pharmaceutically acceptable carrier; wherein the pharmaceutically acceptable carrier comprises albumin.

51. A composition comprising nanoparticles, wherein the nanoparticles comprise a compound selected from: 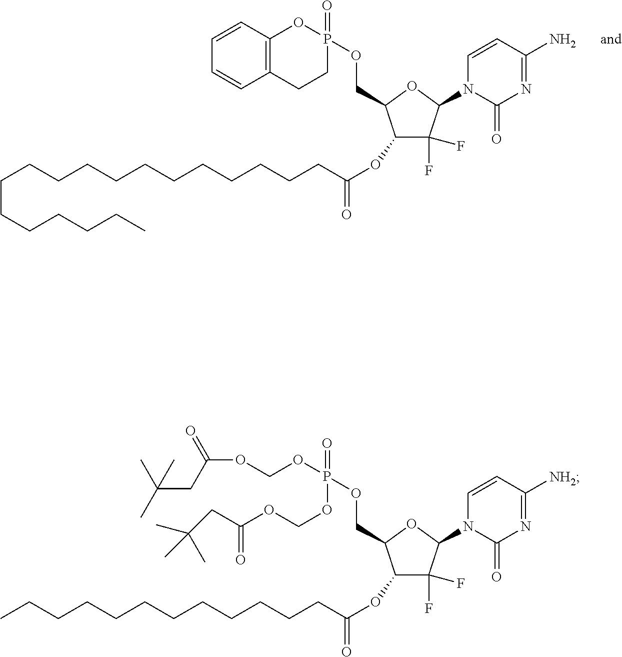 [SMILES: CCCCCCCCCCCCCCCCCCC(=O)O[C@@H]1[C@@H](COP2(=O)CCc3ccccc3O2)O[C@@H](n2ccc(N)nc2=O)C1(F)F ; "and" ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]
and a pharmaceutically acceptable carrier; wherein the pharmaceutically acceptable carrier comprises albumin.

52. A composition comprising nanoparticles, wherein the nanoparticles comprise a compound selected from: 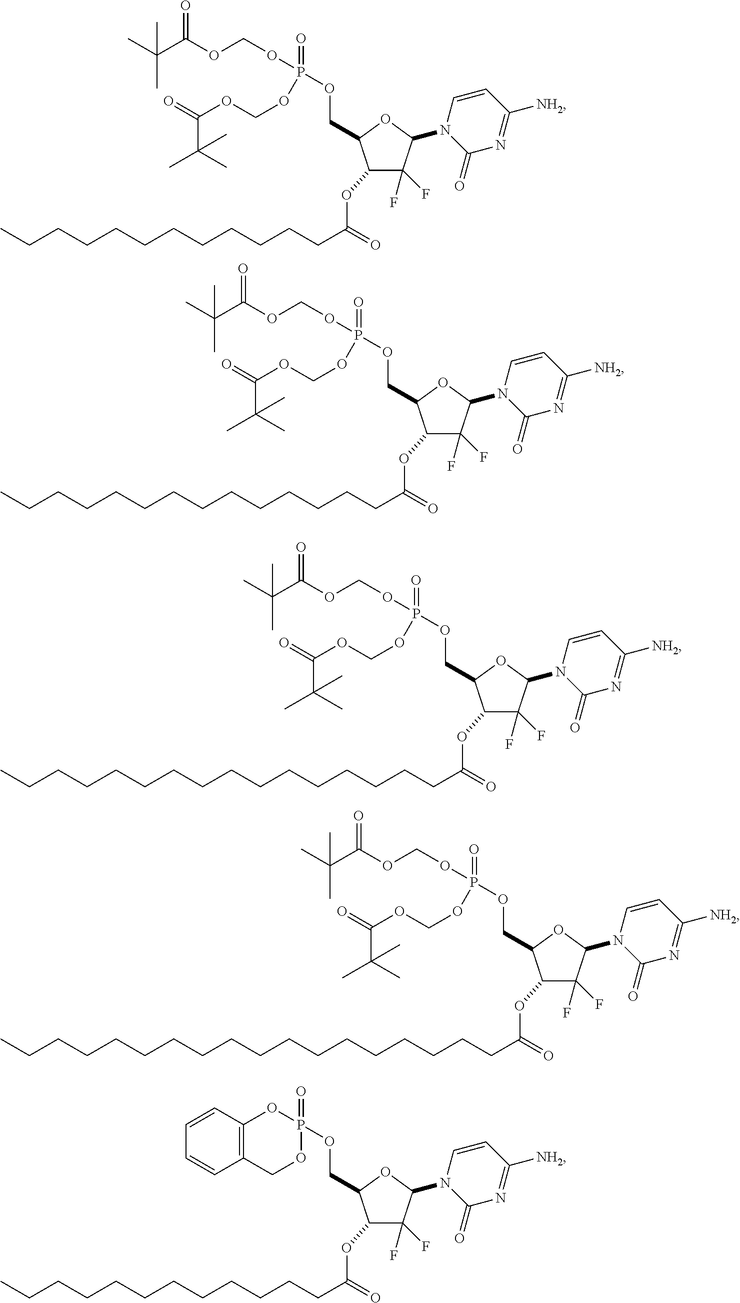 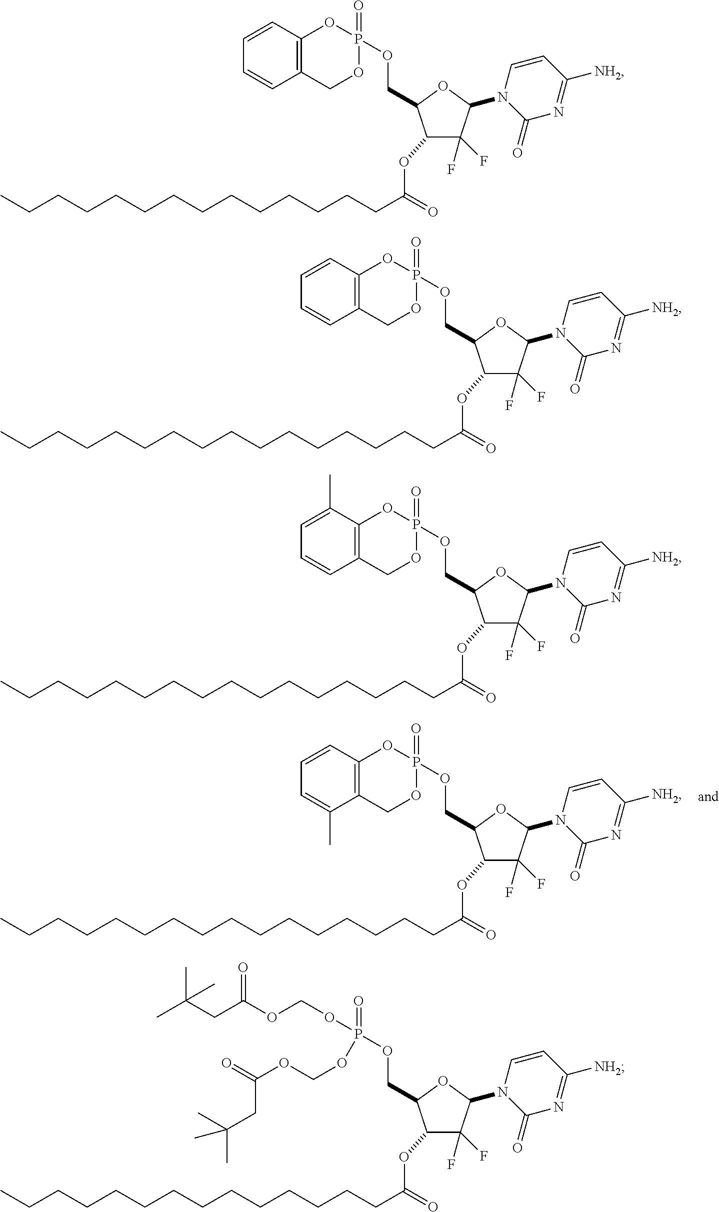
and a pharmaceutically acceptable carrier; wherein the pharmaceutically acceptable carrier comprises albumin.

53. The composition of any of claims 1-52, wherein the nanoparticles have an average diameter of about 1000 nm or less for at least about 15 minutes after nanoparticle formation.

54. The composition of any of claims 1-52, wherein the nanoparticles have an average diameter of about 10 nm or greater for at least about 15 minutes after nanoparticle formation.

55. The composition of any of claims 1-52, the nanoparticles have an average diameter of from about 10 nm to about 1000 nm for at least about 15 minutes after nanoparticle formation.

56. The composition of any of claims 1-52, wherein the nanoparticles have an average diameter of about 1000 nm or less for at least about 4 hours after nanoparticle formation.

57. The composition of any of claims 1-52, wherein the nanoparticles have an average diameter of about 10 nm or greater for at least about 4 hours nanoparticle formation.

58. The composition of any of claims 1-52, the nanoparticles have an average diameter of from about 10 nm to about 1000 nm for at least about 4 hours after nanoparticle formation.

59. The composition of any one of claims 1-58, wherein the nanoparticles have an average diameter of from about 10 nm to about 1000 nm.

60. The composition of claim 59, wherein the nanoparticles have an average diameter of from about 30 nm to about 250 nm.

61. The composition of any of claims 1-60, wherein the albumin is human serum albumin.

62. The composition of any one of claims 1-61, wherein the molar ratio of the compound to pharmaceutically acceptable carrier is from about 1:1 to about 20:1.

63. The composition of claim 62, wherein the molar ratio of the compound to pharmaceutically acceptable carrier is from about 2:1 to about 12:1.

64. The composition of any one of claims 1-63, wherein the nanoparticles are suspended, dissolved, or emulsified in a liquid.

65. The composition of any one of claims 1-64, wherein the composition is sterile filterable.

66. The composition of any one of claims 1-65, wherein the composition is dehydrated.

67. The composition of claim 66, wherein the composition is a lyophilized composition.

68. The composition of claim 66 or 67, wherein the composition comprises from about 0.9% to about 24% by weight of the compound.

69. The composition of claim 68, wherein the composition comprises from about 1.8% to about 16% by weight of the compound.

70. The composition of any one of claims 66-69, wherein the composition comprises from about 76% to about 99% by weight of the pharmaceutically acceptable carrier.

71. The composition of claim 70, wherein the composition comprises from about 84% to about 98% by weight of the pharmaceutically acceptable carrier.

72. The composition of any one of claims 66-71, wherein the composition is reconstituted with an appropriate biocompatible liquid to provide a reconstituted composition.

73. The composition of claim 72, wherein the appropriate biocompatible liquid is a buffered solution.

74. The composition of claim 72, wherein the appropriate biocompatible liquid is a solution comprising dextrose.

75. The composition of claim 72, wherein the appropriate biocompatible liquid is a solution comprising one or more salts.

76. The composition of claim 72, wherein the appropriate biocompatible liquid is sterile water, saline, phosphate-buffered saline, 5% dextrose in water solution, Ringer's solution, or Ringer's lactate solution.

77. The composition of any one of claims 72-76, wherein the nanoparticles have an average diameter of from about 10 nm to about 1000 nm after reconstitution.

78. The composition of claim 77, wherein the nanoparticles have an average diameter of from about 30 nm to about 250 nm after reconstitution.

79. The composition of any one of claims 1-78, wherein the composition is suitable for injection.

80. The composition of any one of claims 1-79, wherein the composition is suitable for intravenous administration.

81. The composition of any one of claims 1-78, wherein the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

82. The composition of any one of claims 1-81, wherein the compound is an anticancer agent.

83. The composition of any one of claims 1-81, wherein the compound is an antiviral agent.

84. A method of treating a disease in a subject in need thereof comprising administering the composition of any one of claims 1-83.

85. The method of claim 84, wherein the disease is cancer.

86. The method of claim 84, wherein the disease is caused by an infection.

87. The method of claim 86, wherein the infection is viral.

88. A method of delivering a compound of Formula (I) or Formula (II) to a subject in need thereof comprising administering the composition of any one of claims 1-83.

89. A process of preparing a composition of any one of claims 1-83 comprising
a) dissolving a compound of Formula (I) or Formula (II) in a volatile solvent to form a solution comprising a dissolved compound of Formula (I) or Formula (II);
b) adding the solution comprising the dissolved compound of Formula (I) or Formula (II) to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the composition of any one of claims 1-83.

90. The process of claim 89, wherein the volatile solvent is a chlorinated solvent, alcohol, ketone, ester, ether, acetonitrile, or any combination thereof.

91. The process of claim 90, wherein the volatile solvent is chloroform, ethanol, methanol, or butanol.

92. The process of any one of claims 89-91, wherein the homogenization is high pressure homogenization.

93. The process of claim 92, wherein the emulsion is cycled through high pressure homogenization for an appropriate amount of cycles.

94. The process of claim 93, wherein the appropriate amount of cycles is from about 2 to about 10 cycles.

95. The process of any one of claims 89-94, wherein the evaporation is accomplished with a rotary evaporator.

96. The process of any one of claims 89-95, wherein the evaporation is under reduced pressure.

97. A compound selected from: 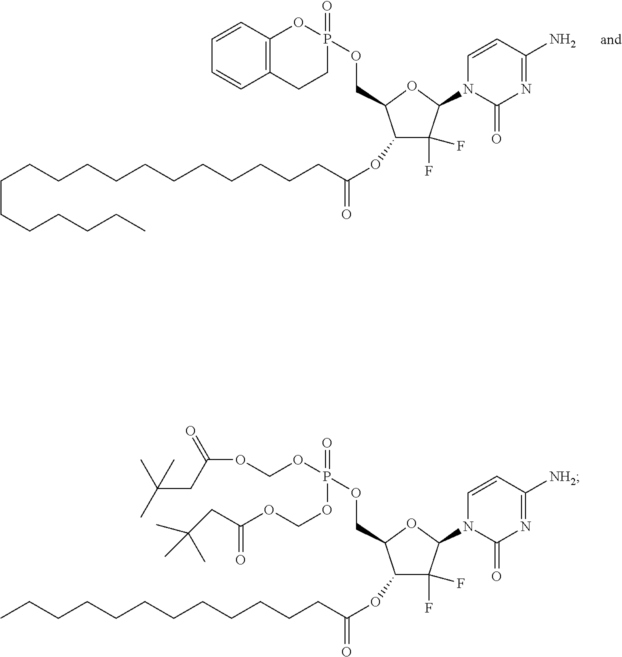 [SMILES: CCCCCCCCCCCCCCCCCCC(=O)O[C@@H]1[C@@H](COP2(=O)CCc3ccccc3O2)O[C@@H](n2ccc(N)nc2=O)C1(F)F ; "and" ; CCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]
or a pharmaceutically acceptable salt thereof.

98. A compound that is: 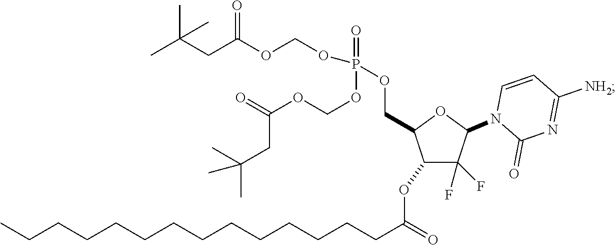 [SMILES: CCCCCCCCCCCCCCC(=O)O[C@@H]1[C@@H](COP(=O)(OCOC(=O)CC(C)(C)C)OCOC(=O)CC(C)(C)C)O[C@@H](n2ccc(N)nc2=O)C1(F)F]
or a pharmaceutically acceptable salt thereof.

99. A pharmaceutical composition comprising a compound of claim 97 or claim 98, or a pharmaceutically acceptable salt thereof, and at least one pharmaceutically acceptable excipient.

100. A method of treating cancer in a patient in need thereof, comprising administering to the patient a therapeutically effective amount of a compound of claim 97 or claim 98, or a pharmaceutically acceptable salt thereof.

101. A method of treating an infectious disease in a patient in need thereof, comprising administering to the patient a therapeutically effective amount of a compound of claim 97 or claim 98, or a pharmaceutically acceptable salt thereof.
